(19) United States
(12) Patent Application Publication
(10) Pub. No.: US 2003/0144570 A1
(43) Pub. Date: Jul. 31, 2003

(54) Compositions and methods for treating disease utilizing a combination of radioactive therapy and cell-cycle inhibitors

(75) Inventors: William L. Hunter, Vancouver (CA); David M. Gravett, Vancouver (CA); Richard T. Liggins, Coquitlam (CA); Troy A.E. Loss, North Vancouver (CA); Arpita Maiti, Vancouver (CA); Philip M. Toleikis, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Angiotech Pharmaceuticals, Inc., Vancouver, BC (CA)

(21) Appl. No.: 10/155,868

(22) Filed: May 24, 2002

Related U.S. Application Data
(63) Continuation-in-part of application No. 09/865,195, filed on May 24, 2001.
Application No. 09/865,195 is a continuation-in-part of application No. 09/712,047, filed on Nov. 13, 2000.
(60) Provisional application No. 60/165,259, filed on Nov. 12, 1999.

(51) Int. Cl. A61N 5/00

(52) U.S. Cl. 600/1

(57) ABSTRACT

Disclosed herein are therapeutic devices, compositions and methods for treating proliferative diseases. For example, within one aspect of the invention therapeutic devices are provided, comprising a device that locally administers radiation and a cell-cycle inhibitor

### CROSS-REFERENCE TO RELATED APPLICATIONS

This application is a continuation-in-part of U.S. patent application Ser. No. 09/865,195, filed May 24, 2001, which application is a continuation-in-part of U.S. patent application Ser. No. 09/712,047, filed Nov. 13, 2000, which application claims priority to U.S. Provisional Application No. 60/165,259, filed Nov. 12, 1999, all of which applications are incorporated by reference in their entirety.

### TECHNICAL FIELD

The present invention relates generally to pharmaceutical compositions, devices and methods, and more specifically, to methods for treating a wide variety of hyperproliferative diseases and conditions utilizing radiation and cell-cycle inhibitors.

### BACKGROUND OF THE INVENTION

Proliferative diseases, such as for example, cancer, represent a tremendous burden to the health-care system. For example, cancer is newly diagnosed in at least 1.4 million patients each year in the U.S., and is the second leading cause of death. Cancer, which is typically characterized by the uncontrolled division of a population of cells frequently results in the formation of a tumor, as well as subsequent metastasize to one or more sites.

Proliferative diseases can result from a number of factors, including for example, exposure to compounds found in the environment or workplace (e.g., exposure to heavy metals, petroleum products, or, asbestos, exposure to the sun or radiation, or, smoking), genetic factors (e.g., BRAC-1 or -2), and, exposure to viruses or other disease causing entities (e.g., retroviruses) (see generally, Cancer: Causes, Occurrence and Control. Edited by L. Tomatis. Oxford University Press, 1990; Cancer Epidemiology and Prevention. Edited by D. Schottenfeld and J. F. Fraumeni, Jr., Oxford University Press, 1996).

Many solid tumors can be treated by resection. However, many patients who present solid tumors clinically also have micrometastases beyond the primary tumor site. If treated with surgery alone, many of these patients will experience recurrence of the cancer. In addition to surgery, many cancers are now also treated with a combination of therapies involving cytotoxic chemotherapeutic drugs (e.g., vincristine, vinblastine, cisplatin, etc.) and/or radiation therapy. One difficulty with this approach, however, is that radiotherapeutic and chemotherapeutic agents are toxic to normal tissues, and often create life-threatening side effects. In addition, these approaches often have extremely high failure/remission rates (up to 90% depending upon the type of cancer).

The present invention discloses novel compositions devices and methods for treating a wide variety of proliferative diseases and conditions, and further provides other related advantages.

### SUMMARY OF THE INVENTION

Briefly stated, the present invention provides compositions and methods for the treatment of a variety of proliferative diseases. For example, within one aspect of the invention therapeutic devices are provided, comprising a device which locally administers radiation, and a cell-cycle inhibitor. Within another aspect of the present invention, compositions are provided, comprising a radioactive source and a cell-cycle inhibitor.

Utilizing the above-noted devices and compositions, a wide variety of diseases or conditions associated with cellular proliferation may be readily treated or prevented. Such methods generally comprise the step of administering to a patient (e.g., a warm-blooded animal such as a human, horse or cow) a therapeutic device as noted above, or alternatively, one or more cell-cycle inhibitors, and one or more sources of radiation. Representative diseases or conditions which may be treated with such devices and compositions include a wide variety of cancers, stenosis or restenosis, adhesions (e.g., surgical adhesions or vascular adhesions), vascular disease, and arthritis. Depending on the disease or condition to be treated, a cell-cycle inhibitor or source of radiation may be placed close to the surface of the body (e.g., applied topically), introduced into a body cavity, or directly administered to a body tissue.

A wide variety of devices (e.g., radioactive devices) may be utilized in this regard, including for example, stents, rods, disks, sutures, and seeds (i.e., a particulate radioactive source that may be of a variety of shapes or sizes). Particularly preferred seeds generally have a diameter of 0.85 mm, and a length of either 5.5 mm or 10.0 mm. Further, the radioactive source or cell-cycle inhibitor may be further formulated to contain, be contained within, or be released by a polymer. Polymers may be non-biodegradable, or, biodegradable (and resorbable). Representative examples include poly rotho esters, poly anhydrides, poly (ethylene-vinyl acetate); polyurethane; poly (caprolactone); poly(glycolic acid), poly(glycolic-co-lactic acid), poly (lactic acid); a copolymer of poly (caprolactone) and poly (lactic acid), polyethylene glycol (PEG), methoxypolyethylene glycol (MePEG), poly(methyl methacrylate) or, poly(ethylmethacrylate). Finally, a wide variety of radioactive sources (e.g., I¹²⁵, Pd¹⁰³ and Ir¹⁹²; Co⁶⁰, Cs¹³⁷, Au¹⁹⁸ and Ru¹⁰⁶) and cell-cycle inhibitors (e.g., polypeptides including peptides and fragments or derivatives thereof that may have modifications such as D-amino acids; taxanes such as paclitaxel, or an analogue or derivative thereof; topoisomerase inhibitors; anti-metabolites; alkylating agents; or vinca alkaloids) may be utilized. Within related embodiments of the invention, the above-noted devices can be made radiopaque, or echogenic. For example, within one embodiment, a device as noted above, can be coated with a radiopaque or echogenic coating.

Within one aspect of the invention, therapeutic devices are provided comprising a device that locally administers radiation, and a cell cycle inhibitor. Within various embodiments the device may release both radiation and a cell cycle inhibitor from a unitary body, or alternatively, release the radiation and a cell cycle inhibitor from different aspects of the device. Representative examples of devices that locally administer radiation include radioactive stents, rods, disks, seeds, fastening devices (e.g., sutures). Within certain embodiments, the devices may be formed of, or further comprised of (e.g., coated with) a carrier such as an ointment, liposome, or, polymer (e.g., biodegradable or non-biodegradable polymers such as poly (ethylene-vinyl acetate); polyurethane; poly (caprolactone); poly(glycolic acid), poly(glycolic-co-lactic acid), poly (lactic acid); a copolymer of poly (caprolactone) and poly (lactic acid), polyethylene glycol (PEG), methoxypolyethylene glycol (MePEG), poly(methyl methacrylate) or, poly(ethylmethacrylate). Within certain embodiments, the carrier (e.g., polymer) may be adapted to release a cell cycle inhibitor and/or the radiation). Within further embodiments, the radiation is from a radioactive source selected from the group consisting of activity I¹²⁵, Pd¹⁰³ and Ir¹⁹²; Au¹⁹⁸, Co⁶⁰, Cs¹³⁷, and Ru¹⁰⁶. Representative examples of cell cycle inhibitors include taxanes such as paclitaxel, antimetabolites, vinca alkaloids, alkylating agents, as well as a variety of proteins, and antisense or ribozymes (as well as gene delivery vehicles or vectors which can be, optionally, utilized to deliver or express the protein(s), antisense or ribozyme sequences. Within various embodiments, the device can be made radiopaque or echogenic in order to enhance visualization.

Within other aspects of the invention, therapeutic devices are provided comprising a radioactive source sized to be positioned into the tissue of a patient adjacent to a site to be treated by locally administered radiation from the radioactive source; and a cell-cycle inhibitor positioned adjacent to the radioactive source. Within one embodiment, the device further comprises a carrier member (e.g., a suture) supporting the radioactive source. Within a further embodiment, the radioactive source is disposed within the suture. Within a further embodiment, the radioactive source comprises a plurality of radioactive seeds, and the seeds are positioned at locations along a length of the suture. Within further embodiments, one or more cell-cycle inhibitors are positioned within the suture. Within another embodiment, a cell-cycle inhibitor is positioned within the suture by being absorbed by or incorporated into or onto the suture prior to positioning of the suture in the tissue. Within a further embodiment, a cell-cycle inhibitor is carried by a carrier material positioned one of within the suture or on an outer surface of the suture, and the carrier material is a material selected to release a cell-cycle inhibitor when the suture is within the tissue. Within another embodiment, the material selected for the carrier material is a polymer. Within further embodiments, a cell-cycle inhibitor is carried by the carrier material by being absorbed by or incorporated into or onto the carrier material prior to positioning of the suture in the tissue. Within other embodiments, a cell-cycle inhibitor is carried by a carrier material positioned one of within the suture or on an outer surface of the suture, and the carrier material is a material selected to elute a cell-cycle inhibitor when the suture is within the tissue. Within another embodiment, the suture has at least a portion of the suture comprised of a material that carries a cell-cycle inhibitor. Within further embodiments a cell-cycle inhibitor is carried by the suture, and the suture is a material selected to release a cell-cycle inhibitor when the suture is within the tissue. Within a further embodiment the material selected for the carrier member is a polymer. Within other embodiments, a cell-cycle inhibitor is carried by the suture by being absorbed by or incorporated into or onto the suture prior to positioning of the suture in the tissue. Within further embodiments, a cell-cycle inhibitor is carried by the suture, and the suture is a material selected to elute a cell-cycle inhibitor when the suture is within the tissue. Within other embodiments, a cell-cycle inhibitor is positioned on an outer surface of the suture prior to positioning of the suture in the tissue. Within another embodiment, the suture has an outer member positioned at least partially about an outer surface of the suture prior to positioning of the suture in the tissue, and a cell-cycle inhibitor is carried by the outer member (e.g., a coating at least partially covering the outer surface of the suture). Within further embodiments the coating is a polymeric material and a cell-cycle inhibitor is within the polymeric material. Within related embodiments, the outer member is a material (e.g., a polymer) selected to release a cell-cycle inhibitor when the suture is within the tissue. Within other embodiments, the outer member is a material selected to elute a cell-cycle inhibitor when the suture is within the tissue. Within another embodiment one or more cell-cycle inhibitors are chemically linked to or coated on the radioactive suture. Within other embodiments, the radioactive source is a radioactive wire, which may, optionally, have a cell-cycle inhibitor is positioned on an outer surface of the wire. Within other embodiments a cell-cycle inhibitor is positioned on an outer surface of the wire prior to positioning of the wire in the tissue. Within further embodiments a cell-cycle inhibitor is carried by a carrier material positioned on an outer surface of the wire, and the carrier material is a material (e.g., a polymer selected to release a cell-cycle inhibitor when the wire is within the tissue. Within further embodiments, a cell-cycle inhibitor is carried by the carrier material by being absorbed by or incorporated into or onto the carrier material prior to positioning of the wire in the tissue. Within various embodiments of the above, the device, source of radiation, and/or cell-cycle inhibitor can be made radiopaque or echogenic, in order to enhance visualization.

Within a further embodiment, a cell-cycle inhibitor can be carried by a carrier material positioned on an outer surface of the wire, and the carrier material is a material selected to elute a cell-cycle inhibitor when the wire is within the tissue. Within related embodiments, the wire has an outer member positioned at least partially about an outer surface of the wire prior to positioning of the wire in the tissue, and a cell-cycle inhibitor is carried by the outer member. Within further embodiments, the outer member is a coating at least partially covering the outer surface of the wire. Within yet other embodiments the coating is a polymeric material and a cell-cycle inhibitor is within the polymeric material. Within other embodiments the outer member is a material (e.g., a polymer) selected to release a cell-cycle inhibitor when the wire is within the tissue. Within other embodiments the outer member is a material selected to release a cell-cycle inhibitor when the wire is within a tissue. Within further embodiments the cell-cycle inhibitor is one of chemically linked to or coated on the wire. Within various embodiments of the above, the cell-cycle inhibitor can be made radiopaque or echogenic, in order to enhance visualization.

Within related embodiments, the radioactive source comprises a plurality of radioactive seeds (i.e., particulate radioactive compounds, elements or compositions of any of a variety of radioactive sources, sizes, and/or shapes). Within one embodiment a cell-cycle inhibitor is positioned on an outer surface of the seeds. Within other embodiments a cell-cycle inhibitor is positioned on an outer surface of the seeds prior to positioning of the seeds in the tissue. Within further embodiments a cell-cycle inhibitor is carried by a carrier material positioned on an outer surface of each of the seeds, and the carrier material is a material selected to release a cell-cycle inhibitor when the seeds are within the tissue. Within one embodiment the carrier member is a polymer. Within further embodiments a cell-cycle inhibitor is carried by the carrier material by being absorbed by or incorporated into or onto the carrier material prior to positioning of the seeds in the tissue. Within yet other embodiments a cell-cycle inhibitor is carried by a carrier material positioned on an outer surface of each of the seeds, and the carrier material is a material selected to elute a cell-cycle inhibitor when the seeds are within the tissue. Within further embodiments the device can include a spacer (which can, optionally, carrier the cell cycle inhibitor) positioned being adjacent ones of the plurality of radioactive seeds. Within other embodiments, the spacer (e.g., a polymer) is a material selected to release a cell-cycle inhibitor when within the tissue. Within related embodiments, a cell-cycle inhibitor is carried by the spacer by being absorbed by or incorporated into or onto the spacer prior to positioning of the spacer in the tissue. Within other embodiments, the spacer is a material selected to elute a cell-cycle inhibitor when within the tissue. Within further embodiments, the spacer is a polymeric material and a cell-cycle inhibitor is within the polymeric material. Within yet further embodiments, a cell-cycle inhibitor is positioned on an outer surface of the spacer. Within other embodiments, a cell-cycle inhibitor is positioned on the outer surface of the spacer prior to positioning of the spacer in the tissue. Within related embodiments, a cell-cycle inhibitor is carried by a carrier material positioned on an outer surface of the spacer, and the carrier material is a material selected to elute a cell-cycle inhibitor when the spacer are within the tissue. Within other embodiments, a cell-cycle inhibitor is carried by the carrier material by being absorbed by or incorporated into or onto the carrier material prior to positioning of the spacer in the tissue. Within further embodiments, the seeds and the spacers positioned between the seeds are sized to be received in a catheter for insertion into the tissue. Within related embodiments, the spacers are elongated with a length and positioned with a lengthwise orientation extending between the adjacent seeds between which positioned, and the spacer length is selected to position and hold the seeds within the tissue in a desired spatial pattern based upon the radiation pattern desired to be administered to the site to be treated. Within other embodiments, the device further includes a spacer positioned between adjacent ones of the plurality of radioactive seeds, the spacers both holding the adjacent seeds spaced apart while in the tissue and holding the plurality of seeds together as part of a continuous thread while being positioned in the tissue. Within yet other embodiments the spacers are formed from a spacer material having a liquid phase and a solid phase, the spacers being formed using the spacer material in the liquid phase immediately prior to the time of positioning of the seeds into the tissue by placing the liquid phase spacer material between adjacent ones of the seeds and then allowing the spacer material to change to the solid phase to form the continuous thread. Within further embodiments, the device includes a spacer positioned between adjacent ones of the plurality of radioactive seeds, the spacers holding the adjacent seeds spaced apart while in the tissue, the spacers being a spacer material having a liquid phase and a solid phase, the spacers being formed using the spacer material in the liquid phase immediately prior to the time of positioning of the seeds into the tissue by placing the liquid phase spacer material between adjacent ones of the seeds and then allowing the spacer material to change to the solid phase prior to positioning of the spacers in the tissue. Within yet other embodiments, the device, for use with a catheter, has seeds which are positioned in the catheter in spaced apart relation and the spacer material in the liquid phase is placed between adjacent ones of the seeds and then allowed to change to the solid phase, after changing to the solid phase and without removing the seeds and the spacers from the catheter, the seeds and the spacers being positioned in the catheter in a molded state ready for positioning in the tissue using the catheter. Within further embodiments, after the spacer material has been allowed to change to the solid phase, the seeds and the spacers are in the form of a continuous thread holding the plurality of seeds together for positioning in the tissue and holding the adjacent seeds spaced apart while in the tissue. Within related embodiments, the spacer material is in the liquid phase when heated to a liquid phase temperature above a body temperature of the patient, and in the solid phase when allowed to cool to a solid phase temperature below the liquid phase temperature. Within further embodiments, a cell-cycle inhibitor is one of chemically linked to or coated on the seeds. Within various embodiments of the above, the radioactive seed, seed spacer, and/or cell-cycle inhibitor can be made radiopaque or echogenic, in order to enhance visualization.

Within other embodiments, the radioactive source comprises at least one radioactive seed and the seed has an outer member positioned at least partially about an outer surface of the seed prior to positioning of the seed in the tissue, and wherein a cell-cycle inhibitor is carried by the outer member. Within related embodiments, the outer member is a coating at least partially covering the outer surface of the seed. As an example, the coating can be a polymeric material and a cell-cycle inhibitor is within the polymeric material. Within further embodiments, the outer member is a material (e.g., a polymer) selected to release a cell-cycle inhibitor when the wire is within the tissue. Within other embodiments, the outer member is a material selected to elute a cell-cycle inhibitor when the wire is within the tissue. Within further embodiments a cell-cycle inhibitor is carried by the outer member by being absorbed by or incorporated into or onto the outer member prior to positioning of the seeds in the tissue. Within yet other embodiments, the radioactive source comprises at least one radioactive seed, and wherein a cell-cycle inhibitor is one of chemically linked to or coated on the seed. Within various embodiments of the above, the seed and/or cell-cycle inhibitor can be made radiopaque or echogenic, in order to enhance visualization.

Within other aspects of the present invention, therapeutic devices are provided comprising a radioactive source sized to be positioned into a pre-existing or created body cavity of a patient adjacent to a site to be treated by locally administered radiation from the radioactive source; and a cell-cycle inhibitor positioned adjacent to the radioactive source. Within one embodiment the radioactive source is a radioactive stent. Within a further embodiment, the radioactive source is a seed, film, mesh, fabric, or gel. Within other embodiments, the stent is formed of a carrier material and the carrier material carries a cell-cycle inhibitor, the carrier material being a material selected to release a cell- cycle inhibitor when the stent is within the body cavity. Within further embodiments, the carrier material is a polymer. Within yet other embodiments, the device further includes a stent sized to be positioned in the body cavity, the stent being formed of a carrier material which carries a cell-cycle inhibitor, the carrier material being a material selected to release a cell-cycle inhibitor when the stent is within the body cavity. Within one embodiment, the carrier material is a polymer. Within other embodiments, a cell-cycle inhibitor is positioned on an outer surface of the stent. Within yet other embodiments, a cell-cycle inhibitor is positioned on an outer surface of the stent prior to positioning of the stent in the body cavity. Within further embodiments, a cell-cycle inhibitor is carried by a carrier material positioned on an outer surface of the stent, and the carrier material is a material selected to release a cell-cycle inhibitor when the stent is within the body cavity. Within related embodiments the material selected for the carrier material is a polymer. Within yet other embodiments, a cell-cycle inhibitor is carried by the carrier material by being absorbed by or incorporated into or onto the carrier material prior to positioning of the stent in the body cavity. Within further embodiments, a cell-cycle inhibitor is carried by a carrier material positioned on an outer surface of the stent, and the carrier material is a material selected to elute a cell-cycle inhibitor when the stent is within the body cavity. Within another embodiment, the stent has an outer member positioned at least partially about an outer surface of the stent prior to positioning of the stent in the body cavity, and a cell-cycle inhibitor is carried by the outer member. Within a related embodiment the outer member is a coating at least partially covering the outer surface of the stent. Within other embodiments the coating is a polymeric material and a cell-cycle inhibitor is within the polymeric material. Within yet other embodiments the outer member is a material selected to release a cell-cycle inhibitor when the stent is within the body cavity. Within further embodiments the material selected for the outer member is a polymer. Within other embodiments a cell-cycle inhibitor is carried by the outer member by being absorbed by or incorporated into or onto the outer member prior to positioning of the stent in the body cavity. Within further embodiments, the outer member is a material selected to elute a cell-cycle inhibitor when the stent is within the body cavity. Within yet further embodiments, a cell-cycle inhibitor is one of chemically linked to or coated on the stent. Within another embodiment, the radioactive source comprises a plurality of radioactive seeds. Within related embodiments a cell-cycle inhibitor is positioned on an outer surface of the seeds. Within other embodiments a cell-cycle inhibitor is positioned on an outer surface of the seeds prior to positioning of the seeds in the body cavity. Within yet other embodiments a cell-cycle inhibitor is carried by a carrier material positioned on an outer surface of each of the seeds, and the carrier material is a material (e.g., a polymer) selected to release a cell-cycle inhibitor when the seeds are in the body cavity. Within one embodiment, a cell-cycle inhibitor is carried by the carrier material by being absorbed by or incorporated into or onto the carrier material prior to positioning of the seeds in the body cavity. Within other embodiments, a cell-cycle inhibitor is carried by a carrier material positioned on an outer surface of each of the seeds, and the carrier material is a material selected to elute a cell-cycle inhibitor when the seeds are in the body cavity. Within further embodiments a cell-cycle inhibitor is one of chemically linked to or coated on the seeds. Within various embodiments of the above, the theapeutic device, carrier, radioactive source, and/or cell-cycle inhibitor can be made radiopaque or echogenic, in order to enhance visualization.

Within yet other aspects of the invention, therapeutic devices are provided comprising a radioactive source; a capsule containing the radioactive source, the capsule being sized to be positioned into a pre-existing or created body cavity of a patient adjacent to a site to be treated by locally administered radiation from the radioactive source; and a cell-cycle inhibitor. Within one embodiment the radioactive source comprises a plurality of radioactive seeds. Within another embodiment a cell-cycle inhibitor is positioned on an outer surface of the capsule. Within other embodiments a cell-cycle inhibitor is positioned on the outer surface of the radioactive source prior to positioning of the radioactive source in the capsule. Within yet other embodiments a cell-cycle inhibitor is positioned within the capsule adjacent to the radioactive source. Within further embodiments a cell-cycle inhibitor is carried by a carrier material selected to release a cell-cycle inhibitor when the capsule is in the body cavity. Within further embodiments a carrier material is positioned on an outer surface of the capsule. Within yet further embodiments, a carrier material is positioned on an outer surface of the capsule prior to positioning of the radioactive source in the capsule. Within another embodiment a carrier material is positioned within the capsule adjacent to the radioactive source. Within further embodiments, the carrier material forms the body of the capsule. Within related embodiments the material selected for the carrier member is a polymer. Within yet other embodiments a cell-cycle inhibitor is carried by the carrier material by being absorbed by or incorporated into or onto the carrier material prior to the capsule being positioning in the body cavity. Within yet other embodiments a cell-cycle inhibitor is carried by a carrier material selected to elute a cell-cycle inhibitor when the capsule is in the body cavity. Within various embodiments of the above, the theapeutic device, capsule, cell-cycle inhibitor and/or carrier can be made radiopaque or echogenic, in order to enhance visualization.

Within yet other aspects of the present invention, therapeutic devices are provided comprising a radioactive source; a body contact member carrying the radioactive source, the body contact member being sized to be positioned against a pre-existing or created surface site of a patient's body to be treated by locally administered radiation from the radioactive source; and a cell-cycle inhibitor. Within one embodiment the body contact member is a sheet. Within other embodiments the device can be used when the site of the patient's body to be treated is curved, wherein the body contact member is sufficiently flexible to be bent to at least partially approximate the curve of the site. Within other embodiments, the device can be used when the site of the patient's body to be treated is curved, wherein the body contact member is contoured to at least partially approximate the curve of the site. Within certain embodiments, the body contact member is molded to the curve of the site. Within other embodiments, the radioactive source comprises a plurality of radioactive wires. Within related embodiments the radioactive wires are arranged about the body contact member in a desired spatial pattern based upon a radiation pattern desired to be administered to the site to be treated. Within other embodiments, the radioactive wires are embedded in the body contact member. Within yet other embodiments, the body contact member includes a plurality of spaced apart recesses sized to receive at least partially therein the radioactive wires. Within further embodiments, the device further includes a retainer member extending over at least a portion of the recesses and retaining the radioactive wires in the recesses. Within related embodiments, the retaining member is a sheet extending over at least a portion of the body contact member and closing at least the portion of the recesses over which the sheet extends. Within certain embodiments, the body contact member is a flexible film. Within related embodiments, the film is scored to form the recesses therein. Within other embodiments, the body contact member is a first flexible film and the radioactive wires are one of embedded in, resident on, or retained upon the first film. Within further embodiments, the first film is selected of a material that can be cut with one of a scalpel or scissors to a desired shape. Within yet further embodiments, the radioactive wires are positioned in a desired spatial pattern with respect to the first film based upon a radiation pattern desired to be administered to the site to be treated. Within other embodiments, the device can further include a second flexible film extending over at least a portion of the first film with the radioactive wires being retained between the first and second films. Within yet other embodiments, the first film includes a plurality of spaced apart recesses sized to receive at least partially therein the radioactive wires, and the second film at least partially closes the recesses to retain the radioactive wires therein. Within further embodiments, the body contact member is a flexible film with a plurality of spaced apart recesses sized to receive at least partially therein the radioactive wires, and the device further includes at least one retainer member positioned to retain the radioactive wires within the recesses. Within other embodiments, the radioactive source comprises a plurality of radioactive seeds. Within further embodiments the radioactive seeds are arranged about the body contact member in a desired spatial pattern based upon a radiation pattern desired to be administered to the site to be treated. Within another embodiment, the radioactive seeds are embedded in the body contact member. Within yet other embodiments the body contact member includes a plurality of spaced apart recesses sized to receive at least partially therein the radioactive seeds. Within other embodiments, the device further includes a retainer member extending over at least a portion of the recesses and retaining the radioactive seeds in the recesses. Within related embodiments the retaining member is a sheet extending over at least a portion of the body contact member and closing at least the portion of the recesses over which the sheet extends. Within other embodiments, the body contact member is a flexible film. Within related embodiments the film is scored to form the recesses therein. Within yet other embodiments the body contact member is a first flexible film and the radioactive seeds are one of embedded in, resident on, or retained upon the first film. In such embodiments the first film is selected of a material which can be cut with one of a scalpel or scissors to a desired shape. Within other embodiments, the radioactive seeds are positioned in a desired spatial pattern with respect to the first film based upon a radiation pattern desired to be administered to the site to be treated. Within yet other embodiments the device further includes a second flexible film extending over at least a portion of the first film with the radioactive seeds being retained between the first and second films. Within another embodiment the device has a first film which includes a plurality of spaced apart recesses sized to receive at least partially therein the radioactive seeds, and the second film at least partially closes the recesses to retain the radioactive seeds therein. Within other embodiments the body contact member is a flexible film with a plurality of spaced apart recesses sized to receive at least partially therein the radioactive seeds, and the device further includes at least one retainer member positioned to retain the radioactive seeds within the recesses. Within yet other embodiments a cell-cycle inhibitor is positioned on an outer surface of the body contact member. Within various embodiments of the above, the theapeutic device, body contact member and/or cell-cycle inhibitor can be made radiopaque or echogenic, in order to enhance visualization.

Within yet other embodiments, the body contact member includes a carrier material which carries a cell-cycle inhibitor, the carrier material being selected to release a cell-cycle inhibitor when the body contact member is against the site to be treated. Within other embodiments, the body contact member includes at least one recess sized to receive at least partially therein the radioactive source. Within further embodiments the device further includes a retainer member extending over at least a portion of the recess and retaining the radioactive source in the recess. Within related embodiments the retaining member is a sheet extending over at least a portion of the body contact member and closing at least the portion of the recess over which the sheet extends. Within various embodiments of the above, the carrier and/or retiner member can be made radiopaque or echogenic, in order to enhance visualization.

Within other embodiments, the body contact member is a flexible film. Within related embodiments the film is scored to form at least one recess therein to receive at least partially therein the radioactive source. Within further embodiments the film has the radioactive sources at least one of embedded in, resident on, or retained upon the film. Within yet other embodiments the radioactive source is positioned with a desired spatial pattern with respect to the film based upon a radiation pattern desired to be administered to the site to be treated. Within a further embodiment the body contact member is formed at least in part from a carrier material which carries a cell-cycle inhibitor, the carrier material being selected to release a cell-cycle inhibitor when the body contact member is against the site to be treated. Within another embodiment, the material selected for the carrier member is a polymer. Within yet another embodiment, a cell-cycle inhibitor is carried by the carrier material by being absorbed by or incorporated into or onto the carrier material prior to the body contact member being positioned against the site to be treated. Within yet another embodiment, the body contact member is formed at least in part from a carrier material which carries a cell-cycle inhibitor, the carrier material being selected to elute a cell-cycle inhibitor when the body contact member is against the site to be treated. Within various embodiments of the above, the body contact member, cell-cycle inhibitor, and/or carrier can be made radiopaque or echogenic, in order to enhance visualization.

Within other aspects of the present invention, therapeutic devices are provided, comprising a radioactive source; a body contact material carrying the radioactive source, the body contact member being applied to a pre-existing or created surface site of a patient's body to be treated by locally administered radiation from the radioactive source; and a cell-cycle inhibitor. In one embodiment, the therapeutic device wherein the body contact material is formed from one of a paste, gel, film or spray applied to the site to be treated. Within various embodiments of the above, the theapeutic device as a whole, or the radioactive source or body contact material can be made radiopaque or echogenic, in order to enhance visualization.

In another aspect, the present invention provides a method of treating cellular proliferation, comprising administering to a patient any one of the aforementioned therapeutic devices.

In yet other aspects, the present invention provides a method for treating cellular proliferation, comprising administering to a patient a cell-cycle inhibitor and a source of radiation. In one embodiment, the present invention provides the aforementioned method for treating cellular proliferation wherein said source of radiation is Pd¹⁰³, Ir¹⁹², Co⁰, Cs¹³⁷, or Ru¹⁰⁶. In another embodiment, the source of radiation is I¹²⁵. In still another embodiment, the source of radiation is formulated along with a polymer. In another embodiment, the aforementioned method wherein said source of radiation is a radioactive stent, rod, disk, seed, or fastening devices (e.g., suture).

In related embodiments, the cell-cycle inhibitor is a taxane (e.g. paclitaxel, or an analogue or derivative thereof, an antimetabolite, an alkylating agent, or, a vinca alkaloid. In another embodiment, the cell-cycle inhibitor is camptothecin, or an analogue or derivative thereof. In still another embodiment, the cell cycle inhibitor is formulated along with a polymer. In yet another embodiment, the polymer comprises poly (ethylene-vinyl acetate), polyurethane poly (caprolactone), poly (lactic acid), or a copolymer of poly (caprolactone) and poly (lactic acid), or comprises MePEG.

In related embodiments, the present invention provides any one of the aforementioned methods wherein the cellular proliferation is due to cancer, stenosis or restenosis, an adhesion, vascular disease, or arthritis.

Within other related embodiments, the present invention provides a method wherein a cell-cycle inhibitor and/or radioactive source is administered close to the surface of the body. In another embodiment, a cell-cycle inhibitor or radioactive source is administered within a body cavity. In still another embodiment, the cell-cycle inhibitor and/or radioactive source is administered directly into a body tissue.

In yet other aspects of the invention, compositions are provided comprising a radioactive source and a cell-cycle inhibitor. In one embodiment, the radioactive source is selected from the group consisting of activity I¹²⁵, Pd¹⁰³ and Ir¹⁹²; Co⁶⁰, Cs¹³⁷, and Ru¹⁰⁶. In another embodiment, the cell-cycle inhibitor is a taxane such as paclitaxel or an analogue or derivative thereof. In still another embodiment, the cell-cycle inhibitor is an anti-metabolite, vinca alkaloid, or alkylating agent. In another, the cell cycle inhibitor is camptothecin, or an analogue or derivative thereof. In a further embodiment, the cell-cycle inhibitor is a polypeptide, which may be a protein or a peptide, including fragments or derivatives thereof and that may have modifications, such as D-amino acids. In yet another embodiment, the aforementioned compositions further comprise a polymer (e.g., poly (ethylene-vinyl acetate), polyurethane, poly (caprolactone), poly (lactic acid), or comprises a copolymer of poly (caprolactone) and poly (lactic acid), or comprises MePEG). Within yet other embodiments, the polymer is made radiopaque or echogenic.

Within other aspects of the present invention, therapeutic devices are provided, comprising a radioactive source; a body contact material carrying the radioactive source, the body contact member being applied to a pre-existing or created surface site of a patient's body to be treated by locally administered radiation from the radioactive source; and a cell-cycle inhibitor. In one embodiment, the therapeutic device wherein the body contact material is formed from one of a paste, gel, film or spray applied to the site to be treated.

In another aspect, the present invention provides a method of treating cellular proliferation, comprising administering to a patient any one of the aforementioned therapeutic devices.

In yet other aspects, the present invention provides a method for treating cellular proliferation, comprising administering to a patient a cell-cycle inhibitor and a source of radiation. In one embodiment, the present invention provides the aforementioned method for treating cellular proliferation wherein said source of radiation is Pd¹⁰³, Ir¹⁹², Co⁶⁰, Cs¹³⁷, Au¹⁹⁸, or Ru¹⁰⁶. In another embodiment, the source of radiation is I¹²⁵. In still another embodiment, the source of radiation is formulated along with a polymer. In another embodiment, the aforementioned method wherein said source of radiation is a radioactive stent, rod, disk, seed, or fastening devices (e.g., suture).

In related embodiments, the cell-cycle inhibitor is a taxane (e.g., paclitaxel, or an analogue or derivative thereof, an antimetabolite, an alkylating agent, or, a vinca alkaloid. In another embodiment, the cell-cycle inhibitor is camptothecin, or an analogue or derivative thereof. In still another embodiment, the cell cycle inhibitor is formulated along with a polymer. In yet another embodiment, the polymer comprises poly (ethylene-vinyl acetate), polyurethane poly (caprolactone), poly (lactic acid), or a copolymer of poly (caprolactone) and poly (lactic acid), or comprises MePEG.

In related embodiments, the present invention provides any one of the aforementioned methods wherein the cellular proliferation is due to cancer, stenosis or restenosis, an adhesion, vascular disease, or arthritis.

Within other related embodiments, the present invention provides a method wherein a cell-cycle inhibitor and/or radioactive source is administered close to the surface of the body. In another embodiment, a cell-cycle inhibitor or radioactive source is administered within a body cavity. In still another embodiment, the cell-cycle inhibitor and/or radioactive source is administered directly into a body tissue.

In yet other aspects of the invention, compositions are provided comprising a radioactive source and a cell-cycle inhibitor. In one embodiment, the radioactive source is selected from the group consisting of activity I¹²⁵, Pd¹⁰³ and Ir¹⁹²; Co⁶⁰, Cs¹³⁷, and Ru¹⁰⁶. In another embodiment, the cell-cycle inhibitor is a taxane such as paclitaxel or an analogue or derivative thereof. In still another embodiment, the cell-cycle inhibitor is an anti-metabolite, vinca alkaloid, or alkylating agent. In another, the cell cycle inhibitor is camptothecin, or an analogue or derivative thereof. In yet another embodiment, the aforementioned compositions further comprising a polymer (e.g., poly (ethylene-vinyl acetate), polyurethane, poly (caprolactone), poly (lactic acid), or comprises a copolymer of poly (caprolactone) and poly (lactic acid), or comprises MePEG).

Within various embodiments of the above invention, the cell-cycle inhibitors and/or radioactive sources provided herein can also be comprised of or made echogenic or radiopaque. For example, the cell-cycle inhibitor and/or radioactive source can have an echogenic or a radiopaque coating. Within one embodiment, a radioactive seed is positioned between echogenic spacers, which allow visualization of the spacers under ultrasound.

Within other aspects of the invention, radioactive polymers are provided comprising a radioactive monomer and a non-radioactive monomer. Within one embodiment the polymer is echogenic.

These and other aspects of the present invention will become evident upon reference to the following detailed description and attached drawings. In addition, various references are set forth herein which describe in more detail certain procedures or compositions (e.g., compounds, proteins, vectors, and their generation, etc.), and are therefore incorporated by reference in their entirety. When PCT applications are referred to it is also understood that the underlying or cited U.S. applications are also incorporated by reference herein.

### BRIEF DESCRIPTION OF THE DRAWINGS

FIG. 1 is a schematic illustration showing sites of action within a biological pathway where Cell Cycle Inhibitors may act to inhibit the cell cycle.

FIG. 2 is a schematic illustration of one representative cell-cycle inhibitor coated radioactive suture.

FIG. 3 is a schematic illustration of one representative cell-cycle inhibitor loaded radioactive suture.

FIG. 4 is a schematic illustration of one representative cell-cycle inhibitor coated radioactive seed.

FIG. 5 is a schematic illustration of one representative cell-cycle inhibitor coated radioactive wire.

FIG. 6 is a schematic illustration of one representative cell-cycle inhibitor loaded spacers.

FIG. 7A is a schematic illustration of one representative cell-cycle inhibitor loaded capsule.

FIG. 7B is a schematic illustration of one representative cell-cycle inhibitor coated capsule.

FIG. 8 is a schematic illustration of a representative surface mold containing or adapted to release a radioactive source.

FIG. 9 is a schematic illustration of one representative cell-cycle inhibitor loaded film containing radioactive seeds.

FIG. 10 is a schematic illustration of one representative cell-cycle inhibitor loaded film containing radioactive wires.

FIG. 11 is a schematic representation of spacer preparation. In A), the rod has been formed in the capillary tube. In B), the capillary tube is inserted through the septum. After insertion through the septum, the assembly is transferred to a water bath. In C) the rod is ejected into the sealed vial.

FIG. 12A shows in vitro profiles of paclitaxel release from radiation seed spacers.

FIG. 12B shows in vitro profiles of paclitaxel release from radiation seed spacers.

FIG. 13 shows in vitro profiles of paclitaxel release from paclitaxel coated brachytherapy seeds.

FIG. 14 shows an in vitro profile of paclitaxel release from a coated wire.

FIG. 15 shows an in vitro profile of paclitaxel release from a semi-solid injectable paste.

FIG. 16 shows the decrease in tumor volume 1 week after treatment with a locally administered Cell Cycle Inhibitor (paclitaxel) in conjunction with a local radiation source (I-125).

FIGS. 17A-E are a series of radioactive devices which may be coated with or adapted to release cell cycle inhibitors, including for example, 17A, a ring shaped device, 17B a horseshoe shaped device, 17C a hollow tube shaped device, 17D a rod with holes perpendicular to the axis of the rod, and 17E a rod with protrusions.

### DETAILED DESCRIPTION OF THE INVENTION

Prior to setting forth the invention, it may be helpful to an understanding thereof to set forth definitions of certain terms that will be used hereinafter.

“Hyperproliferative Disease” as used herein refers to any of a number of diseases which are characterized by excessive and/or inappropriate cell division leading to pathological changes. Neoplasia is a classic example of such a condition whereby abnormal cell division and tissue growth occurs more rapidly than normal and continues after the stimuli that initiated the new growth ceases. Neoplasms show partial or complete lack of structural organization and functional coordination with normal tissue and usually form a distinct mass of tissue which can be either benign (benign tumor) or malignant (cancer). Malignant tumors can occur in virtually any tissue (e.g., breast cancer, prostate cancer, colon cancer, lung cancer, skin cancer, etc.) and are characterized by local invasion of tissue and distant metastasis often leading to death. Benign tumor growth is typically not metastatic or locally invasive, but can lead in certain circumstances (e.g., benign brain tumors) to severe disease and even death due to altered tissue function or tumor growth compressing/damaging adjacent critical structures (e.g., arteries, veins, nerves).

Several other nonmalignant diseases are characterized by hyperproliferation of cells and are amenable to treatment with the described compositions and methods. These include premalignant lesions (e.g., polyps, actinic keratosis, cervical dypslasia, carcinoma in situ, Barrett's syndrome), psoriasis, arthritis, vascular disease (e.g., atherosclerosis, arteriosclerosis, arterial stenosis, venous stenosis, restenosis following angioplasty or stenting, and instent restenosis), surgical adhesions, pulmonary fibrosis, pterygium (and other benign diseases of the eye) and keloids.

“Radioactive Source” as used herein refers to any atomic nucleus capable of spontaneously emitting gamma rays or subatomic particles (alpha and beta rays, neutron rays). Commonly-used gamma emitting particles include radium (Ra²²³, Ra²²⁴, Ra²²⁵, Ra²²⁶, Ra²²⁷, Ra²²⁸), cobalt (Co⁵⁵, Co⁵⁶, Co⁵⁷, Co⁵⁸, Co⁶⁰, Co⁶¹, Co⁶²), cesium (Cs¹²⁰, Cs¹³⁰, Cs¹³¹, Cs¹³², Cs¹³⁴, Cs¹³⁵, Cs¹³⁶, Cs¹³⁷), gold (Au¹⁹⁴, Au¹⁹⁵, Au¹⁹⁶, Au¹⁹⁸, Au¹⁹⁹), iridium (Ir¹⁸⁸, Ir¹⁸⁹, Ir¹⁹⁰, Ir¹⁹²), iodine (I¹²⁰, I¹²¹, I¹²², I¹²³, I²⁴, I¹²⁵, I¹²⁶, I¹²⁸, I¹²⁹, I¹³⁰, I¹³¹, I¹³², I¹³³, I¹³⁴, I¹³⁵) and palladium (Pd¹⁰⁰, Pd¹⁰¹, Pd¹⁰³, Pd¹⁰⁷, Pd¹⁰⁹, Pd¹¹¹, Pd¹¹²). Commonly used beta emitters include phosphorus (P²⁹, P³⁰, P³², P³³), ruthenium (Ru⁹⁵, Ru⁹⁷, Ru¹⁰³, Ru¹⁰⁵, Ru¹⁰⁶), strontium (Sr⁸⁰, Sr⁸¹, Sr⁸², Sr⁸³, Sr⁸⁵, Sr⁸⁹, Sr⁹⁰, Sr⁹¹, Sr⁹²) and yttrium (Y⁸⁵, Y⁸⁶, Y⁸⁷, Y⁸⁸, Y⁹⁰, Y⁹¹, Y⁹², Y⁹³). Californium (Cf²⁴⁸, Cf²⁴⁹, Cf²⁵⁰, Cf²⁵¹, Cf²⁵², Cf²⁵³, Cf²⁵⁴, Cf²⁵⁵) is used as a neutron emitter. It should be noted that any other atomic nucleus capable of delivering a therapeutic dose of radioactivity would be suitable for the purposes of this invention Radioactive sources may be constructed or generated in a variety of forms, including for example, as devices (e.g., seeds, metal ribbons, fastening devices (e.g., sutures), stents, metal sheets or films, artificial joints, or other medical devices), or along with or comprised of polymers.

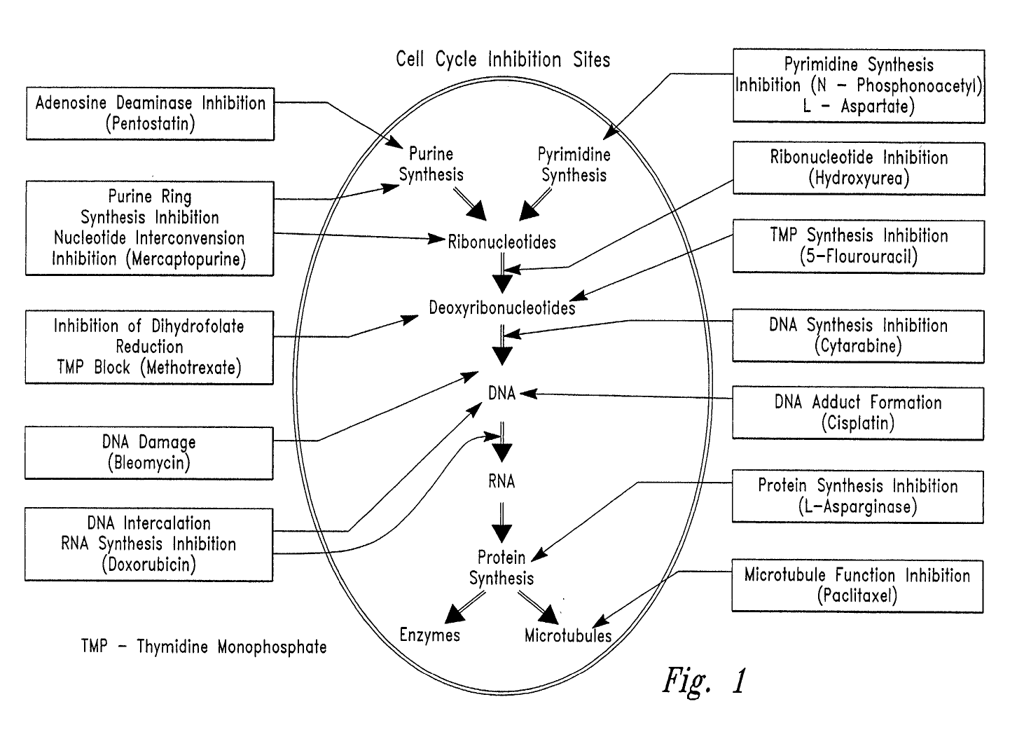

“Cell Cycle Inhibitor” as used herein refers to any protein, peptide, chemical or other molecule which delays or impairs a dividing cell's ability to progress through the cell cycle and replicate. Cell cycle inhibitors which prolong or arrest mitosis (M-phase) or DNA synthesis (S-phase) are particularly effective for the purposes of this invention as they increase the dividing cell's sensitivity to the effects of radiation. A wide variety of methods may be utilized to determine the ability of a compound to inhibit the cell cycle including univariate analysis of cellular DNA content and multiparameter analysis (see the Examples). A Cell Cycle Inhibitor may act to inhibit the cell cycle at any of the steps of the biological pathways shown in FIG. 1, as well as at other possible steps in other biological pathways. In addition, it should be understood that while a single cell cycle agent is often referred to, that this in fact should be understood to include two or more cell cycle agents, as more than one cell cycle agent may be utilized within the compositions, methods and/or devices described herein (e.g., two cell-cycle inhibitors may be selected that act on different steps shown in FIG. 1).

“Echogenic” or “Radiopaque” as used herein refers to a device or composition that has enhanced visualization using ultrasonic or radiologic means. For example, a therapeutic device or composition that is ‘echogenic’ will be more easily identified or seen with ultrasound, as opposed to a therapeutic device or composition which is not echogenic. Therapeutic devices can be made echogenic by, for example, creating a rough surface finish which has numerous acoustic interfaces than a smoother finish. Alternatively, a device or composition can be made with, or, coated with a composition which is echogenic or radiopaque (e.g., made with echogenic or radiopaque with materials such as powdered tantalum, tungsten, barium carbonate, bismuth oxide, barium sulfate, or, by the addition of microspheres or bubbles which present an acoustic interface.

As noted above, the present invention provides methods for treating, preventing, or, inhibiting the development of hyperproliferative diseases comprising the step of delivering to the site of disease at least one cell cycle inhibitor and at least one radioactive source. In related aspects devices are provided for therapeutic applications that can similarly be utilized to treat, prevent, or, inhibit the development of hyperproliferation. Discussed in more detail below are (I) Cell-Cycle Inhibitors; (II) Cell-Cycle Inhibitor Formulations; (III) Cell-Cycle Inhibitor—Radioactive Source/Representative Embodiments; and (IV) Clinical Applications.

### I. CELL-CYCLE INHIBITORS

Briefly, a wide variety of cell cycle inhibitory agents can be utilized, either with or without a carrier (e.g., a polymer or ointment or vector), in order to treat or prevent a hyperproliferative disease. Representative examples of such agents include taxanes (e.g., paclitaxel (discussed in more detail below) and docetaxel) (Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(40): 351-386, 1993), Etanidazole, Nimorazole (B. A. Chabner and D. L. Longo. Cancer Chemotherapy and Biotherapy - Principles and Practice. Lippincott-Raven Publishers, New York, 1996, p.554), perfluorochemicals with hyperbaric oxygen, transfusion, erythropoietin, BW12C, nicotinamide, hydralazine, BSO, WR-2721, IudR, DUdR, etanidazole, WR-2721, BSO, mono-substituted keto-aldehyde compounds (L. G. Egyud.

Keto-aldehyde-amine addition products and method of making same. U.S. Pat. No. 4,066,650, Jan. 3, 1978), nitroimidazole (K. C. Agrawal and M. Sakaguchi. Nitroimidazole radiosensitizers for Hypoxic tumor cells and compositions thereof. U.S. Pat. No. 4,462,992, Jul. 31, 1984), 5-substituted-4-nitroimidazoles (Adams et al., Int. J. Radiat. Biol. Relat. Stud. Phys., Chem. Med. 40(2):153-61, 1981), SR-2508 (Brown et al., Int. J. Radiat. Oncol., Biol. Phys. 7(6):695-703, 1981), 2H-isoindolediones (J. A. Myers, 2H-Isoindolediones, their synthesis and use as radiosensitizers. U.S. Pat. No. 4,494,547, Jan. 22, 1985), chiral [[(2-bromoethyl)-amino]methyl]-nitro- 1 H-imidazole-1-ethanol (V. G. Beylin, et al., Process for preparing chiral [[(2-bromoethyl)-amino]methyl ]-nitro-1H-imidazole-1-ethanol and related compounds. U.S. Pat. No. 5,543,527, Aug. 6, 1996; U.S. Pat. No. 4,797,397; Jan. 10, 1989; U.S. Pat. No. 5,342,959, Aug. 30, 1994), nitroaniline derivatives (W. A. Denny, et al Nitroaniline derivatives and their use as anti-tumor agents. U.S. Pat. No. 5,571,845, Nov. 5, 1996), DNA-affinic hypoxia selective cytotoxins (M.V. Papadopoulou-Rosenzweig. DNA-affinic hypoxia selective cytotoxins. U.S. Pat. No. 5,602,142, Feb. 11, 1997), halogenated DNA ligand (R. F. Martin. Halogenated DNA ligand radiosensitizers for cancer therapy. U.S. Pat. No. 5,641,764, Jun. 24, 1997), 1,2,4 benzotriazine oxides (W. W. Lee et al. 1,2,4-benzotriazine oxides as radiosensitizers and selective cytotoxic agents. U.S. Pat. No. 5,616,584, Apr. 1, 1997; U.S. Pat. No. 5,624,925, Apr. 29, 1997; Process for Preparing 1,2,4 Benzotriazine oxides. U.S. Pat. No. 5,175,287, Dec. 29, 1992), nitric oxide (J. B. Mitchell et al., Use of Nitric oxide releasing compounds as hypoxic cell radiation sensitizers. U.S. Pat. No. 5,650,442, Jul. 22, 1997), 2-nitroimidazole derivatives (M. J. Suto et al. 2-Nitroimidazole derivatives useful as radiosensitizers for hypoxic tumor cells. U.S. Pat. No. 4,797,397, Jan. 10, 1989; T. Suzuki. 2-Nitroimidazole derivative, production thereof, and radiosensitizer containing the same as active ingredient. U.S. Pat. No. 5,270,330, Dec. 14, 1993; T. Suzuki et al. 2-Nitroimidazole derivative, production thereof, and radiosensitizer containing the same as active ingredient. U.S. Pat. No. 5,270,330, Dec 14, 1993; T. Suzuki. 2-Nitroimidazole derivative, production thereof and radiosensitizer containing the same as active ingredient; Patent EP 0 513 351 B1, Jan. 24, 1991), fluorine-containing nitroazole derivatives (T. Kagiya. Fluorine-containing nitroazole derivatives and radiosensitizer comprising the same. U.S. Pat. No. 4,927,941, May 22, 1990), copper (M. J. Abrams. Copper Radiosensitizers. U.S. Pat. No. 5,100,885, Mar. 31, 1992), combination modality cancer therapy (D. H. Picker et al. Combination modality cancer therapy. U.S. Pat. No. 4,681,091, Jul. 21, 1987). 5-CldC or (d)H₄U or 5-halo-2′-halo-2′-deoxy-cytidine or -uridine derivatives (S. B. Greer. Method and Materials for sensitizing neoplastic tissue to radiation. U.S. Pat. No. 4,894,364 Jan. 16, 1990), platinum complexes (K. A. Skov. Platinum Complexes with one radiosensitizing ligand. U.S. Pat. No. 4,921,963. May 1, 1990; K. A. Skov. Platinum Complexes with one radiosensitizing ligand. Patent EP 0 287 317 A3), fluorine-containing nitroazole (T. Kagiya, et al. Fluorine-containing nitroazole derivatives and radiosensitizer comprising the same. U.S. Pat. No. 4,927,941. May 22,1990), benzamide (W. W. Lee. Substituted Benzamide Radiosensitizers. U.S. Pat. No. 5,032,617, Jul. 16, 1991), autobiotics (L.G. Egyud. Autobiotics and their use in eliminating nonself cells in vivo. U.S. Pat. No. 5,147,652. Sep. 15, 1992), benzamide and nicotinamide (W. W. Lee et al Benzamide and Nictoinamide Radiosensitizers. U.S. Pat. No. 5,215,738, Jun. 1, 1993), acridine-intercalator (M. Papadopoulou-Rosenzweig. Acridine Intercalator based hypoxia selective cytotoxins. U.S. Pat. No. 5,294,715, Mar. 15, 1994), fluorine-containing nitroimidazole (T. Kagiya et al. Fluorine containing nitroimidazole compounds. U.S. Pat. No. 5,304,654, Apr. 19, 1994), hydroxylated texaphyrins (J. L. Sessler et al. Hydroxylated texaphrins. U.S. Pat. No. 5,457,183, Oct. 10, 1995), hydroxylated compound derivative (T. Suzuki et al. Heterocyclic compound derivative, production thereof and radiosensitizer and antiviral agent containing said derivative as active ingredient. Publication Number 011106775 A (Japan), Oct. 22,1987; T. Suzuki et al. Heterocyclic compound derivative, production thereof and radiosensitizer, antiviral agent and anti cancer agent containing said derivative as active ingredient. Publication Number 01139596 A (Japan), Nov. 25, 1987; S. Sakaguchi et al. Heterocyclic compound derivative, its production and radiosensitizer containing said derivative as active ingredient; Publication Number 63170375 A (Japan), Jan. 7, 1987), fluorine containing 3-nitro-1,2,4-triazole (T. Kagitani et al. Novel fluorine-containing 3-nitro- 1,2,4-triazole and radiosensitizer containing same compound. Publication Number 02076861 A (Japan), Mar. 31, 1988), 5-thiotretrazole derivative or its salt (E. Kano et al Radiosensitizer for Hypoxic cell. Publication Number 61010511 A (Japan), Jun. 26, 1984), Nitrothiazole (T Kagitani et al. Radiation-sensitizing agent. Publication Number 61167616 A (Japan) Jan. 22, 1985), imidazole derivatives (S. Inayma et al. Imidazole derivative. Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (T. Kagitani et al., Radiosensitizer. Publication Number 62039525 A (Japan), Aug. 15, 1985), 3-nitro-1,2,4-triazole (T. Kagitani et al Radiosensitizer. Publication Number 62138427 A (Japan), Dec. 12, 1985), Carcinostatic action regulator (H. Amagase. Carcinostatic action regulator. Publication Number 63099017 A (Japan), Nov. 21, 1986), 4,5-dinitroimidazole derivative (S. Inayama. 4,5-Dinitroimidazole derivative. Publication Number 63310873 A (Japan) Jun. 9, 1987), nitrotriazole Compound (T. Kagitanil. Nitrotriazole Compound. Publication Number 07149737 A (Japan) Jun. 22, 1993), cisplatin, doxorubin, misonidazole, mitomycin, tiripazamine, nitrosourea, mercaptopurine, methotrexate, flurouracil, bleomycin, vincristine, carboplatin, epirubicin, doxorubicin, cyclophosphamide, vindesine, etoposide (I. F. Tannock. Review Article: Treatment of Cancer with Radiation and Drugs. Journal of Clinical Oncology 14(12):3156-3174, 1996), camptothecin (Ewend M. G. et al. Local delivery of chemotherapy and concurrent external beam radiotherapy prolongs survival in metastatic brain tumor models. Cancer Research 56(22):5217-5223, 1996) and paclitaxel (Tishler R. B. et al. Taxol: a novel radiation sensitizer. International Journal of Radiation Oncology and Biological Physics 22(3):613-617, 1992).

A number of the above-mentioned cell cycle inhibitors also have a wide variety of analogues and derivatives, including, but not limited to, cisplatin, cyclophosphamide, misonidazole, tiripazamine, nitrosourea, mercaptopurine, methotrexate, flurouracil, epirubicin, doxorubicin, vindesine and etoposide. Analogues and derivatives include (CPA)₂Pt[DOLYM] and (DACH)Pt[DOLYM] cisplatin (Choi et al., Arch. Pharmacal Res. 22(2):151-156, 1999), Cis-[PtCl₂(4,7-H-5-methyl-7-oxo]1,2,4[triazolo[1,5-a]pyrimidine)₂] (Navarro et al., J. Med. Chem. 41(3):332-338, 1998), [Pt(cis-1,4-DACH)(trans-Cl₂)(CBDCA)].½MeOH cisplatin (Shamsuddin et al., Inorg. Chem. 36(25):5969-5971, 1997), 4-pyridoxate diammine hydroxy platinum (Tokunaga et al., Pharm. Sci. 3(7):353-356, 1997), Pt(II) . . . Pt(II) (Pt₂[NHCHN(C(CH₂)(CH₃))]₄) (Navarro et al., Inorg. Chem. 35(26):7829-7835, 1996), 254-S cisplatin analogue (Koga et al., Neurol Res. 18(3):244-247, 1996), o-phenylenediamine ligand bearing cisplatin analogues (Koeckerbauer & Bednarski, J. Inorg. Biochem. 62(4):281-298, 1996), trans, cis-[Pt(OAc)₂I₂(en)] (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-[Pt(NH₃)(4-aminoTEMP-O){d(GpG)}] (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), CI-973 cisplatin analogue (Yang et al., Int. J. Oncol. 5(3):597-602, 1994), cis-diamminedichloroplatinum(II) and its analogues cis-1,1-cyclobutanedicarbosylato(2R)-2-methyl-1,4-butanediam-mineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bemays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol 33(l):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman,Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2): 157-65, 1987), JM8 and JM9 cisplatin analogues (Harstrick et al., Int. J. Androl. 10(1); 139-45, 1987), (NPr4)2((PtCL4).cis-(PtCl2-(NH2Me)2)) (Brammer et al., J. Chem. Soc., Chem. Commun. 6:443-5, 1987), aliphatic tricarboxylic acid platinum complexes (EPA 185225), cis-dichloro(amino acid)(tert-butylamine)platinum(II) complexes (Pasini & Bersanetti, Inorg. Chim. Acta 107(4):259-67, 1985); 4-hydroperoxycylcophospharnide (Ballard et al., Cancer Chemother. Pharmacol. 26(6):397-402, 1990), acyclouridine cyclophosphamide derivatives (Zakerinia et al., Helv. Chim. Acta 73(4):912-15, 1990), 1,3,2-dioxa- and —oxazaphosphorinane cyclophosphamide analogues (Yang et al., Tetrahedron 44(20):6305-14, 1988), C5-substituted cyclophosphamide analogues (Spada, University of Rhode Island Dissertation, 1987), tetrahydrooxazine cyclophosphamide analogues (Valente, University of Rochester Dissertation, 1988), phenyl ketone cyclophosphamide analogues (Hales et al., Teratology 39(1):31-7, 1989), phenylketophosphamide cyclophosphamide analogues (Ludeman et al., J. Med. Chem. 29(5):716-27, 1986), ASTA Z-7557 cyclophosphamide analogues (Evans et al., Int. J. Cancer 34(6):883-90, 1984), 3-(1-oxy-2,2,6,6-tetramethyl-4-piperidinyl)cyclophosphamide (Tsui et al., J. Med. Chem. 25(9):1106-10, 1982), 2-oxobis(2-β-chloroethylamino)-4-,6-dimethyl-1,3,2-oxazaphosphorinane cyclophosphamide (Carpenter et al., Phosphorus Sulfir 12(3):287-93, 1982), 5-fluoro- and 5-chlorocyclophosphamide (Foster et al., J. Med. Chem. 24(12):1399-403, 1981), cis- and trans-4-phenylcyclophosphamide (Boyd et al., J. Med. Chem. 23(4):372-5, 1980), 5-bromocyclophosphamide, 3,5-dehydrocyclophosphamide (Ludeman et al., J. Med. Chem. 22(2):151-8, 1979), 4-ethoxycarbonyl cyclophosphamide analogues (Foster, J. Pharm. Sci. 67(5):709-10, 1978), arylaminotetrahydro-2H- 1,3,2-oxazaphosphorine 2-oxide cyclophosphamide analogues (Hamacher, Arch. Pharm. (Weinheim, Ger.) 310(5):J,428-34, 1977), NSC-26271 cyclophosphamide analogues (Montgomery & Struck, Cancer Treat. Rep. 60(4):J381-93, 1976), benzo annulated cyclophosphamide analogues (Ludeman & Zon, J. Med. Chem. 18(12):J1251-3, 1975), 6-trifluoromethylcyclophosphamide (Farmer & Cox, J. Med. Chem. 18(11):J1106-10, 1975), 4-methylcyclophosphamide and 6-methycyclophosphamide analogues (Cox et al., Biochem. Pharmacol. 24(5):J599-606, 1975); FCE 23762 doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Nat'l Cancer Inst. 89(16):1217-1223, 1997), 4-demethoxy-7-O-[2,6-dideoxy-4-O-(2,3,6-trideoxy-3-amino-α-lyxo-hexopyranosyl)-α-L-lyxo-hexopyranosyl]adriamicinone doxorubicin disaccharide analog (Monteagudo et al., Carbohydr. Res. 300(1):11-16, 1997), 2-pyrrolinodoxorubicin (Nagy et al., Proc. Nat'l Acad. Sci. U. S. A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int. J. Cancer 58(1):85-94, 1994), methoxymorpholino doxorubicin derivative (Kuhl et al., Cancer Chemother. Pharmacol 33(l):10-16, 1993), (6-maleimidocaproyl)hydrazone doxorubicin derivative (Willner et al., Bioconjugate Chem. 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. OncoL 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ., 16(Biol. 1):21-7, 1988), 4′-deoxydoxorubicin (Schoelzel et al., Leuk. Res. 10(12):1455-9, 1986), 4-demethyoxy-4′-o-methyldoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (4,314,054), 3′-deamino-3′-(4-mortholinyl) doxorubicin derivatives (4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20, 1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy- 1 -piperidinyl) doxorubicin derivatives (U.S. Pat. No. 4,314,054), doxorubicin-14-valerate, morpholinodoxorubicin (5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277); 4,5-dimethylmisonidazole (Born et al., Biochem. Pharmacol 43(6):1337-44, 1992), azo and azoxy misonidazole derivatives (Gattavecchia & Tonelli, Int. J. Radiat. BioL Relat. Stud. Phys., Chem. Med. 45(5):469-77, 1984); RB90740 (Wardman et al., Br. J. Cancer, 74 Suppl (27):S70-S74, 1996); 6-bromo and 6-chloro-2,3-dihydro-1,4-benzothiazines nitrosourea derivatives (Rai et al., Heterocycl. Commun. 2(6):587-592, 1996), diamino acid nitrosourea derivatives (Dulude et al., Bioorg. Med. Chem. Lett. 4(22):2697-700, 1994; Dulude et al., Bioorg. Med. Chem. 3(2):151-60, 1995), amino acid nitrosourea derivatives (Zheleva et al., Pharmazie 50(1):25-6, 1995), 3′,4′-didemethoxy-3′,4′-dioxo-4-deoxypodophyllotoxin nitrosourea derivatives (Miyahara et al., Heterocycles 39(1):361-9, 1994), ACNU (Matsunaga et al., Immunopharmacology 23(3):199-204, 1992), tertiary phosphine oxide nitrosourea derivatives (Guguva et al., Pharmazie 46(8):603, 1991), sulfamerizine and sulfamethizole nitrosourea derivatives (Chiang et al., Zhonghua Yaozue Zazhi 43(5):401-6, 1991), thymidine nitrosourea analogues (Zhang et al., Cancer Commun. 3(4):119-26, 1991), 1,3-bis(2-chloroethyl)-1-nitrosourea (August et al., Cancer Res. 51(6):1586-90, 1991), 2,2,6,6-tetramethyl-1-oxopiperidiunium nitrosourea derivatives (U.S.S.R. 1261253), 2- and 4-deoxy sugar nitrosourea derivatives (4,902,791), nitroxyl nitrosourea derivatives (U.S.S.R. 1336489), fotemustine (Boutin et al., Eur. J. Cancer Clin. Oncol. 25(9):1311-16, 1989), pyrimidine (II) nitrosourea derivatives (Wei et al., Chung-hua Yao Hsueh Tsa Chih 41(1):19-26, 1989), CGP 6809 (Schieweck et al., Cancer Chemother. Pharmacol. 23(6):341-7, 1989), B-3839 (Prajda et al., In Vivo 2(2):151-4, 1988), 5-halogenocytosine nitrosourea derivatives (Chiang & Tseng, Tai-wan Yao Hsueh Tsa Chih 38(1):37-43, 1986), 1-(2-chloroethyl)-3-isobutyl-3-(β-maltosyl)-1-nitrosourea (Fujimoto & Ogawa, J. Pharmacobio-Dyn. 10(7):341-5, 1987), sulfur-containing nitrosoureas (Tang et al., Yaoxue Xuebao 21(7):502-9, 1986), sucrose, 6-((((2-chloroethyl)nitrosoamino-)carbonyl)amino)-6-deoxysucrose (NS-1C) and 6′-((((2-chloroethyl)nitrosoamino)carbonyl)amino)-6′-deoxysucrose (NS-1D) nitrosourea derivatives (Tanoh et al., Chemotherapy (Tokyo) 33(11):969-77, 1985), CNCC, RFCNU and chlorozotocin (Mena et al., Chemotherapy (Basel) 32(2):131-7, 1986), CNUA (Edanami et al., Chemotherapy (Tokyo) 33(5):455-61, 1985), 1-(2-chloroethyl)-3-isobutyl-3-(β-maltosyl)-1-nitrosourea (Fujimoto & Ogawa, Jpn. J. Cancer Res. (Gann) 76(7):651-6, 1985), choline-like nitrosoalkylureas (Belyaev et al., Izv. Akad. NAUK SSSR, Ser. Khim. 3:553-7, 1985), sucrose nitrosourea derivatives (JP 84219300), sulfa drug nitrosourea analogues (Chiang et al., Proc. Nat'l Sci. Counc., Repub. China, Part A 8(1):18-22, 1984), DONU (Asanuma et al., J. Jpn. Soc. Cancer Ther. 17(8):2035-43, 1982), N,N′-bis (N-(2-chloroethyl)-N-nitrosocarbamoyl)cystamine (CNCC) (Blazsek et al., Toxicol. Appl. Pharmacol. 74(2):250-7, 1984), dimethylnitrosourea (Krutova et al., Izv. Akad. NAUK SSSR, Ser. Biol. 3:439-45, 1984), GANU (Sava & Giraldi, Cancer Chemother. Pharmacol 10(3):167-9, 1983), CCNU (Capelli et al., Med., Biol., Environ. 11(1):111-16, 1983), 5-aminomethyl-2′-deoxyuridine nitrosourea analogues (Shiau, Shih Ta Hsueh Pao (Taipei) 27:681-9, 1982), TA-077 (Fujimoto & Ogawa, Cancer Chemother. Pharmacol. 9(3):134-9, 1982), gentianose nitrosourea derivatives (JP 82 80396), CNCC, RFCNU, RPCNU AND chlorozotocin (CZT) (Marzin et al., INSERM Symp., 19(Nitrosoureas Cancer Treat.):165-74, 1981), thiocolchicine nitrosourea analogues (George, Shih Ta Hsueh Pao (Taipei) 25:355-62, 1980), 2-chloroethyl-nitrosourea (Zeller & Eisenbrand, Oncology 38(l):39-42, 1981), ACNU, (1-(4-amino-2-methyl-5-pyrimidinyl)methyl-3-(2-chloroethyl)-3-nitrosourea hydrochloride) (Shibuya et al., Gan To Kagaku Ryoho 7(8):1393-401, 1980), N-deacetylmethyl thiocolchicine nitrosourea analogues (Lin et al., J. Med. Chem. 23(12):1440-2, 1980), pyridine and piperidine nitrosourea derivatives (Crider et al., J. Med. Chem. 23(8):848-51, 1980), methyl-CCNU (Zimber & Perk, Refu. Vet. 35(l):28, 1978), phensuzimide nitrosourea derivatives (Crider et al., J. Med. Chem. 23(3):324-6, 1980), ergoline nitrosourea derivatives (Crider et al., J. Med. Chem. 22(1):32-5, 1979), glucopyranose nitrosourea derivatives (JP 78 95917), 1-(2-chloroethyl)-3-cyclohexyl-1-nitrosourea (Farner et al., J. Med. Chem. 21(6):514-20, 1978), 4-(3-(2-chloroethyl)-3-nitrosoureid-o)-cis-cyclohexanecarboxylic acid (Drewinko et al., Cancer Treat. Rep. 61(8):J1513-18, 1977), RPCNU (ICIG 1163) (Larnicol et al., Biomedicine 26(3):J176-81, 1977), IOB-252 (Sorodoc et al., Rev. Roum. Med. Virol. 28(1):J55-61, 1977), 1,3-bis(2-chloroethyl)-1-nitrosourea (BCNU) (Siebert & Eisenbrand, Mutat. Res. 42(1):J45-50, 1977), 1-tetrahydroxycyclopentyl-3-nitroso-3-(2-chloroethyl)-urea (4,039,578), d-1-1-(β-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7, 1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7, 1981); indoline ring and a modified ornithine or glutamic acid-bearing methotrexate derivatives (Matsuoka et al., Chem. Pharm. Bull. 45(7): 1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaaminopterin analogues (DeGraw et al., J. Med. Chem. 40(3):370-376, 1997), 5-deazaaminopterin and 5,10-dideazaaminopterin methotrexate analogues (Piper et al., J. Med. Chem. 40(3):377-384, 1997), indoline moiety-bearing methotrexate derivatives (Matsuoka et al., Chem. Pharm. Bull 44(7):1332-1337, 1996), lipophilic amide methotrexate derivatives (Pignatello et al., World Meet. Pharn., Biopharm. Pharm. Technol., 563-4, 1995), L-tbreo-(2S,4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl Chem. 32(1):243-8, 1995), N-(α-aminoacyl) methotrexate derivatives (Cheung et al., Pteridines 3(1-2):101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(12):2400-3, 1991), β,γ-methano methotrexate analogues (Rosowsky et al., Pteridines 2(3):133-9, 1991), 10-deazaaminopterin (10-EDAM) analogue (Braakhuis et al., Chem. Biol Pteridines, Proc. Int. Symp. Pteridines Folic Acid Deriv., 1027-30, 1989), γ-tetrazole methotrexate analogue (Kalman et al., Chem. Biol Pteridines, Proc. Int. Symp. Pteridines Folic Acid Deriv., 1154-7, 1989), N-(L-a-aminoacyl) methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (U.S. Pat. No. 4,725,687), Nδ-acyl-Nα-(4-amino-4-deoxypteroyl)-L-omithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed. Polym.): 311-24, 1987), methotrexate-γ-dimyristoylphophatidylethanolamine (Kinsky et al., Biochim. Biophys. Acta 917(2):211-18, 1987), methotrexate polyglutamate analogues (Rosowsky et al., Chem. Biol. Pteridines, Pteridines Folid Acid Deriv., Proc. Int. Symp. Pteridines Folid Acid Deriv.: Chem., Biol. Clin. Aspects: 985-8, 1986), poly-γ-glutamyl methotrexate derivatives (Kisliuk et al., Chem. Biol. Pteridines, Pteridines Folid Acid Deriv., Proc. Int. Symp. Pteridines Folid Acid Deriv.: Chem., Biol. Clin. Aspects: 989-92, 1986), deoxyuridylate methotrexate derivatives (Webber et al., Chem. Biol. Pteridines, Pteridines Folid Acid Deriv., Proc. Int. Symp. Pteridines Folid Acid Deriv.: Chem., Biol. Clin. Aspects: 659-62, 1986), iodoacetyl lysine methotrexate analogue (Delcamp et al., Chem. Biol. Pteridines, Pteridines Folid Acid Deriv., Proc. Int. Symp. Pteridines Folid Acid Deriv.: Chem., Biol. Clin. Aspects: 807-9, 1986), 2,.omega.-diaminoalkanoid acid-containing methotrexate analogues (McGuire et al., Biochem. Pharmacol 35(15):2607-13, 1986), polyglutamate methotrexate derivatives (Kamen & Winick, Methods Enzymol. 122(Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(l):155-8, 1986), pyrazine methotrexate analogue (Lever & Vestal, J. Heterocycl Chem. 22(l):5-6, 1985), cysteic acid and homocysteic acid methotrexate analogues (4,490,529), y-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.—Chim. Ther. 19(3):267-73, 1984), poly (L-lysine) methotrexate conjugates (Rosowsky et al., J. Med. Chem. 27(7):888-93, 1984), dilysine and trilysine methotrexate derivates (Forsch & Rosowsky, J. Org. Chem. 49(7):1305-9, 1984), 7-hydroxymethotrexate (Fabre et al., Cancer Res. 43(10):4648-52, 1983), poly-γ-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol 17(1):105-10, 1980), halogentated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220); N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9, 1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3):144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-acetyl-3-O-toluyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin (Lanius, Adv. Chemother. Gastrointest. Cancer, (Int. Symp.), 159-67, 1984); N-substituted deacetylvinblastine amide (vindesine) sulfates (Conrad et al., J. Med. Chem. 22(4): 391-400, 1979); and Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

Within one preferred embodiment of the invention, the cell cycle inhibitor is paclitaxel, a compound which disrupts mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216, 1993). “Paclitaxel” (which should be understood herein to include formulations, prodrugs, analogues and derivatives such as, for example, TAXOL®, TAXOTERE®, docetaxel, 10-desacetyl analogues of paclitaxel and 3′N-desbenzoyl-3′N-t-butoxy carbonyl analogues of paclitaxel) may be readily prepared utilizing techniques known to those skilled in the art (see, e.g., Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; W094/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Missouri (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy- 11,12-dihydrotaxol- 10,12(18)-diene derivatives, 10-desacetoxytaxol, Protaxol (2′-and/or 7-O-ester derivatives ), (2′-and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N- dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L- isoleucyl)taxol, 2′-(L-valyl)taxol, 7-(L-valyl)taxol, 2′,7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10- deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site- substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14- beta -hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogs bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the Cell Cycle Inhibitor is a taxane having the formula (C1):

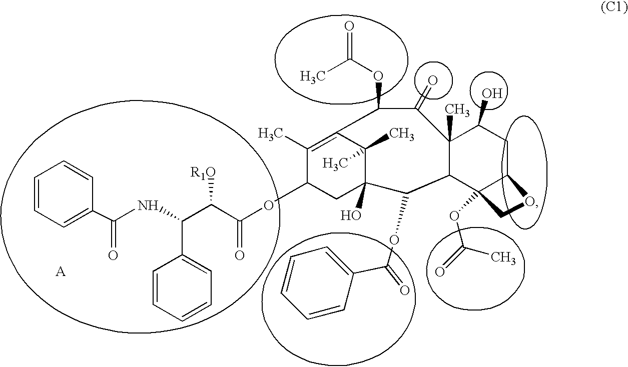

where the gray-highlighted portions may be substituted and the non-highlighted portion is the taxane core. A side-chain (labeled “A” in the diagram) is desirably present in order for the compound to have good activity as a Cell Cycle Inhibitor. Examples of compounds having this structure include paclitaxel (Merck Index entry 7117), docetaxel (Taxotere, Merck Index entry 3458), and 3′-desphenyl-3′-(4-ntirophenyl)-N-debenzoyl-N-(t-butoxycarbonyl)- 1 0-deacetyltaxol.

In one aspect, suitable taxanes such as paclitaxel and its analogs and derivatives are disclosed in U.S. Pat. No. 5,440,056 as having the structure (C2):

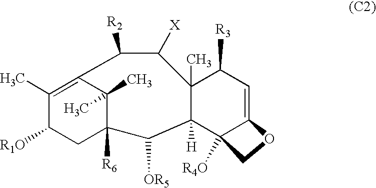

wherein X may be oxygen (paclitaxel), hydrogen (9-deoxy derivatives), thioacyl, or dihydroxyl precursors; R₁ is selected from paclitaxel or taxotere side chains or alkanoyl of the formula (C3)

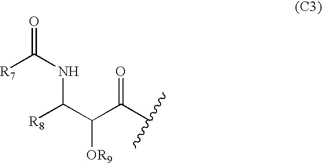

wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogs and derivatives useful as Cell Cycle Inhibitors in the present invention are disclosed in PCT International Patent Application No. WO 93/10076. As disclosed in this publication, the analog or derivative should have a side chain attached to the taxane nucleus at C₁₃, as shown in the structure below (formula C4), in order to confer antitumor activity to the taxane.

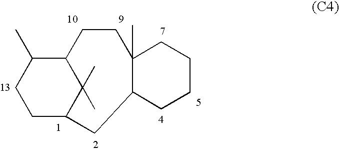

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, the taxane-based Cell Cycle Inhibitor useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aroyl, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Taxanes in general, and paclitaxel is particular, is considered to function as a Cell Cycle Inhibitor by acting as an anti-microtubule agent, and more specifically as a stabilizer. These compounds have been shown useful in the treatment of proliferative disorders, including: non-small cell (NSC) lung; small cell lung; breast; prostate; cervical; endometrial; head and neck cancers.

In another aspect, the Cell Cycle Inhibitor is a vinca alkaloid. Vinca alkaloids have the following general structure. They are indole-dihydroindole dimers.

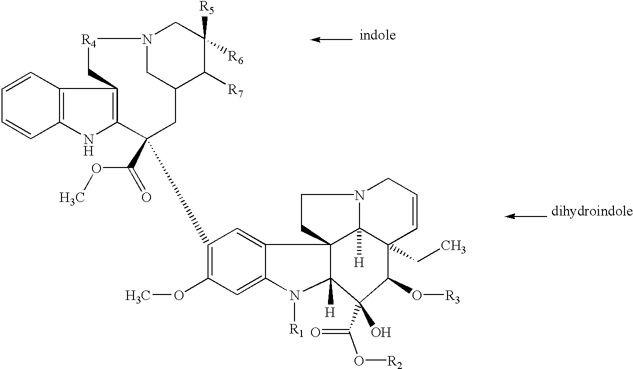

As disclosed in U.S. Pat. Nos. 4,841,045 and 5,030,620, R₁ can be a formyl or methyl group or alternately H. R₁ could also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)-R where R is NH₂, an amino acid ester or a peptide ester. R₃ is typically C(O)CH₃, CH₃ or H. Alternately, a protein fragment may be linked by a bifunctional group such as maleoyl amino acid. R₃ could also be substituted to form an alkyl ester, which may be further substituted. R4 may be —CH₂— or a single bond. R₅ and R₆ may be H, OH or a lower alkyl, typically —CH₂CH₃. Alternatively R₆ and R₇ may together form an oxirane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary vinca alkaloids are vinblastine, vincristine, vincristine sulfate, vindesine, and vinorelbine, having the structures:

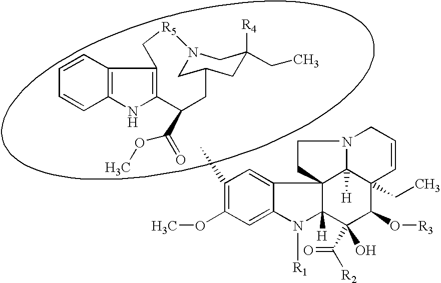

| | R₁ | R₂ | R₃ | R₄ | R₅ |
|---|---|---|---|---|---|
| Vinbiastine: | CH₃ | OCH₃ | C(O)CH₃ | OH | CH₂ |
| Vincristine: | CH₂O | OCH₃ | C(O)CH₃ | OH | CH₂ |
| Vindesine: | CH₃ | NH₂ | H | OH | CH₂ |
| Vinorelbine: | OH₃ | OCH₃ | C(O)CH₃ | 3-double | single |
| | | | | bond | bond |

Analogs typically require the side group (shaded area) in order to have activity. These compounds are thought to act as Cell Cycle Inhibitors by functioning as anti-microtubule agents, and more specifically to inhibit polymerization. These compounds have been shown useful in treating proliferative disorders, including NSC lung; small cell lung; breast; prostate; brain; head and neck; retinoblastoma; bladder; and penile cancers; and soft tissue sarcoma.

In another aspect, the Cell Cycle Inhibitor is Camptothecin, or an anolog or derivative thereof. Camptothecins have the following general structure.

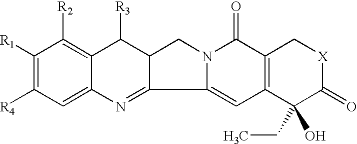

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

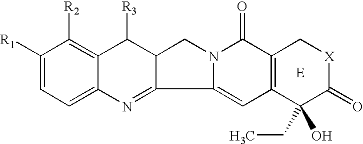

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analog, NH for 21-lactam analogs

Camptothecins have the five rings shown here. The ring labeled E must be intact (the lactone rather than carboxylate form) for maximum activity and minimum toxicity. These compounds are useful to as Cell Cycle Inhibitors, where they function as Topoisomerase I Inhibitors and/or DNA cleavage agents. They have been shown useful in the treatment of proliferative disorders, including, for example, NSC lung; small cell lung; and cervical cancers.

In another aspect, the Cell Cycle Inhibitor is a Podophyllotoxin, or a derivative or an analog thereof. Exemplary compounds of this type are Etoposide or Teniposide, which have the following structures:

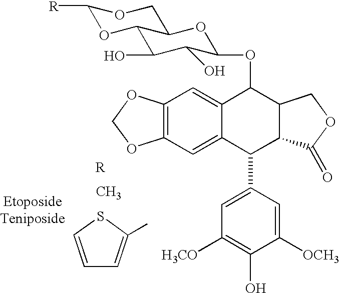

These compounds are thought to function as Cell Cycle Inhibitors by being Topoisomerase II Inhibitors and/or by DNA cleaving agents. They have been shown useful as antiproliferative agents in, e.g., small cell lung, prostate, and brain cancers, and in retinoblastoma.

In another aspect, the Cell Cycle Inhibitor is an anthracycline. Anthracyclines have the following general structure, where the R groups may be a variety of organic groups:

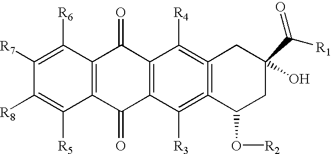

According to U.S. Pat. No. 5,594,158, suitable R groups are as follows: R₁ is CH₃ or CH₂OH; R₂ is daunosamine or H; R₃ and R₄ are independently one of OH, NO₂, NH₂, F, Cl, Br, I, CN, H or groups derived from these; R₅ is hydrogen, hydroxy, or methoxy; and R₆₋₈ are all hydrogen. Alternatively, R₅ and R₆ are hydrogen and R₇ and R₈ are alkyl or halogen, or vice versa, i.e., R₇ and R₈ are hydrogen and R₅ and R₆ are alkyl or halogen.

According to U.S. Pat. No. 5,843,903, R₁ may be a conjugated peptide. According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure:

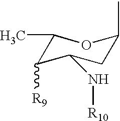

in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxy, mercapto, phenyl, benzyl or methylthio (see U.S. Pat. No. 4,296,105).

Exemplary anthracyclines are Doxorubicin, Daunorubicin, Idarubicin, Epirubicin, Pirarubicin, Zorubicin, and Carubicin. Suitable compounds have the structures:

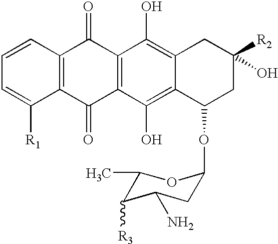

| | R₁ | R₂ | R₃ |
|---|---|---|---|
| Doxorubicin | OCH₃ | C(O)CH₂OH | OH out of ring plane |
| Epirubicin | OCH₃ | C(O)CH₂OH | OH in ring plane |
| (4 epimer of doxorubicin) | | | |
| Daunorubicin | OCH₃ | C(O)CH₃ | OH out of ring plane |
| Idarubicin | H | C(O)CH₃ | OH out of ring plane |
| Pirarubicin | OCH₃ | C(O)CH₂OH | 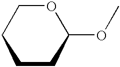 |
| Zorubicin | OCH₃ | C(CH₃)(═N)NHC(O)C₈H₆ | OH |
| Carubicin | OH | C(O)CH₃ | OH out of ring plane |

Other suitable anthracyclines are Anthramycin, Mitoxantrone, Menogaril, Nogalamycin, Aclacinomycin A, Olivomycin A, Chromomycin A₃, and Plicamycin having the structures:

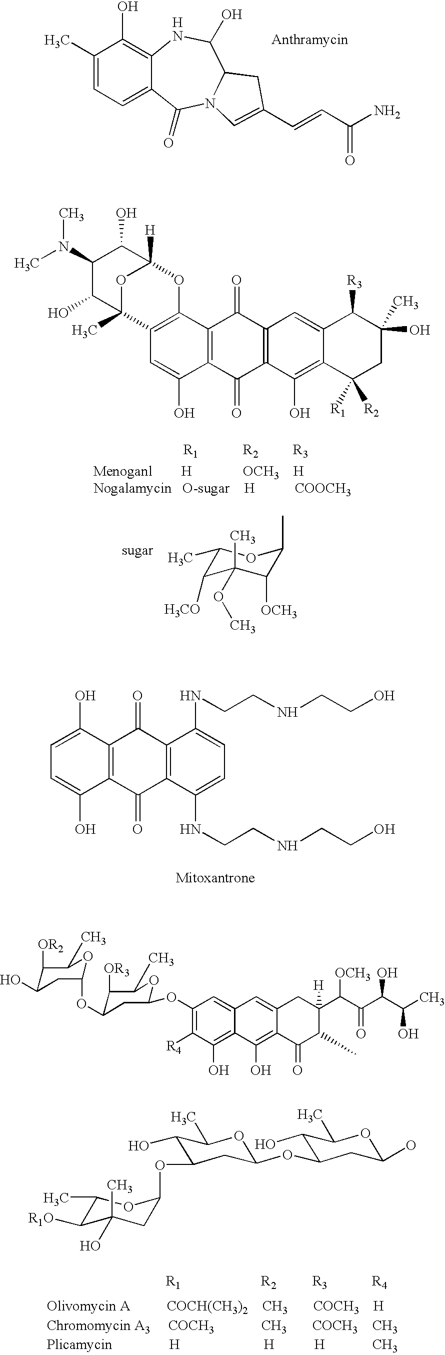

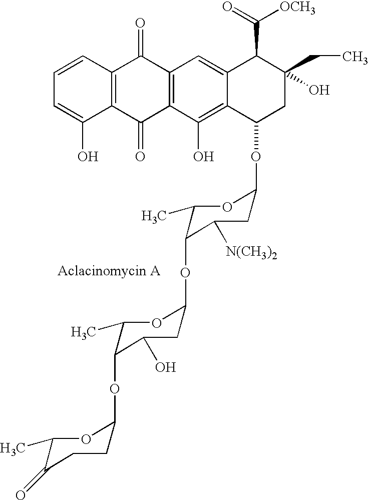

These compounds are thought to function as Cell Cycle Inhibitors by being Topoisomerase Inhibitors and/or by DNA cleaving agents. They have been shown useful in the treatment of proliferative disorders, including small cell lung; breast; endometrial; head and neck; retinoblastoma; liver; bile duct; islet cell; and bladder cancers; and soft tissue sarcoma.

In another aspect, the Cell Cycle Inhibitor is a platinum compound. In general, suitable platinum complexes may be of Pt(II) or Pt(IV) and have this basic structure:

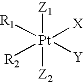

wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

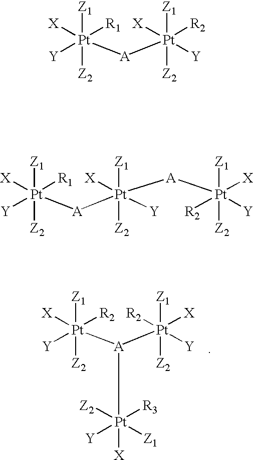

Exemplary platinum compounds are Cisplatin, Carboplatin, Oxaliplatin, and Miboplatin having the structures:

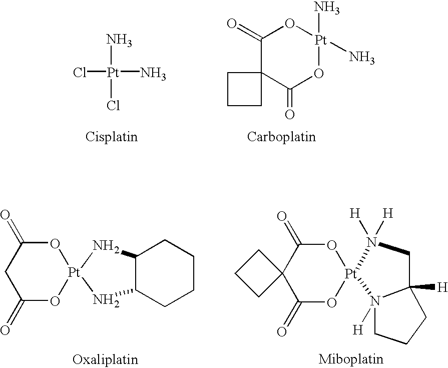

These compounds are thought to function as Cell Cycle Inhibitors by binding to DNA, i.e., acting as alkylating agents of DNA. These compounds have been shown useful in the treatment of cell proliferative disorders, including, e.g., NSC lung; small cell lung; breast; cervical; brain; head and neck; esophageal; retinoblastom; liver; bile duct; bladder; penile; and vulvar cancers; and soft tissue sarcoma.

In another aspect, the Cell Cycle Inhibitor is a nitrosourea. Nitrosoureas have the following general structure (C5), where exemplary compounds include BCNU (Carmustine), Methyl-CCNU, (Semustine), CCNU (Lomustine), Ranimustine, Nimustine, Chlorozotocin, Fotemustine, and Streptozocin, some of which are shown below.

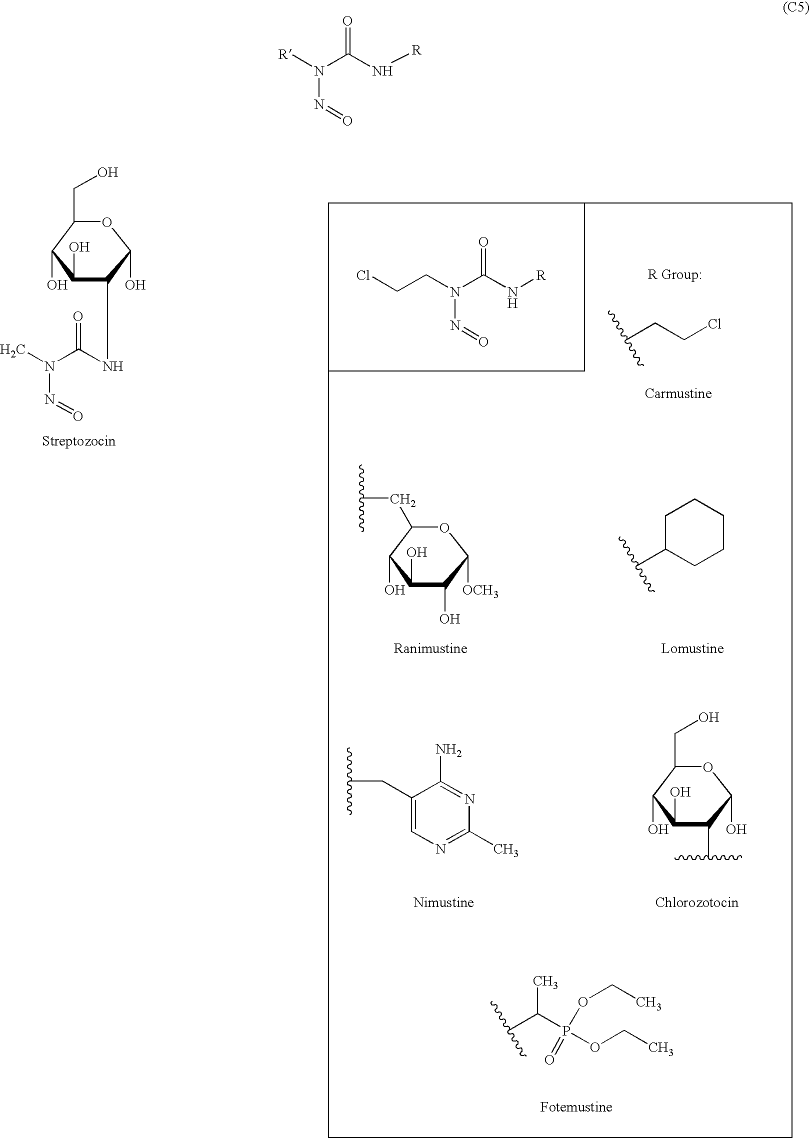

Other suitable R groups include cyclic alkanes, alkanes, halogen substituted groups, sugars, aryl and heteroaryl groups, phosphonyl and sulfonyl groups. As disclosed in U.S. Pat. No. 4,367,239, R may suitably be CH₂—C(X)(Y)(Z), wherein X and Y may be the same or different members of the following groups: phenyl, cyclyhexyl, or a phenyl or cyclohexyl group substituted with groups such as halogen, lower alkyl (C₁₋₄), trifluore methyl, cyano, phenyl, cyclohexyl, lower alkyloxy (C₁₋₄). Z has the following structure: —alkylene—N-R₁R₂, where R₁ and R₂ may be the same or different members of the following group: lower alkyl (C₁₋₄) and benzyl, or together R₁ and R₂ may form a saturated 5 or 6 membered heterocyclic such as pyrrolidine, piperidine, morfoline, thiomorfoline, N-lower alkyl piperazine, where the heterocyclic may be optionally substituted with lower alkyl groups.

As disclosed in U.S. Pat. No. 6,096,923, R and R′ of formula (C5) may be the same or different, where each may be a substituted or unsubstituted hydrocarbon having 1-10 carbons. Substitutions may include hydrocarbyl, halo, ester, amide, carboxylic acid, ether, thioether and alcohol groups. As disclosed in U.S. Pat. No. 4,472,379, R of formula (C5) may be an amide bond and a pyranose structure (e.g., Methyl 2′-[N-[N-(2-chloroethyl)-N-nitroso-carbamoyl]-glycyl]amino-2′-deoxy-α-D-glucopyranoside). As disclosed in U.S. Pat. No. 4,150,146, R of formula (C5) may be an alkyl group of 2 to 6 carbons and may be substituted with an ester, sulfonyl, or hydroxyl group. It may also be substituted with a carboxylic acid or CONH₂ group.

These nitrosourea compounds are thought to function as Cell Cycle Inhibitor by binding to DNA, that is, by functioning as DNA alkylating agents. These Cell Cycle Inhibitors have been shown useful in treating cell proliferative disorders such as, for example, islet cell; small cell lung; melanoma; and brain cancers.

In another aspect, the Cell Cycle Inhibitor is a Nitroimidazole, where exemplary Nitroimidazoles are Metronidazole, Benznidazole, Etanidazole, and Misonidazole, having the structures:

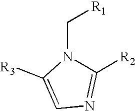

| | R₁ | R₂ | R₃ |
|---|---|---|---|
| Metronidazole | CH₂OH | CH₃ | NO₂ |
| Benznidazole | C(O)NHCH₂-benzyl | NO₂ | H |
| Etanidazole | C(O)NHCH₂CH₂OH | NO₂ | H |
| Misonidazole | CH(OH)CH₂OCH₃ | NO₂ | H |

Suitable nitroimidazole compounds are disclosed in, e.g., U.S. Pat. Nos. 4,371,540 and 4,462,992.

In another aspect, the Cell Cycle Inhibitor is a folic acid antagonist, such as Methotrexate or derivatives or analogs thereof, including Edatrexate, Trimetrexate, Raltitrexed, Piritrexim, Denopterin, Tomudex, and Pteropterin. Methotrexate analogs have the following general structure:

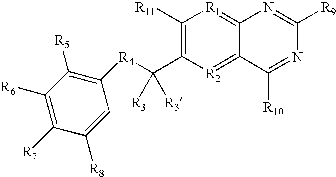

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure:

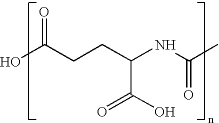

wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

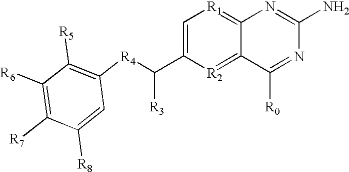

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatrexate | NH₂ | N | N | H | CH(CH₂CH₃) | H | H | A (n = 1) | H |
| Trimetrexate | NH₂ | CH | C(CH₃) | H | NH | H | OCH₃ | OCH₃ | OCH₃ |
| Pteropterin | OH | N | N | H | NH | H | H | A (n = 3) | H |
| Denopterin | OH | N | N | CH₃ | N(CH₃) | H | H | A (n = 1) | H |
| Pentrexim | NH₂ | N | C(CH₃) | H | single bond | OCH₃ | H | H | OCH₃ |

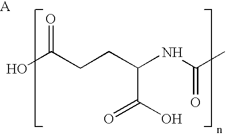

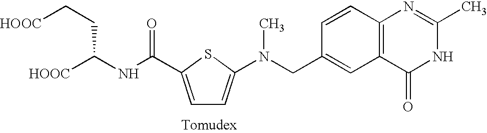

These compounds are thought to function as Cell Cycle Inhibitors by serving as antimetabolites of folic acid. They have been shown useful in the treatment of cell proliferative disorders including, for example, soft tissue sarcoma, small cell lung, breast, brain, head and neck, bladder, and penile cancers.

In another aspect, the Cell Cycle Inhibitor is a cytidine analog, such as Cytarabine or derivatives or analogs thereof, including Enocitabine, FMdC ((E(-2′-deoxy-2′-(fluoromethylene)cytidine), Gemcitabine, 5-Azacitidine, Ancitabine, and 6-Azauridine. Exemplary compounds have the structures:

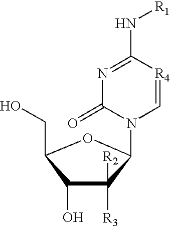

| | R₁ | R₂ | R₃ | R₄ |
|---|---|---|---|---|
| Cytarabine | H | OH | H | CH |
| Enocitabine | C(O)(CH₂)₂₀CH₃ | OH | H | CH |
| Gemcitabine | H | F | F | CH |
| Azacitidine | H | H | OH | N |
| FMdC | H | CH₂F | H | CH |

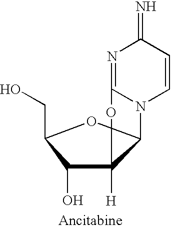

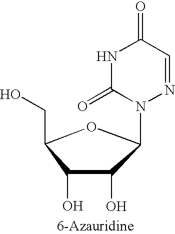

These compounds are thought to function as Cell Cycle Inhibitors as acting as antimetabolites of pyrimidine. These compounds have been shown useful in the treatment of cell proliferative disorders including, for example, pancreatic, breast, cervical, NSC lung, and bile duct cancers.

In another aspect, the Cell Cycle Inhibitor is a pyrimidine analog. In one aspect, the Pyrimidine analogs have the general structure:

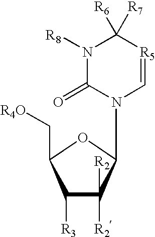

wherein positions 2′,3′ and 5′ on the sugar ring (R₂, R₃ and R4, respectively) can be H, hydroxyl, phosphoryl (see, e.g., U.S. Pat. No. 4,086,417) or ester (see, e.g., U.S. Pat. No. 3,894,000). Esters can be of alkyl, cycloalkyl, aryl or heterocyclo/aryl types. The 2′ carbon can be hydroxylated at either R₂ or R₂′, the other group is H. Alternately, the 2′ carbon can be substituted with halogens e.g., fluoro or difluoro cytidines such as Gemcytabine. Alternately, the sugar can be substituted for another heterocyclic group such as a furyl group or for an alkane, an alkyl ether or an amide linked alkane such as C(O)NH(CH₂)₅CH₃. The 2° amine can be substituted with an aliphatic acyl (R₁) linked with an amide (see, e.g., U.S. Pat. No. 3,991,045) or urethane (see, e.g., U.S. Pat. No. 3,894,000) bond. It can also be further substituted to form a quaternary ammonium salt. R₅ in the pyrimidine ring may be N or CR, where R is H, halogen containing groups, or alkyl (see, e.g., U.S. Pat. No. 4,086,417). R₆ and R₇ can together can form an oxo group or R₆=NH—R₁ and R₇=H. R₈ is H or R₇ and R₈ together can form a double bond or R₈ can be X, where X is:

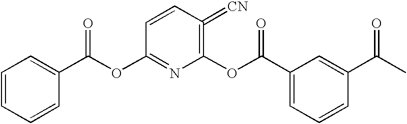

Specific pyrimidine analogs are disclosed in U.S. Pat. No. 3,894,000 (see, e.g., 2′-O-palmityl-ara-cytidine, 3′-O-benzoyl-ara-cytidine, and more than 10 other examples); U.S. Patent No. 3,991,045 (see, e.g., N4-acyl-1-β-D-arabinofuranosylcytosine, and numerous acyl groups derivatives as listed therein, such as palmitoyl.

In another aspect, the Cell Cycle Inhibitor is a fluoro-pyrimidine analog, such as 5-fluorouracil, or an analog or derivative thereof, including Carrnofur, Doxifluridine, Emitefur, Tegafur, and Floxuridine. Exemplary compounds have the structures:

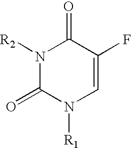

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

A₁

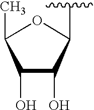

A₂

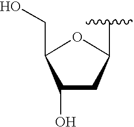

B

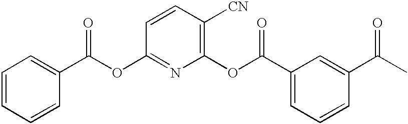

C

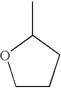

Other suitable fluoropyrimidine analogs include 5-FudR (5-fluoro-5 deoxyuridine), or an analog or derivative thereof, including 5-iododeoxyuridine (5-IudR), 5-bromodeoxyuridine (5-BudR), Fluorouridine triphosphate (5-FUTP), and Fluorodeoxyuridine monophosphate (5-dFUMP). Exemplary compounds have the structures:

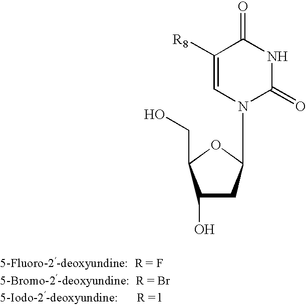

These compounds are thought to function as Cell Cycle Inhibitors by serving as antimetabolites of pyrimidine. These compounds have been shown useful in the treatment of cell proliferative disorders such as breast, cervical, non-melanoma skin, head and neck, esophageal, bile duct, pancreatic, islet cell, penile, and vulvar cancers.

In another aspect, the Cell Cycle Inhibitor is a purine analog. Purine analogs have the following general structure:

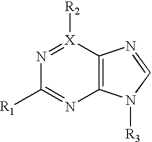

wherein X is typically carbon; R₁ is H, halogen, amine or a substituted phenyl; R₂ is H, a primary, secondary or tertiary amine, a sulfur containing group, typically —SH, an alkane, a cyclic alkane, a heterocyclic or a sugar; R₃ is H, a sugar (typically a furanose or pyranose structure), a substituted sugar or a cyclic or heterocyclic alkane or aryl group. See, e.g., U.S. Pat. No. 5,602,140 for compounds of this type.

In the case of pentostatin, X—R₂ is —CH₂CH(OH)—, i.e., a second carbon atom is inserted in the ring between X and the adjacent nitrogen atom. The X-N double bond becomes a single bond.

U.S. Pat. No. 5,446,139 describes suitable purine analogs of the type shown in the following formula:

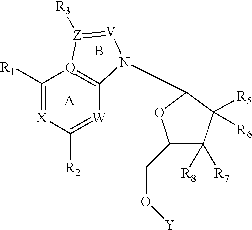

wherein N signifies nitrogen and V, W, X, Z can be either carbon or nitrogen with the following provisos. Ring A may have 0 to 3 nitrogen atoms in its structure. If two nitrogens are present in ring A, one must be in the W position. If only one is present, it must not be in the Q position. V and Q must not be simultaneously nitrogen. Z and Q must not be simultaneously nitrogen. If Z is nitrogen, R₃ is not present. Furthermore, R₁₋₃ are independently one of H, halogen, C₁₋₇ alkyl, C₁₋₇ alkenyl, hydroxyl, mercapto, C₁₋₇ alkylthio, C₁₋₇ alkoxy, C₂₋₇ alkenyloxy, aryl oxy, nitro, primary, secondary or tertiary amine containing group. R₅₋₈ are H or up to two of the positions may contain independently one of OH, halogen, cyano, azido, substituted amino, R₅ and R₇ can together form a double bond. Y is H, a C₁₋₇ alkylcarbonyl, or a mono- di or tri phosphate.

Exemplary suitable purine analogs include 6-mercaptopurine, thioguanosine, Thiamiprine, Cladribine, Fludaribine, Tubercidin, Puromycin, Pentoxyfilline; where these compounds may optionally be phosphorylated. Exemplary compounds have the structures:

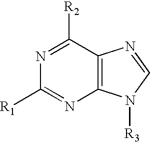

| | R₁ | R₂ | R₃ |
|---|---|---|---|
| Thioguanosine | NH₂ | SH | B₁ |
| Thiamiprine | NH₂ | A | H |
| Cladribine | Cl | NH₂ | B₂ |
| Fludarabine | F | NH₂ | B₃ |
| Puromycin | H | N(CH₃)₂ | B₄ |
| Tubercidin | H | NH₂ | B₁ |

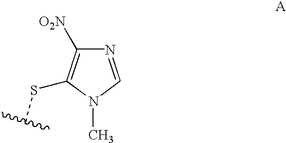

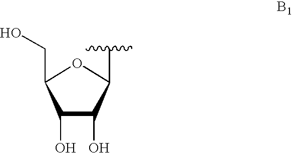

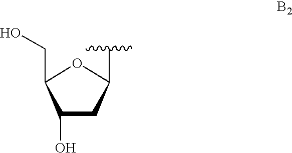

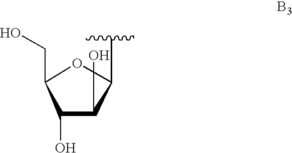

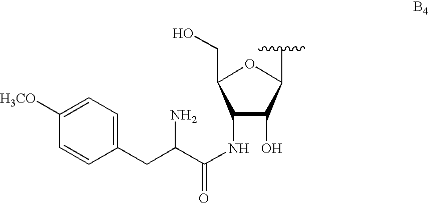

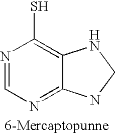

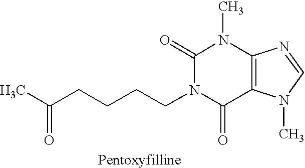

These compounds are thought to function as Cell Cycle Inhibitors by serving as antimetabolites of purine.

In another aspect, the Cell Cycle Inhibitor is a nitrogen mustard. Many suitable nitrogen mustards are known and are suitably used as a Cell Cycle Inhibitor in the present invention. Suitable nitrogen mustards are also known as cyclophosphamides.

A preferred nitrogen mustard has the general structure:

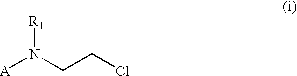

where A is:

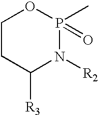

or —CH₃ or other alkane, or chloronated alkane, typically CH₂CH(CH₃)Cl, or a polycyclic group such as B, or a substituted phenyl such as C or a heterocyclic group such as D.

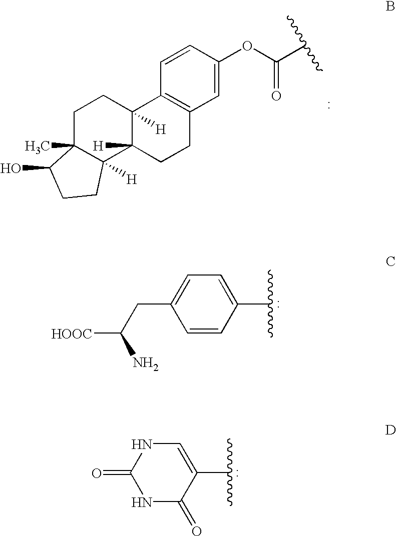

Suitable nitrogen mustards are disclosed in U.S. Pat. No. 3,808,297, wherein A is:

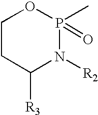

R₁₋₂ are H or CH₂CH₂Cl; R₃ is H or oxygen-containing groups such as hydroperoxy; and R4 can be alkyl, aryl, heterocyclic.

The cyclic moiety need not be intact. See, e.g., U.S. Pat. Nos. 5,472,956, 4,908,356, 4,841,085 that describe the following type of structure:

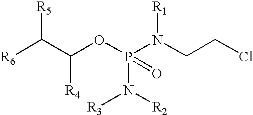

wherein R₁ is H or CH₂CH₂Cl, and R₂₋₆ are various substituent groups.

Exemplary nitrogen mustards include methylchloroethamine, and analogs or derivatives thereof, including methylchloroethamine oxide hydrohchloride, Novembichin, and Mannomustine (a halogenated sugar). Exemplary compounds have the structures:

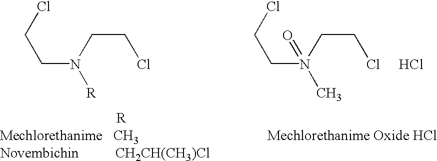

The nitrogen mustard may be Cyclophosphamide, Ifosfamide, Perfosfamide, or Torofosfamide, where these compounds have the structures:

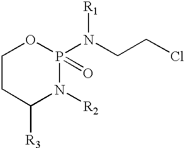

| | R₁ | R₂ | R₃ |
|---|---|---|---|
| Cyclophosphamide | CH₂CH₂Cl | H | H |
| Ifosfamide | H | CH₂CH₂Cl | H |
| Perfosfamide | CH₂CH₂Cl | H | OOH |
| Torofosfamide | CH₂CH₂Cl | CH₂CH₂Cl | H |

The nitrogen mustard may be Estramustine, or an analog or derivative thereof, including Phenesterine, Prednimustine, and Estramustine PO₄. Thus, suitable nitrogen mustard type Cell Cycle Inhibitors of the present invention have the structures:

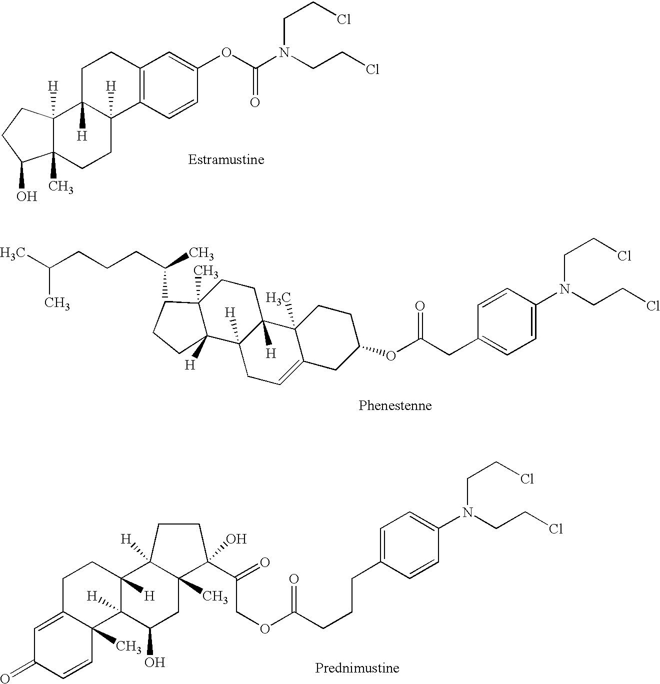

The nitrogen mustard may be Chlorambucil, or an analog or derivative thereof, including Melphalan and Chlormaphazine. Thus, suitable nitrogen mustard type Cell Cycle Inhibitors of the present invention have the structures:

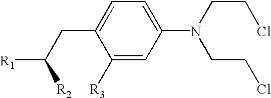

| | R₁ | R₂ | R₃ |
|---|---|---|---|
| Chlorambucil | CH₂COOH | H | H |
| Melphalan | COOH | NH₂ | H |

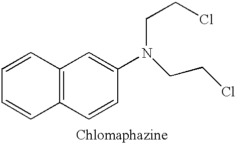

The nitrogen mustard may be uracil mustard, which has the structure:

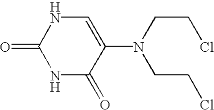

The nitrogen mustards are thought to function as Cell Cycle Inhibitors by serving as alkylating agents for DNA. Nitrogen mustards have been shown useful in the treatment of cell proliferative disorders including, for example, small cell lung, breast, cervical, head and neck, prostate, retinoblastoma, and soft tissue sarcoma.

The Cell Cycle Inhibitor of the present invention may be a hydroxyurea. Hydroxyureas have the following general structure:

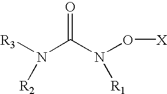

Suitable hydroxyureas are disclosed in, for example, U.S. Pat. No. 6,080,874, wherein R₁ is:

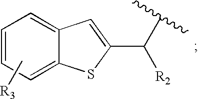

and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-[3-[5-(4-fluorophenylthio)-furyl ]-2-cyclopenten-1-yl]N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with on or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form:

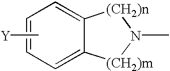

wherein m is 1 or 2, n is 0-2 and Y is an alkyl group.

In one aspect, the hydroxyurea has the structure:

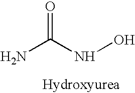

Hydroxyureas are thought to function as Cell Cycle Inhibitors by serving to inhibit DNA synthesis.

In another aspect, the Cell Cycle Inhibitor is a Bleomycin, such as Bleomycin A₂, which have the structures:

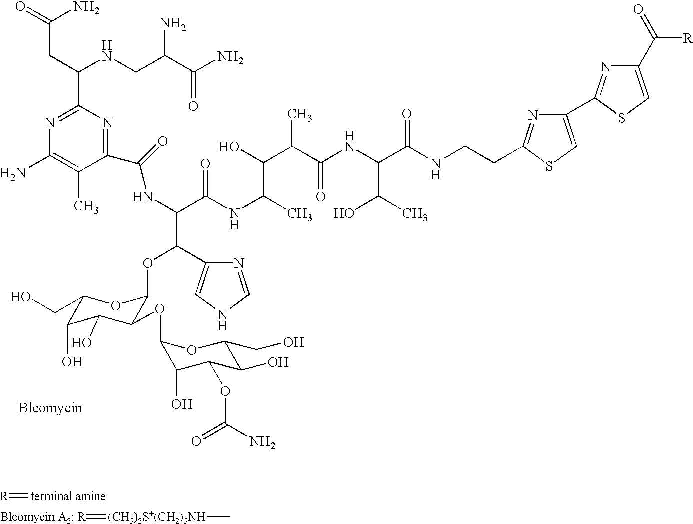

Bleomycins are thought to function as Cell Cycle Inhiitors by cleaving DNA. They have been shown useful in the treatment of cell proliferative disorder such as, e.g., penile cancer.

In another aspect, the Cell Cycle Inhibitor is a Mytomycin, such as Mitomycin C, or an analog or derivative thereof, such as Porfiromycin. Suitable compounds have the structures:

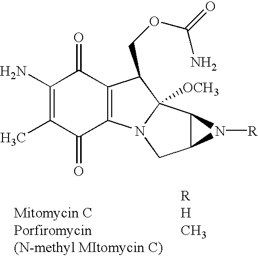

These compounds are thought to function as Cell Cycle Inhibitors by serving as DNA alkylating agents. Mitomycins have been shown useful in the treatment of cell proliferative disorders such as, for example, esophageal, liver, bladder, and breast cancers.

In another aspect, the Cell Cycle Inhibitor is an Alkyl sulfonate, such as Busulfan, or an analog or derivative thereof, such as Treosulfan, Improsulfan, Piposulfan, and Pipobroman. Exemplary compounds have the structures:

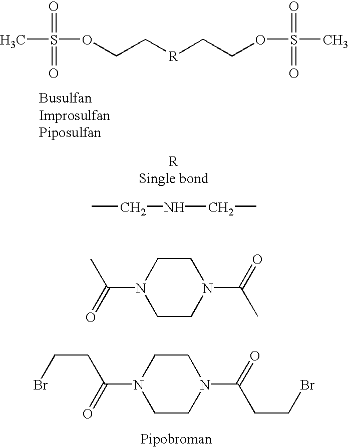

These compounds are thought to function as Cell Cycle Inhibitors by serving as DNA alkylating agents.

In another aspect, the Cell Cycle Inhibitor is a Benzamide. In yet another aspect, the Cell Cycle Inhibitor is a Nicotinamide. These compounds have the basic structure:

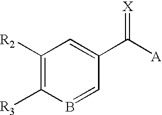

wherein X is either O or S; A is commonly NH₂ or it can be OH or an alkoxy group; B is N or C—R₄, where R₄ is H or an ether-linked hydroxylated alkane such as OCH₂CH₂OH, the alkane may be linear or branched and may contain one or more hydroxyl groups. Alternately, B may be N—R₅ in which case the double bond in the ring involving B is a single bond. R₅ may be H, and alkyl or an aryl group (see, e.g., U.S. Pat. No. 4,258,052); R₂ is H, OR₆, SR₆ or NHR₆, where R₆ is an alkyl group; and R₃ is H, a lower alkyl, an ether linked lower alkyl such as —O—Me or —O—Ethyl (see, e.g., U.S. Pat. No. 5,215,738).

Suitable benzamide compounds have the structures:

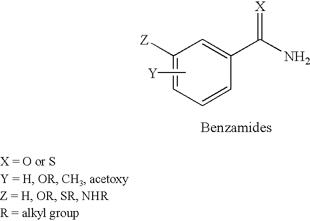

where additional compounds are disclosed in U.S. Pat. No. 5,215,738, (listing some 32 compounds).

Suitable nicotinamide compounds have the structures:

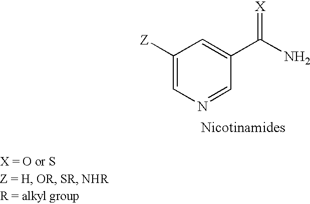

where additional compounds are disclosed in U.S. Pat. No. 5,215,738 (listing some 58 compounds, e.g., 5-OH nicotinamide, 5-aminonicotinamide, 5-(2,3-dihydroxypropoxy) nicotinamide), and compounds having the structures:

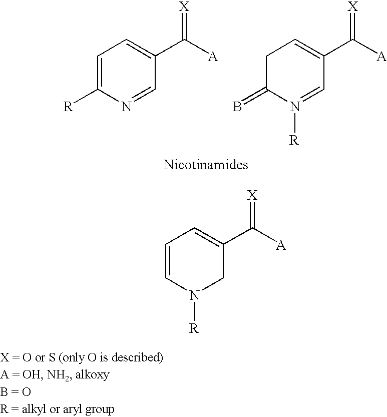

and U.S. Pat. No. 4,258,052 (listing some 46 compounds, e.g., 1-methyl-6-keto-1,6-dihydronicotinic acid).

In one aspect, the Cell Cycle Inhibitor is a tetrazine compound, such as Temozolomide, or an analog or derivative thereof, including Dacarbazine. Suitable compounds have the structures:

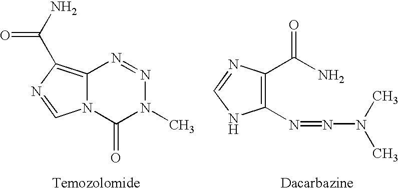

Another suitable tetrazine compound is Procarbazine, including HCl and HBr salts, having the structure:

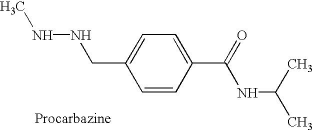

In another aspect, the Cell Cycle Inhibitor is Actinomycin D, or other members of this family, including Dactinomycin, Actinomycin Cl, Actinomycin C₂, Actinomycin C₃, and Actinomycin F₁. Suitable compounds have the structures:

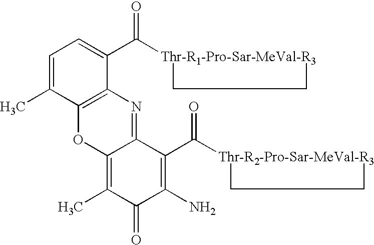

| | R₁ | R₂ | R₂ |
|---|---|---|---|
| Actinomycin D (C₁) | D-Val | D-Val | single bond |
| Actinomycin C₂ | D-Val | D-Alloisoleucine | O |
| Actinomycin C₃ | D-Alloisoleucine | D-Alloisoleucine | O |

In another aspect, the Cell Cycle Inhibitor is an aziridine compound, such as Benzodepa, or an analog or derivative thereof, including Meturedepa, Uredepa, and Carboquone. Suitable compounds have the structures:

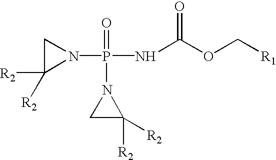

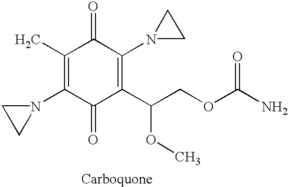

| | R₁ | R₂ |
|---|---|---|
| Benzodepa | phenyl | H |
| Meturedepa | CH₃ | CH₃ |
| Uredepa | CH₃ | H |

In another aspect, the Cell Cycle Inhibitor is halogenated sugar, such as Mitolactol, or an analog or derivative thereof, including Mitobronitol and Mannomustine. Suitable compounds have the structures:

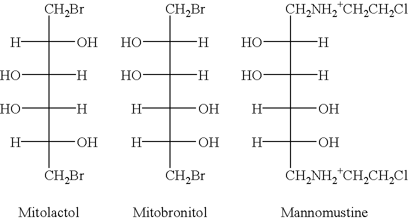

In another aspect, the Cell Cycle Inhibitor is a Diazo compound, such as Azaserine, or an analog or derivative thereof, including 6-diazo-5-oxo-L-norleucine and 5-diazouracil (also a pyrimidine analog). Suitable compounds have the structures:

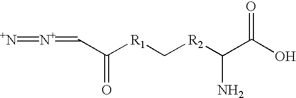

| | R₁ | R₂ |
|---|---|---|
| Azaserine | O | single bond |
| 6-diazo-5-oxo- | single bond | CH₂ |
| L-norleucine | | |

Other compounds that may serve as Cell Cycle Inhibitors according to the present invention are Pazelliptine; Wortmannin; Metoclopramide; RSU; Buthionine sulfoxime; Tumeric; Curcumin; AG337, a thymidylate synthase inhibitor; Levamisole; Lentinan, a polysaccharide; Razoxane, an EDTA analog; Indomethacin; Chlorpromazine; α and β interferon; MnBOPP; Gadolinium texaphyrin; 4-amino-1,8-naphthalimide; Staurosporine derivative of CGP; and SR-2508.

Thus, in one aspect, the Cell Cycle Inhibitor is a DNA alylating agent. In another aspect, the Cell Cycle Inhibitor is an anti-microtubule agent. In another aspect, the Cell Cycle Inhibitor is a Topoisomerase inhibitor. In another aspect, the Cell Cycle Inhibitor is a DNA cleaving agent. In another aspect, the Cell Cycle Inhibitor is an antimetabolite. In another aspect, the Cell Cycle Inhibitor functions by inhibiting adenosine deaminase (e.g., as a purine analog). In another aspect, the Cell Cycle Inhibitor functions by inhibiting purine ring synthesis and/or as a nucleotide interconversion inhibitor (e.g., as a purine analog such as mercaptopurine). In another aspect, the Cell Cycle Inhibitor functions by inhibiting dihydrofolate reduction and/or as a thymidine monophosphate block (e.g., methotrexate). In another aspect, the Cell Cycle Inhibitor functions by causing DNA damage (e.g., Bleomycin). In another aspect, the Cell Cycle Inhibitor functions as a DNA intercalation agent and/or RNA synthesis inhibition (e.g., Doxorubicin). In another aspect, the Cell Cycle Inhibitor functions by inhibiting pyrimidine synthesis (e.g., N-phosphonoacetyl-L-Aspartate). In another aspect, the Cell Cycle Inhibitor functions by inhibiting ribonucleotides (e.g., hydroxyurea). In another aspect, the Cell Cycle Inhibitor functions by inhibiting thymidine monophosphate (e.g., 5-fluorouracil). In another aspect, the Cell Cycle Inhibitor functions by inhibiting DNA synthesis (e.g., Cytarabine). In another aspect, the Cell Cycle Inhibitor functions by causing DNA adduct formation (e.g., platinum compounds). In another aspect, the Cell Cycle Inhibitor functions by inhibiting protein synthesis (e.g., L-Asparginase). In another aspect, the Cell Cycle Inhibitor functions by inhibiting microtubule function (e.g., taxanes). In another aspect, the Cell Cycle Inhibitors acts at one or more of the steps in the biological pathway shown in FIG. 1.

Additional Cell Cycle Inhibitors useful in the present invention, as well as a discussion of their mechanisms of action, may be found in Hardman J. G., Limbird L. E. Molinoff R. B., Ruddon R W., Gilman A. G. editors, Chemotherapy of Neoplastic Diseases in Goodman and Gilman's The Pharmacological Basis of Therapeutics Ninth Edition, McGraw-Hill Health Professions Division, New York, 1996, pages 1225-1287. See also U.S. Pat. Nos. 3,387,001; 3,808,297; 3,894,000; 3,991,045; 4,012,390; 4,057,548; 4,086,417; 4,144,237; 4,150,146; 4,210,584; 4,215,062; 4,250,189; 4,258,052; 4,259,242; 4,296,105; 4,299,778; 4,367,239; 4,374,414; 4,375,432; 4,472,379; 4,588,831; 4,639,456; 4,767,855; 4,828,831; 4,841,045; 4,841,085; 4,908,356; 4,923,876; 5,030,620; 5,034,320; 5,047,528; 5,066,658; 5,166,149; 5,190,929; 5,215,738; 5,292,731; 5,380,897; 5,382,582; 5,409,915; 5,440,056; 5,446,139; 5,472,956; 5,527,905; 5,552,156; 5,594,158; 5,602,140; 5,665,768; 5,843,903; 6,080,874; 6,096,923; and RE030561 (all of which, as noted above, are incorporated by reference in their entirety)

Numerous polypeptides, proteins and peptides, as well as nucleic acids that encode such proteins, can also be used therapeutically as cell cycle inhibitors. This is accomplished by delivery by a suitable vector or gene delivery vehicle which encodes a cell cycle inhibitor (Walther & Stein, Drugs 60(2):249-71, Aug 2000; Kim et al., Archives of Pharmacal Res. 24(1):1-15, Feb 2001; and Anwer et al., Critical Reviews in Therapeutic Drug Carrier Systems 17(4):377-424, 2000. Genes encoding proteins that modulate cell cycle include the INK4 family of genes (U.S. Pat. Nos. 5,889,169; 6,033,847), ARF-pl9 (U.S. Pat. No. 5,723,313), p21 and p27 (WO 9513375; WO 9835022), p₂₇^{KIP1} (WO 9738091), p57^{KIP2} (U.S. Pat. No. 6,025,480), ATM/ATR (WO 99/04266), Gadd 45 (U.S. Pat. No. 5,858,679), Mytl (U.S. Pat. No. 5,744,349), Weel (WO 9949061) smad 3 and smad 4 (U.S. Pat. No. 6,100,032), 14-3-3σ (WO 9931240), GSK3β (Stambolic, V. and Woodgett, J. R., Biochem Journal 303: 701-704, 1994), HDAC-1 (Furukawa, Y. et al., Cytogenet. Cell Genet. 73: 130-133, 1996; Taunton, J. et al., Science 272: 408-411, 1996), PTEN (WO 9902704), p53 (U.S. Pat. No. 5,532,220), p33^{ING1} (U.S. Pat. No. 5,986,078), Retinoblastoma (EPO 390530), and NF-1 (WO 9200387).

A wide variety of gene delivery vehicles may be utilized to deliver and express the proteins described herein, including for example, viral vectors such as retroviral vectors (e.g., U.S. Pat. Nos. 5,591,624, 5,716,832, 5,817,491, 5,856,185, 5,888,502, 6,013,517, and 6,133,029; as well as subclasses of retroviral vectors such as lentiviral vectors (e.g., PCT Publication Nos. WO 00/66759, WO 00/00600, WO 99/24465, WO 98/51810, WO 99/51754, WO 99/31251, WO 99/30742, and WO 99/15641)), alphavirus based vector systems (e.g., U.S. Pat. Nos. 5,789,245, 5,814,482, 5,843,723, and 6,015,686), adeno-associated virus-based system (e.g., U.S. Pat. Nos. 6,221,646, 6,180,613, 6,165,781, 6,156,303, 6,153,436, 6,093,570, 6,040,183, 5,989,540, 5,856,152, and 5,587,308) and adenovirus-based systems (e.g., U.S. Pat. Nos. 6,210,939, 6,210,922, 6,203,975, 6,194,191, 6,140,087, 6,113,913, 6,080,569, 6,063,622, 6,040,174, 6,033,908, 6,033,885, 6,020,191, 6,020,172, 5,994,128, and 5,994,106), herpesvirus based or “arnplicon” systems (e.g., U.S. Pat. Nos. 5,928,913, 5,501,979, 5,830,727, 5,661,033, 4,996,152 and 5,965,441) and, “naked DNA” based systems (e.g., U.S. Pat. Nos. 5,580,859 and 5,910,488) (all of which are, as noted above, incorporated by reference in their entirety).

Within one aspect of the invention, ribozymes or antisense sequences (as well as gene therapy vehicles which can deliver such sequences) can be utilized as cell cycle inhibitors. One representative example of such inhibitors is disclosed in PCT Publication No. WO 00/32765 (which, as noted above, is incorporated by reference in its entirety).

### (II) CELL CYCLE INHIBITOR FORMULATIONS

As noted above, therapeutic cell cycle inhibitory agents described herein may be formulated in a variety of manners, and thus may additionally comprise a carrier. In this regard, a wide variety of carriers may be selected of either polymeric or non-polymeric origin. The polymers and non-polymer based carriers and formulations, which are discussed in more detail below, are provided merely by way of example and not by way of limitation.

Within one embodiment of the invention a wide variety of polymers may be utilized to contain and/or deliver one or more of the therapeutic agents discussed above, including for example both biodegradable and non-biodegradable compositions. Representative examples of biodegradable compositions include albumin, collagen, gelatin, chitosan, hyaluronic acid, starch, cellulose and derivatives thereof (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), alginates, casein, dextrans, polysaccharides, fibrinogen, poly(L-lactide), poly(D,L lactide), poly(L-lactide-co-glycolide), poly(D,L-lactide-co-glycolide), poly(glycolide), poly(trimethylene carbonate), poly(hydroxyvalerate), poly(hydroxybutyrate), poly(caprolactone), poly(alkylcarbonate) and poly(orthoesters), polyesters, poly(hydroxyvaleric acid), polydioxanone, poly(malic acid), poly(tartronic acid), polyanhydrides, polyphosphazenes, poly(amino acids), copolymers of such polymers and blends of such polymers (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986). Representative examples of nondegradable polymers include poly(ethylene-co-vinyl acetate) (“EVA”) copolymers, silicone rubber, acrylic polymers (e.g., polyacrylic acid, polymethylacrylic acid, poly(hydroxyethylmethacrylate), polymethylmethacrylate, polyalkylcyanoacrylate), polyethylene, polyproplene, polyamides (e.g., nylon 6,6), polyurethane (e.g., poly(ester urethanes), poly(ether urethanes), poly(ester-urea), poly(carbonate urethanes)), polyethers (e.g., poly(ethylene oxide), poly(propylene oxide), Pluronics and poly(tetramethylene glycol)) and vinyl polymers [e.g., polyvinylpyrrolidone, poly(vinyl alcohol), poly(vinyl acetate phthalate)]. Polymers may also be developed which are either anionic (e.g., alginate, carrageenin, carboxymethyl cellulose and poly(acrylic acid), or cationic (e.g., chitosan, poly-L-lysine, polyethylenimine, and poly (allyl amine)) (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995). Particularly preferred polymeric carriers include poly(ethylene-co-vinyl acetate), polyurethane, poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly(caprolactone) or poly(lactic acid) with a polyethylene glycol (e.g., MePEG), and blends thereof.

Other representative polymers include carboxylic polymers, polyacetates, polyacrylamides, polycarbonates, polyethers, polyesters, polyethylenes, polyvinylbutyrals, polysilanes, polyureas, polyurethanes, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxy, melamine, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, polyurethane, polyacrylate, natural and synthetic elastomers, rubber, acetal, nylon, polyester, styrene polybutadiene, acrylic resin, polyvinylidene chloride, polycarbonate, homopolymers and copolymers of vinyl compounds, polyvinylchloride, polyvinylchloride acetate.

Representative examples of patents relating to polymers and their preparation include PCT Publication Nos. WO72827, 98/12243, 98/19713, 98/41154, 99/07417, 00/33764, 00/21842, 00/09190, 00/09088, 00/09087, 2001/17575 and 2001/15526 (as well as their corresponding U.S. applications), and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,099,563, 6,106,473, 6,110,483, 6,121,027, 6,156,345, 6,179,817, 6,197,051, 6,214,901, 6,335,029, 6,344,035, which, as noted above, are all incorporated by reference in their entirety.

Polymers can be fashioned in a variety of forms, with desired release characteristics and/or with specific desired properties. For example, polymers can be fashioned to release a therapeutic agent upon exposure to a specific triggering event such as pH (see, e.g., Heller et al., “Chemically Self-Regulated Drug Delivery Systems,” in Polymers in Medicine III, Elsevier Science Publishers B. V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Comejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gumy et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid)-based polymers and derivatives (including, for example, homopolymers such as poly(aminocarboxylic acid), poly(acrylic acid), poly(methyl acrylic acid), copolymers of such homopolymers, and copolymers of poly(acrylic acid) and acrylmonomers such as those discussed above). Other pH sensitive polymers include polysaccharides such as carboxymethyl cellulose, hydroxypropylmethylcellulose phthalate, hydroxypropyl-methylcellulose acetate succinate, cellulose acetate trimellilate, chitosan and alginates. Yet other pH sensitive polymers include any mixture of a pH sensitive polymer and a water soluble polymer.

Likewise, polymers can be fashioned which are temperature sensitive (see, e.g., Chen et al., “Novel Hydrogels of a Temperature-Sensitive Pluronic Grafted to a Bioadhesive Polyacrylic Acid Backbone for Vaginal Drug Delivery,” in Proceed. Intern. Symp. Control. ReL Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffinan, “Thermally Reversible Hydrogels Containing Biologically Active Species,” in Migliaresi et al. (eds.), Polymers in Medicine III, Elsevier Science Publishers B. V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers include homopolymers such as poly(N-methyl-N-n-propylacrylamide), poly(N-n-propylacrylamide), poly(N-methyl-N-isopropylacrylamide), poly(N-n-propylmethacrylamide), poly(N-isopropylacrylamide), poly(N, n-diethylacrylamide), poly(N-isopropylmethacrylamide), poly(N-cyclopropylacrylamide), poly(N-ethylmethyacrylamide), poly(N-methyl-N-ethylacrylamide), poly(N-cyclopropylmethacrylamide) and poly(N-ethylacrylamide). Moreover thermogelling polymers may be made by preparing copolymers between (among) monomers of the above, or by combining such homopolymers with other water soluble polymers such as acrylmonomers (e.g., acrylic acid and derivatives thereof such as methylacrylic acid, acrylate and derivatives thereof such as butyl methacrylate, acrylamide, and N-n-butyl acrylamide).

Other representative examples of thermogelling cellulose ether derivatives such as hydroxypropyl cellulose, methyl cellulose, hydroxypropylmethyl cellulose, ethylhydroxyethyl cellulose, and Pluronics, such as F-1 27, L-122, L-92, L-81, and L-61.

A wide variety of forms may be fashioned by the polymers of the present invention, including for example, rod-shaped devices, pellets, slabs, particulates, micelles, films, molds, sutures, threads, gels, creams, ointments, sprays or capsules (see, e.g., Goodell et al., Am. J. Hosp. Pharm. 43:1454-1461, 1986; Langer et al., “Controlled release of macromolecules from polymers”, in Biomedical Polymers, Polymeric Materials and Pharmaceuticals for Biomedical Use, Goldberg, E. P., Nakagim, A. (eds.) Academic Press, pp. 113-137, 1980; Rhine et al., J. Pharm. Sci. 69:265-270, 1980; Brown et al., J. Pharm. Sci. 72:1181-1185, 1983; and Bawa et al., J. Controlled Release 1:259-267, 1985). Therapeutic agents may be linked by occlusion in the matrices of the polymer, bound by covalent linkages, or encapsulated in microcapsules. Within certain preferred embodiments of the invention, therapeutic compositions are provided in non-capsular formulations, such as microspheres (ranging from nanometers to micrometers in size), pastes, threads or sutures of various size, films and sprays.

Other compositions which may be utilized to carry and/or deliver the cell cycle inhibitors described herein include vitamin-based compositions (e.g., based on vitamins A, D, E and/or K, see, e.g., PCT publication Nos. WO 98/30205 and WO 00/71163) and liposomes (see, U.S. Pat. Nos. 5,534,499, 5,683,715, 5,776,485, 5,882,679, 6,143,321, 6,146,659, 6,200,598, and PCT Publication Nos. WO 98/34597, WO 99/65466, WO 00/01366, WO 00/53231, WO 99/35162, WO 00/117508, WO 00/125223, WO 00/149,268, WO 00/1565438, WO 00/158455.

Preferably, therapeutic compositions of the present invention are fashioned in a manner appropriate to the intended use. Within certain aspects of the present invention, the therapeutic composition should be biocompatible, and release one or more therapeutic agents over a period of several days to months. For example, “quick release” or “burst” therapeutic compositions are provided that release greater than 10%, 20% or 25% (w/v) of a therapeutic agent (e.g., paclitaxel) over a period of 7 to 10 days. Such “quick release” compositions should, within certain embodiments, be capable of releasing chemotherapeutic levels (where applicable) of a desired agent. Within other embodiments, “slow release” therapeutic compositions are provided that release less than 1% (w/v) of a therapeutic agent over a period of 7 to 10 days. Further, therapeutic compositions of the present invention should preferably be stable for several months and capable of being produced and maintained under sterile conditions.

Within certain aspects of the present invention, therapeutic compositions may be fashioned in any size ranging from 50 nm to 500 μm, depending upon the particular use. Alternatively, such compositions may also be readily applied as a “spray” which solidifies into a film or coating. Such sprays may be prepared from microspheres of a wide array of sizes, including for example, from 0.1 μm to 9 μm, from 10 μm to 30 μm and from 30 μm to 100 μm.

Therapeutic compositions of the present invention may also be prepared in a variety of “paste” or gel forms. For example, within one embodiment of the invention, therapeutic compositions are provided which are liquid at one temperature (e.g., temperature greater than 37° C.) and solid or semi-solid at another temperature (e.g., ambient body temperature, or any temperature lower than 37° C). Also included are polymers, such as Pluronic F-127, which are liquid at a low temperature (e.g., 4° C.) and a gel at body temperature. Such “thermopastes” may be readily made given the disclosure provided herein.

Within yet other aspects of the invention, the therapeutic compositions of the present invention may be formed as a film. Preferably, such films are generally less than 5, 4, 3, 2 or 1 mm thick, more preferably less than 0.75 mm or 0.5 mm thick, and most preferably less than 500 μm. Such films are preferably flexible with a good tensile strength (e.g., greater than 50, preferably greater than 100, and more preferably greater than 150 or 200 N/cm²), good adhesive properties (i.e., readily adheres to moist or wet surfaces), and have controlled permeability.

Within further aspects of the invention, the therapeutic compositions may be formulated for topical application. Representative examples include: ethanol; mixtures of ethanol and glycols (e.g. ethylene glycol or propylene glycol); mixtures of ethanol and isopropyl myristate or ethanol, isopropyl myristate and water (e.g., 55:5:40); mixtures of ethanol and eineol or D-limonene (with or without water); glycols (e.g., ethylene glycol or propylene glycol) and mixtures of glycols such as propylene glycol and water, phosphatidyl glycerol, dioleoylphosphatidyl glycerol, Transcutol®, or terpinolene; mixtures of isopropyl myristate and 1-hexyl-2-pyrrolidone, N-dodecyl-2-piperidinone or 1-hexyl-2-pyrrolidone. Other excipients may also be added to the above, including for example, acids such as oleic acid and linoleic acid, and surfactants, such as sodium lauryl sulfate. For a more detailed description of the above, see generally, Hoelgaard et al., J. Contr. Rel. 2:111, 1985; Liu et al., Pharm. Res. 8:938, 1991; Roy et al., J. Pharm. Sci. 83:126, 1991; Ogiso et al., J. Pharm. Sci. 84:482, 1995; Sasaki et al., J. Pharm. Sci. 80:533, 1991; Okabe et al., J. Contr. Rel. 32:243, 1994; Yokomizo et al., J. Contr. Rel. 38:267, 1996; Yokomizo et al., J. Contr. Rel. 42:37, 1996; Mond et al., J. Contr. Rel. 33:72, 1994; Michniak et al., J. Contr. Rel. 32:147, 1994; Sasaki et al., J. Pharm. Sci. 80:533, 1991; Baker & Hadgraft, Pharm. Res. 12:993, 1995; Jasti et al., AAPS Proceedings, 1996; Lee et al., AAPS Proceedings, 1996; Ritschel et al., Skin Pharmacol. 4:235, 1991; and McDaid & Deasy, Int. J. Pharm. 133:71, 1996.

Within certain embodiments of the invention, the therapeutic compositions can also comprise additional ingredients such as surfactants (e.g., Pluronics such as F-127, L-122, L-92, L-81, and L-61).

Within further aspects of the present invention, polymers are provided which are adapted to contain and release a hydrophobic compound, the carrier containing the hydrophobic compound in combination with a carbohydrate, protein or polypeptide. Within certain embodiments, the polymeric carrier contains or comprises regions, pockets or granules of one or more hydrophobic compounds. For example, within one embodiment of the invention, hydrophobic compounds may be incorporated within a matrix which contains the hydrophobic compound, followed by incorporation of the matrix within the polymeric carrier. A variety of matrices can be utilized in this regard, including for example, carbohydrates and polysaccharides, such as starch, cellulose, dextran, methylcellulose, and hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver the therapeutic agents described herein include: hydroxypropyl β-cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), implants (Jampel et al., Invest. Ophthalm. Vis. Science 34(11): 3076-3083, 1993; Walter et al., Cancer Res. 54:22017-2212, 1994), nanoparticles (Violante and Lanzafame PAACR), nanoparticles—modified (U.S. Pat. No. 5,145,684), nanoparticles (surface modified) (U.S. Pat. No. 5,399,363), taxol emulsion/solution (U.S. Pat. No. 5,407,683), micelle (surfactant) (U.S. Pat. No. 5,403,858), synthetic phospholipid compounds (U.S. Pat. No. 4,534,899), gas borne dispersion (U.S. Pat. No. 5,301,664), foam, spray, gel, lotion, cream, ointment, dispersed vesicles, particles or droplets solid- or liquid- aerosols, microemulsions (U.S. Pat. No. 5,330,756), polymeric shell (nano- and micro- capsule) (U.S. Pat. No. 5,439,686), taxoid-based compositions in a surface-active agent (U.S. Pat. No. 5,438,072), liquid emulsions (Tarr et al., Pharm Res. 4:62-165, 1987), nanospheres (Hagan et al., Proc. Intern. Symp. Control ReL Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994) and implants (U.S. Pat. No. 4,882,168).

Within other aspects of the invention, radioactive polymer compositions are provided which may be in the form of a solid, porous material, slurry, gel, spray, or the like. For example, within one embodiment the radioactive polymer comprises a radioactive material or source (e.g., I¹²⁵, Pd¹⁰³, Ir¹⁹², Co⁶⁰, Cs¹³⁷, Au¹⁹⁸ and/or Ru¹⁰⁶) which is incorporated into, or, adapted to be released from a polymer. As noted above, a wide variety of polymers may be utilized in this context, including both biodegradable and non-biodegradable polymers discussed above.

Within one preferred embodiment, the radioactive polymer may be comprised of radioactive monomer(s) and non-radioactive monomer(s), or, of radioactive monomer(s) only. For example, radioactive polymers may be produced from (a) and (bi) or (bii), wherein (a) a non-radioactive component comprising repeating units that may be produced from the reaction of a molecule containing a carbon-carbon double bond (e.g., acrylates or methacrylates such as ethyl methacrylate, methyl methacrylate, 2-hydroxyethyl methacrylate, 2-hydroxyethyl acrylate, methacrylic acid, acrylic acid, or vinyl monomers such as vinyl acetate, styrene and vinyl chloride), and (bi) a radioactive component comprising repeating units that may be produced from the reaction of:

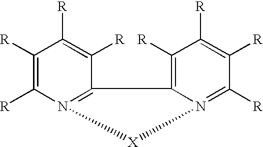

in which X is a radioactive moiety such as ¹⁰³PDY₂, ¹⁰⁶RuY₄, ⁶⁰CoY₄, and ¹⁹²IrY₂, in which Y is Cl, NH₃, or P(C₆H₅)₃ and the R groups are selected independently from H , OH, C₁₋₄ alkyl, —COOH and amino and 1 to 3 R groups contain polymerizable group(s) (e.g., co-bonded C₄₋₂₀ alkenes containing a single carbon-carbon double bond, acylates or methyacrylates (e.g., alkyl acrylate and alkyl methacylate), and alkyl acrylamide groups);

and

(bii) is a radioactive component comprising repeating units that may be produced from the reaction of:

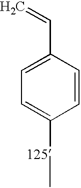

where the repeating units a) and b) are bonded to one another resulting in desaturation of the carbon-carbon double bonds.

Within various embodiments, the non-radioactive component comprises repeating units that may be produced from the reaction of a molecule containing a carbon-carbon double bond (e.g., acrylates or methacrylates such as ethyl methacrylate, methyl methacrylate, 2-hydroxyethyl methacrylate, 2-hydroxyethyl acrylate, methacrylic acid, acrylic acid, or vinyl monomers such as vinyl acetate, styrene and vinyl chloride). Within other embodiments, radioactive component comprises repeating units that may be produced from the reaction of

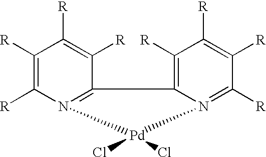

in which one R group is —(CH₂)ₘ—CH=CH₂ and the remaining R groups are H and m is an integer from 4 to 18. Within further embodiments, the radioactive component comprises repeating units that may be produced from the reaction of

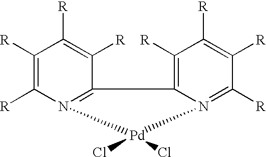

in which two or more R groups are —(CH₂)ₘ—CH=CH₂ and the remaining R groups are H and m is an integer from 4 to 18. Within yet other embodiments, the radioactive and non-radioactive repeating units are in a mole ratio of 1:1 to 1 :10,000.

Within other aspects polymers are provided which contain in its structure a therapeutically active radioactive isotope comprising a radioactive component comprising repeating units that may be produced from the reaction of:

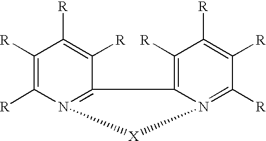

in which X is a radioactive moiety such as ¹⁰³PdY₂, ¹⁰⁶RuY₄, ⁶⁰CoY₄, and ¹⁹²IrY₂, in which Y is Cl, NH₃, or PPh₃ and the R groups are selected independently from H , OH, C₁₋₄ alkyl, and amino and 1 to 3 R groups are polymerizable group(s) (e.g., (ω-bonded C₄₋₂₀ alkenes containing a single carbon-carbon double bond, acylates or methyacrylates (e.g., alkyl acrylate and alkyl methacylate), and alkyl acrylamide group, where the repeating units are bonded to one another resulting in desaturation of the carbon-carbon double bonds

Within various embodiments of the above, the polymer(s) may be formed into a fibre, woven fabric, knitted fabric, sutures, or solid implant (e.g., in the shape of a a cylinder or sphere with one or more holes, a rod, a hollow cylinder, a ring, a U-shape, a rod with holes in it, a rod with protrusions extended from its surface, or a sphere). Representative examples of cell cycle inhibitors that may be used in this regard include taxanes, antimetabolites, topoisomerase inhibitors, platinums, alkylating agents, nitrogen mustards, anthracyclines, or, vinca alkaloids.

Within various embodiments of the above, the formulations can be made echogenic or radiopaque. For example, the compositions described herein may either be made with, made to contain, or coated with a composition which is echogenic or radiopaque (e.g., made with echogenic or radiopaque with materials such as powdered tantalum, tungsten, barium carbonate, bismuth oxide, bariumsulfate, or, made by the addition of microspheres or bubbles which present an acoustic interface. Echogenic materials and methods have been described in a number of patents and patent applications, including for example, U.S. Pat. Nos. 5,201,314, 5,271,928, 5,380,519, 5,413,774, 5,531,980, 5,578,292, 5,658,551, 5,711,933, and 6,106,473, all of which are incorporated by reference in their entirety.

As discussed in more detail below, cell cycle inhibitors of the present invention, which are optionally incorporated within one of the carriers described herein to form an effective composition, may be prepared and utilized to enhance the effects of brachytherapy by sensitizing the hyperproliferating cells that characterize the diseases being treated. Within further embodiments, the devices and compositions provided herein can be sterilized, packaged with preservatives and the like suitable for administration to humans.

### (III) CELL CYCLE INHIBITOR—RADIOACTIVE SOURCE-REPRESENTATIVE EMBODIMENTS

As described in more detail herein, typically the source of irradiation can be placed directly into the tissues (interstitial therapy), within a body cavity (intracavitary therapy), or, close to the surface of the body (surface therapy). The implants can be either permanent or temporary (i.e., removed after the appropriate dose has been delivered). In addition, their placement within/around a desired location (e.g., a tumor) can be determined uniquely for each patient procedure using well defined dose mapping techniques. Within preferred embodiments of the invention, the compositions and devices discussed in more detail below are provided in a sterile form suitable for medical use. In addition, as noted above, within various embodiments the compositions and devices described herein can be made radiopaque or echogenic in order to enhance visualization.

As noted above, cell cycle inhibitors can be deposited directly onto all or a portion of a device or implant (see, e.g., U.S. Pat. Nos. 6,096,070 and 6,299,604), or, admixed with a delivery system or carrier (e.g., a polymer, liposome, or vitamin as discussed above) which is applied to all or a portion of the device (see the patents, patent applications, and references listed above under “Compositions and Formulations.”

Within certain aspects of the invention, cell cycle inhibitors can be attached to a medical implant using non-covalent attachments. For example, for compounds that are relatively sparingly water soluble or water insoluble, the compound can be dissolved in an organic solvent as a specified concentration. The solvent chosen for this application would not result in dissolution or swelling of the polymeric device surface. The medical implant can then be dipped into the solution, withdrawn and then dried (air dry and/or vacuum dry). Alternatively, this drug solution can be sprayed onto the surface of the implant. This can be accomplished using current spray coating technology. The release duration for this method of coating would be relatively short and would be a function of the solubility of the drug in the body fluid in which it was placed.

Alternatively, a cell cycle inhibitor can be dissolved in a solvent that has the ability to swell or partially dissolve the surface of a polymeric implant. Depending on the solvent/implant polymer combination, the implant could be dipped into the drug solution for a period of time such that the drug can diffuse into the surface layer of the polymeric device. Alternatively the drug solution can be sprayed onto all or a part of the surface of the implant. The release profile of the drug depends upon the solubility of the drug in the surface polymeric layer. Using this approach, one would ensure that the solvent does not result in a significant distortion or dimensional change of the medical implant.

If the implant is non-polymeric, or, is composed of materials that do not allow incorporation of a cell cycle inhibitor into the surface layer using the above solvent method, one can treat the surface of the device with a plasma polymerization method such that a thin polymeric layer is deposited onto the device surface. Examples of such methods include parylene coating of devices, and the use of various monomers such hydrocyclosiloxane monomers. One can then use the dip coating or spray coating methods describe above to incorporate the cell cycle inhibitor into the surface of the implant.

For cell cycle inhibitors that have some degree of water solubility, the retention of these compounds on a device are relatively short-term. For cell cycle inhibitors that contain ionic groups, it is possible to ionically complex these agents to oppositely charged compounds that have a hydrophobic component. For example cell cycle inhibitors containing amine groups can be complexed with compounds such as sodium dodecyl sulfate (SDS). Compounds containing carboxylic groups can be complexed with tridodecymethyanunonium chloride (TDMAC). Mitoxantrone, for example has two secondary amine groups and comes as a chloride salt. This compound can be added to sodium dodecyl sulfate in order to form a complex. This complex can be dissolved in an organic solvent which can then be dip coated or spray coated. Doxorubicin has an amine group and could thus also be complexed with SDS. This complex could then be applied to the device by dip coating or spray coating methods. Methotrexate, for example contains 2 carboxylic acid groups and could thus be complexed with TDMAC and then coated onto the medical implant.

Cell cycle inhibitors with available functional groups can be covalently attached to the medical imlant surface using several chemical methods. If the polymeric material used to manufacture the implant has available surface functional groups then these can be used for covalent attachment of the agent. If the implant surface contains carboxylic acid groups, these groups can be converted to activated carboxylic acid groups (e.g acid chlorides, succinimidyl derivatives, 4-nitrophenyl ester derivatives etc). These activated carboxylic acid groups can then be reacted with amine functional groups that are present on the cell cycle inhibitor (e.g. methotrexate, mitoxantrone).

For surfaces that do not contain appropriate functional groups, these groups can be introduced introduced to the polymer surface via a plasma treatment regime. For example, carboxylic acid groups can be introduced via a plasma treatment process that includes CO₂ in the gas mixture. The carboxylic acid groups can also be introduced using acrylic acid or methacrylic acid in the gas stream. These carboxylic acid groups can then be converted to activated carboxylic acid groups (e.g acid chlorides, succinimidyl derivatives, 4-nitrophenyl ester derivatives etc) that can subsequently be reacted with amine functional groups that are present on the cell cycle inhibitor.

In order to further the understanding of the compositions, methods and devices provided herein, representative embodiments of the invention are discussed in more detail below.

A. INTERSTITIAL THERAPY

In interstitial therapeutic embodiments, the cell cycle inhibitor and the radioactive source are placed directly into (within) the hyperproliferative tissue. As discussed in more detail below, the implantation can be permanent or temporary (i.e., removed after a therapeutic dose has been delivered).

Permanent (i.e., non-removed) radioactive sources are implanted into the diseased tissues and allowed to decay completely. Therefore, typically, isotopes with low energy and/or short half-lives are used for this application, such as radioactive iodine (e.g., I¹²⁵), palladium (e.g., Pd¹⁰³) and gold (e.g., Au¹⁹⁸). Permanent implants include, for example, “loose” radioactive “seeds” injected into tissues via needles, catheters, or automated injectors. Radioactive sources contained within sutures are also used as a means of permanently implanting isotopes within tissues. The following describes compositions and methods for the simultaneous permanent interstitial delivery of radioactive sources and cell cycle inhibitors including: Cell Cycle Inhibitor-Coated Radioactive Sutures, Cell Cycle Inhibitor-Loaded Radioactive Sutures, Interstitial Injection of Cell Cycle Inhibitors and Cell Cycle Inhibitor-Coated Radioactive Seeds.

Temporary radioactive sources are implanted interstitially into diseased tissue and subsequently removed after delivering the desired dose of radiotherapy. Catheters can be advanced into the tissue as a means to initially deliver, and later remove, the radioactive source. Higher energy radioactivity can be used under these circumstances since the material does not remain in the tissue indefinitely. These so-called high-dose-rate (HDR) radioactive sources include, for example, high activity ¹²⁵, Pd¹⁰³ and Ir¹⁹², Co⁶⁰, Cs¹³⁷, Ru¹⁰⁶ and Rn²²² as well as several others. The radioactive source can be physically delivered via the catheter as a “seed” or “pellet”, or as a radioactive wire. In this embodiment, introduction catheters that are microscopically or macroscopically porous can be used to deliver aqueous and/or sustained release preparations of cell cycle inhibitors. The following describes compositions and methods for simultaneous temporary interstitial delivery of radioactive sources and cell cycle inhibitors including: Cell Cycle Inhibitor-Coated Radioactive Wires, Cell Cycle Inhibitor-Loaded (or coated) Spacers, Cell Cycle Inhibitor-Loaded Sutures, Cell Cycle Inhibitor-Coated Sutures, and Interstitial Injection of Cell Cycle Inhibitors. As should be readily evident, radioactive sources and cell cycle inhibitors can also be delivered separately (or sequentially).

1. Cell Cycle Inhibitor-Coated Radioactive Fastening Devices —Nonabsorbable or absorbable radioactive fastening devices (e.g., I¹²⁵ sutures, Medic-Physics Inc., Arlington Heights, IL; staples, pins, nails, screws, plates, barbs, anchors or patches such as those described in EPB No. 386757, U.S. Pat. Nos. 5,906,573, 5,897,573, 5,709,644, and PCT Publication Nos. W098/18408, WO 98/57703, WO 98/47432, WO 97/19706) can be interstitially implantated into tissues (e.g., superficial shallow depth tumors or into tumor beds during open surgery). Fastening devices can be made from a variety of materials, including, but not limited to, metals and polymers (e.g., polyesters (e.g., poly(glycolic acid), polypropylene, glycolide/lactide, glycolide/diaxanone/trimethylene carbonate, polydiaxanone, poly(ethylene terephthalate)), nylon, silk, connective tissue, polyviolene, polyglecaprone 25, polygalactin, polyolefin, prolene, poly(tetrafluoroethylene) (ePTFE), silicon, polyurethanes, chitosan, Vicryl (polygalactin) and polyvinylidenefluoride). Within various embodiments of the above, the radioactive fastening devices can be made echogenic in order to enhance visualization.

Within certain embodiments of the invention, a variety of cell cycle inhibitors can be coated directly onto, or, loaded into a composition (e.g., a polymer) that is applied to the surface of the fastening device. Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and etrazines (e.g., dacarbazine and procarbazine).

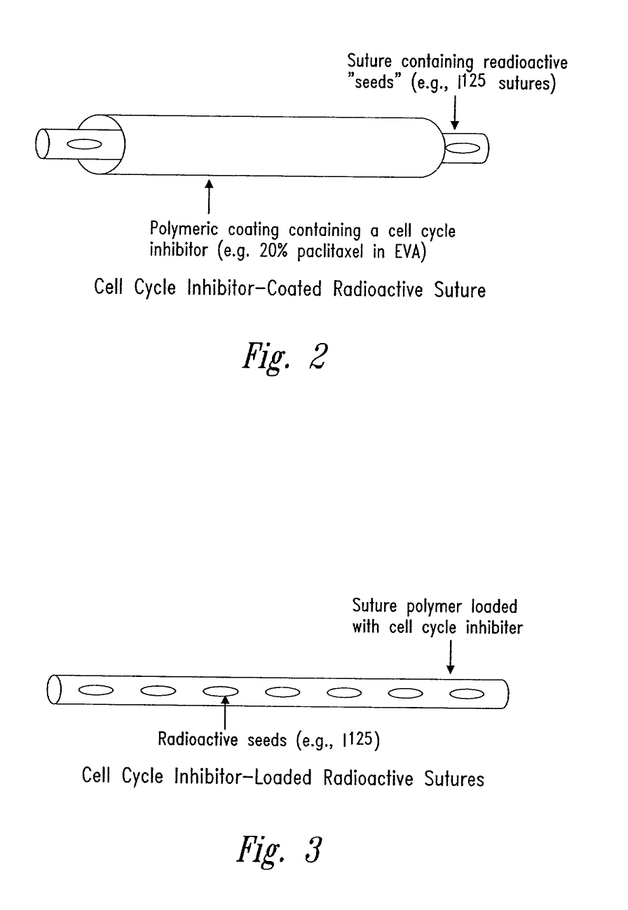

One example of a nonabsorbable suture is 1-30% paclitaxel loaded into EVA, polyurethane (PU) or PLGA applied as a coating (e.g., sprayed, dipped, etc.) onto a suture prior to insertion in the tissue. Conversely, poly(lactide-co-glycolide) can be used as a coating for absorbable radioactive sutures. A representative example is shown below in FIG. 2.

2. Cell Cycle Inhibitor-Loaded Radioactive Fastening Devices—In this embodiment, nonabsorbable or absorbable radioactive fastening devices (e.g., I¹²⁵ sutures, Medic-Physics Inc., Arlington Heights, IL; staples, pins, nails, screws, plates, barbs, 25 anchors or patches such as those described in EPB No. 386757, U.S. Pat. Nos. 5,906,573, 5,897,573, 5,709,644, and PCT Publication Nos. WO 98/18408, WO 98/57703, WO 98/47432, WO 97/19706) can be manufactured to comprise, or otherwise elute a cell cycle inhibitor (e.g., from a constituent polymer; see, as an example FIG. 3). Within various embodiments of the above, the radioactive fastening devices can be made echogenic in order to enhance visualization.

Within certain embodiments of the invention, a variety of cell cycle inhibitors can be applied to the surface of the fastening device (e.g., either by directly coating the cell-cycle inhibitor onto the device, or, through use of polymers, ointments, or the like). Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In one embodiment 1-30% (20% most preferred) paclitaxel is loaded into a polyester, such as poly(glycolide), poly(lactide-co-glycolide) and/or poly(glycolide-co-caprolactone), to produce a resorbable suture also containing a radioactive source (e.g., I¹²⁵ seeds), and polypropylene and/or silicon for nonabsorbable sutures.

Methods for loading cell cycle inhibitors into polymers are described in the following examples. In another preferred embodiment 1-30% paclitaxel (20% most preferred) is loaded into polypropylene to manufacture nonabsorbable radioactive suture (e.g., I¹²⁵) material.

3. Interstitial Injection of Cell Cycle Inhibitors—In this embodiment, the cell cycle inhibitor is injected into the tissue surrounding the site where the radioactive source has been placed. The cell cycle inhibitor is formulated into an aqueous, nanoparticulate, microparticuate or microspheric form as described in the examples. Within certain embodiments of the invention, a variety of cell cycle inhibitors can be loaded into polymers that are applied to the surface of the suture material. Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In a preferred embodiment, 1-30% paclitaxel is loaded into 1-30 μm-sized microspheres composed of a blend of PLA and PLGA (see following examples for manufacturing methods) or paclitaxel is formulated into micelles composed of methoxy poly(ethylene glycol) (MePEG) and poly(D,L-lactide) (PDLLA). The injectable is administered prior to, in conjunction with, or subsequent to implantation of the radioactive source. The injectable can be administered via a needle or via the catheter used for implantation of the radioactive source. If an automated injector is used (e.g., Mick Applicator, Mick Radio-Nuclear Instruments Inc., Bronx, N.Y.; Scott Applicator, Lawrence Soft-Ray Corp., San Jose, Calif.; Quick Seeder System, Mick Radio-Nuclear Instruments Inc., Bronx, N.Y.; Gold Grain Gun, Royal Marsden Hosp.), the injectable cell cycle inhibitor can be administered via this apparatus. Within various embodiments of the above, the cell cycle inhibitor can be formulated into a radiopaque or echogenic composition, in order to enhance visualization.

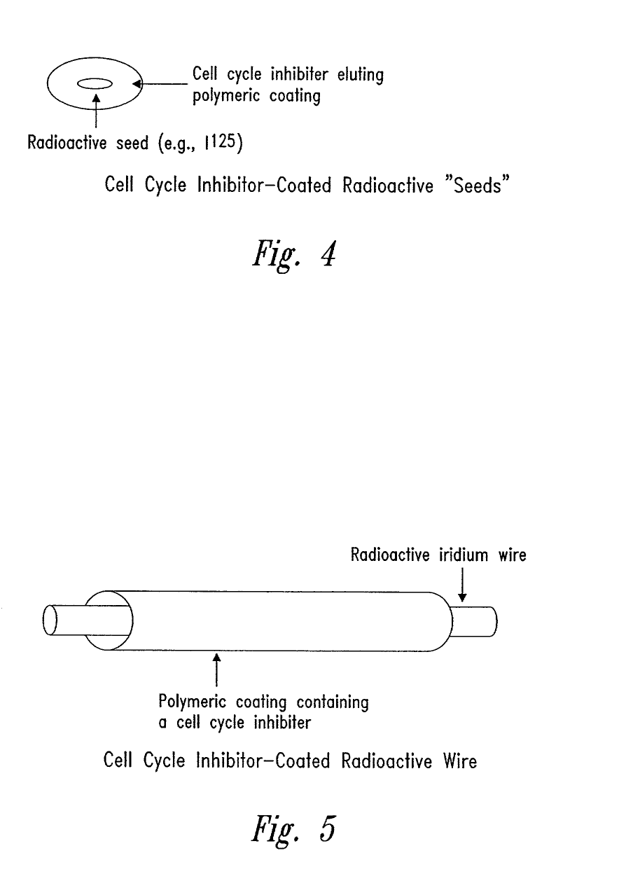

4. Cell Cycle Inhibitor-Coated Radioactive “Seeds”—In this embodiment, the cell cycle inhibitor is directly coated on, or chemically linked to, a radioactive seed used for interstitial implantation (see, as an example, FIG. 4). Representative examples of radioactive seeds, methods for making and deploying such seeds are disclosed in U.S. Pat. Nos. 6,132,359, 6,103,295, 6,095,967, 6,080,099, 6,060,036, 6,007,475, 5,928,130, 5,163,896 and 4,323,055.

Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In one embodiment, 1-30% paclitaxel-loaded EVA (or PU) is used to coat radioactive seeds (e.g., I¹²⁵ seeds, Pd¹⁰³ seeds, Au¹⁹⁸ grains). The polymer/cell cycle inhibitor-coated seeds are then implanted into the tissue via catheters or automated injectors as described previously. Within various embodiments of the above, the cell-cycle inhibitor coated radioactive seeds can be made echogenic in order to enhance visualization.

5. Cell Cycle Inhibitor Coated Radioactive Wires—In this embodiment, when iridium (Ir¹⁹²) or other radioactive wires are placed through the tumor via the skin or during open surgery, a cell cycle inhibitor can be delivered to the therapeutic target (e.g., via a polymeric, drug releasing coating applied to the wire prior to insertion (see the examples; see also, FIG. 5), or by directly coating the cell-cycle inhibitor onto the wire).

A variety of polymeric carriers and cell cycle inhibitors can be utilized in this manner. A preferred embodiment for long-term treatment is 1-30% paclitaxel loaded in poly(ethylene-co-vinyl acetate) (EVA) or polyurethane (PU) applied as a coating (e.g., spray, dipped, etc.) prior to wire insertion. For short-term brachytherapy, the cell cycle inhibitor would need to be released more quickly, so a preferred embodiment would be 1-30% paclitaxel loaded into hyaluronic acid (HA) and/or a cellulose polymer coating. The coating will elute drug into the hyperproliferative tissue and augment the effects of the radioactive portion of the therapy.

Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine). Within various embodiments of the above, the cell-cycle inhibitor coated radioactive seeds can be made echogenic in order to enhance visualization

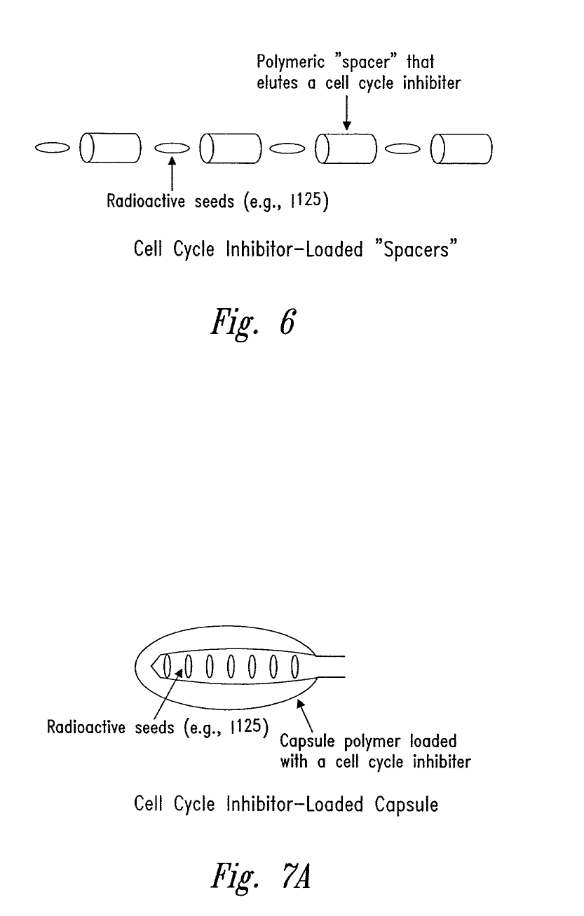

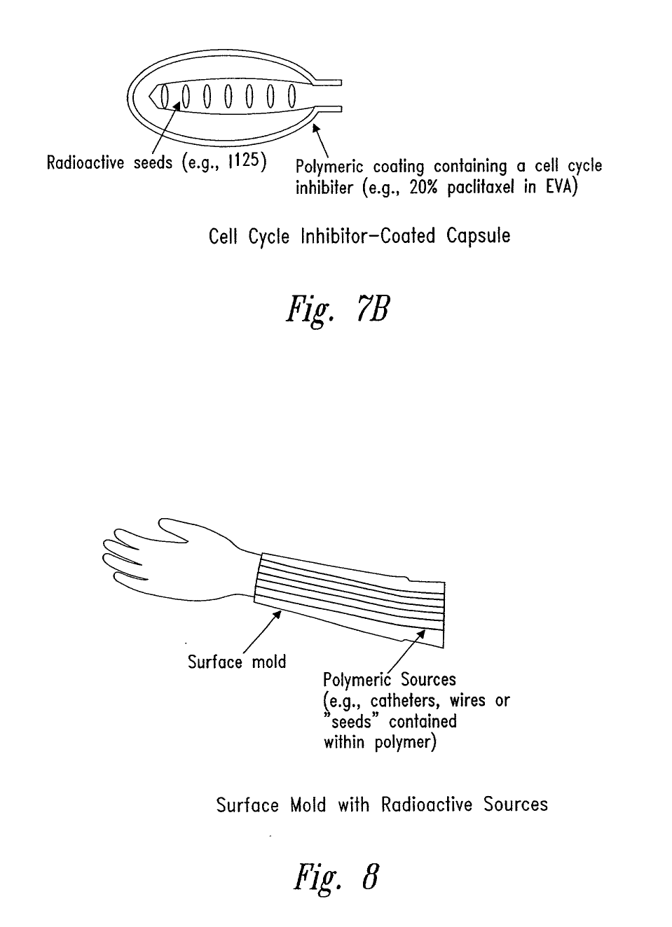

6. Cell Cycle Inhibitor-Loaded “Spacers”—In interstitial therapy catheters are advanced into (and through) the hyperproliferative tissue. Radioactive seeds (e.g., I¹²⁵) are placed into the catheter and plastic “spacers” (often 1 cm long) are placed between seeds to ensure proper spacing within the catheter. In this embodiment, the “spacer” is a polymeric carrier that elutes a cell cycle inhibitor (see, as an example, FIG. 6).

In one embodiment, the spacer is made of 1-30% paclitaxel loaded into a resorbable polymer (e.g., poly(glycolide), poly(lactide-co-glycolide), poly(glycolide-co-caprolactone)) or a nonresorbable polymer [e.g., poly(propylene)] depending upon the indication. Methods for loading a cell cycle inhibitor into an absorbable or nonabsorbable polymer are contained in the examples. The drug loaded polymer cylinders (sized to fit into the administration catheter) can be cut into lengths (e.g., 0.5 cm, 1.0 cm, 1.5 cm) for use as “spacers”. Alternatively, commercially available spacers can be coated with a cell cycle inhibitor eluting polymer coating (as described for Cell Cycle Inhibitor-Coated Wires).

In yet another embodiment, the spacers can be created at the time of insertion. A bisected catheter is laid on a flat surface and the radioactive seeds are placed in it at the appropriate spacing interval. Molten polymer (i.e., liquid phase polymer which will solidify (see “Thermopaste” and “Aquapaste” examples) is injected into the catheter “mold” to create drug loaded spacers between radioactive sources. In a preferred embodiment of this invention, 1-30% paclitaxel is loaded into a polycaprolactone-methoxy polyethylene-glycol polymer blend (“Thermopaste”). The material is heated to approximately 60° C.; prior to use and injected into the prepared catheter mold as described above. The material is allowed to cool to room temperature, at which point it solidifies to form a continuous polymeric “thread” with the radioactive sources separated by the appropriate distance. The entire material is now suitable for interstitial therapeutic use.

In yet another embodiment, the spacers are elongated with a length and positioned with a lengthwise orientation extending between the adjacent seeds between which positioned, and the spacer length is selected to position and hold the seeds within the tissue in a desired spatial pattern based upon the radiation pattern desired to be administered to the site to be treated.

In yet another embodiment, the device further includes a spacer positioned between adjacent ones of the plurality of radioactive seeds, the spacers both holding the adjacent seeds spaced apart while in the tissue and holding the plurality of seeds together as part of a continuous thread while being positioned in the tissue. Optionally, the spacers are formed from a spacer material having a liquid phase and a solid phase, the spacers being formed using the spacer material in the liquid phase immediately prior to the time of positioning of the seeds into the tissue by placing the liquid phase spacer material between adjacent ones of the seeds and then allowing the spacer material to change to the solid phase to form the continuous thread.

In yet another embodiment, the device further includes a spacer positioned between adjacent ones of the plurality of radioactive seeds, the spacers holding the adjacent seeds spaced apart while in the tissue, the spacers being a spacer material having a liquid phase and a solid phase, the spacers being formed using the spacer material in the liquid phase immediately prior to the time of positioning of the seeds into the tissue by placing the liquid phase spacer material between adjacent ones of the seeds and then allowing the spacer material to change to the solid phase prior to positioning of the spacers in the tissue. Within related embodiments, seed spacers can be made from, coated by, or otherwise designed to contain a variety of echogenic or radiopaque materials. The seed spacers can be made from either biodegradable (e.g., either natural or synthetic polymers) or non-biodegradable materials. One example of a natural material used to make the seed spacers is cat gut, while biodegradable polyesters (e.g. PLG) are often used as the synthetic material to make the seed spacers. Non-degradable spacers can be made from polymers such as poly(methyl methacrylate), polyurethane, poly(ethylene-co-vinyl acetate), polyethylene, polypropylene, blends and copolymers thereof. The seed spacers can contain materials that are incorporated into the spacer (or spacer coating) that improve scattering of the ultrasound waves. These incorporated materials can include particles of metal and/or glass. Coatings of this nature are described in US Pat. No. 5,201,314 which is incorporated in its entirety as a reference. These polymer coatings can also contain gas bubbles and/or pores and/or channels such that air or another gas is entrapped in the polymeric coating once it is inserted into the desired tissue. US Pat. No. 6,106,473 describes a polymeric coating that enhances the visibility of coated medical devices when viewed using ultrasonography. This patent is incorporated as reference.

The seed spacers can also be made from a solid piece of materials, a porous material or it can be made from a tubular structure. The tubular structure can have open ends or the ends can be sealed. The sealing of the ends can be accomplished by dipping the open ends in to a molten polymer or a polymer solution such that the open ends become sealed with a thin polymeric plug. One can also bone wax to seal the ends of the tubes. Alternatively one could use a non-polymeric material to seal the open ends of the tubes. These materials can include low melting aliphatic or aromatic alcohols, carboxylic acids, esters and/or amides. For example, one could use a molten solution of stearyl alcohol to seal the ends of the tubes.

For a porous material, one can use ePTFE material to prepare the spacers. The ePTFE has a network of pores within the material and is sufficiently hydrophobic as to prevent water from entering the pores. Therefore, once implanted the air remains trapped within the material thereby providing good visibility under ultrasound visualization methods. These spacers can be used as is or they can be coated with a coating that enhances the echogenicty of the spacer, as described above.

The spacers described herein can also contain a biologically active agent. These agents can include hormones, chemotherapeutic agents, radiation sensitizing agents, oligonucleotides or proteins.

In yet another embodiment, the device may be used with a catheter, wherein the seeds are positioned in the catheter in spaced apart relation and the spacer material in the liquid phase is placed between adjacent ones of the seeds and then allowed to change to the solid phase, after changing to the solid phase and without removing the seeds and the spacers from the catheter, the seeds and the spacers being positioned in the catheter in a molded state ready for positioning in the tissue using the catheter. Optionally, after the spacer material has been allowed to change to the solid phase, the seeds and the spacers are in the form of a continuous thread holding the plurality of seeds together for positioning in the tissue and holding the adjacent seeds spaced apart while in the tissue. As another option, the spacer material is in the liquid phase when heated to a liquid phase temperature above a body temperature of the patient, and in the solid phase when allowed to cool to a solid phase temperature below the liquid phase temperature.

Representative examples of cell cycle inhibitors that can be utilized in this regard include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

6. Cell Cycle Inhibitor Coated or loaded radioactive fabrics—In this embodiment, a radioactive fabric is prepared by coating a fabric with a radioactive substance, or, by interweaving radioactive fibre(s) to form a radioactive cloth. Similarly, the cell cycle inhibitor can be coated onto a fabric, or, the fabric itself can be composed of or interwoven with cell cycle inhibitor fibers. Within certain embodiments, the fabric may be coated with or interwoven with a composition of fiber(s) which contain or comprise both a radioactive substance and a cell cycle inhibitor.

Representative examples of cell cycle inhibitors that can be utilized in this regard include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

Within various embodiments of the above, the cell-cycle inhibitor coated or loaded fabrics can be made echogenic in order to enhance visualization

7. Coating of a Radioactive Medical Device—In this embodiment, a radioactive medical device is coated with polymer(s) such as acrylates (e.g., polyacrylic acid, or a methacrylate such as polymethylmethacylate), cellulose (e.g, ethyl cellulose), polysaccharide (e.g., hyaluronic acid), vinyls (e.g., polyvinyl acetate), ethers (e.g., polyoxyethylene), styrenes (e.g., polystyrene), or amino acids (e.g., polyaspartic acid or albumin). Within certain embodiments, the polymer(s) can be cross-linked by reaction with a compatible crosslinker.

As an example, a polymer at 10% is dissolved in a compatible solvent such as dichloromethane for polymethylmethacrylate or water for hyaluronic acid. The radioactive device, such as a fastening device, seed, wire, or the like is then dipped into the solution and then transferred to a dryer to remove the solvent by mild heating to 45° C. with a high vacuum. The coated device is dried to constant weight. A dried device has less then a 1% change in weight in three consecutive measurements of mass after 6 hours of drying time.

As noted above, the polymer coating can include a cell cycle inhibitor as well. This is accomplished by dissolving the cell cycle inhibitor and polymer in a mass ratio of 1:9 into the compatible solvent. In another method, the cell cycle inhibitor is micronized by milling, a particle size fraction of 10-100 μm is collected by sieving and this fraction is suspended by stirring for 30 minutes in a 30% polymer solution. Representative examples of cell cycle inhibitors that can be utilized in this regard include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

Within various further embodiments of the above, the device may also include a glidant, wax, magnetic resonance responsive (e.g. a Gadolinium III chelate), X-ray responsive (e.g. tantalum), or ultrasound responsive material. This material is loaded in the same manner as described for the inclusion of drugs. Within various embodiments of the above, the device can be made echogenic in order to enhance visualization

B. INTRACAVITARY THERAPY

In intracavitary therapeutic embodiments, the cell cycle inhibitor and the radioactive source are placed within a body cavity. Body cavities include the female reproductive tract (vagina, cervix, uterus, fallopian tubes), nasopharynx, oral cavity, respiratory tract (trachea, bronchi, bronchioles, alveoli), gastrointestinal tract (esophagus, stomach, duodenum, small intestine, colon, rectum), biliary tract, urinary tract (uterus, urethra (including prostatic urethra), bladder), male reproductive tract, sinuses and vascular system (arteries, veins). Cavities can also be created during surgical procedures (e.g., tumor resection site), while other cavities can be accessed during open, endoscopic or radiologic procedures, such as the thoracic and abdominal (peritoneal) cavity. In intracavitary therapy, implantation of the radioactive source can be permanent or temporary.

Specialized applicators are frequently used for intracavitary placement of radioactive sources in the female reproductive tract, including the Rectangular Handle Fletcher-Suit Afterloading Applicator, the Round Handle Fletcher-Suit-Delclos Afterloading Applicator, the Delclos Miniovoid Afterloading Applicator, the Henschke Afterloading Applicator (Fletcher et al., American Journal of Roentgenology, 68:935-947, 1952) and vaginal cylinders. These are typically used to temporarily deliver cesium (e.g., Cs¹³⁷), radium (Ra²²⁶), iridium (Ir¹⁹²), iodine (I¹²⁵) or other isotopes as “seeds”, or to deliver specialized carriers (e.g., Simon-Heyman Capsules; U.S. Pat. No. 3,750,653).

For the placement of radioactive sources into deeper body cavities (e.g., GI tract, biliary tract, urinary tract, respiratory tract, vascular system) specialized catheters are used in combination with endoscopy (e.g., GI, respiratory, and biliary tracts) or radiographic guidance (e.g., vascular system) for proper placement. The following describes compositions and methods for simultaneous temporary intracavitary delivery of radioactive sources and cell cycle inhibitors including: Cell Cycle Inhibitor-Coated Radioactive Seeds, Cell Cycle Inhibitor-Coated Capsules, Cell Cycle Inhibitor-Loaded Capsules, Administration of Cell Cycle Inhibitors to the Cavity Surface and Injection of Cell Cycle Inhibitors.

Permanent intracavitary therapy can also be performed as part of implantation of a medical device. Catheters, balloons and stents are often used to open obstructed body cavities. Malignant diseases (e.g., esophageal cancer, colon cancer, biliary cancer) and non-malignant hyperproliferative diseases (e.g., atherosclerosis, restenosis, benign prostatic hypertrophy) are frequently treated in this manner. A catheter is advanced across the obstruction, a balloon is inflated to dilate the passageway and a stent is implanted to physically hold the lumen open. Radioactive catheters (e.g.,, Beta-Cath, Novoste Corporation, U.S. Pat. No. 5,899,882, see also EPA 832670, U.S. Pat. Nos. 5,938,582, 5,916,143, 5,899,882, 5,891,091, 5,851,171, 5,840,008, 5,816,999, 5,803,895, 5,782,740, 5,720,717, 5,653,683, 5,618,266, 5,540,659, 5,267,960, 5,199,939, 4,998,932, 4,963,128, 4,862,887, 4,588,395, WO 99/42162, WO 99/42149, WO 99/40974, WO 99/40973, WO 99/40972, WO 99/40971, WO 99/40962, WO 99/29370, WO 99/24116, WO 99/22815, WO 98/36790, WO 97/48452), balloon devices (see, e.g., EPA 904799, EPA 904798, EPA 879614, EPA 858815, EPA 853957, EPA 829271, EPA 325836, EPA 311458, EPB 805703, U.S. Pat. Nos. 5,913,813, 5,882,290, 5,879,282, 5,863,285, WO 99/32192, WO 99/15225, WO 99/04856, WO 98/47309, WO 98/39062, WO 97/40889) and radioactive stents (see, e.g., EPA 857470, EPA 810004, EPA 722702, EPA 539165, EPA 497495, EPB 433011, U.S. Pat. No. 5,919,126, 5,873,811, 5,871,437, 5,843,163, 5,840,009, 5,730,698, 5,722,984, 5,674,177, 5,653,736, 5,354,257, 5,213,561, 5,183,455, 5,176,617, 5,059,166, 4,976,680, WO 99/42177, WO 99/39765, WO 99/29354, WO 99/22670, WO 99/03536, WO 99/02195, WO 99/02194 and WO 98/48851). In this embodiment, compositions and methods are described for delivery of cell cycle inhibitors from catheters and balloons. In another embodiment, the cell cycle inhibitor is applied as coatings for a radioactive stent.

Within various embodiments of the above, the medical device or composition which is utilized in the intracavitary therapy can be made echogenic or radiopaque in order to enhance visualization

1. Cell Cycle Inhibitor-Coated Radioactive Seeds—This embodiment has been described above in the detailed description of interstitial therapy. Briefly, a cell cycle inhibitor is coated in a polymer capable of sustained release [such as poly(ethylene-co-vinyl acetate) (EVA) or polyurethane (PU)] and is applied to a radioactive “seed” (e.g., Cd¹³⁷, Ra²²⁶, Ir¹⁹², I¹²⁵). Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

A preferred embodiment is 1-30% paclitaxel by weight in EVA or PU applied as a coating on the radioactive source. The cell cycle inhibitor-coated radioactive source is then delivered to the tissue via any of the specialized applicators described above. In some instances, the applicator must be modified to be porous (microscopically or macroscopically) to allow the cell cycle inhibitor to elute from the “seeds” into the target tissue.

2. Cell Cycle Inhibitor-Coated Radioactive Capsules and Cell Cycle Inhibitor-Loaded Radioactive Capsules—As described above, for some intracavitary applicators specialized “capsules” are used to deliver the radioactive source to the hyperproliferative tissue (e.g., Simon-Heyman Capsules). These capsules can be coated as described above. The cell cycle inhibitor is formulated into an eluting polymer (e.g., EVA or PU) and applied to the outer surface of the capsule. Alternatively, the cell cycle inhibitor is contained in a polymer used to house the radioactive source within the polymer. Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In one preferred embodiment, 1-30% paclitaxel is loaded into EVA which is applied as a coating to the capsules. In a second preferred embodiment, 1-30% paclitaxel is a polycaprolactone-MePEG blend to heated to molten state (>60° C.). As the polymer begins to cool and solidify, radioactive sources are added in the appropriate geometry to form a cell cycle inhibitor-loaded capsule which contains radioactive seeds.

The capsules are then delivered by an applicator which is porous (i.e., allows the passage of drug through it) to allow simultaneous delivery of the cell cycle inhibitor and the therapeutic radioactive dose.

3. Administration of Cell Cycle Inhibitors to the Cavitary Surface—In another embodiment, the cell cycle inhibitor can be applied to the cavitary surface. Cell cycle inhibitors can be formulated into topical compositions suitable for administration to a cavity surface. Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In one embodiment, 1-30% paclitaxel is formulated in a gel (e.g., Pluronic F-127), that is applied as a liquid and forms a gel at body temperature, and applied to the cavity surface. Suitable indications include topical application to the vaginal mucosa, the vaginal surface of the cervix, the endocervix (or cervical canal) or the endometrium for gynecological applications. Topical application can also be easily achieved on the oral mucosa, rectal mucosa, the nasal mucosa and the surface of the nasopharynx. With the aid of endoscopy, the epithelial surface of the esophagus, stomach, duodenum, colon, trachea and bronchi can be accessed. Endoscopy can also allow access to the peritoneal surface ((abdominal cavity, the pleural space (thoracic cavity)) and the pericardial sac (thoracic cavity) for delivery of cell cycle inhibitors to these areas. Here, the preferred embodiment is a gel formulation delivered via endoscopy. For example, 1-30% paclitaxel in gel (e.g., Pluronic F-127) can be applied to the epithelial surface via endoscopy. Alternatively, an aqueous solution (e.g., “micellar paclitaxel”—1-30% paclitaxel in a diblock copolymer of polylactic acid and methoxypolyethylene glycol) can be administered via the delivery port of the endoscope. The radioactive source is then delivered according to the needs of the particular procedure. For example, the vagina or uterus is fitted with specialized applicators and a radioactive source is administered. In endoscopic applications, a catheter is maneuvered into place via the accessory port; the catheter is held or sutured in place and high-dose-rate brachytherapy is placed in the catheter. A catheter under radiographic (or endoscopic) guidance can also be used to deploy a radioactive stent capable of delivering intracavitary and radiotherapy. Regardless of the manner in which the radioactive source is applied, in this embodiment a cell cycle inhibitor is applied topically or injected into/beneath the epithelial surface to achieve local tissue levels of the agent during the radiotherapy treatment.

4. Intracavitary Injection of Cell Cycle Inhibitors—In yet another embodiment, the cell cycle inhibitor is injected into or under the cavity surface. An aqueous, nanoparticulate, microparticulate or gel formulation of a cell cycle inhibitor can be used in this manner. Injection can be accomplished directly for superficial sites (e.g., oral cavity, rectum, nasal cavity, oropharynx, nasopharynx, vagina, cervix) or via endoscope (or other specialized access device) for deeper body cavities. In a preferred embodiment, 1-30% paclitaxel in PLGA microspheres 1-20 μm in size are injected into or beneath the surface of the body cavity.

The radioactive source is then delivered according to the needs of the particular procedure. For example, the vagina or uterus is fitted with specialized applicators and a radioactive source is administered. In endoscopic applications, a catheter is maneuvered into place via the accessory port, the catheter is held or sutured in place and a high-dose-rate brachytherapy source is placed in the catheter. In medical device applications, a catheter and balloon under radiographic (or endoscopic) guidance can be used to deploy a radioactive stent capable of delivering intracavitary radiotherapy. Regardless of the manner in which the radioactive source is administered, in this embodiment a cell cycle inhibitor is applied topically or injected into/beneath the epithelial surface to achieve local tissue levels of the agent during the radiotherapy treatment.

Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

5. Cell Cycle Inhibitor-Coated Radioactive Stents—A variety of radioactive stents have been described previously (see, e.g., EPA 857470, EPA 810004, EPA 722702, EPA 539165, EPA 497495, EPB 433011, U.S. Pat. Nos. 5,919,126, 5,873,811, 5,871,437, 5,843,163, 5,840,009, 5,730,698, 5,722,984, 5,674,177, 5,653,736, 5,354,257, 5,213,561, 5,183,455, 5,176,617, 5,059,166, 4,976,680, WO 99/42177, WO 99/39765, WO 99/29354, WO 99/22670, WO 99/03536, WO 99/02195, WO 99/02194 and WO 98/48851). These devices are implanted to treat malignant obstruction of body passageways (e.g., esophageal cancer, cholangiocarcinoma, rectal cancer, lung cancer, colonic cancer) or nonmalignant hyperproliferative obstructions of body passageways (e.g., atherosclerosis, arteriosclerosis, venous stenosis, restenosis, in-stent restenosis, biliary sclerosis, benign prostatic hypertrophy). Briefly, a catheter is advanced across the obstruction under radiographic or endoscopic guidance. Typically, a balloon is inflated to dilate the obstruction and a stent is deployed (either balloon expanded or self-expanded) to physically hold open the obstructed passageway. Radioactive isotopes, such as P³², Au¹⁹⁸, Ir¹⁹², Co⁶⁰, I¹²⁵ and Pd¹⁰³, are incorporated into the stent to provide local emission of radiotherapy.

In this embodiment, a cell cycle inhibitor is linked to, coated on, or adapted to be released from the stent (e.g., the cell-cycle can be incorporated into a polymeric carrier applied to the surface of the stent or incorporated into the stent material itself).

In one embodiment, paclitaxel at 1-30% loading by weight is incorporated into polyurethane and applied as a coating to the surface of the stent. In a second embodiment, 10 μg to 2 mg of paclitaxel in a crystalline form is dried onto the surface of stent. A polymeric coating may then be placed over the drug to help control release of the cell cycle inhibitor into the tissue. In a third embodiment, 1-30% paclitaxel by weight is incorporated into a polymer which composes part of the stent's structure. Such polymeric stents have been described previously (e.g., U.S. Pat. Nos. 5,762,625, 5,670,161, WO 95/26762, EPA 420541, U.S. Pat. Nos. 5,464,450, 5,551,954) and cell cycle inhibitors and radioactive sources (e.g., I¹²⁵) can be easily incorporated as described herein. For example, paclitaxel can be incorporated into poly(lactide-co-caprolactone) (PLC), polyurethane (PU) and/or poly(lactic acid) (PLA); radioactive “seeds” can be physically incorporated into the polymer matrix prior to solidification as part of the casting and manufacturing of the stent.

Alternatively, the radioactive source can be delivered via a catheter, as has been described previously (e.g., Beta-Cath®, RadioCath) and the cell cycle inhibitor is delivered via the stent as described above.

6. Cell Cycle Inhibitor Delivered via Drug Delivery Balloons—Numerous balloons have been described for the delivery of pharmacologic agents (Transport®, Crescendo®, Channel®). In this embodiment, the cell cycle inhibitor is delivered via such a balloon in conjunction with a radioactive source. Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In a preferred embodiment, 1-30% micellar (aqueous) paclitaxel (MePeg-PDLLA) is infused via balloon. Alternatively, a 1-30% paclitaxel-loaded microparticulate or microspheric formulation (e.g., PLGA) of the cell cycle inhibitor can be utilized.

The radioactive source is delivered via the catheter (see above), via the stent or via the balloon. In another preferred embodiment, a balloon capable of microinjection into the wall of body passageways is deployed (e.g., Channel® balloon). Here a radioactive seed is coated with a cell cycle inhibitor and injected via the balloon into the wall of the obstructed passageway. Cell cycle inhibitor-coated radioactive seeds have been described previously.

7. Cell Cycle Inhibitor Delivered via Catheter—Numerous drug delivery catheters have been described for the local delivery of pharmacologic agents, e.g., radioactive catheters (EPA 832670, U.S. Pat. Nos. 5,938,582, 5,916,143, 5,899,882, 5,891,091, 5,851,171, 5,840,008, 5,816,999, 5,803,895, 5,782,740, 5,720,717, 5,653,683, 5,618,266, 5,540,659, 5,267,960, 5,199,939, 4,998,932, 4,963,128, 4,862,887, 4,588,395, WO 99/42162, WO 99/42149, WO 99/40974, WO 99/40973, WO 99/40972, WO 99/40971, WO 99/40962, WO 99/29370, WO 99/24116, WO 99/22815, WO 98/36790, WO 97/48452) and balloon devices (EPA 904799, EPA 904798, EPA 879614, EPA 858815, EPA 853957, EPA 829271, EPA 325836, EPA 311458, EPB 805703, U.S. Pat. Nos. 5,913,813, 5,882,290, 5,879,282, 5,863,285, WO 99/32192, WO 99/15225, WO 99/04856, WO 98/47309, WO 98/39062, WO 97/40889). Here aqueous, nanoparticulate and microparticulate formulations (all described above) can be infised via such a device. The therapy is then delivered via the catheter, the stent or the balloon.

Representative examples of cell cycle inhibitors that can be delivered in this manner include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

8. Cell Cycle Inhibitor Coated or loaded radioactive fabrics—In this embodiment, a radioactive fabric is prepared by coating a fabric with a radioactive substance, or, by interweaving radioactive fibre(s) to form a radioactive cloth. Similarly, the cell cycle inhibitor can be coated onto a fabric, or, the fabric itself can be composed of or interwoven with cell cycle inhibitor fibers. Within certain embodiments, the fabric may be coated with or interwoven with a composition of fiber(s) which contain or comprise both a radioactive substance and a cell cycle inhibitor.

Representative examples of cell cycle inhibitors that can be utilized in this regard include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

9. Coating of a Radioactive Medical Device—In this embodiment, a radioactive medical device is coated with polymer(s) such as acrylates (e.g., polyacrylic acid, or a methacrylate such as polymethylmethacylate), cellulose (e.g, ethyl cellulose), polysaccharide (e.g., hyaluronic acid), vinyls (e.g., polyvinyl acetate), ethers (e.g., polyoxyethylene), styrenes (e.g., polystyrene), or amino acids (e.g., polyaspartic acid or albumin). Within certain embodiments, the polymer(s) can be cross-linked by reaction with a compatible crosslinker.

As an example, a polymer at 10% is dissolved in a compatible solvent such as dichloromethane for polymethylmethacrylate or water for hyaluronic acid. The radioactive device, such as a fastening device, seed, wire, or the like is then dipped into the solution and then transferred to a dryer to remove the solvent by mild heating to 45° C. with a high vacuum. The coated device is dried to constant weight. A dried device has less then a 1% change in weight in three consecutive measurements of mass after 6 hours of drying time.

As noted above, the polymer coating can include a cell cycle inhibitor as well. This is accomplished by dissolving the cell cycle inhibitor and polymer in a mass ratio of 1:9 into the compatible solvent. In another method, the cell cycle inhibitor is micronized by milling, a particle size fraction of 10-100 μm is collected by sieving and this fraction is suspended by stirring for 30 minutes in a 30% polymer solution. Representative examples of cell cycle inhibitors that can be utilized in this regard include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

Within various further embodiments of the above, the device may also include a glidant, wax, magnetic resonance responsive (e.g. a Gadolinium III chelate), X-ray responsive (e.g. tantalum), or ultrasound responsive material. This material is loaded in the same manner as described for the inclusion of drugs.

C. SURFACE THERAPY

In surface therapeutic embodiments, the cell cycle inhibitor and the radioactive source are placed on the surface of a hyperproliferative tissue. The principle applications are for the treatnent of superficial hyperproliferative skin diseases and the surfaces of tumor surgical resection sites.

Within various embodiments of the above, the medical device or composition which is utilized for surface therapy can be made echogenic or radiopaque in order to enhance visualization.For dermal applications, when brachytherapy is administered, it is typically in the form of interstitial therapy (described previously) or given via custom-made surface “molds” which contain radioactive wires (e.g., iridium wires) or catheters fitted with a radioactive source. The following describes compositions and methods for simultaneous surface delivery of cell cycle inhibitors and radioactive sources including: Topical Cell Cycle Inhibitor Administration, Surface Molds Containing Cell Cycle Inhibitors and a Radioactive Source and Intradernal Injection of Cell Cycle Inhibitors.

Briefly, tumor resection is the primary therapeutic option for the majority of patients diagnosed with a solid tumor. Complete surgical removal of the mass offers the best opportunity for cure and is undertaken wherever possible. Unfortunately, in a significant number of patients, complete excision of the mass is not possible as the disease has grossly spread into critical structures which cannot be removed. In others, pathological examination reveals microscopic evidence of the disease remaining at the tumor resection margins. While in still many other patients, local recurrence of the tumor occurs within centimeters of the tumor resection site despite gross and microscopic evidence taken at the time of surgery indicating that the tumor had been completely excised. Therefore, there remains a considerable clinical need to develop therapies that will attack tumor tissue left behind (grossly, microscopically or occultly) after attempted tumor excision surgery.

To address this problem, permanent surface brachytherapy placement can be performed during surgical resection of a tumorous mass. An open, or endoscopic, procedure is undertaken to access a naturally occurring (e.g., visceral surface of organs, such as the heart, lungs, small bowel, stomach, liver or colon; the pleural, pericardial or peritoneal cavities; and the surface of arteries, veins, nerves, muscles and tendons) or artificially created (e.g., tumor resection “beds”) internal body surface. The delivery of permanent surface brachytherapy is initiated by fabricating a custom-made mold (usually made using dental alginates) to obtain an impression of the surface anatomy. An implant is then constructed from the mold and a radioactive source (e.g., “seeds”, catheters or wires) is placed within it. The radioactive implant is then inserted onto the internal surface to deliver permanent local brachytherapy. The following embodiments describe surgical “paste”, “gel”, “film” and “spray” compositions and methods of administration for locally delivering cell cycle inhibitors and radiotherapy. These embodiments have two distinct advantages over conventional therapies: (1) simultaneous local delivery of both a cell cycle inhibitor and radiotherapy; and (2) one-step application (i.e., a “mold” is not required; the paste, gel, film or spray conforms to the body cavity and the radioactive source is placed within it, thereby eliminating a step in the administration of the therapy). This can significantly reduce treatment administration time, which, in turn, can greatly reduce the period the surgical wound remains open. Decreasing the duration of the surgery and the time the wound remains open can reduce the morbidity and mortality associated with complicated tumor resection surgeries.

1. Topical Cell Cycle Inhibitor Administration—In this embodiment, a topical formulation of the cell cycle inhibitor is administered in conjunction with brachytherapy. For dermal applications, the cell cycle inhibitor is formulated in a vehicle such that the agent penetrates through the full thickness of the skin. Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In a preferred embodiment, 1-30% paclitaxel (or analogues or derivatives thereof) by weight is administered in a topical gel formulation based on Transcutol® and hydroxyethylcellulose to the skin surface. The topical paclitaxel formulation is applied 1-4 times daily over the affected area. Radiotherapy is then applied as surface brachytherapy or interstitial brachytherapy to compliment the topical administration of the cell cycle inhibitor.

2. Surface Molds Containing a Cell Cycle Inhibitor and a Radioactive Source—In this embodiment, a surface mold is fabricated which houses a radioactive source and elutes a cell cycle inhibitor for the management of hyperproliferative dermal diseases. Briefly, in surface brachytherapy, molds containing radioactive seeds, catheters or wires are fabricated for placement over the hyperproliferative skin lesion (Crook J. M. et al, Brachytherapy for Skin Cancer, In: Principles and Practices of Brachytherapy, Editor: Subir Nag, Futura Publishing Co., 1997). Representative examples of cell cycle inhibitors include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In one embodiment, 1-30% paclitaxel is loaded into polyurethane and fabricated into a surface mold into which a radioactive source is inserted (see FIG. 8).

3. Intradermal Injection of Cell Cycle Inhibitors—In this embodiment, the cell cycle inhibitor is formulated in an aqueous, nanoparticulate or microparticulate form for intradermal injections. Such compositions have been described previously. Briefly, the cell cycle inhibitor formulated in a sustained-release vehicle is injected intradermally or subcutaneously. The formulation is designed to provide sustained release of the cell cycle inhibitor for the duration of the radiotherapy. The radiotherapy is delivered as surface brachytherapy or interstitial brachytherapy.

Representative examples of cell cycle inhibitors that can be administered in this manner include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

4. Surgical “Pastes” Containing Cell Cycle Inhibitors and a Radioactive Source—In this embodiment, a cell cycle inhibitor and a radioactive source are applied to an internal body surface during an open or endoscopic surgical procedure. Specific clinical indications are described elsewhere herein, but typically this will be performed as part of tumor resection surgery.

Since the anatomy of any given tumor resection site is highly variable and impossible to anticipate prior to the surgical procedure, it is important that the surgical embodiments be able to conform to the resection cavity. Surgical pastes possess this property. In a surgical “paste”, the cell cycle inhibitor is contained in a polymer that is in a liquid or molten state at application temperature and forms a solid or semisolid at body temperature (37° C.) in situ.

Representative examples of cell cycle inhibitors that can be delivered in this manner include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In one embodiment, the cell cycle inhibitor is contained in a “thermopaste” polymer composed of polycaprolactone and MePEG. This surgical “thermopaste” is molten at 55-60° C. For example, 1-30% paclitaxel is loaded into thermopaste (see example) and the mixture is gently heated to 60° C. The cell cycle inhibitor-loaded thermopaste can then be injected via a syringe into the resection cavity and spread by the surgeon to cover the entire resection margin (the formulation is a viscous liquid at this temperature). As the thermopaste begins to cool to body temperature (37° C.), it gradually begins to solidify in the shape of the resection cavity. During this time interval, the radioactive source can be inserted into the paste in the correct geometry to also deliver radiotherapy. Radioactive catheters, wires or seeds can be placed in the molten liquid which subsequently hardens to fix the radioactive source in place. The cell cycle inhibitor is released gradually over time from the polymer and the radioactive source decays over time to deliver a therapeutic dose. The result is delivery of a cell cycle inhibitor and brachytherapy directly to the entire resection margin—all accomplished in a single administration step.

A related embodiment is a cell cycle inhibitor contained within “cryopaste”. Here the Pluronic F-127 carrier polymer is liquid at 4° C. The cell cycle inhibitor, for example 1-30% paclitaxel cryopaste (see example), is applied to the tumor resection margin. As the composition warms to 37° C., it slowly begins to solidify. In the same manner as described for thermopaste, it is during this time interval that a radioactive source can be added to create the finished product. Radioactive seeds, wires or catheters are placed in the cryopaste to deliver the correct dosimetry to the resection margin.

As should be readily evident, thermogelling polymers are appropriate for this application. In particular, most biocompatible polymers or polymer blends which are fluid or semisolid above or below body temperature, but solid at body temperature can be used for this embodiment. Similarly, the radioactive source can be evenly dispersed within the liquid prior to application (as opposed to being added after placement in the resection surface).

5. Surgical Gels Containing a Cell Cycle Inhibitor and a Radioactive Source—In this embodiment, the cell cycle inhibitor and the radioactive source are contained within a gel that is applied to the resection margin. Representative examples of cell cycle inhibitors that can be delivered in this manner include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In a preferred embodiment, the gel is composed of hyaluronic acid loaded with 1-30% paclitaxel by weight. The gel is applied by the surgeon directly to the entire resection margin during open procedures or via endoscopy. The radioactive sources, preferably “seeds”, are then placed into the gel in the appropriate spacing.

6. Surgical “Films” Containing a Cell Cycle Inhibitor and a Radioactive Source—In this embodiment, the cell cycle inhibitor and the radioactive source are contained within a flexible film appropriate for application at a tumor resection site. Ideal polymeric delivery vehicles for this application include polyurethane (PU) and poly(ethylene-co-vinyl acetate) (EVA) (see examples). However, any polymer that is flexible and biocompatible is suitable for use in this embodiment.

Representative examples of cell cycle inhibitors that can be delivered in this manner include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, cannustine and lomustine), estramustine, tamoxifen, leucovorin, floxuridine, ethyleneimines (e.g., thiotepa); and tetrazines (e.g., dacarbazine and procarbazine).

In a preferred embodiment, 1-30% paclitaxel by weight is incorporated in polyurethane. The cell cycle inhibitor-loaded film is fabricated in one of two ways:

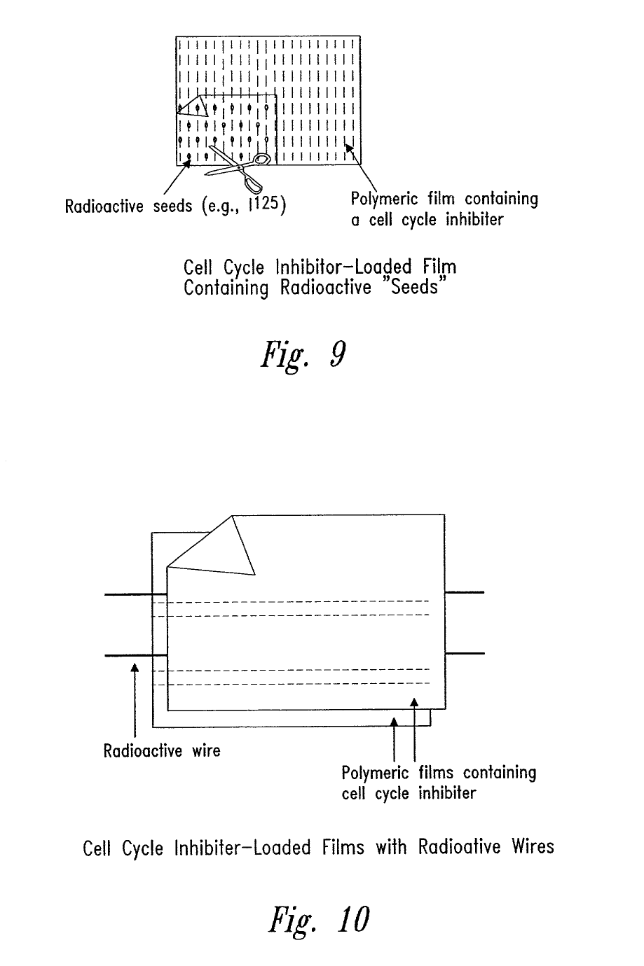

(a) The surface of the film is scored to contain 0.1 cm×0.5 cm×0.1 cm wells (i.e., I¹²⁵ and Pd¹⁰³ seeds are about this size (the size of a grain of rice)) spaced 0.5 or 1.0 cm apart (see, e.g., FIG. 9). The wells are sized such that a radioactive “seed” (e.g. U.S. Pat. No. 4,323,055) can be placed within it. The “wells” are spaced 0.5 cm or 1.0 cm apart (in all directions) depending on the application to allow for even dosimetry of the brachytherapy. The advantage of PU and EVA is that both polymer films can be cut with a scalpel or scissors and both are very flexible. Therefore, the surgeon can cut the film to the ideal size and shape which covers the cavity surface. Radioactive “seeds” are then placed in the wells to achieve the desired dosimetry. The seeds can then be “sealed” in the wells by applying a molten polymer over the seeds which solidifies in place (see Surgical Paste section for a more detailed description of formulations). Alternatively, a second polymer film can be applied over the wells to ensure seed placement is maintained. The cell cycle inhibitor-loaded film containing the radioactive seeds is then placed in the resection cavity and can be sutured in place, if required.

(b) The surface of the film is scored to contain radioactive wires (see, e.g., FIG. 10. Two sheets of cell cycle inhibitor-loaded polymeric films are fabricated for placement on either side of radioactive wires.

In a preferred embodiment, 1-30% paclitaxel is loaded into PU and solvent-casted into “sheets” with or without depressions (to aid in wire placement). Again, the sheets can be cut to size and the entire composition (drug-loaded polymer and radioactive wires) are placed into the resection cavity.

7. Surgical “Sprays” Containing a Cell Cycle Inhibitor and a Radioactive Source—In this embodiment, the cell cycle inhibitor and the radioactive source are contained within a spray which is delivered to the tumor resection margin. Representative examples of cell cycle inhibitors that can be delivered in this manner include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

In a preferred embodiment, 1-30% paclitaxel is formulated into an aerosol into which radioactive seeds are dispersed. Microparticulate radioactive sources are preferred (e.g., gold grains). The cell cycle inhibitor-loaded radioactive spray is then applied to the resection margin. This is particularly effective for endoscopic procedures, since this embodiment can be delivered via the side port of the endoscope.

8. Cell Cycle Inhibitor Coated or loaded radioactive fabrics—In this embodiment, a radioactive fabric is prepared by coating a fabric with a radioactive substance, or, by interweaving radioactive fibre(s) to form a radioactive cloth. Similarly, the cell cycle inhibitor can be coated onto a fabric, or, the fabric itself can be composed of or interwoven with cell cycle inhibitor fibers. Within certain embodiments, the fabric may be coated with or interwoven with a composition of fiber(s) which contain or comprise both a radioactive substance and a cell cycle inhibitor.

Representative examples of cell cycle inhibitors that can be utilized in this regard include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

9. Coating of a Radioactive Medical Device—In this embodiment, a radioactive medical device is coated with polymer(s) such as acrylates (e.g., polyacrylic acid, or a methacrylate such as polymethylmethacylate), cellulose (e.g, ethyl cellulose), polysaccharide (e.g., hyaluronic acid), vinyls (e.g., polyvinyl acetate), ethers (e.g., polyoxyethylene), styrenes (e.g., polystyrene), or amino acids (e.g., polyaspartic acid or albumin). Within certain embodiments, the polymer(s) can be cross-linked by reaction with a compatible crosslinker.

As an example, a polymer at 10% is dissolved in a compatible solvent such as dichloromethane for polymethylmethacrylate or water for hyaluronic acid. The radioactive device, such as a fastening device, seed, wire, or the like is then dipped into the solution and then transferred to a dryer to remove the solvent by mild heating to 45° C. with a high vacuum. The coated device is dried to constant weight. A dried device has less then a 1% change in weight in three consecutive measurements of mass after 6 hours of drying time.

As noted above, the polymer coating can include a cell cycle inhibitor as well. This is accomplished by dissolving the cell cycle inhibitor and polymer in a mass ratio of 1:9 into the compatible solvent. In another method, the cell cycle inhibitor is micronized by milling, a particle size fraction of 10-100 μm is collected by sieving and this fraction is suspended by stirring for 30 minutes in a 30% polymer solution. Representative examples of cell cycle inhibitors that can be utilized in this regard include taxanes (e.g., paclitaxel and docetaxel), topoisomerase inhibitors (e.g., ironotecan and topotecan), vinca alkaloids (e.g., vinblastine, vincristine and vinorelbine), platinum (e.g., cisplatin and carboplatin), mitomycin, gemcitabine, alkalating agents (e.g., cyclophosphamide, flouropyrimidine, capecitabine, and 5-FU), anthracylines (e.g., doxorubicin mitoxantrone and epirubicin), nitrogen mustards (e.g., ifosfamide and melphalan), antimetabolites (e.g., methotrexate), nitrosoureas (e.g., CCNU, streptozocin, carmustine and lomustine), estramustine, tamoxifen, leucovorin, floxuridine, ethyleneimines (e.g., thiotepa); and tetrazines (e.g., dacarbazine and procarbazine).

Within various further embodiments of the above, the device may also include a glidant, wax, magnetic resonance responsive (e.g. a Gadolinium III chelate), X-ray responsive (e.g. tantalum), or ultrasound responsive material. This material is loaded in the same manner as described for the inclusion of drugs.

### (IV) CLINICAL APPLICATIONS

In order to further the understanding of the compositions and methods for the treatment of hyperproliferative diseases, representative clinical applications are discussed in more detail below. As utilized herein, it should be understood that the term “treatment” refers to the therapeutic administration of a desired device, composition, or compound, in an amount and/or for a time sufficient to treat, inhibit, or prevent at least one aspect or marker of a disease, in a statistically significant manner.

Hyperproliferative Diseases of the Prostate

Prostate cancer is the most common malignancy of men (>300,000 new cases per year in the U.S.) and benign prostatic hypertrophy (BPH) affects an increasing number of individuals as they grow older (it is estimated that BPH affects 80% of men over the age of 80). As a result, more effective therapies for hyperproliferative diseases of the prostate are greatly needed.

An effective therapy for prostate cancer would stop or slow tumor growth and/or prevent the spread of the disease into adjacent or distant organs. Since the disease affects older individuals, treatments that do not require surgery are preferred as many patients have concurrent illnesses that make them poor surgical candidates.

An effective therapy for BPH would reduce the symptoms associated with urinary obstruction (e.g., poor urine stream, terminal dribbling, nocturia) and improve voiding.

For hyperproliferative lesions within the prostate, transperineal or transrectal, ultrasound-guided, permanent brachytherapy is the most commonly employed form of treatment. Usually, I¹²⁵ or Pd¹⁰³ seeds are implanted, although Au¹⁹⁸ and Rn²²² are occasionally employed. The patients treated usually have Stage A or B (occasionally C) prostate cancer with no evidence of distant metastases. The recommended dose of brachytherapy is 115-120 Gy for Pd¹⁰³ and 150-160 Gy for I¹²⁵, although this can vary somewhat between individual patients. Although any interstitial, intracavitary, or surface therapy described previously can be utilized, preferred embodiments include:

1. Cell Cycle Inhibitor-Loaded Spacers

2. Cell Cycle Inhibitor-Coated Radioactive Seeds

3. Cell Cycle Inhibitor-Coated Radioactive Sutures

4. Cell Cycle Inhibitor-Loaded Radioactive Sutures

5. Interstitial Injection of Cell Cycle Inhibitors

6. Cell Cycle Inhibitor-Coated Radioactive Wires

7. Cell Cycle Inhibitor-Coated Radioactive Urethral Stents

8. Transurethral Delivery of Cell Cycle Inhibitors via Drug-Delivery Balloons or Catheters

9. Cell Cycle Inhibitor-Loaded Surgical Pastes, Films, or Sprays

Within particularly preferred embodiments of the above, the therapeutic device or compositions which are utilized in the above therapy (e.g., radioactive seed, seed spacers, carriers, or cell-cycle inhibitors) can be made radiopaque or echogenic in order to enhance visualizaiton.

In one embodiment, a cycle inhibitor is loaded into a resorbable [(e.g., poly (glycolide), poly (lactide-co-glycolide), poly (glycolide-co-caprolactone), albumin, hyaluronic acid, gelatin, and/or Carbopol)] or nonresorbable [(e.g., polypropylene, silicone, EVA, polyurethane, and/or polyethylene] polymer(s) and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted through a template and into the hyperproliferative tissue in the prostate. Under general or spinal anesthesia, a template is placed over the perineum (e.g. Syed-Neblett Template, Martinez Universal Perineal Interstitial Template) and needles/catheters are inserted through holes in the template under ultrasonic or fluoroscopic guidance until the entire prostate is implanted with needles 0.5 to 1.0 cm apart. Although any cell cycle inhibitor could be incorporated into a polymeric spacer, taxanes, topoisomerase inhibitors, vinca alkaloids and/or estramustine are preferred. For example, 0.1-₄₀% w/w paclitaxel incorporated into a resorbable or non-resorbable polymeric spacer is an ideal embodiment. Docetaxel at 0.1-40% w/w, 0.1-40% w/w etoposide, 0.1-40% w/w vinblastine, and/or 0.1-40% w/w estramustine are also preferred embodiments. It should be obvious to one of skill in the art that analogues or derivatives of the above compounds (as described previously) given at similar, or biologically equivalent, dosages would also be suitable for the above invention.

In a second embodiment, a cell cycle inhibitor-coated radioactive seed can be utilized. Here the cell cycle inhibitor is coated directly onto the radioactive seed (e.g. I¹²⁵or Pd¹⁰³) either prior to, or at the time of, implantation into the prostate. Once again preferred cell cycle inhibitors include taxanes, topoisomerase inhibitors, vinca alkaloids and/or estramustine. For example, 0.1-40% w/w paclitaxel or 0.1-₄₀% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Similarly 0.1-40% w/w etoposide, 0.1-40% w/w vinblastine and/or 0.1-40% w/w estramustine can be incorporated into poly (glycolide), poly (lactide-co-glycolide), poly (glycolide -co-caprolactone), albumin, hyaluronic acid, gelatin, Carbopol, polypropylene, silicone, EVA, polyurethane, polyethylene and coated onto a brachytherapy seed. The cell cycle inhibitor-coated radioactive seed is then implanted into the prostate via needles or catheters (as described previously) or via specialized applicators (e.g. Mick Applicator). The Mick Applicator, for example, can implant cell cycle inhibitor-coated seeds at 1 cm intervals in the prostate and their position can be verified by fluoroscopy.

In a third embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the prostate percutaneously or during open surgery. A cell cycle inhibitor can be loaded into a polymeric carrier applied to the surface of the suture material prior to, or during, implantation. Preferred cell cycle inhibitors for non-absorbable sutures are taxanes, topoisomerase inhibitors, vinca alkaloids and/or estramustine loaded into EVA, polyurethane (PU), PLGA, silicone, gelatin, and/or dextran. The polymer-cell cycle inhibitor formulation is then applied as a coating (e.g. sprayed, dipped, “painted” on) onto the radioactive suture prior to insertion in the prostate. Examples of specific, preferred agents include 0.1-40% w/w, paclitaxel, 0.1-40% w/w docetaxel, 0.1-40% w/w, etoposide, 0.1-40% w/w, vinblastine, and/or 0.1-40% w/w estramustine loaded into one (or a combination of) the above polymers and applied as a coating to a radioactive suture. Conversely, incorporation of the above agents in poly(lactide-co-glycolide), poly(glycolide) or dextran would be the preferred coating for absorbable radioactive sutures.

In a fourth embodiment, the cell cycle inhibitor is loaded into a radioactive suture (i.e., the cell cycle inhibitor-polymer composition is a constituent component of the suture). In a preferred embodiment, a taxane, topoisomerase inhibitor, vinca alkaloid and/or estramustine is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵or Pd¹⁰³). Particularly, preferred cell cycle inhibitors for this purpose include 0.1-40% w/w paclitaxel, 0.1-40% w/w docetaxel, 0.1-40% w/w etoposide, 0.1-40% w/w vinblastine, and/or 0.1-40% w/w estramustine. If a nonabsorbable suture is desired, the above agents can be loaded into polypropylene or silicone. In both cases the radioactive source is evenly spaced (e.g. 1 cm apart) within the suture see FIG. 3.

A fifth embodiment for the treatment of hyperproliferative diseases of the prostate is infiltration of the prostate with interstitial injections of cell cycle inhibitor formulations (aqueous, nanoparticulates, microspheres, pastes, gels, etc.) prior to, or at the time of brachytherapy treatment. Taxanes, topoisomerase inhibitors, vinca alkaloids and/or estramustine compounds are preferred for this embodiment. For example, paclitaxel, docetaxel, etoposide, vinblastine and/or estramustine can be incorporated into a polymeric carrier as described previously. The resulting formulation—whether aqueous, nano or microparticulate, gel, or paste in nature—must be suitable for injection through a needle or catheter. The polymer-cell cycle inhibitor formulation is then injected into the prostate gland such that therapeutic drug levels are reached in the diseased tissues. A brachytherapy source is also administered interstitially by any of the methods as described previously. While also suitable for use with permanent low dose brachytherapy sources, this treatment form is best suited for use with temporary high dose rate (HDR) brachytherapy. For example, the prostate can be infiltrated by interstitial injection of the cell cycle inhibitor in combination with high energy I¹⁹², administered via a template, which remains in place for 50-80 minutes before being removed. Interstitial injection of the cell cycle inhibitor is ideal for HDR therapy since, unlike some of the other interstitial embodiments, it does not require attachment of the cell cycle inhibitor to the brachytherapy source—important since the brachytherapy source is ultimately removed in HDR.

In a sixth embodiment, a cell cycle inhibitor is coated onto a radioactive wire. In this application, radioactive wires (e.g. Ir¹⁹²) are placed through the tumor via the skin (percutaneously) or during open surgery. If the wire is to remain in place permanently, a variety of polymeric carriers are suitable for administration of the cell cycle inhibitor including EVA, polyurethane and silicone. The cell cycle inhibitor-polymer coating can be applied as a spray or via a dipped coating process either in advance of, or at the time of, insertion. A “sheet” of cell cycle inhibitor-polymer material (e.g. EVA, Polyurethane) can also be wrapped around the wire prior to insertion. If temporary high dose brachytherapy is employed, the wire must be directly coated with a cell cycle inhibitor (i.e., the drug is dried on to the surface of the wire or directly attached to the wire) or the cell cycle inhibitor must be loaded into a polymer capable of rapid drug release, such as polyethylene glycol, dextran and hyaluronic acid (this is necessary since most of the drug must be released within a 1-2 hour period). Regardless of the form of brachytherapy performed, ideal cell cycle inhibitors for use as wire coatings in the treatment of hyperproliferative diseases of the prostate include taxanes, topoisomerase inhibitors, vinca alkaloids and/or estramustine. For example, 0.1-40% w/w paclitaxel, 0.1-40% w/w docetaxel, 0.1-40% w/w etoposide 0.1-40% w/w vinblastine, and/or 0.1-40% w/w estramustine can be loaded into fast release polymeric formulations such as polyethylene glycol, dextran and hyaluronic acid for coating onto temporary HDR brachytherapy wires.

In a seventh embodiment, a cell cycle inhibitor can be coated onto a radioactive stent [EPA 857470; EPA 810004; EPA 722702; EPA 539165; EPA 497495; EPB 433011; U.S. Pat. Nos. 5,919,216; 5,873,811; 5,871,437; 5,843,163; 5,840,009; 5,730,698; 5,722,984; 5,674,177; 5,653,736; 5,354,257; 5,213,561; 5,183,455; 5,176,617; 5,059,166; 4,976,680; WO 99/42177; WO 99/39765; WO 99/29354; WO 99/22670; WO 99/03536; WO 99/02195; WO 99/02194; WO 98/48851]. A cell cycle inhibitor-coated radioactive stent can be implanted in the prostatic urethra for treatment of BPH or malignant obstruction of the urethra. Briefly, a catheter is advanced across the obstruction under radiographic or endoscopic guidance, a balloon is inflated to dilate the obstruction, and a stent is deployed (either balloon expanded or self expanded). Radioactive isotopes, such as P³², Au¹⁹⁸, Ir¹⁹², Co⁶⁰, I¹²⁵, and Pd¹⁰³ are contained within the stent to provide a source of radioactivity. A cell cycle inhibitor is linked to the surface of the stent, incorporated into a polymeric carrier applied to the surface of the stent (or as a “sleeve” which surrounds the stent), or is incorporated into the stent material itself. Cell cycle inhibitors ideally suited to this embodiment include taxanes, topoisomerase inhibitors, vinca alkaloids and/or estramustine. For example, 0.01 - 10% w/w paclitaxel, 0.01 - 10% w/w docetaxel, 0.01-10% w/w etoposide 0.01-10% w/w vinblastine, and/or 0.01-10% w/w estramustine can be incorporated into silicone, polyurethane and/or EVA, which is applied as a coating to the radioactive stent. Alternatively, 10 μg-10 mg paclitaxel, 10 μg-10 mg docetaxel, 10μg-10 mg etoposide, 10 μg-10 mg vinblastine, and/or 10 μg-10 mg estramustine in a crystalline form can be dried onto the surface of the stent. A polymeric coating may be applied over the cell cycle inhibitor to help control the release of the agent into the surrounding tissue. A third alternative is to incorporate 0.01-10% w/w paclitaxel, 0.01-10% w/w docetaxel, 0.01-10% w/w etoposide, 0.01-10% w/w vinblastine, and/or 0.01-10% w/w estramustine into a polymer (U.S. Pat. Nos. 5,762,625; 5,670,161; WO 95/26762; EPA 420541; U.S. Pat. Nos. 5,464,450; 5,551,954) which comprises part of the stent structure. For example, the cell cycle inhibitor can be incorporated into a polymer such as poly (lactide-co-caprolactone), polyurethane, and/or polylactic acid in combination with a radioactive source (e.g. I¹²⁵, P³²) prior to solidification as part of the casting and manufacturing of the stent. A final alternative involves delivering the brachytherapy source via a catheter (e.g. Beta-Cath®, RadioCath®, etc.) while the cell cycle inhibitor is delivered via the stent.

In an eighth embodiment, the cell cycle inhibitor can be delivered into (or through) the prostatic urethra via specialized balloons (e.g. Transport®; Crescendo®, Channel®; and see EPA 904799; EPA 904798; EPA 879614; EPA 858815; EPA 853957; EPA 829271; EPA 325836; EPA 311458; EPB 805703; U.S. Pat. Nos. 5,913,813; 5,882,290; 5,879,282; 5,863,285; WO 99/32192; WO 99/15225; WO 99/04856; WO 98/47309; WO 98/39062; WO 97/40889) or delivery catheters (EPA 832670; U.S. Pat. Nos. 5,938,582; 5,916,143; 5,899,882; 5,891,091; 5,851,171; 5,840,008; 5,816,999; 5,803,895; 5,782,740; 5,720,717; 5,653,683; 5,618,266; 5,540,659; 5,267,960; 5,199,939; 4,998,932; 4,963,128; 4,862,887; 4,588,395; WO 99/42162; WO 99/42149; WO 99/40974; WO 99/40973; WO 99/40972; WO 99/40971; WO 99/40962; WO 99/29370; WO 99/24116; WO 99/22815; WO 98/36790; WO 97/48452). Here a cell cycle inhibitor formulated into an aqueous, non-aqueous, nanoparticulate, microsphere and/or gel formulation can be delivered by such a device. Preferred cell cycle inhibitors include taxanes (e.g. paclitaxel, docetaxel), topoisomerase inhibitors (e.g. etoposide), vinca alkaloids (e.g. vinblastine) and/or estramustine at appropriate therapeutic doses. The brachytherapy is delivered via the catheter, balloon or stent.

In a ninth embodiment, the cell cycle inhibitor and the radioactive source are delivered intraoperatively as part of tumor resection surgery. Resection of a malignant prostate mass is the primary therapeutic option for many patients diagnosed with prostate cancer. Unfortunately, for many patients complete removal of the mass is not possible and malignant cells remain in adjacent tissues. To address this problem, a cell cycle inhibitor can be combined with a radioactive source and applied to the surface of the tumor resection margin. Surgical pastes, gels and films containing taxanes, topoisomerase inhibitors, vinca alkaloids and/or estramustine are ideally suited for treatment of prostate tumor resection beds. In a surgical paste, 0.1-40% w/w paclitaxel, 0.1-40% w/w docetaxel, 0.1-40% w/w etoposide, 0.1-40% w/w vinblastine, and/or 0.1-40% w/w estramustine is incorporated into polymeric or non-polymeric paste incorporated into a formulation (refer to examples). The cell cycle inhibitor-loaded paste is injected via a syringe into the resection cavity and spread by the surgeon to cover the desired area. For thermally responsive pastes, as the formulation cools (thermopastes: cold-sensitive) or heats (cryopastes: heat-sensitive) to body temperature (37° C.) it gradually solidifies. During this time interval, radioactive sources (e.g., iridium wires, I¹²⁵ seeds, Pd¹⁰³ seeds) are inserted into the molten formulation in the correct geometry to deliver the desired dosimetry. The paste will then completely harden in the shape of the resection margin while also fixing the radioactive source in place. Alternatively, a particulate radioactive source can be added to the thermopaste or cryopaste prior to administration when precise dosimetry is not required. A gel composed of a cell cycle inhibitor contained in hyaluronic acid can be used in the same manner as described for cryopaste and thermopastes.

Surgical films containing a cell cycle inhibitor and a radioactive source can also be used in the management of prostate tumor resection margins. Ideal polymeric vehicles for surgical films include flexible non-degradable polymers such as polyurethane, EVA, silicone and resorbable polymers such as poly (glycolide), poly (lactide-co- glycolide), poly (glycolide-co-caprolactone), albumin, hyaluronic acid, gelatin, and Carbopol. The surface of the film can be modified to hold I¹²⁵, Pd¹⁰³ seeds at regular intervals or to hold radioactive wires (see FIG. 10) for a more detailed description). In a preferred embodiment, the surgical film is loaded with a taxane, topoisomerase inhibitor, vinca alkaloid and/or estramustine. For example, 0.1-40% w/w paclitaxel, 0.1-40 w/w docetaxel, 0.1-40% w/w etoposide,0.1-40% w/w vinblastine, and/or 0.1-40% w/w estramustine is incorporated in to the film. The radioactive seeds or wires are placed in the film and can be sealed in place with either another piece of cell cycle inhibitor-loaded film or molten polymer containing a cell cycle inhibitor (described above) which hardens in place. The cell cycle inhibitor-loaded film containing the radioactive source is then placed in the resection cavity as required.

A surgical spray loaded with a cell cycle inhibitor and a brachytherapy source is also suitable for use in the treatment of prostate tumor resection margins. For this embodiment, taxanes, topoisomerase inhibitors, vinca alkaloids and/or estramustine are formulated into an aerosol into which a radioactive source is incorporated. In a preferred embodiment, paclitaxel, docetaxel, etoposide, vinblastine, and or estramustine is formulated into an aerosol which also contains an aqueous radioactive source (or microparticulate such as gold grains). This is sprayed onto the resection margin during open or endoscopic surgery interventions to help prevent tumor recurrence.

Hyperproliferative Diseases of the Anorectum

Anorectal area cancer is readily accessible to local treatment interventions. Early stage rectal adenocarcinoma is typically treated by excision, electrocoagulation or external beam radiotherapy. However, patients with more advanced disease or recurrent disease can benefit from brachytherapy and cell cycle inhibitor therapy. In general, both intracavitary and interstitial therapies can be administered to patients with anorectal area cancer including:

1. Administration of a Cell Cycle Inhibitor to the Rectal Mucosa in Combination with Placement of an Intracavitary Source of Radiation.

2. Cell Cycle Inhibitor-Coated Radioactive Capsules.

3. Cell Cycle Inhibitor-Loaded Radioactive Capsules.

4. Cell Cycle Inhibitor-Loaded Spacers.

5. Cell Cycle Inhibitor-Coated Radioactive Seeds.

6. Cell Cycle Inhibitor-Coated Radioactive Sutures.

7. Cell Cycle Inhibitor-Loaded Radioactive Sutures.

8. Interstitial Injection of Cell Cycle Inhibitors.

9. Cell Cycle Inhibitor-Coated Radioactive Wires.

For intracavitary therapy, at least three embodiments of the present invention can be utilized. In the first, a topical formulation of a cell cycle inhibitor is applied to the anal and rectal surface. Taxanes, alkylating agents, platinum, topoisomerase inhibitors, mitomycin and/or leucovorin are preferred agents for this purpose. For example 0.1-40% w/w paclitaxel, 0.1-40% w/w docetaxel, 0.1-40% w/w 5-Fluorouracil, 0.1-40% w/w cisplatin, 0.1-40% w/w irinotecan, 0.1-40% w/w mitomycin, and/or 0.1-40% w/w leucovorin are formulated into topical carriers such as a petrolatum based ointment, or a bioadhesive gel and applied to the anal and/or rectal surface. A rectal cylinder is then inserted and a central radioactive source (e.g. Ir¹⁹² wire) is placed in the cylinder for the appropriate time period to deliver a therapeutic dose of radiotherapy.

In the second and third embodiments, a porous rectal cylinder is inserted (i.e., a cylinder which readily allows passage of therapeutic agents through the wall). The cylinder must be macroporated and/or microporated. Cell cycle inhibitor-coated radioactive capsules and/or cell cycle inhibitor-loaded radioactive capsules (described previously) are then placed within the cylinder to deliver both pharmacologic and radiographic therapy. Taxanes, alkylating agents, platinum, topoisomerase inhibitors, mitomycin and/or leucovorin are preferred agents for these two embodiments. Specifically, 0.1-40% w/w paclitaxel, 0.1-40% w/w docetaxel, 0.1-40% w/w 5-Fluorouracil, 0.1-40% w/w cisplatin, 0.1-40% w/w irinotecan, 0.1-40% w/w mitomycin, and/or 0.1-40% w/w leucovorin are formulated into a polymer and applied as a coating to a radioactive capsule, or formulated into a polymer which are constituent components of the radioactive capsule.

The remaining six embodiments are suitable for interstitial treatment of anorectal malignancy. Here the interstitial embodiments are inserted percutaneously via the perineum using specialized templates (see prostate clinical applications for a more detailed description) or inserted through the anal or rectal mucosa (transrectally) into the tumor tissue under ultrasonic guidance. Intracavitary therapy can be used concurrently with interstitial therapy if clinically warranted.

In a fourth embodiment, a cell cycle inhibitor is loaded into a resorbable [(e.g., poly (glycolide), poly (lactide-co-glycolide), poly (glycolide-co-caprolactone), albumin, hyaluronic acid, gelatin, Carbopol)] or nonresorbable [(e.g., polypropylene, silicone, EVA, polyurethane, polyethylene] polymer(s) and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted through a perineal template or transrectally under ultrasound or fluoroscopic guidance until the entire tumorous area is implanted with needles 0.5 to 1.0 cm apart. Although any cell cycle inhibitor could be incorporated into a polymeric spacer, taxanes, alkylating agents, platinum, topoisomerase inhibitors, mitomycin and/or leucovorin are preferred. For example, 0.1-⁴0% w/w paclitaxel incorporated into a resorbable or non-resorbable polymeric spacer is an ideal embodiment. Docetaxel at 0.1-40% w/w, 0.1-40% w/w, 5-Fluorouracil, 0.1-40% w/w cisplatin, 0.1-40% w/w irinotecan, 0.1-40% w/w mitomycin, and/or 0.1-40% w/w leucovorin are also preferred embodiments.

In a fifth embodiment, a cell cycle inhibitor-coated seed can be utilized. Here the cell cycle inhibitor is coated directly onto the radioactive seed (e.g. I¹²⁵or Pd¹⁰³) either prior to, or at the time of, implantation into the anorectal area. Once again preferred cell cycle inhibitors include taxanes, alkylating agents, platinum, topoisomerase inhibitors, mitomycin and/or leucovorin. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Similarly 0.1-40% w/w 5-Fluorouracil, 0.1-40% w/w cisplatin, 0.1-40% w/w ironotecan, 0.1-40% w/w mitomycin, and/or 0.1-40w/w leucovorin can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the anorectal area via needles or catheters (as described above) or via specialized applicators (e.g. Mick Applicator). The Mick Applicator, for example, can implant cell cycle inhibitor-coated seeds at 1 cm intervals in the anorectal area and their position can be verified by fluoroscopy.

In a sixth embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the anorectal area percutaneously or during open surgery. A cell cycle inhibitor can be loaded into a polymeric carrier applied to the surface of the suture material prior to, or during, implantation. Preferred cell cycle inhibitor for non-absorbable sutures are taxanes, alkylating agents, platinum, topoisomerase inhibitors, mitomycin and/or leucovorin loaded into EVA, polyurethane (PU) or PLGA silicone, gelatin, and/or dextran. The polymer-cell cycle inhibitor formulation is then applied as a coating (e.g. sprayed, dipped, “painted” on) prior to insertion in the anorectal area. Examples of specific, preferred agents include 0.1-40% w/w paclitaxel, 0.1-40% w/w docetaxel, 0.1-40% w/w 5-Fluorouracil, 0.1-40% w/w cisplatin, 0.1-40% w/w ironotecan, 0.1-40% w/w mitomycin, and/or 0.1-40% w/w leucovorin loaded into one (or a combination of) the above polymers and applied as a coating to a radioactive suture. Conversely, incorporation of the above agents in poly(lactide-co-glycolide), poly(glycolide) and/or dextran would be the preferred coating for absorbable radioactive sutures.

In a seventh embodiment, the cell cycle inhibitor is loaded into a radioactive suture (i.e., the cell cycle inhibitor-polymer composition is a constituent component of the suture). In a preferred embodiment, a taxane, topoisomerase inhibitor, vinca alkaloid and/or estramustine is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵ or Pd¹⁰³). Particularly, preferred cell cycle inhibitors for this purpose include 0.1-40% w/w paclitaxel, 0.1-40% w/w docetaxel, 0.1-40% w/w 5-Fluorouracil, 0.1-40% w/w cisplatin, 0.1-40% w/w irinotecan, 0.1-40% w/w mitomycin, and/or 0.1-40% w/w leucovorin. If a nonabsorbable suture is desired, the above agents can be loaded into polypropylene or silicone. In both cases the radioactive source is evenly spaced (e.g. 1 cm apart) within the suture (see FIG. 3).

An eighth embodiment for the treatment of hyperproliferative diseases of the anorectal area is infiltration of the anorectal area with interstitial injections of cell cycle inhibitor formulations (aqueous, nanoparticulates, microspheres, pastes, gels, etc.) prior to, or at the time of brachytherapy treatment. Taxanes, alkylating agents, platinum, topoisomerase inhibitors, mitomycin and/or leucovorin compounds are preferred for this embodiment. For example, paclitaxel, docetaxel, 5-Fluorouracil, cisplatin, irinotecan, mitomycin, and/or leucovorin can be incorporated into a polymeric carrier as described previously. The resulting formulation - whether aqueous, nano or microparticulate, gel, or paste in nature—must be suitable for injection through a needle or catheter. The polymer-cell cycle inhibitor formulation is then injected transrectally or percutaneously into the anorectal area such that therapeutic drug levels are reached in the diseased tissues. A brachytherapy source is then administered interstitially or intracavitarily (within the anus or rectum) by any of the methods as described previously. While also suitable for use with permanent low dose brachytherapy sources, this treatment form is best suited for use with temporary high dose rate (HDR) brachytherapy. For example, the anorectal area can be infiltrated by interstitial injection of the cell cycle inhibitor in combination with high energy I¹⁹², which remains in place for 50-80 minutes before being removed. Interstitial injection of the cell cycle inhibitor is ideal for HDR therapy since, unlike some of the other interstitial embodiments, it does not require attachment of the cell cycle inhibitor to the brachytherapy source—important since the brachytherapy source is ultimately removed in HDR.

In a ninth embodiment, a cell cycle inhibitor is coated onto a radioactive wire. In this application, radioactive wires (e.g. Ir¹⁹²) are placed through the tumor via the skin (percutaneously), via the rectum, or during open surgery. If the wire is to remain in place permanently, a variety of polymeric carriers are suitable for administration of the cell cycle inhibitor including EVA, polyurethane and silicone. The cell cycle inhibitor-polymer coating can be applied as a spray or via a dipped coating process either in advance of or at the time of insertion. A “sheet” of cell cycle inhibitor-polymer material (e.g. EVA, Polyurethane) can also be wrapped around the wire prior to insertion. If temporary high dose brachytherapy is employed, the wire must be coated directly with a cell cycle inhibitor (i.e., the cell cycle inhibitor is dried onto or directly linked to the wire) or the cell cycle inhibitor must be loaded into a polymer capable of rapid drug release, such as polyethylene glycol, dextran and/or hyaluronic acid (since most of the drug must be released within a 1-2 hour period). Regardless of the form of brachytherapy performed, ideal cell cycle inhibitors for use as wire coatings in the treatment of hyperproliferative diseases of the anorectal area include taxanes, alkylating agents, platinum, topoisomerase inhibitors, mitomycin and/or leucovorin. For example, 0.1-40% w/w paclitaxel, 0.1-40% W/w docetaxel, 0.1-40% w/w 5-Fluorouracil, 0.1-40% w/w cisplatin, 0.1-40% w/w ironotecan, 0.1-40% w/w mitomycin, and/or 0.1-40% w/w leucovorin can be loaded into fast release polymeric formulations such as polyethylene glycol, dextran and/or hyaluronic acid for coating onto temporary HDR brachytherapy wires.

Hyperproliferative Diseases of the Bladder

Tumors of the bladder and urinary tract account for 4.2% of all cancer cases, and there are 51,200 new cases reported each year in the United States. Unfortunately, the patient often does not present until the disease is quite advanced and the morbidity and mortality rates attributable to this condition are quite high. There exists a significant unmet medical need to develop new therapeutic options for patients with bladder cancer.

An effective treatment for bladder cancer would stop or slow tumor growth and/or prevent the spread of the disease into adjacent or distant organs. In patients in whom a curative procedure is impossible, an effective treatment will reduce the incidence or severity of symptoms such as pain, dysuria, frequency, urgency, hematuria and nocturia. If surgical resection of the tumor is attempted, and effective adjuvent therapy will reduce the size of the tumor prior to resection (to make the surgical procedure easier or more effective). Intraoperative placement of the described embodiments during tumor excision surgery can also reduce the incidence of local recurrence of the disease in the postoperative period.

Interstitial brachytherapy is the most common form of local radiotherapy employed in the management of bladder or urethral cancer. Permanent interstitial brachytherapy implants (such as I¹²⁵ seeds, radioactive gold grains, or radioactive radon seeds) are placed directly into the tumor via cystoscope, directly during open surgery, percutaneously inserted via a suprapubic approach, or inserted via the vagina. Temporary (high-dose-rate) brachytherapy implants include radium, cobalt or tantalum needles or iridium wires (typical dose is 14.5-29 μGy/hr). Temporary interstitial implants are usually placed percutaneously or transvaginally, but can also be placed during open surgery. Interstitial embodiments suitable for the treatment of bladder cancer include:

1. Cell Cycle Inhibitor-Loaded Spacers

2. Cell Cycle Inhibitor-Coated Radioactive Seeds

3. Cell Cycle Inhibitor-Coated Radioactive Sutures

4. Cell Cycle Inhibitor-Loaded Radioactive Sutures

5. Interstitial Injection of Cell Cycle Inhibitors

6. Cell Cycle Inhibitor-Coated Radioactive Wires

In one embodiment, a cycle inhibitor is loaded into a resorbable [(e.g., poly (glycolide), poly (lactide-co-glycolide), poly (glycolide-co-caprolactone), albumin, hyaluronic acid, gelatin, and/or Carbopol)] or nonresorbable [(e.g., polypropylene, silicone, EVA, polyurethane, and/or polyethylene] polymer(s) and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted until the entire bladder tumor is implanted with needles 0.5 to 1.0 cm apart. Although any cell cycle inhibitor could be incorporated into a polymeric spacer, taxanes, anthracyclines, antimetabolites, vinca alkaloids, platinum and/or mitomycin-C are preferred. For example, 0.1-40% w/w paclitaxel (by weight) incorporated into a resorbable or non-resorbable polymeric spacer is an ideal embodiment. Docetaxel at 0.1-40% w/w, 0.1-40% w/w thiotepa, 0.1-40% w/w doxorubicin, 0.1-40% w/w methotrexate, 0 1-40% w/w vinblastine, 0.1-40% w/w cisplatin and/or 0.1-40% w/w mitomycin-C are also preferred embodiments. It should be obvious to one of skill in the art that analogues or derivatives of the above compounds (as described previously) given at similar or biologically equivalent dosages would also be suitable for the above invention.

In a second embodiment, a cell cycle inhibitor-coated seed can be utilized. Here the cell cycle inhibitor is coated directly onto the radioactive seed (e.g. I¹²⁵or Pd¹⁰³) either prior to, or at the time of, implantation into the bladder. Once again preferred cell cycle inhibitors include taxanes, ethyleneimine, anthracyclines, antimetabolites, vinca alkaloids, platinum and/or mitomycin-C. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Similarly 0.1-40% w/w thiotepa, 0.1-40% w/w doxorubicin, 0.1-40% w/w methotrexate, 0.1-40% w/w vinblastine, 0.1-40% w/w cisplatin and/or 0.1-40% w/w mitomycin-C can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the bladder via needles or catheters (as described previously) or via specialized applicators.

In a third embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the bladder percutaneously or during open surgery. A cell cycle inhibitor can be loaded into a polymeric carrier applied to the surface of the suture material prior to, or during, implantation. Preferred cell cycle inhibitor for non-absorbable sutures are taxanes, ethyleneimine, anthracyclines, antimetabolites, vinca alkaloids, platinum and/or mitomycin-C loaded into EVA, polyurethane (PU), PLGA, silicone, gelatin, and/or dextran. The polymer-cell cycle inhibitor formulation is then applied as a coating (e.g. sprayed, dipped, “painted” on) prior to insertion in the bladder. Examples of specific, preferred agents include 0.1-40% w/w paclitaxel, 0.1-40% w/w docetaxel, 0.1-40% w/w thiotepa, 0.1-40% w/w doxorubicin, 0.1-40% w/w methotrexate, 0.1-40% w/w vinblastine, 0.1-40% w/w cisplatin and/or 0.1-40% w/w mitomycin-C loaded into one (or a combination of) the above polymers and applied as a coating to a radioactive suture. Conversely, incorporation of the above agents in poly(lactide-co-glycolide), poly(glycolide) and/or dextran would be the preferred coating for absorbable radioactive sutures.

In a fourth embodiment, the cell cycle inhibitor is loaded into a radioactive suture (i.e., the cell cycle inhibitor-polymer composition is a constituent component of the suture). In a preferred embodiment, a taxane, topoisomerase inhibitor, vinca alkaloid and/or estramustine is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵or Pd¹⁰³). Particularly preferred cell cycle inhibitors for this purpose include 0.1-40% w/w paclitaxel, 0.1-40% w/w docetaxel, 0.1-40% w/w thiotepa, 0.1-40% w/w doxorubicin, 0.1-40% w/w methotrexate, 0.1-40% w/w vinblastine, 0.1-40% w/w cisplatin and/or 0.1-40% w/w mitomycin-C. If a nonabsorbable suture is desired, the above agents can be loaded into polypropylene or silicone. In both cases the radioactive source is evenly spaced (e.g. 1 cm apart) within the suture (see FIG. 3).

A fifth embodiment for the treatment of bladder cancer is infiltration of the bladder with interstitial injections of cell cycle inhibitor formulations (aqueous, nanoparticulates, microspheres, pastes, gels, etc.) prior to, or at the time of brachytherapy treatment. Taxanes, anthracyclines, antimetabolites, vinca alkaloids, platinum and/or mitomycin-C compounds are preferred for this embodiment. For example, paclitaxel, docetaxel, thiotepa, doxorubicin, methotrexate, vinblastine, cisplatin and/or mitomycin-C can be incorporated into a polymeric carrier as described previously. The resulting formulation whether aqueous, micro or nanoparticulate, gel, or paste in nature, must be suitable for injection through a needle or catheter. The polymer-cell cycle inhibitor formulation is then injected into the bladder wall (e.g. via cystoscope or percutaneously) such that therapeutic drug levels are reached in the diseased tissues. A brachytherapy source is also administered by any of the methods described previously. While also suitable for use with permanent low dose brachytherapy sources, this treatment form is best suited for use with temporary high dose rate (HDR) brachytherapy.

In a sixth embodiment, a cell cycle inhibitor is coated onto a radioactive wire. In this application, radioactive wires (e.g. Ir¹⁹²) are placed through the tumor via the skin (percutaneously) or during open surgery. If the wire is to remain in place permanently, a variety of polymeric carriers are suitable for administration of the cell cycle inhibitor including EVA, polyurethane and silicone. The cell cycle inhibitor-polymer coating can be applied as a spray or via a dipped coating process either in advance of, or at the time of insertion. A “sheet” of cell cycle inhibitor-polymer material (e.g. EVA or polyurethane) can also be wrapped around the wire prior to insertion. If temporary high dose brachytherapy is employed, the wire must be directly coated with a cell cycle inhibitor or coated with a cell cycle inhibitor loaded into a polymer capable of rapid drug release, such as polyethylene glycol, dextran and/or hyaluronic acid since most of the drug must be released within a 1-2 hour period. Regardless of the form of brachytherapy performed, ideal cell cycle inhibitors for use as wire coatings in the treatment of bladder cancer include taxanes, ethyleneimine, anthracyclines, antimetabolites, vinca alkaloids, platinum and/or mitomycin-C. For example, 0.1-40% w/w paclitaxel, 0.1-40% w/w docetaxel, 0.1-40% w/w thiotepa, 0.1-40% w/w doxorubicin, 0.1-40% w/w methotrexate, 0.1-40% w/w vinblastine, 0.1-40% w/w cisplatin and/or 0.1-40% w/w mitomycin-C can be loaded into fast release polymeric formulations such as polyethylene glycol, dextran and hyaluronic for coating onto temporary HDR brachytherapy wires.

Hyperproliferative Diseases of the Eye

Although relatively rare, ocular tumors can have devastating clinical consequences. Uveal melanoma (1500 new cases per year in the U.S.) and retinoblastoma (300-350 cases per year in the U.S.; primarily children) often require enucleation (removal of the affected eye) to effectively treat the disease. The object of the local therapies described below is to destroy the tumor and while preserving visual acuity. In addition, the non-malignant hyperproliferative eye disease pterygia can also be treated with these embodiments. Pterygia is the growth of proliferative fibrovascular tissue that originates from the canthus and grows towards the limbus and cornea. The tissue is non-transparent and can cause obstruction of vision. Although it can be treated by surgical excision, recurrence following resection is common. Embodiments of the present invention suitable for the treatment of hyperproliferative diseases of the eye include:

1. Surface Eye Molds Containing a Cell Cycle Inhibitor and a Radioactive Source

2. Intravitreal Injection of Cell Cycle Inhibitors

3. Cell Cycle Inhibitor Surgical Pastes, Gels, Films and Sprays.

Eye “plaques” or “molds” have been developed for the delivery of brachytherapy to the eye. For example, eye plaques can be fabricated in gold in the shape of the eye surface. I¹²⁵ seeds are attached to the gold plate, a polymer insert is placed on the inner surface, and the plaque is placed on the eye for 3-5 days. Seed carrier eye inserts are also manufactured by Trachsell Dental Studio Inc. (Rochester, Mass.). These are designed so that the brachytherapy seeds and the sterile surface of the plaque are separated by 1 mm of plastic (called COMS plaques).

In the first embodiment, the plaques or molds can be fabricated with a polymer which releases a cell cycle inhibitor. A “contact lens” structure can be manufactured containing a cell cycle inhibitor and an eye plaque containing a brachytherapy source is placed over top of it as described above. Alternatively, a polymer coating can be applied to the inner surface of an eye mold or plaque which contains regularly spaced (0.5-1.0 cm apart) indentations designed to hold brachytherapy seeds. Typically I¹²⁵ seeds are used, but Pd¹⁰³, Co⁶⁰, Ru¹⁰⁶, Ir¹⁹² and Ru¹⁰⁶/Rh¹⁰⁶ brachytherapy sources can also be administered. Taxanes, vinca alkaloids, alkylating agents, anthracyclines, platinum, nitrogen mustards and/or topoisomerase inhibitors can be incorporated into “fast release” polymers such as dextran which are suitable for application to the surface of the eye. The brachytherapy seeds are then placed in the depressions on the posterior surface of the polymer formulation (i.e., the one in contact with the mold/plaque, not the surface in contact with the eye) prior to placement on the eye. Preferred cell cycle inhibitor formulations include 0.1-40% w/w paclitaxel, 0.1-40% w/w docetaxel, 0.1-40% w/w vincristine, 0.1-40% w/w cyclophosphamide, 0.1-40% w/w doxorubicin, 0.1-40% w/w idarubicin, 0.1-40% w/w carboplatin, 0.1-40% w/w ifosfamide, and/or 0.1-40% w/w etoposide incorporated into the polymers described above. It should be noted that a topical eye drop formulation of a cell cycle inhibitor would also be suitable for use in this embodiment.

In a second embodiment, the cell cycle inhibitor is injected into the vitreous prior to, or at the time of, administration of the brachytherapy with. Intravitreal injections of cell cycle inhibitor formulations (aqueous, nanoparticulates, microspheres, pastes, gels, etc.) containing taxanes, vinca alkaloids, alkylating agents, anthracyclines, platinum, nitrogen mustards and/or topoisomerase inhibitor compounds prior to, or at the time of brachytherapy treatment are preferred embodiments. For example, paclitaxel, docetaxel, vincristine, cyclophosphamide, doxorubicin, idarubicin, carboplatin, ifosfamide, and/or etoposide can be incorporated into a polymeric carrier as described previously. The resulting formulation—whether aqueous, nano or microparticulate, gel, or paste in nature —must be suitable for injection through a needle or catheter. The polymer-cell cycle inhibitor formulation is then injected into the vitreous of the eye such that therapeutic drug levels are reached. A brachytherapy source is also administered either topically (described above) or via injection in the vitreous. While also suitable for use with permanent low dose brachytherapy sources, this treatment form is well suited for use with temporary high dose rate (HDR) brachytherapy

In a third embodiment, a cell cycle inhibitor-loaded surgical paste, gel, film or spray can be used during surgical resection of hyperproliferative tissue. Although useful in cancer surgery, this would be particularly effective in the management of pterygia. Here the cell cycle inhibitor-loaded surgical paste, gel, film or spray is applied to the cut surface of pterygia. A radioactive source is also delivered intraoperatively during resection of the pyterygia. Surgical pastes, gels and films containing taxanes, vinca alkaloids, alkylating agents, anthracyclines, platinum, nitrogen mustards and/or topoisomerase inhibitors are ideally suited for treatment of eye tumor resection beds and pyterygia. In a surgical paste (0.1-40% w/w paclitaxel, 0.1-40% w/w docetaxel, 0.1-40% w/w vincristine, 0.1-40% w/w cyclophosphamide, 0.1-40% w/w doxorubicin, 0.1-40% w/w idarubicin, 0.1-40% w/w carboplatin, 0.1-40% w/w ifosfamide, and/or 0.1-40% w/w etoposide is incorporated into polymeric or non-polymeric paste formulation (refer to examples). The cell cycle inhibitor-loaded paste is injected via a syringe into the resection cavity or the cut surface of the pterygium and spread by the surgeon to cover the desired area. For thermally responsive pastes, as the formulation cools (cold-sensitive) or heats (heat-sensitive) to body temperature (37° C.) it gradually solidifies. During this time interval, radioactive sources (e.g., I¹²⁵ seeds, Pd¹⁰³ seeds) are inserted into the molten formulation in the correct geometry to deliver the desired dosimetry. The paste will then completely harden in the shape of the resection margin while also fixing the radioactive source in place. Alternatively, a particulate radioactive source can be added to the thermopaste or cryopaste prior to administration when precise dosimetry is not required. A gel composed of a cell cycle inhibitor and a brachytherapy source contained in hyaluronic acid can be used in the same manner as described for cryopaste and thermopastes.

Surgical films containing a cell cycle inhibitor and a radioactive source can also be used in the management of eye tumor resection margins and pterygium. Ideal polymeric vehicles for surgical films include flexible non-degradable polymers such as polyurethane, EVA silicone and resorbable polymers such as poly (glycolide), poly (lactide-co-glycolide), poly (glycolide-co-caprolactone), albumin, hyaluronic acid, gelatin, and/or Carbopol. The surface of the film can be modified to hold I¹²⁵, Pd¹⁰³ seeds at regular intervals (see FIG. 9 for a more detailed description). In a preferred embodiment, the surgical film is loaded with taxanes, vinca alkaloids, alkylating agents, anthracyclines, platinum, nitrogen mustards and/or topoisomerase inhibitors. For example, 0.1-40% w/w paclitaxel, 0.1-40% w/w docetaxel, 0.1-40% w/w vincristine, 0.1-40% w/w cyclophosphamide, 0.1-40% w/w doxorubicin, 0.1-40% w/w idarubicin, 0.1-40%W/w carboplatin, 0.1-40% w/w ifosfamide, and/or 0.1-40% w/w etoposide is incorporated in to the film. The radioactive seeds are placed in the film and can be sealed in place with either another piece of cell cycle inhibitor-loaded film or molten polymer containing a cell cycle inhibitor (described above) which hardens in place. The cell cycle inhibitor-loaded film containing the radioactive source is then placed on the resection margin as required.

A surgical spray loaded with a cell cycle inhibitor and a brachytherapy source is also suitable for use in the treatment of eye tumor and pterygium resection margins. For this embodiment, taxanes, vinca alkaloids, alkylating agents, anthracyclines, platinum, nitrogen mustards and/or topoisomerase inhibitors are formulated into an aerosol which also incorporates a radioactive source. In a preferred embodiment, paclitaxel, docetaxel, vincristine, cyclophosphamide, doxorubicin, idarubicin, carboplatin, ifosfamide, and/or etoposide is formulated into an aerosol which also contains an aqueous radioactive source (or microparticulate, such as gold grains). This is sprayed onto the resection margin during interventions to help prevent local recurrence of the disease.

Hyperproliferative Diseases of the Brain

Brachytherapy is used in the management of malignant glioma, astrocytoma, skull base tumors, craniopharyngioma, pediatric tumors and tumors which have metastasized to the brain. Interstitial and surgical paste embodiments of cell cycle inhibitors are ideally suited to this illness due to its clinical course. Malignant gliomas rarely metastasize, therefore, the morbidity and mortality associated with this condition is almost universally due to an inability to control local spread of the disease (approximately 80% of treatment failures occur within 2 cm of the primary tumor). A second consideration is that the treatment of brain tumors requires the administration of relatively high doses of radiotherapy. Thus, the use of local brachytherapy vs. external beam radiotherapy reduces the amount of brain tissue exposed to ionizing radiation (thereby decreasing damage to surrounding normal brain tissue), while the concurrent administration of a cell cycle inhibitor can decrease the dose of radiotherapy required.

An effective therapy for brain tumors would stop or slow tumor growth and/or prevent the spread of the disease into adjacent brain tissue. If surgical resection is attempted, an effective therapy will reduce the local recurrence of the tumor—perhaps the single most important problem in the management of this condition.

Preferred embodiments include:

1. Cell Cycle Inhibitor-Loaded Spacers

2. Cell Cycle Inhibitor-Coated Radioactive Seeds

3. Cell Cycle Inhibitor-Coated Radioactive Sutures

4. Cell Cycle Inhibitor-Loaded Radioactive Sutures

5. Interstitial Injection of Cell Cycle Inhibitors

6. Cell Cycle Inhibitor-Loaded Surgical Pastes, Films, or Sprays

In the interstitial treatment of the brain tumors, a stereotatic base ring is affixed to the patient's skull under local anesthesia. A CT Scan is performed and a treatment plan is developed. Several catheters (usually 2-6) are placed through the skin and skull (the skin is incised under local anesthetic, holes are drilled in the skull) and into the tumor tissue. A template attached to the base ring can be used to assist with proper placement. Radioactive sources (often I¹²⁵) are inserted via the catheters into the tumor to deliver a therapeutic dose (0.4-0.6 Gy/hr).

In one embodiment, a cycle inhibitor is loaded into a resorbable [(e.g., poly (glycolide), poly (lactide-co-glycolide), poly (glycolide-co-caprolactone), albumin, hyaluronic acid, gelatin, and/or Carbopol)] or nonresorbable [(e.g., polypropylene, silicone, EVA, polyurethane, and/or polyethylene] polymers and formed into a cylindrical spacer 1-5 mm in diameter and 0.5 cm or 1.0 cm in length. I²⁵ or Pd¹⁰³ seeds are placed in the catheter and separated from each other by the cell cycle inhibitor-loaded spacers (i.e., seed-spacer-seed-spacer, etc.) of the appropriate length. The needles or catheters are then inserted through a template and into the hyperproliferative tissue in the brain (as described above). Although any cell cycle inhibitor could be incorporated into a polymeric spacer, taxanes, nitrosureas, tetrazine, vinca alkaloids, platinum, topoisomerase inhibitors, antimetabolites, and/or leucovorin are preferred. For example, 0.1-40% w/w paclitaxel (by weight) incorporated into a resorbable or non-resorbable polymeric spacer is an ideal embodiment. Docetaxel at 0.1-40% w/w, 0.1-40% w/w CCNU, 0.1-40% w/w carmustine (BCNU), 0.1-40% w/w procarbazine, 0.1-40% w/w vincristine, 0.1-40% w/w cisplatin, 0.1-40% w/w etoposide, 0.1-40% w/w methotrexate, and/or 0.1-40% w/w leucovorin are also preferred embodiments. It should be obvious to one of skill in the art that analogues or derivatives of the above compounds (as described previously) given at similar or biologically equivalent dosages would also be suitable for the above invention.

In a second embodiment, a cell cycle inhibitor-coated seed can be utilized. Here the cell cycle inhibitor is coated directly onto the radioactive seed (e.g. I¹²⁵or Pd¹⁰³) either prior to, or at the time of, permanent implantation into the brain. Once again preferred cell cycle inhibitors include taxanes, nitrosureas, tetrazine, vinca alkaloids, platinum, topoisomerase inhibitors, antimetabolites, and/or leucovorin. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Similarly 0.1-40% w/w CCNU, 0.1-40% w/w cannustine (BCNU), 0.1-40% w/w procarbazine, 0.1-40% w/w vincristine, 0.1-40% w/w cisplatin, 0.1-40% w/w etoposide, 0.1-40% w/w methotrexate, and/or 0.1-40% w/w leucovorin can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the brain via catheters (as described previously.

In a third embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the brain percutaneously, via catheters or during open surgery. A cell cycle inhibitor can be loaded into a polymeric carrier applied to the surface of the suture material prior to, or during, implantation. Preferred cell cycle inhibitor for non-absorbable sutures are taxanes, nitrosureas, tetrazine, vinca alkaloids, platinum, topoisomerase inhibitors, antimetabolites, and/or leucovorin loaded into EVA, polyurethane (PU) or PLGA silicone, gelatin, and/or dextran. The polymer-cell cycle inhibitor formulation is then applied as a coating (e.g. sprayed, dipped, “painted” on) prior to insertion in the brain. Examples of specific, preferred agents include 0.1-40% w/w paclitaxel, 0.1-40% w/w docetaxel, 0.1-40% w/w CCNU, 0.1-40% w/w carmustine (BCNU), 0.1-40% w/w procarbazine, 0.1-40% w/w vincristine, 0.1-40% w/w cisplatin, 0.1-40% w/w etoposide, 0.1-40% w/w methotrexate, and/or 0.1-40% w/w leucovorin loaded into one (or a combination of) the above polymers and applied as a coating to a radioactive suture. Conversely, incorporation of the above agents in poly(lactide-co-glycolide), poly(glycolide) or dextran would be the preferred coating for absorbable radioactive sutures.

In a fourth embodiment, the cell cycle inhibitor is loaded into a radioactive suture (i.e., the cell cycle inhibitor-polymer composition is a constituent component of the suture) for administration (as described above). In a preferred embodiment, a taxane, nitrosurea, tetrazine, vinca alkaloid, platinum, topoisomerase inhibitor, antimetabolite, and/or leucovorin is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵ or Pd¹⁰³). Particularly, preferred cell cycle inhibitors for this purpose include 0.1-40% w/w paclitaxel, 0.1-40% w/w docetaxel, 0.1-40% w/w CCNU, 0.1-40% w/w carmustine (BCNU), 0.1-40% w/w procarbazine, 0.1-40% w/w vincristine, 0.1-40% w/w cisplatin, 0.1-40% w/w etoposide, 0.1-40% w/w methotrexate, and/or 0.1-40% w/w leucovorin. If a nonabsorbable suture is desired, the above agents can be loaded into polypropylene or silicone. In both cases the radioactive source is evenly spaced (e.g. 1 cm apart) within the suture (see FIG. 3).

A fifth embodiment for the treatment of hyperproliferative diseases of the brain is infiltration of the brain with interstitial injections of cell cycle inhibitor formulations (aqueous, nanoparticulates, microspheres, pastes, gels, etc.) prior to, or at the time of brachytherapy treatment. Taxanes, nitrosureas, tetrazine, vinca alkaloids, platinum, topoisomerase inhibitors, antimetabolites, and/or leucovorin compounds are preferred for this embodiment. For example, 0.1-40% w/w paclitaxel, 0.1-40% w/w docetaxel, 0.1-40% w/w CCNU, 0.1-40% w/w carmustine (BCNU), 0.1-40% w/w procarbazine, 0.1-40% w/w vincristine, 0.1-40% w/w cisplatin, 0.1-40% w/w etoposide, 0.1-40% w/w methotrexate, and/or 0.1-40% w/w leucovorin can be incorporated into a polymeric carrier as described previously. The resulting formulation—whether aqueous, nano or microparticulate, gel, or paste in nature —must be suitable for injection through a catheter. The polymer-cell cycle inhibitor formulation is then injected into the brain via a catheter (as described above) such that therapeutic drug levels are reached in the diseased tissues. A brachytherapy source is also administered interstitially via the catheter.

In a sixth embodiment, the cell cycle inhibitor and the radioactive source are delivered intraoperatively part of tumour resection surgery. Resection of a malignant brain mass is the primary therapeutic option for many patients diagnosed with brain cancer. Unfortunately, for many patients complete removal of the mass is not possible and malignant cells remain in adjacent tissues. To address this problem, a cell cycle inhibitor can be combined with a radioactive source and applied to the surface of the tumor resection margin. Surgical pastes, gels and films containing taxanes, nitrosureas, tetrazine, vinca alkaloids, platinum, topoisomerase inhibitors, antimetabolites and/or leucovorin are ideally suited for treatment of brain tumor resection beds. In a surgical paste, 0.1-40% w/w paclitaxel, 0.1-40% w/w docetaxel, 0.1-40% w/w CCNU, 0.1-40% w/w carmustine (BCNU), 0.1-40% w/w procarbazine, 0.1-40% w/w vincristine, 0.1-40% w/w cisplatin, 0.1-40% w/w etoposide, 0.1-40% w/w methotrexate, and/or 0.1-40% w/w leucovorin is incorporated into polymeric or non-polymeric paste formulation (refer to examples). The cell cycle inhibitor-loaded paste is injected via a syringe into the resection cavity and spread by the surgeon to cover the desired area. For thermally responsive pastes, as the formulation cools (cold-sensitive) or heats (heat-sensitive) to body temperature (37° C.) it gradually solidifies. During this time interval, radioactive sources (e.g., iridium wires, I¹²⁵ seeds, Pd¹⁰³ seeds) are inserted into the molten formulation in the correct geometry to deliver the desired dosimetry. The paste will then completely harden in the shape of the resection margin while also fixing the radioactive source in place. Alternatively, a particulate radioactive source can be added to the thermopaste or cryopaste prior to administration when precise dosimetry is not required. A gel composed of a cell cycle inhibitor and a brachytherapy source contained in hyaluronic acid can be used in the same manner as described for cryopaste and thermopastes.

Surgical films containing a cell cycle inhibitor and a radioactive source can also be used in the management of brain tumor resection margins. Ideal polymeric vehicles for surgical films include flexible non-degradable polymers such as polyurethane, EVA, silicone and resorbable polymers such as poly (glycolide), poly (lactide-co-glycolide), poly (glycolide-co-caprolactone), albumin, hyaluronic acid, gelatin, and/or Carbopol. The surface of the film can be modified to hold I¹²⁵, Pd¹⁰³ seeds at regular intervals (see FIG. 9). In a preferred embodiment, the surgical film is loaded with a taxane, topoisomerase inhibitor, vinca alkaloid and/or estramustine.

For example, 0.1-40% w/w paclitaxel, 0.1-40 w/w docetaxel, 0.1-40% w/w CCNU, 0.1-40% w/w carmustine (BCNU), 0.1-40% w/w procarbazine, 0.1-40% w/w vincristine, 0.1-40% w/w cisplatin, 0.1-40% w/w etoposide, 0.1-40% w/w methotrexate, and/or 0.1-40% w/w leucovorin is incorporated into the film. The radioactive seeds or wires are placed in the film and can be sealed in place with either another piece of cell cycle inhibitor-loaded film or molten polymer containing a cell cycle inhibitor (described above) which hardens in place. The cell cycle inhibitor-loaded film containing the radioactive source is then placed in the resection cavity as required.

A surgical spray loaded with a cell cycle inhibitor and a brachytherapy source is also suitable for use in the treatment of brain tumor resection margins. For this embodiment, taxanes, nitrosureas, tetrazine, vinca alkaloids, platinum, topoisomerase inhibitors, antimetabolites and/or leucovorin are formulated into an aerosol into which a radioactive source is incorporated. In a preferred embodiment, paclitaxel, docetaxel, CCNU, carmustine (BCNU), procarbazine, vincristine, cisplatin, etoposide, methotrexate, and/or leucovorin is formulated into an aerosol that also contains an aqueous radioactive source (or microparticulate such as gold grains). This is sprayed onto the resection margin during open or endoscopic surgery interventions to help prevent tumor recurrence.

Hyperproliferative Diseases of the Breast

Breast cancer is one of the most common malignancies in women affecting close to 1 in 10 women in their lifetime. Although many new treatments have been developed, the morbidity and mortality associated with this disease remains high and more effective therapies need to be made available.

Lumpectomy, with or without adjunct external beam radiotherapy, is widely accepted as the primary therapeutic modality for most breast cancer patients. However, in many patients, the tumor is incompletely removed during surgery and the patient is at high risk for local or metastatic recurrence of their disease. For many patients, the risk of local recurrence of their breast cancer is related to gross, microscopic, or occult tumor tissue remaining in adjacent breast tissue and lymph nodes after lumpectomy. Interstitial brachytherapy has been used clinically in patients who are at high risk for local recurrence.

An effective cell cycle inhibitor and brachytherapy treatment would stop or slow breast tumor growth, prevent the spread of the disease into the adjacent or distant tissues and/or reduce the rate of local or metastatic recurrence of the disease.

Implantation of low-dose-rate (LDR) interstitial brachytherapy (typically utilizing Ir¹⁹² or I¹²⁵) is used in the management of breast cancer patients. The brachytherapy source can be implanted directly during lumpectomy surgery or percutaneously in the post-operative period (usually 7-10 days after the lumpectomy). Stainless steel trocars (17 g) are inserted into the breast tissue intraoperatively or percutaneously (with or without use of a template) at 1.0 to 1.5 cm intervals. Afterloading tubes are pulled through the breast as the trocars are removed and are used to deliver the radioactive source.

For breast cancer, ideal therapeutic embodiments are interstitial treatments and surgical implants including:

1. Cell Cycle Inhibitor-Loaded Spacers

2. Cell Cycle Inhibitor-Coated Radioactive Seeds

3. Cell Cycle Inhibitor-Coated Radioactive Sutures

4. Cell Cycle Inhibitor-Loaded Radioactive Sutures

5. Interstitial Injection of Cell Cycle Inhibitors

6. Cell Cycle Inhibitor-Coated Radioactive Wires

7. Cell Cycle Inhibitor-Loaded Surgical Pastes, Films, or Sprays

In one embodiment, a cycle inhibitor is loaded into a resorbable [(e.g., poly (glycolide), poly (lactide-co-glycolide), poly (glycolide-co-caprolactone), albumin, hyaluronic acid, gelatin, and/or Carbopol)] or nonresorbable [(e.g., polypropylene, silicone, EVA, polyurethane, and/or polyethylene] polymers and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted through a template and into the breast (as described above). Although any cell cycle inhibitor could be utilized, taxanes, anthracyclines, alkylating agents, antimetabolites, vinca alkaloids, platinum, nitrogen mustards, gemcitabine, and/or mitomycin-C are preferred. For example, 0.1-40% w/w paclitaxel (by weight) incorporated into a resorbable or non-resorbable polymeric spacer is an ideal embodiment. Docetaxel at 0.1-40% w/w, 0.1-40% w/w doxorubicin, 0.1-40% w/w epirubicin, 0.1-40% w/w mitoxantrone, 0.1-40% w/w cyclophosphamide, 0.1-40% w/w 5-FU, 0.1-40% w/w capecitabine, 0.1-40% w/w methotrexate, 0.1-40% w/w vinorelbine, 0.1-40% w/w vinblastine, 0.1-40% w/w vincristine, 0.1-40% w/w carboplatinum, 0.1-40% w/w cisplatin, 0.1-40% w/w gemcitabine, 0.1-40% w/w mitomycin-C, 0.1-40% w/w ifosfamide, and/or 0.1-40% w/w melphalan are also preferred embodiments. It should be obvious to one of skill in the art that analogues or derivatives of the above compounds (as described previously) given at similar or biologically equivalent dosages would also be suitable for the above invention.

In a second embodiment, a cell cycle inhibitor-coated seed can be utilized. Here the cell cycle inhibitor is coated directly onto the radioactive seed (e.g. I¹²⁵or Pd¹⁰³) either prior to, or at the time of, implantation into the breast. Once again preferred cell cycle inhibitors include taxanes, anthracyclines, alkylating agents, antimetabolites, vinca alkaloids, platinum, nitrogen mustards, gemcitabine, and/or mitomycin-C. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Similarly 0.1-40% w/w doxorubicin, 0.1-40% w/w epirubicin, 0.1-40% w/w mitoxantrone, 0.1-40% w/w cyclophosphamide, 0.1-40% w/w 5-FU, 0.1-40% w/w capecitabine, 0.1-40% w/w methotrexate, 0.1-40% w/w vinorelbine, 0.1-40% w/w vinblastine, 0.1-40% w/w vincristine, 0.1-40% w/w carboplatinum, 0.1-40% w/w cisplatin, 0.1-40% w/w gemcitabine, 0.1-40% w/w mitomycin-C, 0.1-40% w/w ifosfamide, and/or 0.1-40% w/w melphalan can be incorporated into poly (glycolide), poly (lactide-co-glycolide), poly (glycolide -co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the breast via needles or catheters (as described previously) or via specialized applicators.

In a third embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the breast percutaneously or during open surgery. A cell cycle inhibitor can be loaded into a polymeric carrier applied to the surface of the suture material prior to, or during, implantation. Preferred cell cycle inhibitor for non-absorbable sutures are taxanes, anthracyclines, alkylating agents, antimetabolites, vinca alkaloids, platinum, nitrogen mustards, gemcitabine, and/or mitomycin-C loaded into EVA, polyurethane (PU), PLGA silicone, gelatin, and/or dextran. The polymer-cell inhibitor formulation is then applied as a coating (e.g. sprayed, dipped, “painted” on) prior to insertion in the breast. Examples of specific, preferred agents include 0.1-40% w/w paclitaxel, 0.1-40% w/w docetaxel, 0.1-40% w/w doxorubicin, 0.1-40% w/w epirubicin, 0.1-40% w/w mitoxantrone, 0.1-40% w/w cyclophosphamide, 0.1-40% w/w, 5-FU, 0.1-40% w/w capecitabine, 0.1-40% w/w methotrexate, 0.1-40% w/w vinorelbine, 0.1-40% w/w vinblastine, 0.1-40% w/w vincristine, 0.1-40% w/w carboplatinum, 0.1-40% w/w cisplatin, 0.1-40% w/w gemcitabine, 0.1-40% w/w mitomycin-C, 0.1-40% w/w ifosfamide, and/or 0.1-40% w/w melphalan loaded into one (or a combination of) the above polymers and applied as a coating to a radioactive suture. Conversely, incorporation of the above agents in poly(lactide-co-glycolide), poly(glycolide) and/or dextran would be the preferred coating for absorbable radioactive sutures.

In a fourth embodiment, the cell cycle inhibitor is loaded into a radioactive suture (i.e., the cell cycle inhibitor—polymer composition is a constituent component of the suture). In a preferred embodiment, a taxane, anthracycline, alkylating agent, antimetabolite, vinca alkaloid, platinum, nitrogen mustard, gemcitabine and/or mitomycin-C is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I ¹²⁵or Pdl⁰³). Particularly, preferred cell cycle inhibitors for this purpose include 0.1-40% w/w paclitaxel, 0.1-40% w/w docetaxel, 0.1-40% w/w doxorubicin, 0.1-40% w/w epirubicin, 0.1-40% w/w mitoxantrone, 0.1-40% w/w cyclophosphamide, 0.1-40% w/w 5-FU, 0.1-40% w/w capecitabine, 0.1-40% w/w methotrexate, 0.1-40% w/w vinorelbine, 0.1-40% w/w vinblastine, 0.1-40% w/w vincristine, 0.1-40% w/w carboplatinum, 0.1-40% w/w cisplatin, 0.1-40% w/w gemcitabine, 0.1-40% w/w mitomycin-C, 0.1-40% w/w ifosfamide, and/or 0.1-40% w/w melphalan. If a nonabsorbable suture is desired, the above agents can be loaded into polypropylene or silicone. In both cases the radioactive source is evenly spaced (e.g 1 cm apart) within the suture (see FIG. 3).

A fifth embodiment for the treatment of breast cancer is infiltration of the breast with interstitial injections of cell cycle inhibitor formulations (aqueous, nanoparticulates, microspheres, pastes, gels, etc.) prior to, or at the time of brachytherapy treatment. Taxanes, anthracyclines, alkylating agents, antimetabolites, vinca alkaloids, platinum, nitrogen mustards, gemcitabine, and/or mitomycin-C compounds are preferred for this embodiment. For example, paclitaxel, docetaxel, doxorubicin, epirubicin, mitoxantrone, cyclophosphamide, 5-FU, capecitabine, methotrexate, vinorelbine, vinblastine, vincristine, carboplatinum, cisplatin, gemcitabine, mitomycin-C, ifosfamide, and/or melphalan can be incorporated into a polymeric carrier as described previously. The resulting formulation—whether aqueous, nano or microparticulate, gel, or paste in nature —must be suitable for injection through a needle or catheter. The polymer-cell cycle inhibitor formulation is then injected into the breast gland such that therapeutic drug levels are reached in the diseased tissues. A brachytherapy source is also administered interstitially by the methods described previously. While also suitable for use with permanent low dose brachytherapy sources, this treatment form is best suited for use with temporary high dose rate (HDR) brachytherapy. For example, the breast can be infiltrated by interstitial injection of the cell cycle inhibitor in combination with high energy I¹⁹² wires, which remain in place for 50-80 minutes before being removed. Interstitial injection of the cell cycle inhibitor is ideal for HDR therapy since, unlike some of the other interstitial embodiments, it does not require attachment of the cell cycle inhibitor to the brachytherapy source—important since the brachytherapy source is ultimately removed in HDR.

In a sixth embodiment, a cell cycle inhibitor is coated onto a radioactive wire. In this application, radioactive wires (e.g. Ir¹⁹²) are placed through the tumor via the skin (percutaneously) or during open surgery. Since temporary high dose brachytherapy is employed, the wire must be directly coated with a cell cycle inhibitor (i.e., the drug is directly attached to, or dried on to the wire surface) or the cell cycle inhibitor must be loaded into a polymer capable of rapid drug release, such as polyethylene glycol, dextran and/or hyaluronic acid since most of the drug must be released within a 1-2 hour period. Ideal cell cycle inhibitors for use as wire coatings in the treatment of hyperproliferative diseases of the breast include taxanes, anthracyclines, alkylating agents, antimetabolites, vinca alkaloids, platinum, nitrogen mustards, gemcitabine and/or mitomycin-C. For example, 0.1-40% w/w paclitaxel, 0.1-40% w/w docetaxel, 0.1-40% w/w doxorubicin, 0.1-40% w/w epirubicin, 0.1-40% w/w mitoxantrone, 0.1-40% w/w cyclophosphamide, 0.1-40% w/w 5-FU, 0.1-40% w/w capecitabine, 0.1-40% w/w methotrexate, 0.1-40% w/w vinorelbine, 0.1-40% w/w vinblastine, 0.1-40% w/w vincristine, 0.1-40% w/w carboplatinum, 0.1-40% w/w cisplatin, 0.1-40% w/w gemcitabine, 0.1-40% w/w mitomycin-C, 0.1-40% w/w ifosfamide, and/or 0.1-40% w/w melphalan can be loaded into fast release polymeric formulations such as polyethylene glycol, dextran and hyaluronic for coating onto temporary HDR brachytherapy wires.

In a seventh embodiment, the cell cycle inhibitor and the radioactive source are delivered intraoperatively as part of tumour resection surgery lumpectomy. Resection of a malignant breast mass is the primary therapeutic option for many patients diagnosed with breast cancer. Unfortunately, for many patients complete removal of the mass is not possible and malignant cells remain in adjacent tissues. To address this problem, a cell cycle inhibitor can be combined with a radioactive source and applied to the surface of the tumor resection margin. Surgical pastes, gels and films containing taxanes, anthracyclines, alkylating agents, antimetabolites, vinca alkaloids, platinum, nitrogen mustards, gemcitabine and/or mitomycin-C are ideally suited for treatment of breast tumor resection beds. In a surgical paste, 0.1-40% w/w paclitaxel, 0.1-40% w/w docetaxel, 0.1-40% w/w doxorubicin, 0.1-40% w/w epirubicin, 0.1-40% w/w mitoxantrone, 0.1-40% w/w cyclophosphamide, 0.1-40% w/w 5-FU, 0.1-40% w/w capecitabine, 0.1-40% w/w methotrexate, 0.1-40% w/w vinorelbine, 0.1-40% w/w vinblastine, 0.1-40% w/w vincristine, 0.1-40% w/w carboplatinum, 0.1-40% w/w cisplatin, 0.1-40% w/w gemcitabine, 0.1-40% w/w mitomycin-C, 0.1-40% w/w ifosfamide, and/or 0.1-40% w/w melphalan is incorporated into polymeric or non-polymeric paste formulation (refer to examples). The cell cycle inhibitor-loaded paste is injected via a syringe into the resection cavity and spread by the surgeon to cover the desired area. For thermally responsive pastes, as the formulation cools (cold-sensitive) or heats (heat-sensitive) to body temperature (37° C.) it gradually solidifies. During this time interval, radioactive sources (e.g., I¹²⁵ seeds, Pd¹⁰³ seeds) are inserted into the molten formulation in the correct geometry to deliver the desired dosimetry. The paste will then completely harden in the shape of the resection margin while also fixing the radioactive source in place. Alternatively, a particulate radioactive source can be added to the thermopaste or cryopaste prior to administration when precise dosimetry is not required. A gel composed of a cell cycle inhibitor and a brachytherapy source contained in hyaluronic acid can be used in the same manner as described for cryopaste and thermopastes.

Surgical films containing a cell cycle inhibitor and a radioactive source can also be used in the management of breast tumor resection margins. Ideal polymeric vehicles for surgical films include flexible non-degradable polymers such as polyurethane, EVA silicone and resorbable polymers such as poly (glycolide), poly (lactide-co-glycolide), poly (glycolide-co-caprolactone), albumin, hyaluronic acid, gelatin, and/or Carbopol. The surface of the film can be modified to hold I¹²⁵, Pd¹⁰³ seeds at regular intervals (see FIG. 9 for a more detailed description). In a preferred embodiment, the surgical film is loaded with a taxane, anthracycline, alkylating agent, antimetabolite, vinca alkaloid, platinum, nitrogen mustard, gemcitabine and/or mitomycin-C. For example, 0.1-40% w/w paclitaxel, 0.1-40 w/w docetaxel, 0.1-40% w/w doxorubicin, 0.1-40% w/w epirubicin, 0.1-40% w/w mitoxantrone, 0.1-40% w/w cyclophosphamide, 0.1-40% w/w 5-FU, 0.1-40% w/w capecitabine, 0.1-40% w/w methotrexate, 0.1-40% w/w vinorelbine, 0.1-40% w/w vinblastine, 0.1-40% w/w vincristine, 0.1-40% w/w carboplatinum, 0.1-40% w/w, cisplatin, 0.1-40% w/w gemcitabine, 0.1-40% w/w mitomycin-C, 0.1-40% w/w ifosfamide, and/or 0.1-40% w/w melphalan is incorporated in to the film. The radioactive seeds or wires are placed in the film and can be sealed in place with either another piece of cell cycle inhibitor-loaded film or molten polymer containing a cell cycle inhibitor (described above) which hardens in place. The cell cycle inhibitor-loaded film containing the radioactive source is then placed in the resection cavity as required.

A surgical spray loaded with a cell cycle inhibitor and a brachytherapy source is also suitable for use in the treatment of breast tumor resection margins. For this embodiment, taxanes, anthracyclines, alkylating agents, antimetabolites, vinca alkaloids, platinum, nitrogen mustards, gemcitabine and/or mitomycin-C are formulated into an aerosol into which a radioactive source is incorporated. In a preferred embodiment, paclitaxel, docetaxel, doxorubicin, epirubicin, mitoxantrone, cyclophosphamide, 5-FU, capecitabine, methotrexate, vinorelbine, vinblastine, vincristine, carboplatinum, cisplatin, gemcitabine, and/or mitomycin-C, ifosfamide, and/or is formulated into an aerosol which also contains an aqueous radioactive source (or microparticulate such as gold grains). This is sprayed onto the resection margin during surgical interventions to help prevent tumor recurrence.

Hyperproliferative Diseases of the Esophagus

Esophageal cancer is a particularly difficult tumor to treat and most patients have very poor 5-year survival rates. Esophageal tumors are well suited for treatment with the present inventions for several reasons. First, they are easily accessible via minimally invasive techniques such as endoscopy. Secondly, local and regional tumor control is a significant clinical problem. In one study, it was estimated that 74% of patients died as a result of local and regional tumor effects, while only 18% of patients died due to metastatic spread of the disease. Therefore, the embodiments described below which are designed to improve local control of the disease, are particularly useful clinically.

An effective therapy for esophageal cancer would reduce or inhibit tumor growth and decrease local and metastatic spread of the disease. Effective local tumor control can also result in decreased patient morbidity by improving pain, dysphagia, reflux, emesis and hematemesis.

Endoscopically delivered therapies are particularly useful in the management of esophageal cancer, including:

1. Cell Cycle Inhibitor-Coated Radioactive Stents, and

2. Delivery of Cell Cycle Inhibitors via Drug-Delivery Balloons or

Catheters

The first embodiment, a cell cycle inhibitor is coated onto a radioactive stent (see, e.g., EPA 857470; EPA 810004; EPA 722702; EPA 539165; EPA 497495; EPB 433011; U.S. Pat. Nos. 5,919,216; 5,873,811; 5,871,437; 5,843,163; 5,840,009; 5,730,698; 5,722,984; 5,674,177; 5,653,736; 5,354,257; 5,213,561; 5,183,455; 5,176,617; 5,059,166; 4,976,680; WO 99/42177; WO 99/39765; WO 99/29354; WO 99/22670; WO 99/03536; WO 99/02195; WO 99/02194; and WO 98/48851). A cell cycle inhibitor-coated radioactive stent can be endoscopically implanted in the esophagus for treatment of malignant obstruction of the esophagus. Briefly, a catheter is advanced across the obstruction under or endoscopic guidance, a balloon is inflated to dilate the obstruction, and a stent is deployed (either balloon expanded or self expanded). Radioactive isotopes, such as p³², Au¹⁹⁸, Ir¹⁹², Co⁶⁰, I¹²⁵ and Pd 103 are contained within the stent to provide a source of radioactivity. A cell cycle inhibitor is linked to the surface of the stent, incorporated into a polymeric carrier applied to the surface of the stent (or as a “sleeve” which surrounds the stent), or is incorporated into the stent material itself. Cell cycle inhibitors ideally suited to this embodiment include taxanes, alkylating agents, platinum and/or mitomycin-C. For example, 0.01- 10% w/w paclitaxel, 0.01-10% w/w docetaxel, 0.01-10% w/w 5-Fluorouracil, 0.01 - 10% w/w cisplatin, and/or 0.01-10% w/w mitomycin-C can be incorporated into silicone, polyurethane and/or EVA, which is applied as a coating to the radioactive stent. Alternatively, 10 mg-500 mg paclitaxel, 10 mg-500 mg docetaxel, 10 mg-500 mg 5-Fluorouracil, 10 mg-500 mg cisplatin, and/or 10 mg-500 mg mitomycin-C in a crystalline form can be dried onto the surface of the stent. A polymeric coating may be applied over the cell cycle inhibitor to help control the release of the agent into the surrounding tissue. A third alternative is to incorporate, 1-30% w/w paclitaxel, 1-30% w/w docetaxel, 1-30% w/w 5-Fluorouracil, 1-30% w/w cisplatin, and/or 1-30% w/w mitomycin-C into a polymer (U.S. Pat. Nos. 5,762,625; 5,670,161; WO 95/26762; EPA 420541; U.S. Pat. Nos. 5,464,450; 5,551,954) which comprises part of the stent's structure. For example, the cell cycle inhibitor can be incorporated into a polymer such as poly (lactide-co-caprolactone), polyurethane, and/or polylactic acid in combination with a radioactive source (e.g. I¹²⁵, P³²) prior to solidification as part of the casting and manufacturing of the stent. A final alternative involves delivering the brachytherapy source via a catheter (e.g. Beta-Cath®, RadioCath®, etc.) while the cell cycle inhibitor is delivered via the stent.

In the second embodiment, the cell cycle inhibitor is delivered via specialized balloons (e.g. Transport®; Crescendo®, Channel®; EPA 904799; EPA 904798; EPA 879614; EPA 858815; EPA 853957; EPA 829271; EPA 325836; EPA 311458; EPB 805703; U.S. Pat. Nos. 5,913,813; 5,882,290; 5,879,282; 5,863,285; WO 99/32192; WO 99/15225; WO 99/04856; WO 98/47309; WO 98/39062; WO 97/40889) or delivery catheters (EPA 832670; U.S. Pat. Nos. 5,938,582; 5,916,143; 5,899,882; 5,891,091; 5,851,171; 5,840,008; 5,816,999; 5,803,895; 5,782,740; 5,720,717; 5,653,683; 5,618,266; 5,540,659; 5,267,960; 5,199,939; 4,998,932; 4,963,128; 4,862,887; 4,588,395; WO 99/42162; WO 99/42149; WO 99/40974; WO 99/40973; WO 99/40972; WO 99/40971; WO 99/40962; WO 99/29370; WO 99/24116; WO 99/22815; WO 98/36790; WO 97/48452). Here a cell cycle inhibitor formulated into an aqueous, non-aqueous, nanoparticulate, microsphere and/or gel formulation can be delivered by such a device. Preferred cell cycle inhibitors include taxanes (e.g. paclitaxel, docetaxel), alkylating agents, platinum and/or mitomycin-C at appropriate therapeutic doses. The brachytherapy is delivered via the catheter, balloon or stent.

Genital Tract Tumors

Genital tract tumors include cancer of the penis in men and vaginal cancer in women. Although both conditions are relatively uncommon, embodiments described below would be suitable for treating these conditions.

An effective therapy for the treatment of genital tract tumors would stop or slow tumor growth and/or prevent the spread of the disease into adjacent or distant organs. In patients undergoing surgical resection of the tumorous mass, an effective embodiment would reduce the incidence of local recurrence of the disease in adjacent tissues. In patients in whom a complete response is not possible, an effective treatment will reduce the morbidity associated with their illness by decreasing symptoms such as pain, bleeding, dysuria, fistula formation with adjacent organs (e.g. rectovaginal fistulas, vesicovaginal fistulas), and pain with intercourse. Ideally, an effective therapy will eliminate the need for surgery or limit the amount of surgical resection required in order to preserve fertility and/or sexual function.

Interstitial therapy is commonly employed in cancer of the penis. The most common form of brachytherapy is Ir¹⁹² wires inserted percutaneously to deliver 60-70 Gy over a 4 to 8 day period.

Both interstitial and intracavitary brachytherapy are used in the management of vaginal cancer. Typically 6000 cGy (1000 cGy/day) is administered intravaginally (for a more detailed description see “Hyperproliferative Diseases of the Uterus”); the vagina is filled with a vaginal cylinder and a brachytherapy source is inserted (Cs¹³⁷, Ir¹⁹²). In more advanced disease intravaginal brachytherapy is supplemented with interstitial brachytherapy (i. e., catheters are inserted percutaneously across the perineum using a perineal template).

Interstitial and intracavitary therapies useful for the treatment of genital tract tumors include:

1. Cell Cycle Inhibitor-Loaded Spacers

2. Cell Cycle Inhibitor-Coated Radioactive Seeds

3. Cell Cycle Inhibitor-Coated Radioactive Sutures

4. Cell Cycle Inhibitor-Loaded Radioactive Sutures

5. Interstitial Injection of Cell Cycle Inhibitors

6. Cell Cycle Inhibitor-Coated Radioactive Wires

7. Cell Cycle Inhibitor-Loaded Surgical Pastes, Films, or Sprays

In one embodiment, a cycle inhibitor is loaded into a resorbable [(e.g., poly (glycolide), poly (lactide-co-glycolide), poly (glycolide-co-caprolactone), albumin, hyaluronic acid, gelatin, and/or Carbopol)] or nonresorbable [(e.g., polypropylene, silicone, EVA, polyurethane, and/or polyethylene] polymers and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted through a template and into the tumor. Under general or spinal anesthesia, a template is placed over the perineum (e.g. Syed-Neblett Template, Martinez Universal Perineal Interstitial Template) and needles/catheters are inserted under ultrasound or fluoroscopic guidance until the entire tumor is implanted with needles 0.5 to 1.0 cm apart. Although any cell cycle inhibitor could be incorporated into a polymeric spacer, taxanes, vinca alkaloids, antimetabolites, platinum and/or alkylating agents are preferred. For example, 0.1-40% w/w paclitaxel (by weight) incorporated into a resorbable or non-resorbable polymeric spacer is an ideal embodiment. Docetaxel at 0.1-40% w/w, 0.1-40% w/w vincristine, 0.1-40% w/w methotrexate, 0.1-40% w/w cisplatin, and/or 0.1-40% w/w 5-FU are also preferred embodiments. It should be obvious to one of skill in the art that analogues or derivatives of the above compounds (as described previously) given at similar or biologically equivalent dosages would also be suitable for the above invention.

In a second embodiment, a cell cycle inhibitor-coated seed can be utilized. Here the cell cycle inhibitor is coated directly onto the radioactive seed (e.g. I¹²⁵or Pd¹⁰³) either prior to, or at the time of, implantation into the genital tract tumor. Once again preferred cell cycle inhibitors include taxanes, vinca alkaloids, antimetabolites, platinum and/or alkylating agents. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Similarly, 0.1-40% w/w vincristine, 0.1-40% w/w methotrexate, 0.1-40% w/w cisplatin, and/or 0.1-40% w/w 5-FU can be incorporated into poly (glycolide), poly (lactide-co-glycolide), poly (glycolide -co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the genital tract tumor via needles or catheters (as described previously) or via specialized applicators (e.g. Mick Applicator). The Mick Applicator, for example, can implant cell cycle inhibitor-coated seeds at 1 cm intervals in the genital tract tumors and their position can be verified by fluoroscopy.

In a third embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the genital tract tumor percutaneously or during open surgery. A cell cycle inhibitor can be loaded into a polymeric carrier applied to the surface of the suture material prior to, or during, implantation. Preferred cell cycle inhibitors for non-absorbable sutures are taxanes, vinca alkaloids, antimetabolites, platinum and/or alkylating agents loaded into EVA, polyurethane (PU), PLGA, silicone, gelatin, and/or dextran. The polymer-cell cycle inhibitor formulation is then applied as a coating (e.g. sprayed, dipped, “painted” on) prior to insertion in the genital tract tumors. Examples of specific, preferred agents include 0.1-40% w/w paclitaxel, 0.1-40% w/w docetaxel, 0.1-40% w/w vincristine, 0.1-40% w/w methotrexate, 0.1-40% w/w cisplatin, and/or 0.1-40% w/w 5-FU loaded into one (or a combination of) the above polymers and applied as a coating to a radioactive suture. Conversely, incorporation of the above agents in poly(lactide-co-glycolide), poly(glycolide) and/or dextran would be the preferred coating for absorbable radioactive sutures.

In a fourth embodiment, the cell cycle inhibitor is loaded into a radioactive suture (i.e., the cell cycle inhibitor-polymer composition is a constituent component of the suture). In a preferred embodiment, a taxane, vinca alkaloid, antimetabolite, platinum and/or alkylating agent loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵ or Pd¹⁰³). Particularly, preferred cell cycle inhibitors for this purpose include 0.1-40% w/w paclitaxel, 0.1-40% w/w docetaxel, 0.1-40% w/w vincristine, 0.1-40% w/w methotrexate, 0.1-40% w/w cisplatin, and/or 0.1-40% w/w 5-FU. If a nonabsorbable suture is desired, the above agents can be loaded into polypropylene or silicone. In both cases the radioactive source is evenly spaced (e.g. 1 cm apart) within the suture (see FIG. 3).

A fifth embodiment for the treatment of genital tract tumors is infiltration of the tumor with interstitial injections of cell cycle inhibitor formulations (aqueous, nanoparticulates, microspheres, pastes, gels, etc.) prior to, or at the time of brachytherapy treatment. Taxanes, vinca alkaloids, antimetabolites, platinum and/or alkylating agents are preferred for this embodiment. For example, paclitaxel, docetaxel, vincristine, methotrexate, cisplatin, and/or 5-FU can be incorporated into a polymeric carrier as described previously. The resulting formulation—whether aqueous, nano or microparticulate, gel, or paste in nature—must be suitable for injection through a needle or catheter. The polymer-cell cycle inhibitor formulation is then injected into the tumor such that therapeutic drug levels are reached in the diseased tissues. A brachytherapy source is also administered interstitially or intracavitarily by any of the methods described previously. While also suitable for use with permanent low dose brachytherapy sources, this treatment form is best suited for use with temporary high dose rate (HDR) brachytherapy. For example, the genital tract tumors can be infiltrated by interstitial injection of the cell cycle inhibitor in combination with high energy I¹⁹², administered via a template or intravaginally, which remains in place for 50-80 minutes before being removed. Interstitial injection of the cell cycle inhibitor is ideal for HDR therapy since, unlike some of the other interstitial embodiments, it does not require attachment of the cell cycle inhibitor to the brachytherapy source - important since the brachytherapy source is ultimately removed in HDR.

In a sixth embodiment, a cell cycle inhibitor is coated onto a radioactive wire. In this application, radioactive wires (e.g. Ir¹⁹²) are placed through the tumor via the skin (percutaneously), transvaginally, or during open surgery. The cell cycle inhibitor-polymer coating can be applied as a spray or via a dipped coating process either in advance of or at the time of insertion. A “sheet” of cell cycle inhibitor-polymer material (e.g. EVA, Polyurethane) can also be wrapped around the wire prior to insertion. In temporary high dose brachytherapy, the wire must be directly coated with a cell cycle inhibitor (i.e., dried on to the surface of the wire or attached to the wire without a carrier) or the cell cycle inhibitor can be loaded into a polymer capable of rapid drug release, such as polyethylene glycol, dextran and/or hyaluronic acid since most of the drug must be released within a 1-2 hour period. Ideal cell cycle inhibitors for use as wire coatings in the treatment of genital tract tumors include taxanes, vinca alkaloids, antimetabolites, platinum and/or alkylating agents. For example, 0.1-40% w/w paclitaxel, 0.1-40% w/w docetaxel, 0.1-40% w/w vincristine, 0.1-40% w/w methotrexate, 0.1-40% w/w cisplatin, and/or 0.1-40% w/w 5-FU can be loaded into fast release polymeric formulations such as polyethylene glycol, dextran and/or hyaluronic acid for coating onto temporary HDR brachytherapy wires.

In a seventh embodiment, the cell cycle inhibitor and the radioactive source are delivered intraoperatively part of tumour resection surgery. Resection of a malignant genital tract tumor is the primary therapeutic option for many patients. Unfortunately, for many patients complete removal of the mass is not possible and malignant cells remain. in adjacent tissues. To address this problem, a cell cycle inhibitor can be combined with a radioactive source and applied to the surface of the tumor resection margin. Surgical pastes, gels and films containing taxanes, vinca alkaloids, antimetabolites, platinum and/or alkylating agents are ideally suited for treatment of genital tract tumor resection beds. In a surgical paste, 0.1-40% w/w paclitaxel, 0.1-40% w/w docetaxel, 0.1-40% w/w vincristine, 0.1-40% w/w methotrexate, 0.1-40% w/w cisplatin, and/or 0.1-40% w/w 5-FU is incorporated into polymeric or non-polymeric paste formulations (refer to examples). The cell cycle inhibitor-loaded paste is injected via a syringe into the resection cavity and spread by the surgeon to cover the desired area. For thermally responsive pastes, as the formulation cools (cold-sensitive) or heats (heat-sensitive) to body temperature (37° C.) it gradually solidifies. During this time interval, radioactive sources (e.g., iridium wires, I¹²⁵ seeds, Pd¹⁰³ seeds) are inserted into the molten formulation in the correct geometry to deliver the desired dosimetry. The paste will then completely harden in the shape of the resection margin while also fixing the radioactive source in place. Alternatively, a particulate radioactive source can be added to the thermopaste or cryopaste prior to administration when precise dosimetry is not required. A gel composed of a cell cycle inhibitor contained in hyaluronic acid can be used in the same manner as described for cryopaste and thermopastes.

Surgical films containing a cell cycle inhibitor and a radioactive source can also be used in the management of genital tract tumor resection margins. Ideal polymeric vehicles for surgical films include flexible non-degradable polymers such as polyurethane, EVA, silicone and resorbable polymers such as poly (glycolide), poly (lactide-co-glycolide), poly (glycolide-co-caprolactone), albumin, hyaluronic acid, gelatin, and/or Carbopol. The surface of the film can be modified to hold I¹²⁵ or Pd¹⁰³ seeds at regular intervals or to hold radioactive wires (see FIG. 9 for a more detailed description). In a preferred embodiment, the surgical film is loaded with a taxane, vinca alkaloid, antimetabolite, platinum and/or alkylating agent. For example, 0.1-40% w/w paclitaxel, 0.1-40 w/w docetaxel, 0.1-40% w/w vincristine, 0.1-40% w/w methotrexate, 0.1-40% w/w cisplatin, and/or 0.1-40% w/w 5-FU is incorporated into the film. The radioactive seeds or wires are placed in the film and can be sealed in place with either another piece of cell cycle inhibitor-loaded film or molten polymer containing a cell cycle inhibitor (described above) which hardens in place. The cell cycle inhibitor-loaded film containing the radioactive source is then placed in the resection cavity as required.

A surgical spray loaded with a cell cycle inhibitor and a brachytherapy source is also suitable for use in the treatment of genital tract tumor resection margins. For this embodiment, taxanes, vinca alkaloids, antimetabolites, platinum and/or alkylating agents are formulated into an aerosol into which a radioactive source is incorporated. In a preferred embodiment, paclitaxel, docetaxel, vincristine, methotrexate, cisplatin, and/or 5-FU is formulated into an aerosol which also contains an aqueous radioactive source (or microparticulate such as gold grains). This is sprayed onto the resection margin during open or endoscopic surgery interventions to help prevent tumor recurrence.

Hyperproliferative Diseases of the Uterus

Tumors of the uterus and cervix are among the most common cancers in women. Endometrial cancer is the most common gynecological malignancy with 32,000 new cases per year. Non-malignant tumors of the uterus, specifically uterine fibroids, are extremely common benign tumors. Both of these hyperproliferative diseases of the uterus are frequently treated surgically by hysterectomy; making this the most common surgical procedure performed in women. Cervical cancer is also a widespread gynecological hyperproliferative disease of the female reproductive tract. Although surgical resection of the affected tissue remains the mainstay of therapy for these three conditions, there is a significant clinical need for nonsurgical treatments for patients with advanced disease, tumors not amenable to surgical resection, women with concurrent illnesses which make them poor surgical candidates, or younger women wishing to preserve fertility.

An effective therapy for the treatment of malignant uterine tumors would stop or slow tumor growth and/or prevent the spread of the disease into adjacent or distant organs. In patients undergoing surgical resection of the tumorous mass, an effective embodiment would reduce the incidence of local recurrence of the disease in adjacent tissues. In patients in whom a complete response is not possible, an effective treatment will reduce the morbidity associated with their illness by decreasing symptoms such as pain, vaginal bleeding, and fistula formation with adjacent organs (e.g. rectovaginal fistulas, vesicovaginal fistulas). And finally, effective treatment of uterine fibroids using the described embodiments would decrease pain, improve dysmenorrhea, reduce menorrhagia and prevent pain with intercourse.

Suitable embodiments for the treatment of hyperproliferative diseases of the uterus include:

1. Cell Cycle Inhibitor-Coated Radioactive Capsules

2. Cell Cycle Inhibitor-Loaded Radioactive Capsules

3. Administration for the Cell Cycle Inhibitor to the Surface of the Cervix or Endometrium

4. Cell Cycle Inhibitor-Loaded Spacers

5. Cell Cycle Inhibitor-Coated Radioactive Seeds

6. Cell Cycle Inhibitor-Coated Radioactive Sutures

7. Cell Cycle Inhibitor-Loaded Radioactive Sutures

8. Interstitial Injection of Cell Cycle Inhibitors

9. Cell Cycle Inhibitor-Loaded Surgical Pastes, Gels, Films, or Sprays

In one embodiment, the cell cycle inhibitor is coated onto a radioactive capsule suitable for intra-cavitary placement in the vagina or uterus. Several commercially available capsules are available for this purpose (e.g. Simon-Heyman Capsules) which are loaded with a radioactive source (usually cesium¹³⁷ or radium²²⁶). A cell cycle inhibitor is formulated into a polymer such as silicone, gelatin, polyurethane, or polylactide-co-glycolide which is applied as a coating to the surface of the capsule. Cell cycle inhibitors such as taxanes, platinum, alkylating agents, nitrogen mustards, topoisomerase inhibitors, anthracyclines and/or estramustine are preferred. Specifically, 0.1-40% w/w paclitaxel, 0.1-40% w/w docetaxel, 0.1-40% w/w cisplatin, 0.1-40% w/w 5-Fluorouracil, 0.1-40% w/w ifosfamide, 0.1-40% w/w irinotecan, 0.1-40% w/w doxorubicin, and/or 0.1-40% w/w gemcitabine formulated in polyurethane and applied as a surface coating to a radioactive capsule are particularly preferred embodiment.

In a second embodiment, the cell cycle inhibitor is incorporated into a polymer which is a constituent component of the radioactive capsule. For example cell cycle inhibitors such as taxanes, platinum, alkylating agents, nitrogen mustards, topoisomerase inhibitors, anthracyclines, and/or estramustine are formulated into a molten polymer (e.g. polycaprolactone at 60°, polyethyleneglycol which is allowed to cool/heat as required to solidify. During the solidification process, a radioactive source (e.g. Ce¹³⁷, Co⁶⁰, Ir¹⁹², I¹²⁵, Pd¹⁰³) is added in the appropriate geometry. Preferred cell cycle inhibitors for use in this embodiment include 0.1-40% w/w paclitaxel, 0.1-40% w/w docetaxel, 0.1-40% w/w cisplatin, 0.1-40% w/w 5-Fluorouracil, 0.1-40% w/w ifosfamide, 0.1-40% w/w irinotecan, 0.1-40% w/w doxorubicin, and/or 0.1-40% w/w gemcitabine.

The cell cycle inhibitor-coated radioactive capsules or cell cycle inhibitor-loaded radioactive capsules are administered in a similar manner. Over 100 different applications are available worldwide to administer capsules such as these (e.g. Fletcher-Suit-Deleos Colpostats, Fletcher Intrauterine Tandems, Vaginal Cylinders). The applicator used should be porous to allow passage of the cell cycle inhibitor into the cervical or endometrial tissue. Under general or spinal anesthesia, the patient is placed in the dorsal lithotomy position, a weighted speculum is inserted and the uterine canal is sounded. The cervical is dilated and a tandem is inserted into the cervix and ovoids are placed on the external surface of the cervix. The cell cycle inhibitor-coated or cell cycle inhibitor-loaded capsules are then delivered via the applicator or required to achieve the appropriate dosimetry to the endometrium and/or cervix.

In a third embodiment, the cell cycle inhibitor is administered to the surface of the cervix or endometrium. Topical preparations such as taxanes, platinum, alkylating agents, nitrogen mustards, topoisomerase inhibitors, anthracyclines and/or estramustines formulated with a mucoadhesive polymer are ideally suited for this embodiment. For example, 0.1-40% w/w paclitaxel, 0.1-40% w/w docetaxel, 0.1-40% w/w cisplatin, 0.1-40% w/w 5-Fluorouracil, 0.1-40% w/w ifosfamide, 0.1-40% w/w irinotecan, 0.1-40% w/w doxorubicin, and/or 0.1-40% w/w gemcitabine are formulated into a topical carrier and applied to the surface of the cervix or endometrium. A radioactive source (such as Simon-Heyman Capsule with or without a cell cycle inhibitor coating) is inserted into the cervix or vagina as described above.

For some patients, transperineal implantation of interstitial brachytherapy is preferred over, or is used in combination with, intracavitary brachytherapy. Often a perineal template (e.g. Martinez Perineal Interstitial Template, Syed-Neblett Transperineal Template) is used to aid in placement of the radioactive source. The template is often sutured in place on the perineum and has an array of small holes (1 cm apart) that serve as trocar guides which allow insertion of needles in parallel horizontal planes. Typically, I¹²⁵, Cs¹³⁷, or I¹⁹² radioactive sources are used to deliver a dose of brachytherapy (usually 50-80 cGy/hr). Interstitial brachytherapy—cell cycle inhibitor formulations can also be placed directly during surgical procedures.

Embodiments 4 through 8 describe interstitial cell cycle inhibitor —brachytherapy inventions suitable for administration in this manner.

In a fourth embodiment, a cycle inhibitor is loaded into a resorbable [(e.g., poly (glycolide), poly (lactide-co-glycolide), poly (glycolide-co-caprolactone), albumin, byaluronic acid, gelatin, and/or Carbopol)] or nonresorbable [(e.g., polypropylene, silicone, EVA, polyurethane, and/or polyethylene] polymer(s) and formed into a cylindrical spacer 1-5 mm in diameter and 0.5 cm or 1.0 cm in length. 1125 or Pdl seeds are placed in a needle (or catheter) and separated from each other by the cell cycle inhibitor-loaded spacers (i.e., seed-spacer-seed-spacer, etc.) of the appropriate length. The needles or catheters are then inserted through a template and into the hyperproliferative tissue in the uterus. Under general or spinal anesthesia, a template is placed over the perineum (e.g. Syed-Neblett Template, Martinez Universal Perineal Interstitial Template) and needles/catheters are inserted under ultrasound or fluoroscopic guidance until the tumorous uterine tissue is implanted with needles 0.5 to 1.0 cm apart. Although any cell cycle inhibitor could be incorporated into a polymeric spacer, taxanes, platinum, alkylating agents, nitrogen mustards, topoisomerase inhibitors, anthracyclines and/or estramustines are preferred. For example, 0.1-40% w/w paclitaxel (by weight) incorporated into a resorbable or non-resorbable polymeric spacer is an ideal embodiment. Docetaxel at 0.1-₄₀% w/w, 0.1-40% w/w cisplatin, 0.1-40% w/w 5-Fluorouracil, 0.1-40% w/w ifosfamide, 0.1-40% w/w irinotecan, 0.1-40% w/w doxorubicin, and/or 0.1-40% w/w gemcitabine are also preferred embodiments.

In a fifth embodiment, a cell cycle inhibitor-coated seed can be utilized. Here the cell cycle inhibitor is coated directly onto the radioactive seed (e.g. I¹²⁵ or Pd¹⁰³) either prior to, or at the time of, implantation into the uterus. Once again preferred cell cycle inhibitors include taxanes, platinum, alkylating agents, nitrogen mustards, topoisomerase inhibitors, anthracyclines and/or gemcitabine. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Specifically, 0.1-40% w/w cisplatin, 0.1-40% w/w 5-Fluorouracil, 0.1-40% w/w ifosfamide, 0.1-40% w/w irinotecan, 0.1-40% w/w doxorubicin, and/or 0.1-40% w/w gemcitabine can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the uterus via needles or catheters (as described previously) or via specialized applicators (e.g. Mick Applicator). The Mick Applicator, for example, can implant cell cycle inhibitor-coated seeds at 1 cm intervals in the uterus and their position can be verified by fluoroscopy.

In a sixth embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the uterus percutaneously or during open surgery. A cell cycle inhibitor can be loaded into a polymeric carrier applied to the surface of the suture material prior to, or during, implantation. Preferred cell cycle inhibitors for non-absorbable sutures are taxanes, platinum, alkylating agents, nitrogen mustards, topoisomerase inhibitors, anthracyclines and/or gemcitabine loaded into EVA, polyurethane (PU) or PLGA silicone, gelatin, and/or dextran. The polymer-cell inhibitor formulation is then applied as a coating (e.g. sprayed, dipped, “painted” on) prior to insertion in the uterus. Examples of specific, preferred agents include 0.1-40% w/w paclitaxel, 0.1-40% w/w docetaxel, 0.1-40% w/w cisplatin, 0.1-40% w/w 5-Fluorouracil, 0.1-40% w/w ifosfamide, 0.1-40% w/w irinotecan, 0.1-40% w/w doxorubicin, and/or 0.1-40% w/w gemcitabine loaded into one (or a combination of) the above polymers and applied as a coating to a radioactive suture. Conversely, incorporation of the above agents in poly(lactide-co-glycolide), poly(glycolide) and/or dextran would be the preferred coating for absorbable radioactive sutures.

In a seventh embodiment, the cell cycle inhibitor is loaded into a radioactive suture (i.e., the cell cycle inhibitor - polymer composition is a constituent component of the suture). In a preferred embodiment, a taxane, topoisomerase inhibitor, vinca alkaloid and/or estramustine is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵or Pd¹⁰³). Particularly, preferred cell cycle inhibitors for this purpose include 0.1-40% w/w paclitaxel, 0.1-40% w/w docetaxel, 0.1-40% w/w cisplatin, 0.1-40% w/w 5-Fluorouracil, 0.1-40% w/w ifosfamide, 0.1-40% w/w irinotecan, 0.1-40% w/w doxorubicin, and/or 0.1-40% w/w gemcitabine. If a nonabsorbable suture is desired, the above agents can be loaded into polypropylene or silicone. In both cases the radioactive source is evenly spaced (e.g. 1 cm apart) within the suture (see FIG. 3).

An eighth embodiment for the treatment of hyperproliferative diseases of the uterus is infiltration of the uterus with interstitial injections of cell cycle inhibitor formulations (aqueous, nanoparticulates, microspheres, pastes, gels, etc.) prior to, or at the time of brachytherapy treatment. Taxanes, platinum, alkylating agents, nitrogen mustards, topoisomerase inhibitors, anthracyclines and/or gemcitabine compounds are preferred for this embodiment. For example, paclitaxel, docetaxel, etoposide, vinblastine and/or estramustine can be incorporated into a polymeric carrier as described previously. The resulting formulation—whether aqueous, nano or microparticulate, gel, or paste in nature —must be suitable for injection through a needle or catheter. The polymer-cell cycle inhibitor formulation is then injected into the uterus such that therapeutic drug levels are reached in the diseased tissues. A brachytherapy source is also administered interstitially by any of the methods as described previously. While also suitable for use with permanent low dose brachytherapy sources, this treatment form is best suited for use with temporary high dose rate (HDR) brachytherapy. For example, the uterus can be infiltrated by interstitial injection of the cell cycle inhibitor in combination with high energy I¹⁹², administered via a template, which remains in place for 50-80 minutes before being removed. Interstitial injection of the cell cycle inhibitor is ideal for HDR therapy since, unlike some of the other interstitial embodiments, it does not require attachment of the cell cycle inhibitor to the brachytherapy source—important since the brachytherapy source is ultimately removed in HDR.

In a ninth embodiment, the cell cycle inhibitor and the radioactive source are delivered intraoperatively part of tumour resection surgery. Resection of a malignant uterus mass is the primary therapeutic option for many patients diagnosed with uterus cancer. Unfortunately, for many patients complete removal of the mass is not possible and malignant cells remain in adjacent tissues. To address this problem, a cell cycle inhibitor can be combined with a radioactive source and applied to the surface of the tumor resection margin. Surgical pastes, gels, and sprays containing taxanes, platinum, alkylating agents, nitrogen mustards, topoisomerase inhibitors, anthracyclines and/or gemcitabine are ideally suited for treatment of uterus tumor resection beds. In a surgical paste, 0.1-40% w/w paclitaxel, 0.1-40%W/w docetaxel, 0.1-40% w/w cisplatin, 0.1-40% w/w 5-Fluorouracil, 0.1-40% w/w ifosfamide, 0.1-40% w/w irinotecan, 0.1-40% w/w doxorubicin, and/or 0.1-40% w/w gemcitabine is incorporated into polymeric or non-polymeric paste formulation (refer to examples). The cell cycle inhibitor-loaded paste is injected via a syringe into the resection cavity and spread by the surgeon to cover the desired area. For thermally responsive pastes, the formulation cools (cold-sensitive) or heats (heat-sensitive) to body temperature (37° C.) it gradually solidifies. During this time interval, radioactive sources (e.g., iridium wires, I¹²⁵ seeds, Pd¹⁰³ seeds) are inserted into the molten formulation in the correct geometry to deliver the desired dosimetry. The paste will then completely harden in the shape of the resection margin while also fixing the radioactive source in place. Alternatively, a particulate radioactive source can be added to the thermopaste or cryopaste prior to administration when precise dosimetry is not required. A gel composed of a cell cycle inhibitor contained in hyaluronic acid can be used in the same manner as described for cryopaste and thermopastes.

Surgical pastes, gels and sprays as described are also well suited for intracavitary use. The uterine cavity, cervical canal, or vagina can be infused with a paste, gel or spray loaded with a cell cycle inhibitor under direct vision (patient in dorsal lithotomy position with a speculum in place). A intracavitary radioactive source is then placed in the vagina, cervix, or uterus to provide a local source of radiotherapy.

It should be obvious to one of skill in the art that analogues or derivatives of the above compounds (as described previously) given at similar or biologically equivalent dosages would also be suitable for the above invention.

Hyperproliferative Diseases of the Liver and Bile Duct

Primary hepatic tumors are more common in Asia and regions of the world with a high incidence of hepatitis B infections. Primary biliary tumors cause morbidity and mortality due to local manifestations (i.e., obstruction of the cystic duct) as opposed to systemic complications. Biliary or hepatic malignancies can both result in biliary obstruction which predisposes the patient to cholangitis, sepsis and liver failure. Therefore, local control of the disease is an important part of the treatment of patients with these conditions.

Endoscopic retrograde cholangiopancreatography (ERCP) has allowed access to the biliary system without open surgery. This allows direct placement of intracavity and interstitial therapeutic embodiments. These embodiments can also be placed percutaneously into the biliary tree under radiographic guidance. A third method of administration involves direct placement of cell cycle inhibitors and brachytherapy sources during open or laparoscopic surgery. Therefore, there are several methods of administration available to one wishing to practice the inventions described below. Common brachytherapy sources for use in these embodiments include low and high activity Ir¹⁹² and Co⁶⁰.

An effective therapy would slow or inhibit tumor growth and prolong patency of the biliary system. By preventing or delaying the obstruction of bile flow, an effective therapy will reduce or eliminate jaundice. Clinically, this will prevent the development of cholangitis, sepsis, liver damage (and potentially liver failure) and death.

Although any interstitial, intracavitary, or surface therapy described previously can be utilized, preferred embodiments include:

1. Cell Cycle Inhibitor-Loaded Spacers

2. Cell Cycle Inhibitor-Coated Radioactive Seeds

3. Cell Cycle Inhibitor-Coated Radioactive Sutures

4. Cell Cycle Inhibitor-Loaded Radioactive Sutures

5. Interstitial Injection of Cell Cycle Inhibitors

6. Cell Cycle Inhibitor-Coated Radioactive Wires

7 . Cell Cycle Inhibitor-Coated Radioactive Stents

8. Delivery of Cell Cycle Inhibitor s via Drug-Deliver y Balloons or Catheters

9. Cell Cycle Inhibitor-Loaded Surgical Pastes, Films, or Sprays

In one embodiment, a cycle inhibitor is loaded into a resorbable [(e.g., poly (glycolide), poly (lactide-co-glycolide), poly (glycolide-co-caprolactone), albumin, hyaluronic acid, gelatin, and/or Carbopol)] or nonresorbable [(e.g., polypropylene, silicone, EVA, polyurethane, and/or polyethylene] polymer(s) and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted percutaneous in the liver or biliary tree. Although any cell cycle inhibitor could be incorporated into a polymeric spacer, taxanes, anthracylines, platinum, alkylating agents, gemcitabine, mitomycin, and/or floxuridine (FUDR) are preferred. For example, 0.1-40% w/w paclitaxel (by weight) incorporated into a resorbable or non-resorbable polymeric spacer is an ideal embodiment. Docetaxel at 0.1-40% w/w, 0.1-40% w/w adriamycin, 0.1-40% w/w doxorubicin, 0.1-40% w/w epirubicin, 0.1-40% w/w cisplatin, 0.1-40% w/w 5-FU, 0.1-40% w/w mitomycin, and/or 0.1-40% w/w FUDR are also preferred embodiments. It should be obvious to one of skill in the art that analogues or derivatives of the above compounds (as described previously) given at similar or biologically equivalent dosages would also be suitable for the above invention.

In a second embodiment, a cell cycle inhibitor-coated radioactive seed can be utilized. Here the cell cycle inhibitor is coated directly onto the radioactive seed (e.g. I¹²⁵or Pd¹⁰³) either prior to, or at the time of, implantation into the liver or bile duct. Once again preferred cell cycle inhibitors include taxanes, anthracylines, platinum, alkylating agents, gemcitabine, mitomycin, and/or floxuridine (FUDR). For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Similarly 0.1-40% w/w adriamycin, 0.1-40w/w doxorubicin, 0.1-40% w/w epirubicin, 0.1-40% w/w cisplatin, 0.1-40% w/w 5-FU, 0.1- 40% w/w mitomycin, and/or 0.1-40% w/w FUDR can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the liver or bile duct via needles or catheters (as described previously) or via specialized applicators.

In a third embodiment, a cell cycle inhibitor can be coated onto a radioactive suture. Nonabsorbable or absorbable radioactive sutures (e.g. I¹²⁵ Sutures, Medic-Physics Inc., Arlington Heights I1; EPB 386757; U.S. Pat. Nos. 5,906,573; 5,897,573; 5,709,644; WO 98/57703; WO 98/47432; WO 97/19706) can be implanted into the liver and bile duct percutaneously or during open surgery. A cell cycle inhibitor can be loaded into a polymeric carrier applied to the surface of the suture material prior to, or during, implantation. Preferred cell cycle inhibitors for non-absorbable sutures are taxanes, anthracylines, platinum, alkylating agents, gemcitabine, mitomycin, and/or floxuridine (FUDR) loaded into EVA, polyurethane (PU) or PLGA silicone, gelatin, and/or dextran. The polymer-cell inhibitor formulation is then applied as a coating (e.g. sprayed, dipped, “painted” on) prior to insertion in the liver and bile duct. Examples of specific, preferred agents include 0.1-40% w/w paclitaxel, 0.1-40% w/w docetaxel, 0.1-40% w/w adriamycin, 0.1-40% w/w doxorubicin, 0.1-40% w/w epirubicin, 0.1-40% w/w cisplatin, 0.1-40% w/w 5-FU, 0.1-40% w/w mitomycin, and/or 0.1-40% w/w FUDR loaded into one (or a combination of) the above polymers and applied as a coating to a radioactive suture. Conversely, incorporation of the above agents in poly(lactide-co-glycolide), poly(glycolide)or dextran would be the preferred coating for absorbable radioactive sutures.

In a fourth embodiment, the cell cycle inhibitor is loaded into a radioactive suture (i.e., the cell cycle inhibitor—polymer composition is a constituent component of the suture). In a preferred embodiment, a taxane, anthracycline, platinum, alkylating agent, gemcitabine, mitomycin, and/or floxuridine (FUDR) is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵or Pd¹⁰³). Particularly, preferred cell cycle inhibitors for this purpose include 0.1-40% w/w paclitaxel, 0.1-40% w/w docetaxel, 0.1-40% w/w adriamycin, 0.1-40% w/w doxorubicin, 0.1-40% w/w epirubicin, 0.1-40% w/w cisplatin, 0.1-40% w/w 5-FU, 0.1-40% w/w mitomycin, and/or 0.1-40% w/w FUDR. If a nonabsorbable suture is desired, the above agents can be loaded into polypropylene or silicone. In both cases the radioactive source is evenly spaced (e.g. 1 cm apart) within the suture (see FIG. 3).

A fifth embodiment for the treatment of malignancies of the liver and bile duct is infiltration of the liver and bile duct with interstitial injections of cell cycle inhibitor formulations (aqueous, nanoparticulates, microspheres, pastes, gels, etc.) prior to, or at the time of brachytherapy treatment. Taxanes, anthracylines, platinum, alkylating agents, gemcitabine, mitomycin, and/or floxuridine (FUDR) compounds are preferred for this embodiment. For example, paclitaxel, docetaxel, adriamycin, doxorubicin, epirubicin, cisplatin, 5-FU, mitomycin, and/or FUDR can be incorporated into a polymeric carrier as described previously. The resulting formulation—whether aqueous, nano or microparticulate, gel, or paste in nature—must be suitable for injection through a needle or catheter. The polymer-cell cycle inhibitor formulation is then injected percutaneously or via endoscope into the liver and bile duct such that therapeutic drug levels are reached in the diseased tissues. A brachytherapy source is also administered interstitially by any of the methods as described previously. While also suitable for use with permanent low dose brachytherapy sources, this treatment form is best suited for use with temporary high dose rate (HDR) brachytherapy. For example, the liver and bile duct can be infiltrated by interstitial injection of the cell cycle inhibitor in combination with high-energy I¹⁹² wires which remain in place for 50-80 minutes before being removed. Interstitial injection of the cell cycle inhibitor is ideal for HDR therapy since, unlike some of the other interstitial embodiments, it does not require attachment of the cell cycle inhibitor to the brachytherapy source—important since the brachytherapy source is ultimately removed in HDR.

In a sixth embodiment, a cell cycle inhibitor is coated onto a radioactive wire. In this application, radioactive wires (e.g. Ir¹⁹²) are placed through the tumor via the skin (percutaneously) or during open surgery. If the wire is to remain in place permanently, a variety of polymeric carriers are suitable for administration of the cell cycle inhibitor including EVA, polyurethane and silicone. The cell cycle inhibitor-polymer coating can be applied as a spray or via a dipped coating process either in advance of or at the time of insertion. A “sheet” of cell cycle inhibitor-polymer material (e.g. EVA, Polyurethane) can also be wrapped around the wire prior to insertion. If temporary high dose brachytherapy is employed, the wire must be directly coated with a cell cycle inhibitor (i.e., dried onto or attached to the wire) or the cell cycle inhibitor must be loaded into a polymer capable of rapid drug release, such as polyethylene glycol, dextran and/or hyaluronic acid since most of the drug must be released within a 1-2 hour period. Regardless of the form of brachytherapy performed, ideal cell cycle inhibitors for use as wire coatings in the treatment of malignancies of the liver and bile duct include taxanes, anthracylines, platinum, alkylating agents, gemcitabine, mitomycin, and/or floxuridine (FUDR). For example, 0.1-40% w/w paclitaxel, 0.1-40% w/w docetaxel, 0.1-40% w/w adriamycin, 0.1-40% w/w doxorubicin, 0.1-40% w/w epirubicin, 0.1-40% w/w cisplatin, 0.1-40% w/w 5-FU, 0.1-40% w/w mitomycin, and/or 0.1-40% w/w FUDR can be loaded into fast release polymeric formulations such as polyethylene glycol, dextran and/or hyaluronic acid for coating onto temporary HDR brachytherapy wires.

In a seventh embodiment, a cell cycle inhibitor can be coated onto a radioactive stent (see, e.g., EPA 857470; EPA 810004; EPA 722702; EPA 539165; EPA 497495; EPB 433011; U.S. Pat. Nos. 5,919,216; 5,873,811; 5,871,437; 5,843,163; 5,840,009; 5,730,698; 5,722,984; 5,674,177; 5,653,736; 5,354,257; 5,213,561; 5,183,455; 5,176,617; 5,059,166; 4,976,680; WO 99/42177; WO 99/39765; WO 99/29354; WO 99/22670; WO 99/03536; WO 99/02195; WO 99/02194; WO 98/48851]. A cell cycle inhibitor-coated radioactive stent can be implanted in the bile duct for treatment of primary sclerosing cholangitis or cholangiocarcinoma. Briefly, a catheter is advanced across the obstruction under radiographic or endoscopic guidance (ERCP), a balloon is inflated to dilate the obstruction, and a stent is deployed (either balloon expanded or self expanded). Radioactive isotopes, such as P³², Au¹⁹⁸, Ir¹⁹², Co⁶⁰, I¹²⁵ and Pd¹⁰³ and Pd¹⁰³ are contained within the stent to provide a source of radioactivity. A cell cycle inhibitor is linked to the surface of the stent, incorporated into a polymeric carrier applied to the surface of the stent (or as a “sleeve” which surrounds the stent), or is incorporated into the stent material itself. Cell cycle inhibitors ideally suited to this embodiment include taxanes, anthracylines, platinum, alkylating agents, gemcitabine, mitomycin, and/or floxuridine (FUDR). For example, 0.1-30% w/w paclitaxel, 0.1- 30% w/w docetaxel, 0.1-30% w/w adriamycin, 0.1- 30% w/w doxorubicin, 0.1- 30% w/w epirubicin, 0.1-30% w/w cisplatin, 0.1-30% w/w 5-FU, 0.1-30% w/w mitomycin, and/or 0.1-30% w/w FUDR can be incorporated into silicone, polyurethane and EVA, which is applied as a coating to the radioactive stent. Alternatively, 10 μg -10 mg paclitaxel, 10 μg-10 mg docetaxel, 10 g-10 mg adriamycin, 10 μg-10 mg doxorubicin, 10 μg-10 mg epirubicin, 10 g-10 mg cisplatin, 10 g-10 mg 5-FU, 10 μg-10 mg mitomycin, and/or 10 g-10 mg FUDR in a crystalline form can be dried onto the surface of the stent. A polymeric coating may be applied over the cell cycle inhibitor to help control the release of the agent into the surrounding tissue. A third alternative is to incorporate, 0.1-30% w/w paclitaxel, 0.1-30% w/w docetaxel, 0.1-30% w/w adriamycin, 0.1-30% w/w doxorubicin, 0.1- 30% w/w epirubicin, 0.1-30% w/w cisplatin, 0.1- 30% w/w 5-FU, 0.1- 30% w/w mitomycin, and/or 0.1- 30% w/w FUDR into a polymer (U.S. Pat. Nos. 5,762,625; 5,670,161; WO 95/26762; EPA 420541; U.S. Pat. Nos. 5,464,450; 5,551,954) which comprises part of the stent's structure. For example, the cell cycle inhibitor can be incorporated into a polymer such as poly (lactide-co-caprolactone), polyurethane, and/or polylactic acid in combination with a radioactive source (e.g. I¹²⁵, P³²) prior to solidification as part of the casting and manufacturing of the stent. A final alternative involves delivering the brachytherapy source via a catheter (e.g. Beta-Cath®, RadioCath®, etc.) while the cell cycle inhibitor is delivered via the stent.

In an eighth embodiment, the cell cycle inhibitor can be delivered into the bile duct via specialized balloons (e.g. Transport®; Crescendo®, Channel®; EPA 904799; EPA 904798; EPA 879614; EPA 858815; EPA 853957; EPA 829271; EPA 325836; EPA 311458; EPB 805703; U.S. Pat. Nos. 5,913,813; 5,882,290; 5,879,282; 5,863,285; WO 99/32192; WO 99/15225; WO 99/04856; WO 98/47309; WO 98/39062; WO 97/40889) or delivery catheters (EPA 832670; U.S. Pat. No. 5,938,582; 5,916,143; 5,899,882; 5,891,091; 5,851,171; 5,840,008; 5,816,999; 5,803,895; 5,782,740; 5,720,717; 5,653,683; 5,618,266; 5,540,659; 5,267,960; 5,199,939; 4,998,932; 4,963,128; 4,862,887; 4,588,395; WO 99/42162; WO 99/42149; WO 99/40974; WO 99/40973; WO 99/40972; WO 99/40971; WO 99/40962; WO 99/29370; WO 99/24116; WO 99/22815; WO 98/36790; WO 97/48452). Here a cell cycle inhibitor formulated into an aqueous, non-aqueous, nanoparticulate, microsphere and/or gel formulation, which may be delivered by such a device. Preferred cell cycle inhibitors include taxanes (e.g. paclitaxel, docetaxel), anthracylines, platinum, alkylating agents, gemcitabine, mitomycin, and/or floxuridine (FUDR) at appropriate therapeutic doses. The brachytherapy is delivered via the catheter, balloon or stent.

In a ninth embodiment, the cell cycle inhibitor and the radioactive source are delivered intraoperatively part of tumour resection surgery. Resection of a malignant liver or bile duct mass is a therapeutic option for some patients diagnosed with hepatic or cholangiocarcinoma. Unfortunately, for many patients complete removal of the mass is not possible and malignant cells remain in adjacent tissues. To address this problem, a cell cycle inhibitor can be combined with a radioactive source and applied to the surface of the tumor resection margin. Surgical pastes, gels and films containing taxanes, anthracylines, platinum, alkylating agents, gemcitabine, mitomycin, and/or floxuridine (FUDR) are ideally suited for treatment of liver and bile duct tumor resection beds. In a surgical paste, 0.1-40% w/w paclitaxel, 0.1-40% w/w docetaxel, 0.1-40% w/w adriamycin, 0.1-40% w/w doxorubicin, 0.1-40% w/w epirubicin, 0.1-40% w/w cisplatin, 0.1-40% w/w 5-FU, 0.1-40% w/w mitomycin, and/or 0.1-40% w/w FUDR is incorporated into polymeric or non-polymeric paste formulation (refer to examples). The cell cycle inhibitor-loaded paste is injected via a syringe into the resection cavity and spread by the surgeon to cover the desired area. For thermally responsive pastes, as the formulation cools (cold-sensitive) or heats (heat- sensitive) to body temperature (37° C.) it gradually solidifies. During this time interval, radioactive sources (e.g., iridium wires, I¹²⁵ seeds, Pd¹⁰³ seeds) are inserted into the molten formulation in the correct geometry to deliver the desired dosimetry. The paste will then completely harden in the shape of the resection margin while also fixing the radioactive source in place. Alternatively, a particulate radioactive source can be added to the thermopaste or cryopaste prior to administration when precise dosimetry is not required. A gel composed of a cell cycle inhibitor contained in hyaluronic acid can be used in the same manner as described for cryopaste and thermopastes.

Surgical films containing a cell cycle inhibitor and a radioactive source can also be used in the management of liver and bile duct tumor resection margins. Ideal polymeric vehicles for surgical films include flexible non-degradable polymers such as polyurethane, EVA silicone and resorbable polymers such as poly (glycolide), poly (lactide-co-glycolide), poly (glycolide-co-caprolactone), albumin, hyaluronic acid, gelatin, and/or Carbopol. The surface of the film can be modified to hold I¹²⁵, Pd¹⁰³ seeds at regular intervals or to hold radioactive wires (see FIG. 10 for a more detailed description). In a preferred embodiment, the surgical film is loaded with a taxanes, anthracylines, platinum, alkylating agents, gemcitabine, mitomycin, and/or floxuridine (FUDR). For example, 0.1-40% w/w paclitaxel, 0.1-40 w/w docetaxel, 0.1-40% w/w adriamycin, 0.1-40% w/w doxorubicin, 0.1-40% w/w epirubicin, 0.1-40% w/w cisplatin, 0.1-40% w/w 5-FU, 0.1-40% w/w mitomycin, and/or 0.1-40% w/w FUDR is incorporated in to the film. The radioactive seeds or wires are placed in the film and can be sealed in place with either another piece of cell cycle inhibitor-loaded film or molten polymer containing a cell cycle inhibitor (described above) which hardens in place. The cell cycle inhibitor-loaded film containing the radioactive source is then placed in the resection cavity as required.

A surgical spray loaded with a cell cycle inhibitor and a brachytherapy source is also suitable for use in the treatment of liver and bile duct tumor resection margins. For this embodiment, taxanes, anthracylines, platinum, alkylating agents, gemcitabine, mitomycin, and/or floxuridine (FUDR) are formulated into an aerosol into which a radioactive source is incorporated. In a preferred embodiment, paclitaxel, docetaxel, anthracyclines, doxorubicin, epirubicin, cisplatin, 5-FU, mitomycin, and/or FUDR is formulated into an aerosol which also contains an aqueous radioactive source (or microparticulate such as gold grains). This is sprayed onto the resection margin during open or endoscopic surgery interventions to help prevent tumor recurrence.

Hyperproliferative Diseases of the Lung

Lung cancer affects over 160,000 patients per year in the U.S. and has a mortality rate in excess of 80%. As a result of this, lung cancer remains a significant health problem.

Surgical resection of the mass is the preferred form of treatment for patients with localized disease. Unfortunately, many patients have advanced disease at the time of presentation to a physician. Cell cycle inhibitor and brachytherapy combination treatments are ideally suited to placement during surgical resection of a mass to help prevent recurrence of the disease. For those in whom complete resection is impossible, these therapies can be used to reduce the morbidity associated with local growth of the tumor. Approximately 30-50% of patients experience significant problems due to local tumor expansion, including severe cough, dyspnea, pain, and hemoptysis. Interstitial embodiments and embodiments delivered via a bronchoscope are ideally suited to local control of tumor growth designed to improve the quality of life of lung cancer patients. The following treatment modalities can be delivered in a variety of ways including direct placement during open surgical procedures and during minimally invasive procedures.

An effective therapy for lung cancer would stop or slow tumor growth and/or prevent the spread of the disease into adjacent or distant organs (metastasis). Locally effective therapies can also reduce the incidence of local recurrence following tumor excision. And finally, effective palliative local therapies will decrease morbidity and improve the patient's quality of life by reducing pain, cough, dyspnea and hemoptysis.

Preferred embodiments for the treatment of lung cancer include:

1. Cell Cycle Inhibitor-Loaded Surgical Pastes, Films, or Sprays

2. Cell Cycle Inhibitor-Coated Radioactive Stents

3. Delivery of Cell Cycle Inhibitors via Drug-Delivery Balloons or Catheters

4. Cell Cycle Inhibitor-Loaded Spacers

5. Cell Cycle Inhibitor-Coated Radioactive Seeds

6. Cell Cycle Inhibitor-Coated Radioactive Sutures

7. Cell Cycle Inhibitor-Loaded Radioactive Sutures

8. Interstitial Injection of Cell Cycle Inhibitors

9. Cell Cycle Inhibitor—Coated Radioactive Wires

In one embodiment, the cell cycle inhibitor and the radioactive source are delivered intraoperatively part of lung tumour resection surgery. Resection of a malignant lung mass is the primary therapeutic option for many patients diagnosed with lung cancer. Unfortunately, for many patients (particularly those with large mediastinal or chest wall tumors) complete removal of the mass is not possible and malignant cells remain in adjacent tissues. To address this problem, a cell cycle inhibitor can be combined with a radioactive source and applied to the surface of the tumor resection margin. Surgical pastes, gels and films containing taxanes, topoisomerase inhibitors, vinca alkaloids, platinum, alkylating agents, anthracyclines, nitrogen mustards, antimetabolites, nitrosureas, mitomycin, and/or gemcitabine are ideally suited for treatment of lung tumor resection beds. In a surgical paste, 0.1-40% w/w paclitaxel, 0.1-40% w/w docetaxel, 0.1-40% w/w etoposide, 0.1-40% w/w topotecan, 0.1-40% w/w irinotecan, 0.1-40% w/w vinblastine, 0.1-40% w/w vincristine, 0.1-40% w/w vinorelbine, 0.1-40% w/w carboplatin, 0.1-40% w/w cisplatin, 0.1-40% w/w cyclophosphamide, 0.1-40% w/w doxorubicin, 0.1-40% w/w ifosfamide, 0.1-40% w/w methotrexate, 0.1-40% w/w lomustine, 0.1-40% w/w mitomycin, and/or 0.1-40% w/w gemcitabine is incorporated into polymeric or non-polymeric paste formulation (refer to examples). The cell cycle inhibitor-loaded paste is injected via a syringe into the resection cavity and spread by the surgeon to cover the desired area. For thermally responsive pastes, the formulation cools (cold-sensitive) or heats (heat-sensitive) to body temperature (37° C.) it gradually solidifies. During this time interval, radioactive sources (e.g., iridium wires, I¹²⁵ seeds, Pd¹⁰³ seeds) are inserted into the molten formulation in the correct geometry to deliver the desired dosimetry. The paste will then completely harden in the shape of the resection margin while also fixing the radioactive source in place. Alternatively, a particulate radioactive source can be added to the thermopaste or cryopaste prior to administration when precise dosimetry is not required. A gel composed of a cell cycle inhibitor contained in hyaluronic acid can be used in the same manner as described for cryopaste and thermopastes. These embodiments are also ideal for placement on the pleural surface, within the mediastinum or in proximity to vital structures such as the aorta.

Surgical films containing a cell cycle inhibitor and a radioactive source can also be used in the management of lung tumor resection margins. Ideal polymeric vehicles for surgical films include flexible non-degradable polymers such as polyurethane, EVA and/or silicone and resorbable polymers such as poly (glycolide), poly (lactide-co-glycolide), poly (glycolide-co-caprolactone), albumin, hyaluronic acid, gelatin, and/or Carbopol. The surface of the film can be modified to hold I¹²⁵, Pd¹⁰³ seeds at regular intervals or to hold radioactive wires (see FIG. 10 for a more detailed description). In a preferred embodiment, the surgical film is loaded with a taxane, topoisomerase inhibitor, vinca alkaloid, platinum, alkylating agent, anthracycline, nitrogen mustard, antimetabolite, nitrosurea, mitomycin, and/or gemcitabine. For example, 0.1-40% w/w paclitaxel, 0.1-40% w/w docetaxel, 0.1-40% w/w etoposide, 0.1-40% w/w topotecan, 0.1-40% w/w irinotecan, 0.1-40% w/w vinblastine, 0.1-40% w/w vincristine, 0.1-40% w/w vinorelbine, 0.1-40% w/w carboplatin, 0.1-40% w/w cisplatin, 0.1-40% w/w cyclophosphamide, 0.1-40% w/w doxorubicin, 0.1-40% w/w ifosfamide, 0.1-40% w/w methotrexate, 0.1-40% w/w lomustine, 0.1-40% w/w mitomycin, and/or 0.1-40% w/w gemcitabine is incorporated in to the film. The radioactive seeds or wires are placed in the film and can be sealed in place with either another piece of cell cycle inhibitor-loaded film or molten polymer containing a cell cycle inhibitor (described above) which hardens in place. The cell cycle inhibitor-loaded film containing the radioactive source is then placed in the resection cavity as required (see surgical pastes above).

A surgical spray loaded with a cell cycle inhibitor and a brachytherapy source is also suitable for use in the treatment of lung tumor resection margins. For this embodiment, taxanes, topoisomerase inhibitors, vinca alkaloids, platinum, alkylating agents, anthracyclines, nitrogen mustards, antimetabolites, nitrosureas, mitomycin, and/or gemcitabine are formulated into an aerosol into which a radioactive source is incorporated. In a preferred embodiment, paclitaxel, docetaxel, etoposide, topotecan, irinotecan, vinblastine, vincristine, vinorelbine, carboplatin, cisplatin, cycophosphamide, doxorubicin, ifosfamide, methotrexate, lomustine, mitomycin, and/or gemcitabine is formulated into an aerosol which also contains an aqueous radioactive source (or microparticulate such as gold grains). This is sprayed onto the resection margin during open or endoscopic surgery interventions to help prevent tumor recurrence.

In a second embodiment, a cell cycle inhibitor can be coated onto a radioactive stent [EPA 857470; EPA 810004; EPA 722702; EPA 539165; EPA 497495; EPB 433011; U.S. Pat. Nos. 5,919,216; 5,873,811; 5,871,437; 5,843,163; 5,840,009; 5,730,698; 5,722,984; 5,674,177; 5,653,736; 5,354,257; 5,213,561; 5,183,455; 5,176,617; 5,059,166; 4,976,680; WO 99/42177; WO 99/39765; WO 99/29354; WO 99/22670; WO 99/03536; WO 99/02195; WO 99/02194; WO 98/48851]. A cell cycle inhibitor-coated radioactive stent can be implanted in the bronchial tree for treatment of malignant obstruction. Briefly, a catheter is advanced across the endobronchial obstruction under endoscopic guidance (bronchoscope), a balloon may be inflated to dilate the obstruction, and a stent is deployed (either balloon expanded or self expanded). Radioactive isotopes, such as P³², Au¹⁹⁸, Ir¹⁹², CO⁶⁰, I¹²⁵ and Pd¹⁰³ are contained within the stent to provide a source of radioactivity. A cell cycle inhibitor is linked to the surface of the stent, incorporated into a polymeric carrier applied to the surface of the stent (or as a “sleeve” which surrounds the stent), or is incorporated into the stent material itself. Cell cycle inhibitors ideally suited to this embodiment include taxanes, topoisomerase inhibitors, vinca alkaloids, platinum, alkylating agents, anthracyclines, nitrogen mustards, antimetabolites, nitrosureas, mitomycin, and/or gemcitabine.

For example, 0.1-30% w/w paclitaxel, 0.1-30% w/w docetaxel, 0.1-30% w/w etoposide, 0.1-30% w/w topotecan, 0.1-30% w/w irinotecan, 0.1-30% w/w vinblastine, 0.1-30% w/w vincristine, 0.1-30% w/w vinorelbine, 0.1-30% w/w carboplatin, 0.1-30% w/w cisplatin, 0.1-30% w/w cyclophosphamide, 0.1-30% w/w doxorubicin, 0.1-30% w/w ifosfamide, 0.1-30% w/w methotrexate, 0.1-30% w/w lomustine, 0.1-30% w/w mitomycin, and/or 0.1-30% w/w gemcitabine can be incorporated into silicone, polyurethane and EVA, which is applied as a coating to the radioactive stent. Alternatively, 100 μg -50 mg paclitaxel, 100 μg-50mg docetaxel, 100 μg-50mg etoposide, 100 μg-50 mg topotecan, 100 μg-50mg irinotecan, 1 OOpg- 50mg vinblastine, 100 μg-50 mg vincristine, 100 μg-50 mg vinorelbine, 100 μg-50mg carboplatin, 100 μg-50 mg cisplatin, 100 μg-50 mg cyclophosphamide, 100 μg-50 mg doxorubicin, 100 μg-50 mgifosfamide, 100 μg-50 mgmethotrexate, 100 μg-50 mg lomustine, 100 μg-50 mg mitomycin, and/or 100 μg-50 mg gemcitabine in a crystalline form can be dried onto the surface of the stent. A polymeric coating may be applied over the cell cycle inhibitor to help control the release of the agent into the surrounding tissue. A third alternative is to incorporate 0.1-30% w/w paclitaxel, 0.1-30% w/w docetaxel, 0.1-30% w/w etoposide, 0.1-30% w/w topotecan, 0.1-30% w/w irinotecan, 0.1-30% w/w vinblastine, 0.1-30% w/w vincristine, 0.1-30% w/w vinorelbine, 0.1-30% w/w carboplatin, 0.1-30% w/w cisplatin, 0.1-30% w/w cyclophosphamide, 0.1-30% w/w doxorubicin, 0.1-30% w/w ifosfamide, 0.1-30 % w/w methotrexate, 0.1-30 % w/w lomustine, 0.1-30% w/w mitomycin, and/or

0.1-30% w/w gemcitabine into a polymer (U.S. Pat. Nos. 5,762,625; 5,670,161; WO 95/26762; EPA 420541; U.S. Pat. Nos. 5,464,450; 5,551,954) which comprises part of the stent's structure. For example, the cell cycle inhibitor can be incorporated into a polymer such as poly (lactide-co-caprolactone), polyurethane, and/or polylactic acid in combination with a radioactive source (e.g. I¹²⁵, P³²) prior to solidification as part of the casting and manufacturing of the stent. A final alternative involves delivering the brachytherapy source via a catheter (e.g. Beta-Cath®, RadioCath®, etc.) while the cell cycle inhibitor is delivered via the stent.

In a third embodiment, the cell cycle inhibitor can be delivered into (or through) the bronchial wall via specialized balloons (e.g. Transport®; Crescendo®, Channel®; EPA 904799; EPA 904798; EPA 879614; EPA 858815; EPA 853957; EPA 829271; EPA 325836; EPA 311458; EPB 805703; U.S. Pat. Nos. 5,913,813; U.S. Pat. Nos. 5,882,290; 5,879,282; 5,863,285; WO 99/32192; WO 99/15225; WO 99/04856; WO 98/47309; WO 98/39062; WO 97/40889) or delivery catheters (EPA 832670; U.S. Pat. Nos. 5,938,582; 5,916,143; 5,899,882; 5,891,091; 5,851,171; 5,840,008; 5,816,999; 5,803,895; 5,782,740; 5,720,717; 5,653,683; 5,618,266; 5,540,659; 5,267,960; 5,199,939; 4,998,932; 4,963,128; 4,862,887; 4,588,395; WO 99/42162; WO 99/42149; WO 99/40974; WO 99/40973; WO 99/40972; WO 99/40971; WO 99/40962; WO 99/29370; WO 99/24116; WO 99/22815; WO 98/36790; WO 97/48452). Here a cell cycle inhibitor formulated into an aqueous, non-aqueous, nanoparticulate, microsphere and/or gel formulation can be delivered by such a device. Preferred cell cycle inhibitors include taxanes (e.g. paclitaxel, docetaxel), topoisomerase inhibitors (e.g. etoposide), vinca alkaloids (e.g. vinblastine), platinum, alkylating agents, anthracyclines, nitrogen mustards, antimetabolites, nitrosureas, mitomycin, and/or gemcitabine at appropriate therapeutic doses. The brachytherapy is delivered via the catheter, balloon or stent.

In a fourth embodiment, a cycle inhibitor is loaded into a resorbable [(e.g., poly (glycolide), poly (lactide-co-glycolide), poly (glycolide-co-caprolactone), albumin, hyaluronic acid, gelatin, and/or Carbopol)] or nonresorbable [(e.g., polypropylene, silicone, EVA, polyurethane, and/or polyethylene] polymers and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted into the lung tumor during open surgery. Although any cell cycle inhibitor could be incorporated into a polymeric spacer, taxanes, topoisomerase inhibitors, vinca alkaloids, platinum, alkylating agents, anthracyclines, nitrogen mustards, antimetabolites, nitrosureas, mitomycin, and/or gemcitabine are preferred. For example, 0.1-40% w/w paclitaxel (by weight) incorporated into a resorbable or non-resorbable polymeric spacer is an ideal embodiment. Docetaxel at 0.1-40% w/w, 0.1-40% w/w etoposide, 0.1-40% w/w topotecan, 0.1-40% w/w irinotecan, 0.1-40% w/w vinblastine, 0.1-40% w/w vincristine, 0.1-40% w/w vinorelbine, 0.1-40% w/w carboplatin, 0.1-40% w/w cisplatin, 0.1-40% w/w cyclophosphamide, 0.1-40% w/w doxorubicin, 0.1-40% w/w ifosfamide, 0.1-40% w/w methotrexate, 0.1-40% w/w lomustine, 0.1-40% w/w mitomycin, and/or 0.1-40% w/w gemcitabine are also preferred embodiments. It should be obvious to one of skill in the art that analogues or derivatives of the above compounds (as described previously) given at similar or biologically equivalent dosages would also be suitable for the above invention.

In a fifth embodiment, a cell cycle inhibitor-coated seed can be utilized. Here the cell cycle inhibitor is coated directly onto the radioactive seed (e.g. I¹²⁵ or Pd¹⁰³) either prior to, or at the time of, implantation into the lung. Once again preferred cell cycle inhibitors include taxanes, topoisomerase inhibitors, vinca alkaloids, platinum, alkylating agents, anthracyclines, nitrogen mustards, antimetabolites, nitrosureas, mitomycin, and/or gemcitabine. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Similarly 0.1-40% w/w etoposide, 0.1-40% w/w topotecan, 0.1-40% w/w irinotecan, 0.1-40% w/w vinblastine, 0.1-40% w/w vincristine, 0.1-40% w/w vinorelbine, 0.1-40% w/w carboplatin, 0.1-40% w/w cisplatin, 0.1-40% w/w cyclophosphamide, 0.1-40% w/w doxorubicin, 0.1-40% w/w ifosfamide, 0.1-40% w/w methotrexate, 0.1-40% w/w lomustine, 0.1-40% w/w mitomycin, and/or 0.1-40% w/w gemcitabine can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the lung tumor via needles or catheters (as described previously) or via specialized applicators.

In a sixth embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the lung during open surgery. A cell cycle inhibitor can be loaded into a polymeric carrier applied to the surface of the suture material prior to, or during, implantation. Preferred cell cycle inhibitor for non-absorbable sutures are taxanes, topoisomerase inhibitors, vinca alkaloids, platinum, alkylating agents, anthracyclines, nitrogen mustards, antimetabolites, nitrosureas, mitomycin, and/or gemcitabine loaded into EVA, polyurethane (PU), PLGA, silicone, gelatin, and/or dextran. The polymer-cell inhibitor fonnulation is then applied as a coating (e.g. sprayed, dipped, “painted” on) prior to insertion in the lung. Examples of specific, preferred agents include 0.1-40% w/w paclitaxel, 0.1-40% w/w docetaxel, 0.1-40% w/w, 0.1-40% w/w etoposide, 0.1-40% w/w topotecan, 0.1-40% w/w irinotecan, 0.1-40% w/w vinblastine, 0.1-40% w/w vincristine, 0.1-40% w/w vinorelbine, 0.1-40% w/w carboplatin, 0.1-40% w/w cisplatin, 0.1-40% w/w cyclophosphamide, 0.1-40% w/w doxorubicin, 0.1-40% w/w ifosfamide, 0.1-40% w/w methotrexate, 0.1-40% w/w lomustine, 0.1-40% w/w mitomycin, and/or 0.1-40% w/w gemcitabine loaded into one (or a combination of) the above polymers and applied as a coating to a radioactive suture. Conversely, incorporation of the above agents in poly(lactide-co-glycolide), poly(glycolide) or dextran would be the preferred coating for absorbable radioactive sutures.

In a seventh embodiment, the cell cycle inhibitor is loaded into a radioactive suture (i.e., the cell cycle inhibitor—polymer composition is a constituent component of the suture). In a preferred embodiment, a taxane, topoisomerase inhibitor, vinca alkaloid, platinum, alkylating agent, anthracycline, nitrogen mustard, antimetabolite, nitrosurea, mitomycin, and/or gemcitabine is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵ or Pd¹⁰³). Particularly, preferred cell cycle inhibitors for this purpose include 0.1-40% w/w paclitaxel, 0.1-40% w/w docetaxel, 0.1-40% w/w etoposide, 0.1-40% w/w topotecan, 0.1-40% w/w irinotecan, 0.1-40% w/w vinblastine, 0.1-40% w/w vincristine, 0.1-40% w/w vinorelbine, 0.1-40% w/w carboplatin, 0.1-40% w/w cisplatin, 0.1-40% w/w cyclophosphamide, 0.1-40% w/w doxorubicin, 0.1-40% w/w ifosfamide, 0.1-40% w/w methotrexate, 0.1-40% w/w lomustine, 0.1-40% w/w mitomycin, and/or 0.1-40% w/w gemcitabine. If a nonabsorbable suture is desired, the above agents can be loaded into polypropylene or silicone. In both cases the radioactive source is evenly spaced (e.g. 1 cm apart) within the suture (see FIG. 3) and the suture is implanted in the lung tumor during open surgery.

An eight embodiment for the treatment of hyperproliferative diseases of the lung is infiltration of the lung with interstitial injections of cell cycle inhibitor formulations (aqueous, nanoparticulates, microspheres, pastes, gels, etc.) prior to, or at the time of brachytherapy treatment. Taxanes, topoisomerase inhibitors, vinca alkaloids and/or estramustine compounds are preferred for this embodiment. For example, paclitaxel, docetaxel, etoposide, vinblastine and/or estramustine can be incorporated into a polymeric carrier as described previously. The resulting formulation—whether aqueous, nano or microparticulate, gel, or paste in nature—must be suitable for injection through a needle or catheter. The polymer-cell cycle inhibitor formulation is then injected into the lung during open surgery or via bronchoscope such that therapeutic drug levels are reached in the tumor tissue. A brachytherapy source is also administered interstitially by any of the methods as described previously

In a ninth embodiment, a cell cycle inhibitor is coated onto a radioactive wire. In this application, radioactive wires (e.g. Ir¹⁹²) are placed through the tumor and out through the skin during open surgery. The cell cycle inhibitor-polymer coating can be applied as a spray or via a dipped coating process either in advance of or at the time of insertion. A “sheet” of cell cycle inhibitor-polymer material (e.g. EVA, Polyurethane) can also be wrapped around the wire prior to insertion. If temporary high dose brachytherapy is employed, the wire must be directly coated with a cell cycle inhibitor (i.e., dried on to or linked to the wire) or the cell cycle inhibitor must be loaded into a polymer capable of rapid drug release, such as polyethylene glycol, dextran and/or hyaluronic acid since most of the drug must be released within a 1-2 hour period. Regardless of the form of brachytherapy performed, ideal cell cycle inhibitors for use as wire coatings in the treatment of hyperproliferative diseases of the lung include taxanes, topoisomerase inhibitors, vinca alkaloids and estramustine. For example, 0.1-40% w/w paclitaxel, 0.1-40% w/w docetaxel, 0.1-40% w/w, 0.1-40% w/w etoposide, 0.1-40% w/w topotecan, 0.1-40% w/w irinotecan, 0.1-40% w/w vinblastine, 0.1-40% w/w vincristine, 0.1-40% w/w vinorelbine, 0.1-40% w/w carboplatin, 0.1-40% w/w cisplatin, 0.1-40% w/w cyclophosphamide, 0.1-40% w/w doxorubicin, 0.1-40% w/w ifosfamide, 0.1-40% w/w methotrexate, 0.1-40% w/w lomustine, 0.1-40% w/w mitomycin, and/or 0.1-40% w/w gemcitabine can be loaded into fast release polymeric formulations such as polyethylene glycol, dextran and/or hyaluronic for coating onto temporary HDR brachytherapy wires. The wires and the catheters are removed following completion of the treatment.

It should be obvious to one of skill in the art that any of the previously mentioned cell cycle inhibitors and derivatives or analogues, thereof, can be combined with any of the previously described polymers and brachytherapy sources to create variation of the above compositions without deviating from the spirit and scope of the invention.

Hyperproliferative Diseases of the Pancreas

Pancreatic cancer is the fifth leading cause of cancer death in the U.S. Unfortunately, surgery and chemotherapy have little effect on survival and external beam radiotherapy often damages critical nearby structures (liver, kidney, spinal cord and GI tract). Therefore, there exists a significant clinical need for new therapies to treat this devastating condition.

An effective treatment for pancreatic cancer would stop or slow tumor growth and/or prevent the spread of the disease into adjacent (liver, bile duct, GI tract) or distant organs. In patients in whom a curative procedure is impossible, an effective treatment will reduce the incidence or severity of symptoms such as pain, depression, jaundice, cholangitis, sepsis, diabetes, and small bowel obstruction. If surgical resection of the tumor is attempted, an effective adjuvent therapy will reduce the size of the tumor prior to resection (to make the surgical procedure easier or more effective). Intraoperative placement of the described embodiments during tumor excision surgery can also reduce the incidence of local recurrence of the disease in the postoperative period.

Typically, brachytherapy is used for unresectable, locally advanced disease. Intraoperative, permanent interstitial placement of brachytherapy sources is the most widely used treatment. Usually, a Mick Applicator is used intraoperatively to insert I¹²⁵ (or Pd¹⁰³) seeds in parallel arrays (1 to 1.5 cm apart) throughout the tumor.

Interstitial embodiments suitable for use in the management of pancreatic cancer include:

1. Cell Cycle Inhibitor-Loaded Spacers

2. Cell Cycle Inhibitor-Coated Radioactive Seeds

3. Cell Cycle Inhibitor-Coated Radioactive Sutures

4. Cell Cycle Inhibitor-Loaded Radioactive Sutures

5. Interstitial Injection of Cell Cycle Inhibitors

In one embodiment, a cycle inhibitor is loaded into a resorbable [(e.g., poly (glycolide), poly (lactide-co-glycolide), poly (glycolide-co-caprolactone), albumin, byaluronic acid, gelatin, and/or Carbopol)] or nonresorbable [(e.g., polypropylene, silicone, EVA, polyurethane, and/or polyethylene] polymer(s) and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted into the pancreatic tumor. Although any cell cycle inhibitor could be incorporated into a polymeric spacer, taxanes, alkylating agents, nitrosureas, anthracyclines and/or gemcitabine are preferred. For example, 0.1-40% w/w paclitaxel (by weight) incorporated into a resorbable or non-resorbable polymeric spacer is an ideal embodiment. Docetaxel at 0.1-40% w/w, 0.1-40% w/w 5-FU, 0.1-40% w/w doxorubicin, 0.1-₄₀% w/w streptozotocin, and/or 0.1-₄₀% w/w gemcitabine are also preferred embodiments. It should be obvious to one of skill in the art that analogues or derivatives of the above compounds (as described previously) given at similar or biologically equivalent dosages would also be suitable for the above invention.

In a second embodiment, a cell cycle inhibitor-coated seed can be utilized. Here the cell cycle inhibitor is coated directly onto the radioactive seed (e.g. I¹²⁵ or Pd¹⁰³) either prior to, or at the time of, implantation into the pancreatic tumor. Once again, preferred cell cycle inhibitors include taxanes, alkylating agents, nitrosureas, anthracyclines and/or gemcitabine. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Specifically, 0.1-40% w/w 5-FU, 0.1-40% w/w doxorubicin, 0.1-40% w/w streptozotocin, and/or 0.1-40% w/w gemcitabine can be incorporated into poly (glycolide), poly (lactide-co-glycolide), poly (glycolide -co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the pancreas via needles or catheters (as described previously) or via specialized applicators (e.g. Mick Applicator). The Mick Applicator, for example, can implant cell cycle inhibitor-coated seeds at 1 cm intervals in the pancreas and their position can be verified by fluoroscopy.

In a third embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the pancreas during open surgery. A cell cycle inhibitor can be loaded into a polymeric carrier applied to the surface of the suture material prior to, or during, implantation. Preferred cell cycle inhibitors applied as coatings for non-absorbable sutures are taxanes, alkylating agents, nitrosureas, anthracyclines and/or gemcitabine loaded into EVA, polyurethane (PU), PLGA, silicone, gelatin, and/or dextran. The polymer-cell inhibitor formulation is then applied as a coating (e.g. sprayed, dipped, “painted” on) prior to insertion in the pancreas. Examples of specific, preferred agents include 0.1-40% w/w paclitaxel, 0.1-40% w/w docetaxel, 0.1-40% w/w 5-FU, 0.1-40% w/w doxorubicin, 0.1-40% w/w streptozotocin, and/or 0.1-40% w/w gemcitabine loaded into one (or a combination of) the above polymers and applied as a coating to a radioactive suture. Conversely, incorporation of the above agents in poly(lactide-co-glycolide), poly(glycolide) and/or dextran would be the preferred coating for absorbable radioactive sutures.

In a fourth embodiment, the cell cycle inhibitor is loaded into a radioactive suture (i.e., the cell cycle inhibitor—polymer composition is a constituent component of the suture). In a preferred embodiment, a taxane, alkylating agent, nitrosurea, anthracycline and/or gemcitabine is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵ or Pd¹⁰³). Particularly, preferred cell cycle inhibitors for this purpose include 0.1-40% w/w paclitaxel, 0.1-40% w/w docetaxel, 0.1-40% w/w 5-FU, 0.1-40% w/w doxorubicin, 0.1-40% w/w streptozotocin, and/or 0.1-40% w/w gemcitabine. If a nonabsorbable suture is desired, the above agents can be loaded into polypropylene or silicone. In both cases the radioactive source is evenly spaced (e.g. 1 cm apart) within the suture (see FIG. 3).

A fifth embodiment for the treatment of pancreatic cancer is infiltration of the pancreas with interstitial injections of cell cycle inhibitor formulations (aqueous, nanoparticulates, microspheres, pastes, gels, etc.) prior to, or at the time of brachytherapy treatment. Taxanes, alkylating agents, nitrosureas, anthracyclines and/or gemcitabine compounds are preferred for this embodiment. For example, paclitaxel, docetaxel, 0.1-40% w/w 5-FU, 0.1-40% w/w doxorubicin, 0.1-40% w/w streptozotocin, and/or 0.1-40% w/w gemcitabine can be incorporated into a polymeric carrier as described previously. The resulting formulation—whether aqueous, nano or microparticulate, gel, or paste in nature —must be suitable for injection through a needle or catheter. The polymer-cell cycle inhibitor formulation is then injected into the pancreas intraoperatively such that therapeutic drug levels are reached in the diseased tissues. A brachytherapy source is administered interstitially by any of the methods as described previously.

Soft Tissue Sarcomas

These rare tumors affect 2 in 100,000 people in the U.S. and encompass many different pathological types. Although surgical resection of the tumor is the mainstay of therapy, local recurrence of the illness is common. Due to the infiltrating nature of the tumors, they frequently surround vital structures or expand beyond visible tumor margins making complete resection difficult or impossible.

The most common form of brachytherapy employed in the treatment of sarcomas is implantation of interstitial radioactive sources during tumor resection surgery. Catheters are threaded through the skin and tumor bed intraoperatively. This allows Ir¹⁹² wires to be inserted into the tumor resection bed in the postoperative period (usually 5-7 days after surgery) to deliver a dose of approximately 1000 cGy/day.

Interstitial therapeutic embodiments suitable for use in the treatment of soft tissue sarcomas include:

1. Cell Cycle Inhibitor-Loaded Spacers

2. Cell Cycle Inhibitor-Coated Radioactive Seeds

3. Cell Cycle Inhibitor-Coated Radioactive Sutures

4. Cell Cycle Inhibitor-Loaded Radioactive Sutures

5. Interstitial Injection of Cell Cycle Inhibitors

6. Cell Cycle Inhibitor-Coated Radioactive Wires

7. Cell Cycle Inhibitor-Loaded Surgical Pastes, Films, or Sprays

In one embodiment, a cycle inhibitor is loaded into a resorbable [(e.g., poly (glycolide), poly (lactide-co-glycolide), poly (glycolide-co-caprolactone), albumin, hyaluronic acid, gelatin, and/or Carbopol)] or nonresorbable [(e.g., polypropylene, silicone, EVA, polyurethane, and/or polyethylene] polymer(s) and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted into the tumor resection bed as described above. Although any cell cycle inhibitor could be incorporated into a polymeric spacer, taxanes, anthracyclines, nitrogen mustards, tetrazine, platinum, antimetabolites and/or vinca alkaloids are preferred. For example, 0.1-40% w/w paclitaxel (by weight) incorporated into a resorbable or non-resorbable polymeric spacer is an ideal embodiment. Docetaxel at 0.1-40% w/w, 0.1-40% w/w doxorubicin, 0.1-40% w/w ifosfamide, 0.1-40% w/w dacarbazine, 0.1-40% w/w cisplatin, 0.1-40% w/w methotrexate and/or 0.1-40% w/w vinorelbine are also preferred embodiments. It should be obvious to one of skill in the art that analogues or derivatives of the above compounds (as described previously) given at similar or biologically equivalent dosages would also be suitable for the above invention.

In a second embodiment, a cell cycle inhibitor-coated seed can be utilized. Here the cell cycle inhibitor is coated directly onto the radioactive seed (e.g. I¹²⁵ or Pd¹⁰³) either prior to, or at the time of, implantation into the soft tissue sarcoma. Once again, preferred cell cycle inhibitors include taxanes, anthracyclines, nitrogen mustards, tetrazine, platinum, antimetabolites and/or vinca alkaloids. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Specifically, 0.1-40% w/w doxorubicin, 0.1-40% w/w ifosfamide, 0.1-40% w/w dacarbazine, 0.1-40% w/w cisplatin, 0.1-40% w/w methotrexate and/or 0.1-40% w/w vinorelbine can be incorporated into poly (glycolide), poly (lactide-co-glycolide), poly (glycolide -co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the soft tissue sarcoma via needles or catheters (as described previously) or via specialized applicators.

In a third embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the soft tissue sarcoma during open surgery. A cell cycle inhibitor can be loaded into a polymeric carrier applied to the surface of the suture material prior to, or during, implantation. Preferred cell cycle inhibitors for non-absorbable sutures are taxanes, anthracyclines, nitrogen mustards, tetrazine, platinum, antimetabolites and/or vinca alkaloids loaded into EVA, polyurethane (PU), PLGA, silicone, gelatin, and/or dextran. The polymer-cell inhibitor formulation is then applied as a coating (e.g. sprayed, dipped, “painted” on) prior to insertion in the soft tissue sarcoma or resection margins. Examples of specific, preferred agents include 0.1-₄₀% w/w paclitaxel, 0.1-40% w/w docetaxel, 0.1-40% w/w doxorubicin, 0.1-40% w/w ifosfamide, 0.1-40% w/w dacarbazine, 0.1-40% w/w cisplatin, 0.1-40% w/w methotrexate and/or 0.1-40% w/w vinorelbine loaded into one (or a combination of) the above polymers and applied as a coating to a radioactive suture. Conversely, incorporation of the above agents in poly(lactide-co-glycolide), poly(glycolide)or dextran would be the preferred coating for absorbable radioactive sutures.

In a fourth embodiment, the cell cycle inhibitor is loaded into a radioactive suture (i.e., the cell cycle inhibitor—polymer composition is a constituent component of the suture). In a preferred embodiment, a taxane, anthracycline, nitrogen mustard, tetrazine, platinum, antimetabolite and/or vinca alkaloid is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵ or Pd¹⁰³). Particularly, preferred cell cycle inhibitors for this purpose include 0.1-40% w/w paclitaxel, 0.1-40% w/w docetaxel, 0.1-40% w/w doxorubicin, 0.1-40% w/w ifosfamide, 0.1-40% w/w dacarbazine, 0.1-40% w/w cisplatin, 0.1-40% w/w methotrexate and/or 0.1-40% w/w vinorelbine. If a nonabsorbable suture is desired, the above agents can be loaded into polypropylene or silicone. In both cases the radioactive source is evenly spaced (e.g. 1 cm apart) within the suture (see FIG. 3).

A fifth embodiment for the treatment of soft tissue sarcoma is infiltration of the soft tissue sarcoma with interstitial injections of cell cycle inhibitor formulations (aqueous, nanoparticulates, microspheres, pastes, gels, etc.) prior to, or at the time of brachytherapy treatment. Taxanes, anthracyclines, nitrogen mustards, tetrazine, platinum, antimetabolites and/or vinca alkaloids compounds are preferred for this embodiment. For example, paclitaxel, docetaxel, etoposide, vinblastine and/or estramustine can be incorporated into a polymeric carrier as described previously. The resulting formulation —whether aqueous, nano or microparticulate, gel, or paste in nature—must be suitable for injection through a needle or catheter. The polymer-cell cycle inhibitor formulation is then injected into the soft tissue sarcoma such that therapeutic drug levels are reached in the diseased tissues. A brachytherapy source is also administered interstitially by any of the methods as described previously. While also suitable for use with permanent low dose brachytherapy sources, this treatment form is best suited for use with temporary high dose rate (HDR) brachytherapy. For example, the soft tissue sarcoma can be infiltrated by interstitial injection of the cell cycle inhibitor in combination with high energy I¹⁹² wires administered via catheters inserted through the skin during surgery (see above), which remain in place temporarily before being removed. Interstitial injection of the cell cycle inhibitor is ideal for HDR therapy since, unlike some of the other interstitial embodiments, it does not require attachment of the cell cycle inhibitor to the brachytherapy source —important since the brachytherapy source is ultimately removed in HDR.

In a sixth embodiment, a cell cycle inhibitor is coated onto a radioactive wire. In this application, radioactive wires (e.g. Ir¹⁹²) are placed into the tumor bed via catheters placed during open surgery. The cell cycle inhibitor-polymer coating can be applied as a spray or via a dipped coating process either in advance of or at the time of insertion. A “sheet” of cell cycle inhibitor-polymer material (e.g. EVA, Polyurethane) can also be wrapped around the wire prior to insertion. In temporary high dose brachytherapy, the wire must be coated directly with a cell cycle inhibitor (i.e dried onto the wire or affixed to the wire without a polymer carrier) or the cell cycle inhibitor must be loaded into a polymer capable of rapid drug release (such as polyethylene glycol, dextran and/or hyaluronic acid) since most of the drug must be released within a 1-2 hour period. Ideal cell cycle inhibitors for use as wire coatings in the treatment of soft tissue sarcoma include taxanes, anthracyclines, nitrogen mustards, tetrazine, platinum, antimetabolites and/or vinca alkaloids. For example, 0.1-40% w/w paclitaxel, 0.1-40% w/w docetaxel, 0.1-40% w/w doxorubicin, 0.1-40% w/w ifosfamide, 0.1-40% w/w dacarbazine, 0.1-40% w/w cisplatin, 0.1-40% w/w methotrexate and/or 0.1-40% w/w vinorelbine can be loaded into fast release polymeric formulations such as polyethylene glycol, dextran and/or hyaluronic acid for coating onto temporary HDR brachytherapy wires.

In a seventh embodiment, the cell cycle inhibitor and the radioactive source are delivered intraoperatively as part of tumor resection surgery. Resection of a malignant soft tissue sarcoma is the primary therapeutic option for most patients diagnosed with this condition. Unfortunately, for many patients complete removal of the mass is not possible and malignant cells remain in adjacent tissues. To address this problem, a cell cycle inhibitor can be combined with a radioactive source and applied to the surface of the tumor resection margin. Surgical pastes, gels and films containing taxanes, anthracyclines, nitrogen mustards, tetrazine, platinum, antimetabolites and/or vinca alkaloids are ideally suited for treatment of soft tissue sarcoma tumor resection beds. In a surgical paste, 0.1-40% w/w paclitaxel, 0.1-40% w/w docetaxel 0.1-40% w/w doxorubicin, 0.1-40% w/w ifosfamide, 0.1-40% w/w dacarbazine, 0.1-40% w/w cisplatin, 0.1-40% w/w methotrexate and/or 0.1-40% w/w vinorelbine is incorporated into polymeric or non-polymeric paste formulation (refer to examples). The cell cycle inhibitor-loaded paste is injected via a syringe into the resection cavity and spread by the surgeon to cover the desired area. For thermally responsive pastes, as the formulation cools (cold-sensitive) or heats (heat-sensitive) to body temperature (37° C.) it gradually solidifies. During this time interval, radioactive sources (e.g., iridium wires, I¹²⁵ seeds, Pd¹⁰³ seeds) are inserted into the molten formulation in the correct geometry to deliver the desired dosimetry. The paste will then completely harden in the shape of the resection margin while also fixing the radioactive source in place. Alternatively, a particulate radioactive source can be added to the thermopaste or cryopaste prior to administration when precise dosimetry is not required. A gel composed of a cell cycle inhibitor contained in hyaluronic acid can be used in the same manner as described for cryopaste and thermopastes.

Surgical films containing a cell cycle inhibitor and a radioactive source can also be used in the management of soft tissue sarcoma tumor resection margins. Ideal polymeric vehicles for surgical films include flexible non-degradable polymers such as polyurethane, EVA silicone and resorbable polymers such as poly (glycolide), poly (lactide-co-glycolide), poly (glycolide-co-caprolactone), albumin, hyaluronic acid, gelatin, and/or Carbopol. The surface of the film can be modified to hold I¹²⁵, Pd¹⁰³ seeds at regular intervals or to hold radioactive wires (see FIG. 10 for a more detailed description). In a preferred embodiment, the surgical film is loaded with a polypeptide, taxane, anthracycline, nitrogen mustard, tetrazine, platinum, antimetabolite and/or vinca alkaloid. For example, 0.1-40% w/w paclitaxel, 0.1-40w/w docetaxel, 0.1-40% w/w doxorubicin, 0.1-40% w/w ifosfamide, 0.1-40% w/w dacarbazine, 0.1-40% w/w cisplatin, 0.1-40% w/w methotrexate and/or 0.1-40% w/w vinorelbine is incorporated in to the film. The radioactive seeds or wires are placed in the film and can be sealed in place with either another piece of cell cycle inhibitor-loaded film or molten polymer containing a cell cycle inhibitor (described above) which hardens in place. The cell cycle inhibitor-loaded film containing the radioactive source is then placed in the resection cavity as required.

A surgical spray loaded with a cell cycle inhibitor and a brachytherapy source is also suitable for use in the treatment of soft tissue sarcoma tumor resection margins. For this embodiment, taxanes, anthracyclines, nitrogen mustards, tetrazine, platinum, antimetabolites and/or vinca alkaloids are formulated into an aerosol into which a radioactive source is incorporated. In a preferred embodiment, paclitaxel, docetaxel, doxorubicin, ifosfamide, dacarbazine, cisplatin, methotrexate and vinorelbine is formulated into an aerosol which also contains an aqueous radioactive source (or microparticulate such as gold grains). This is sprayed onto the resection margin during open surgery interventions to help prevent tumor recurrence.

Hyperproliferative Diseases of the Skin

Utilizing the agents, compositions and methods provided herein, a wide variety of hyperproliferative skin diseases can be readily treated or prevented. Benign tumors of the skin include epidermal nevi, seborrheic keratoses, keratoacanthoma, acrokeratosis verruciformis of Hopf, hyperkeratosis lenticularis perstans (Flegel's disease), clear cell acanthoma, and keloids. The most common premalignant skin lesions are actinic keratosis and atypical moles (dysplastic nevus). Skin malignancies include basal cell carcinoma [the most common malignancy in humans (500,000 new cases annually in the U.S.)] squamous cell carcinoma, Merkel cell carcinoma, xeroderma pigmentosum, malignant melanoma, Kaposi's sarcoma and tumors of the hair follicles, sebaceous glands and sweat glands. Nonmalignant, nontumorous hyperproliferative diseases of the skin include psoriasis and warts. All of the above conditions feature a hyperproliferative cell type (e.g., keratinocyte, and melanocyte) which produces a mass (tumor) or results in thickening of the epidermis.

Utilizing the compositions of the invention, hyperproliferative skin lesions are treated by administration of a cell cycle inhibiting agent in combination with a radioactive source. Suitable cell cycle inhibitory agents are described in detail above and include, for example, taxanes, alkylating agents, tetrazine and nitrosureas. Suitable radioactive sources are described in detail above and include, for example, radioactive isotopes of radium, cobalt, cesium, gold, iridium, iodine, palladium, phosphorus, ruthenium, strontium, yttrium and californium, as well as any other atomic nucleus capable of delivering therapeutic doses of radioactivity. The cell cycle inhibitor and/or the radioactive source may, within certain embodiments, be delivered as a composition along with a polymeric carrier, or in a liposome, cream, gel or ointment formulation as discussed in more detail both above and below. An effective therapy for hyperproliferative tumorous skin diseases will achieve at least on of the following: (1) decrease the size of a tumorous mass, (2) eliminate a tumorous mass, and/or (3) prevent recurrence of the mass after effective treatment or removal. For nontumorous hyperproliferative diseases (e.g., psoriasis and warts), it will achieve one of the following: (1) decrease the number and severity of skin lesions, (2) decrease the frequency or duration of active disease exacerbations or (3) increase the amount of time spent in remission (i.e., periods when the patient is symptom-free), and/or (4) reduce cutaneous symptoms (pain, burning, bleeding). Pathologically, the therapy will result in inhibition of cell proliferation of the affected cells (e.g. transformed cells, keratinocytes, melanocytes, basal cells, and vascular cells).

The cell cycle inhibitor can be administered in any manner sufficient to achieve the above end points, but preferred methods include:

1. Topical Administration of Cell Cycle Inhibitors.

2. Surface Molds Containing a Cell Cycle Inhibitor and a Radioactive Source.

3. Subcutaneous or Intradermal Injection of Cell Cycle Inhibitors

4. Cell Cycle Inhibitor-Loaded Spacers

5. Cell Cycle Inhibitor-Coated Radioactive Seeds

6. Cell Cycle Inhibitor-Coated Radioactive Sutures

7. Cell Cycle Inhibitor-Loaded Radioactive Sutures

8. Cell Cycle Inhibitor-Coated Radioactive Wires

In one embodiment, surface high-dose-rate brachytherapy is used for flat anatomical skin surfaces. The cell cycle inhibitor is applied as a topical cream, ointment or emollient prior to or during brachytherapy treatment. For example, a topical cream containing taxanes, alkylating agents, tetrazine, and/or nitrosureas is applied 1-4 times daily beginning 1 -10 days prior to initiation of radiotherapy and continuing for the duration of the treatment. For tumorous hyperproliferative disease, the preferred dose is 0.1-40% w/w paclitaxel, 0.1-40% w/w docetaxel, 0.1-40% w/w 5-FU, 0.1-40% w/w dacarbazine, 0.1-40% w/w carmustine, and/or 0.1-40% w/w lomustine by weight applied topically twic e daily. For nontumorous disease (e.g. psoriasis), the preferred dose is 0.1-40% w/w paclitaxel, 0.1-40% w/w docetaxel , 0.1-40% w/w 5- FU, 0.1-40% w/w dacarbazine, 0.1-40% w/w caustine, and/or 0.1-40% w/w lomustine by weight applied 1-4 times daily. The radiation dose will be determined by lesion size and duration of treatment.

A second suitable embodiment is a surface mold containing a cell cycle inhibitor and a radioactive source. Several polymers, such as polyurethane (flexible mold), or polycaprolactone (rigid mold), are suitable for manufacturing a mold containing a cell cycle inhibitor which houses a radioactive source (typically radioactive “seeds” or wires). Taxanes, alkylating agents, tetrazine, and/or nitrosureas capable of topical absorption are ideally suited for this embodiment. In specific, 0.1-40% w/w paclitaxel, 0.1-40% w/w docetaxel, 0.1-40% w/w 5-FU, 0.1-40% w/w dacarbazine, 0.1-40% w/w carmustine, and/or 0.1-40% w/w lomustine in a sustained released form (capable of topical absorption) are preferred agents. The mold also would contain a brachytherapy source such as I¹²⁵ seeds or Pd¹⁰³ seeds and/or Ir¹⁹² wires aligned to deliver the ideal dosimetry.

In a third embodiment, the cell cycle inhibitor can be injected subcutaneously or intradermally. Taxanes, alkylating agents, tetrazine, and/or nitrosureas compounds are preferred for this embodiment. For example, paclitaxel, docetaxel, 5-FU, dacarbazine, carmustine, and/or lomustine can be incorporated into a polymeric carrier as described previously. The resulting formulation—whether aqueous, nano or microparticulate, gel, or paste in nature—must be suitable for injection through a needle or catheter. The polymer-cell cycle inhibitor formulation is then injected into the skin such that therapeutic drug levels are reached in the diseased tissues. A brachytherapy source is also administered interstitially or topically by any of the methods described previously. While also suitable for use with permanent low dose brachytherapy sources, this treatment form is best suited for use with temporary high dose rate (HDR) brachytherapy. For example, the skin can be infiltrated by interstitial injection of the cell cycle inhibitor in combination with high energy I¹⁹², administered topically (to the skin surface), which remains in place for 50-80 minutes before being removed. Interstitial injection of the cell cycle inhibitor is ideal for HDR therapy since, unlike some of the other interstitial embodiments, it does not require attachment of the cell cycle inhibitor to the brachytherapy source—important since the brachytherapy source is ultimately removed in HDR.

In a fourth embodiment, a cycle inhibitor is loaded into a resorbable [(e.g., poly (glycolide), poly (lactide-co-glycolide), poly (glycolide-co-caprolactone), albumin, hyaluronic acid, gelatin, and/or Carbopol)] or nonresorbable [(e.g., polypropylene, silicone, EVA, polyurethane, and/or polyethylene] polymers and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted through the skin and into the hyperproliferative tissue. Although any cell cycle inhibitor could be incorporated into a polymeric spacer, taxanes, alkylating agents, tetrazine, and/or nitrosureas are preferred. For example, 0.1-40% w/w paclitaxel (by weight) incorporated into a resorbable or non-resorbable polymeric spacer is an ideal embodiment. Docetaxel at 0.1-40% w/w 0.1-40% w/w 5-FU, 0.1-40% w/w dacarbazine, 0.1-40% w/w carmustine, and/or 0.1-40% w/w lomustine are also preferred embodiments. It should be obvious to one of skill in the art that analogues or derivatives of the above compounds (as described previously) given at similar or biologically equivalent dosages would also be suitable for the above invention.

In a fifth embodiment, a cell cycle inhibitor-coated seed can be utilized. Here the cell cycle inhibitor is coated directly onto the radioactive seed (e.g. I¹²¹⁵ or Pd¹⁰³) either prior to, or at the time of, implantation into the skin. Once again preferred cell cycle inhibitors include taxanes, alkylating agents, tetrazine, and/or nitrosureas. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Similarly, 0.1-40% w/w 5-FU, 0.1-40% w/w dacarbazine, 0.1-40% w/w carmustine, and/or 0.1-40% w/w lomustine can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene and coated onto a brachytherapy seed. The cell cycle inhibitor-coated seed is then implanted into the skin via needles or catheters (as described previously) or via specialized applicators.

In a sixth embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the skin percutaneously or during tumor resection surgery. A cell cycle inhibitor can be loaded into a polymeric carrier applied to the surface of the suture material prior to, or during, implantation. Preferred cell cycle inhibitors for non-absorbable sutures are polypeptides, taxanes, alkylating agents, tetrazine, and/or nitrosureas loaded into EVA, polyurethane (PU) or PLGA silicone, gelatin, and/or dextran. The polymer-cell inhibitor formulation is then applied as a coating (e.g. sprayed, dipped, “painted” on) prior to insertion in the skin. Examples of specific, preferred agents include 0.1-40% w/w paclitaxel, 0.1-40% w/w docetaxel, 0.1-40% w/w 5-FU, 0.1-40% w/w dacarbazine, 0.1-40% w/w carmustine, and/or 0.1-40% w/w lomustine loaded into one (or a combination of) the above polymers and applied as a coating to a radioactive suture. Conversely, incorporation of the above agents in poly(lactide-co-glycolide), poly(glycolide) and/or dextran would be the preferred coating for absorbable radioactive sutures.

In a seventh embodiment, the cell cycle inhibitor is loaded into a radioactive suture (i.e., the cell cycle inhibitor—polymer composition is a constituent component of the suture). In a preferred embodiment, a taxane, alkylating agent, tetrazine, and/or nitrosureas is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵or Pd¹⁰³). Particularly, preferred cell cycle inhibitors for this purpose include 0.1-40% w/w paclitaxel, 0.1-40% w/w docetaxel, 10.1-40% w/w 5-FU, 0.1-40% w/w dacarbazine, 0.1-40% w/w carmustine, and/or 0.1-40% w/w lomustine. If a nonabsorbable suture is desired, the above agents can be loaded into polypropylene or silicone. In both cases the radioactive source is evenly spaced (e.g. 1 cm apart) within the suture (see FIG. 3).

In an eighth embodiment, a cell cycle inhibitor is coated onto a radioactive wire. In this application, radioactive wires (e.g. Ir¹⁹²) are placed through the tumor via the skin (percutaneously) or during open surgery. The cell cycle inhibitor-polymer coating can be applied as a spray or via a dipped coating process either in advance of or at the time of insertion. A “sheet” of cell cycle inhibitor-polymer material (e.g. EVA, Polyurethane) can also be wrapped around the wire prior to insertion. If temporary high dose brachytherapy is employed, the wire must be directly coated with a cell cycle inhibitor (i.e., dried on to, or linked to the radioactive wire) or the cell cycle inhibitor must be loaded into a polymer capable of rapid drug release, such as polyethylene glycol, dextran and/or hyaluronic acid since most of the drug must be released within a 1-2 hour period. Regardless of the form of brachytherapy performed, ideal cell cycle inhibitors for use as wire coatings in the treatment of hyperproliferative diseases of the skin include taxanes, alkylating agents, tetrazine, and/or nitrosureas. For example, 0.1-40% w/w paclitaxel, 0.1-40% w/w docetaxel, 0.1-40% w/w 5-FU, 0.1-40% w/w dacarbazine, 0.1-40% w/w carmustine, and/or 0.1-40% w/w lomustine can be loaded into fast release polymeric formulations such as polyethylene glycol, dextran and/or hyaluronic acid for coating onto temporary HDR brachytherapy wires.

Hyperproliferative Diseases of the Head and Neck

The use of brachytherapy is well established for the treatment of tumors of the tongue, floor of the mouth, lip, tonsil, nasopharynx, hypopharynx, oropharynx and larynx. Both permanent and temporary interstitial brachytherapy are used as intracavitary temporary HDR brachytherapy is used. The preferred isotopes are Ir¹⁹² and I¹²⁵ depending upon the indication.

An effective therapy for head and neck tumors would reduce or inhibit tumor growth and/or decrease local and metastatic spread of the disease. Local recurrence of the disease following tumor resection surgery is a significant clinical problem. Therefore, treatments that reduce the incidence of local tumor recurrence are particularly desirable. For patients in whom palliation is the best possible clinical outcome, an effective therapy would decrease symptoms, such as pain, dysphagia, hemoptysis, epitaxis, cough, hoarseness and dyspnea.

Although any interstitial, intracavitary, or surface therapy described previously can be utilized, preferred embodiments include:

1. Cell Cycle Inhibitor-Loaded Spacers.

2. Cell Cycle Inhibitor-Coated Radioactive Seeds.

3. Cell Cycle Inhibitor-Coated Radioactive Sutures.

4. Cell Cycle Inhibitor-Loaded Radioactive Sutures.

5. Interstitial Injection of Cell Cycle Inhibitors.

6. Cell Cycle Inhibitor-Coated Radioactive Wires.

In one embodiment, a cycle inhibitor is loaded into a resorbable [(e.g., poly (glycolide), poly (lactide-co-glycolide), poly (glycolide-co-caprolactone), albumin, hyaluronic acid, gelatin, and/or Carbopol)] or nonresorbable [(e.g., polypropylene, silicone, EVA, polyurethane, and/or polyethylene] polymers and formed into a cylindrical spacer 1-5 mm in diameter and 0.5 cm or 1.0 cm in length. I¹²⁵ or Pd¹⁰³ seeds are placed in a needle (or catheter) and separated from each other by the cell cycle inhibitor-loaded spacers (i.e., seed-spacer-seed-spacer, etc.) of the appropriate length. The needles or catheters are then inserted through a template and into the hyperproliferative tissue in the head and neck. Under general or spinal anesthesia, a template is placed over the perineum (e.g. Syed-Neblett Template, Martinez Universal Perineal Interstitial Template) and needles/catheters are inserted under ultrasound or fluoroscopic guidance until the entire head and neck is implanted with needles 0.5 to 1.0 cm apart. Although any cell cycle inhibitor could be incorporated into a polymeric spacer, taxanes, antimetabolites, platinum, alkylating agents, nitrogen mustards, anthracyclines, and/or vinca alkaloids are preferred. For example, 0.1-40% w/w paclitaxel (by weight) incorporated into a resorbable or non-resorbable polymeric spacer is an ideal embodiment. Docetaxel at 0.1-40% w/w, 0.1-40% w/w methotrexate, 0.1-40% w/w cisplatin, 0.1-40% w/w carboplatin, 0.1-40% w/w 5-FU, 0.1-40% w/w ifosfamide, 0.1-40% w/w doxorubicin, and/or 0.1-40% w/w vinorelbine are also preferred embodiments. It should be obvious to one of skill in the art that analogues or derivatives of the above compounds (as described previously) given at similar or biologically equivalent dosages would also be suitable for the above invention.

In a second embodiment, a cell cycle inhibitor-coated seed can be utilized. Here the cell cycle inhibitor is coated directly onto the radioactive seed (e.g. I¹²⁵ or Pd¹⁰³) either prior to, or at the time of, implantation into the head and neck. Once again preferred cell cycle inhibitors include taxanes, antimetabolites, platinum, alkylating agents, nitrogen mustards, anthracyclines, and/or vinca alkaloids. For example, 0.1-40% w/w paclitaxel or 0.1-40% w/w docetaxel can be incorporated into poly (glycolide), poly (lactide-co-glycolide), poly (glycolide-co-caprolactone), albumin, hyaluronic acid, gelatin, Carbopol, polypropylene, silicone, EVA, polyurethane, and/or polyethylene which are applied as a coating on the brachytherapy seed. Similarly 0.1-40% w/w methotrexate, 0.1-40% w/w cisplatin, 0.1-40% w/w carboplatin, 0.1-40% w/w 5-FU, 0.1-40% w/w ifosfamide, 0.1-40% w/w doxorubicin, and/or 0.1-40% w/w vinorelbine can be incorporated into poly (glycolide), poly (lactide-co-glycolide), poly (glycolide -co-caprolactone), albumin, hyaluronic acid, gelatin, and/or Carbopol, polypropylene, silicone, EVA, polyurethane, and/or polyethylene and coated onto a brachytherapy seed. The cell cycle inhibitor-coated seed is then implanted into the head and neck via needles or catheters (as described previously) or via specialized applicators (e.g. Mick Applicator). The Mick Applicator, for example, can implant cell cycle inhibitor-coated seeds at 1 cm intervals in the head and neck and their position can be verified by fluoroscopy.

In a third embodiment, a cell cycle inhibitor can be coated onto a radioactive suture. Nonabsorbable or absorbable radioactive sutures (e.g. I¹²⁵ Sutures, Medic-Physics Inc., Arlington Heights Ill.; EPB 386757; U.S. Pat. Nos. 5,906,573; 5,897,573; 5,709,644; WO 98/57703; WO 98/47432; WO 97/19706) can be implanted into the head and neck percutaneously or during open surgery. A cell cycle inhibitor can be loaded into a polymeric carrier applied to the surface of the suture material prior to, or during, implantation. Preferred cell cycle inhibitors for non-absorbable sutures are polypeptides, taxanes, antimetabolites, platinum, alkylating agents, nitrogen mustards, anthracyclines, and/or vinca alkaloids loaded into EVA, polyurethane (PU) or PLGA silicone, gelatin, and dextran. The polymer-cell inhibitor formulation is then applied as a coating (e.g. sprayed, dipped, “painted” on) prior to insertion in the head and neck. Examples of specific, preferred agents include 0.1-40% w/w paclitaxel, 0.1-40% w/w docetaxel, 0.1-40% w/w methotrexate, 0.1-40% w/w cisplatin, 0.1-40% w/w carboplatin, 0.1-40% w/w 5-FU, 0.1-40% w/w ifosfamide, 0.1-40% w/w doxorubicin, and/or 0.1-40% w/w vinorelbine loaded into one (or a combination of) the above polymers and applied as a coating to a radioactive suture. Conversely, incorporation of the above agents in poly(lactide-co-glycolide), poly(glycolide)or dextran would be the preferred coating for absorbable radioactive sutures.

In a fourth embodiment, the cell cycle inhibitor is loaded into a radioactive suture (i.e., the cell cycle inhibitor—polymer composition is a constituent component of the suture). In a preferred embodiment, a polypeptide, taxane, antimetabolite, platinum, alkylating agent, nitrogen mustard, anthracycline, and/or vinca alkaloid is loaded into a polyester [such as poly (glycolide), poly (lactide-co-glycolide), poly (glycolide-co-caprolactone), albumin, hyaluronic acid, gelatin and/or Carbopol] to produce a resorbable suture which also contains a radioactive source (e.g., I¹²⁵or Pd¹⁰³). Particularly, preferred cell cycle inhibitors for this purpose include 0.1-40% w/w paclitaxel, 0.1-40% w/w docetaxel, 0.1-40% w/w methotrexate, 0.1-40% w/w cisplatin, 0.1-40% w/w carboplatin, 0.1-40% w/w 5-FU, 0.1-40% w/w ifosfamide, 0.1-40% w/w doxorubicin, and/or 0.1-40% w/w vinorelbine. If a nonabsorbable suture is desired, the above agents can be loaded into polypropylene or silicone. In both cases the radioactive source is evenly spaced (e.g. 1 cm apart) within the suture (see FIG. 3).

A fifth embodiment for the treatment of hyperproliferative diseases of the head and neck is infiltration of the head and neck with interstitial injections of cell cycle inhibitor formulations (aqueous, nanoparticulates, microspheres, pastes, gels, etc.) prior to, or at the time of brachytherapy treatment. Polypeptides, taxanes, antimetabolites, platinum, alkylating agents, nitrogen mustards, anthracyclines, and/or vinca alkaloids compounds are preferred for this embodiment. For example, paclitaxel, docetaxel, methotrexate, cisplatin, carboplatin, 5-FU, ifosfamide, doxorubicin, and/or vinorelbine can be incorporated into a polymeric carrier as described previously. The resulting formulation—whether aqueous, nano or microparticulate, gel, or paste in nature—must be suitable for injection through a needle or catheter. The polymer-cell cycle inhibitor formulation is then injected into the head and neck tumor tissue such that therapeutic drug levels are reached in the diseased tissues. A brachytherapy source is also administered interstitially by any of the methods as described previously. While also suitable for use with permanent low dose brachytherapy sources, this treatment form is best suited for use with temporary high dose rate (HDR) brachytherapy. For example, the head and neck tumor can be infiltrated by interstitial injection of the cell cycle inhibitor in combination with high energy I¹⁹², administered via a template, which remains in place for 50-80 minutes before being removed. Interstitial injection of the cell cycle inhibitor is ideal for HDR therapy since, unlike some of the other interstitial embodiments, it does not require attachment of the cell cycle inhibitor to the brachytherapy source—important since the brachytherapy source is ultimately removed in HDR.

In a sixth embodiment, a cell cycle inhibitor is coated onto a radioactive wire. In this application, radioactive wires (e.g., Ir¹⁹²) are placed through the tumor via the skin (percutaneously) or during open surgery. If the wire is to remain in place permanently, a variety of polymeric carriers are suitable for administration of the cell cycle inhibitor including EVA, polyurethane and silicone. The cell cycle inhibitor-polymer coating can be applied as a spray or via a dipped coating process either in advance of or at the time of insertion. A “sheet” of cell cycle inhibitor-polymer material (e.g., EVA, Polyurethane) can also be wrapped around the wire prior to insertion. If temporary high dose brachytherapy is employed, the wire must be coated with a cell cycle inhibitor loaded into a polymer capable of rapid drug release, such as polyethylene glycol, dextran and hyaluronic since most of the drug must be released within a 1-2 hour period. Regardless of the form of brachytherapy performed, ideal cell cycle inhibitors for use as wire coatings in the treatment of hyperproliferative diseases of the head and neck include taxanes, antimetabolites, platinum, alkylating agents, nitrogen mustards, anthracyclines, and/or vinca alkaloids. For example, 0.1-40% w/w paclitaxel, 0.1-40% w/w docetaxel, 0.1-40% w/w methotrexate, 0.1-40% w/w cisplatin, 0.1-40% w/w carboplatin, 0.1-40% w/w 5-FU, 0.1-40% w/w ifosfamide, 0.1-40% w/w doxorubicin, and/or 0.1-40% w/w vinorelbine can be loaded into fast release polymeric formulations such as polyethylene glycol, dextran and hyaluronic for coating onto temporary HDR brachytherapy wires.

It should be obvious to one of skill in the art that any of the previously mentioned cell cycle inhibitors and derivatives or analogues, thereof, can be combined with any of the previously described polymers and brachytherapy sources to create variation of the above compositions without deviating from the spirit and scope of the invention.

### EXAMPLES

### Example 1

### Fluorescence Activated Cell Sorting Analysis To Determine Cell Cycle Position

A. Univariate Analysis of Cellular DNA Content

Progression through S phase and completion of mitosis (cytokinesis) result in changes in cellular DNA content. The cells' position in the major phases (G_{0.1} versus S versus G₂/M) of the cycle, therefore can be estimated based on DNA content measurement.

To carry out the procedure, admix 0.2 ml of cell suspension (10⁵ to 10⁶ cells, either directly withdrawn from tissue culture or prefixed in suspension in 70% ethanol, then rinsed and suspended in buffered saline) with 2 ml staining solution. The staining solution consists of Triton X-100, 0.1% (v/v); MgCl₂, 2 mM; NaCl, 0.1 M; PIPES buffer, 10 mM (pH 6.8); and 4′,6′-diamidino-2-phenylindone (DAPI), 1 μg/ml (2.85 μM) (final concentrations).

Transfer the sample to the flow cytometer and measure cell fluorescence. Maximum excitation of DAPI, bound to DNA, is at 359 nm and emission is at 461 nm. For fluorescence excitation, use the available UV light laser line at the wavelength nearest to 359 nm. When a mercury arc lamp serves as the excitation source, use a UGI excitation filter. A combination of appropriate dichroic mirrors and emission filters should be used to measure cell fluorescence at wavelength between 450 nm and 500 nm.

The data acquisition software of most flow cytometers/sorters allows one to record fluorescence intensities (the electronic area of the pulse signal) of 10⁴ or more cells per sample. Data are presented as DNA content frequency histograms. The data analysis software can be used to estimate the percentage of cells in G_{0.1} (generally represented by the first peak on the histograms, which these programs integrate under the assumption of the Gaussian distribution), S, and G₂+M (the second peak).

The protocol described above can be modified to accommodate different dyes and can be applied to numerous types of cells.

B. Multiparameter Analysis

Nuclear chromatin undergoes condensation during the cell cycle. In mitosis, the chromatin is maximally condensed, whereas the most decondensation is observed at the time of entrance to the S phase. The chromatin of Go cells is highly condensed, although less so than in mitosis. These changes in chromatin condensation are detected by altered DNA in situ sensitivity to denaturation.

The solutions required for the assay are metachromatic fluorochrome acridine orange (AO) stock solution and the staining solution. To prepare the AO stock solution, dissolve 1 mg AO in 1 ml of distilled water. AO of the highest purity should be used. This solution of AO is stable for several months when kept at 4° C. in the dark. To prepare the staining solution, combine 90 ml of 0.1 M citric acid with 10 ml of 0.2 M Na₂HPO₄ and add 0.6ml of the AO stock solution (final AO concentration is 6 μg/ml, i.e., approximately 20 μM, pH 2.6).

The protocol for the assay is as follows: fix the cells in suspension in 70% ethanol for at least 2 hours. Then centrifuge cells at 300g for 5 minutes. Resuspend cell pellet (10⁶ to 2×10⁶ cells) in 1 ml of phosphate buffered saline (PBS) and add 100 μg of DNase-free RNase A. Incubate at 37° C. for 1 hour. Centrifuge and resuspend in 0.5 ml of PBS. Add 0.2 ml of this suspension to 0.5 ml of 1.0 M HCl, at room temperature. After 30 seconds, add 2 ml of the staining solution at room temperature.

Transfer the sample to the flow cytometer and measure cell fluorescence. Optimal excitation of AO fluorescence is with blue light (457 or 488 nm laser lines, or BG 12 excitation filter in the case of illumination with a mercury arc lamp). Measure the green fluorescence of AO, reflecting the interaction of this dye with double-stranded DNA, at a bandwidth between 515 and 545 nm. The red fluorescence, representing AO binding to denatured DNA, is measured with a long-pass filter above 640 nm.

Data can be transformed to represent total cell fluorescence (red and green) versus αₜ, where total fluorescence is proportional to total DNA content in the cell and αₜ is the fraction of denatured DNA.

Cells are most sensitive to the effects of radiation when they are in the M or S phase of the cell cycle. Either of these two assays can be used to determine what phase a group of cells in currently in.

### Example 2

### Cell Cycle Inhibitor Determination Assay

Examples of human tumor cell lines that can be used for this assay include human melanoma, cervical carcinoma and astrocytoma. These cell lines can be cultured in slide flasks, 60 mm dishes or 100 mm dishes. Asynchronously growing populations are plated out for 24 hours for attachment and growth, after which different concentration-time combinations of the drug may be used, followed by irradiation as appropriate. Mitotic cell accumulations and cellular morphology can be evaluated microscopically, with the fraction of cells cycling being monitored by bromodeoxyuridine (BrdUrd) uptake (5 μM) into DNA, fixation in situ and fluorescence examination of a fluorescein-tagged monoclonal antibody against BrdUrd-substituted DNA. Mitotic indices can be determined by counting 1000 cell samples and determining the proportion of rounded, chromatin-condensed mitotic cells in relation to all cells. Flow cytometry is then undertaken on propidium iodine-stained cells and DNA profiles generated.

Clonogenicity studies are undertaken in 100 mm dishes with cells being replated at appropriate cell numbers to generate 70 to 100 clones per dish. Colony formation in complete medium or complete medium plus the drug for a continuous exposure should take place over 14 to 20 days, following which the medium is discarded and fixative (cold methanol, 3 parts: acetic acid, 1 part) added. After at least a 1 hour fixation, the fixative is discarded, dishes rinsed and Giemsa stain added. Macroscopically visible colonies of greater than 50 cells are counted and related to the number of cells plated. Results should be expressed relative to the controls.

Ideally, in initiating combined modality protocols involving a drug and ionizing radiations, the effectiveness of the two agents should at least be additive and preferably superadditive with combinations of relatively low doses resulting in a sensitizing response. The drug should result in the accumulation of the cells in the late G2 phase and not allowing or slowing the continued cycling and progression of cells through mitosis will lead to cells in the most radiosensitive phase of the cell cycle. There is also an optimal radiation dose where cells are delayed, accumulated and rendered susceptible to lethally induced damage. This effect of selective accumulation and killing of cells in the sensitive G2 phase of the cell cycle is indicative of an agent that would be classified as a cell cycle inhibitor.

These assays can be used to determine whether a compound can be classified as cell cycle inhibitor. Together with the assays outlined in Example 1, one would be able to determine whether the compounds not only arrests cells, but also arrests them in either the M or S phase of the cell cycle.

### Example 3

### Manufacture Of Topical Formulations Of Cell Cycle Inhibitors

Cell cycle inhibitors can be applied topically as a therapy in conjunction with locally administered radiation. Topical formulations of cell cycle inhibitors can be gels, creams, or ointments.

A: Gel Formulation

A topical gel was prepared as follows. A cell cycle inhibitor (e.g., paclitaxel) was incorporated into the topical gel at a concentration of 1%. An active phase was produced by mixing 250 g ethoxydiglycol with 500 mg methylparaben and 250 mg propylparaben, while continuously stirring at 200 rpm. When all components were completely dissolved, 5 g of paclitaxel was added and mixed for an additional 20 minutes at 200 rpm. The mixture was covered with parafilm and set aside.

A gum phase was prepared by mixing 82.2 g of ethoxydiglycol with 7.5 g hydroxyethylcellulose. The cellulose was added slowly over a 5 minute period with stirring at 200 rpm. Once the hydroxyethylcellulose was added, the mixing speed was increased to 400 rpm for 40 minutes. Water (155 ml) was slowly added and thoroughly mixed for 60 minutes

To prepare the gel, 20 ml of the active phase was added to the gum phase while mixing at a stirrer setting of 200 rpm over 15 minute time interval. The remaining active phase was added over 45 minutes, while mixing. The speed was increased to 400 rpm and mixing continued for 5 hours. This process yielded approximately 500 g of a 1% paclitaxel-loaded gel. This process can be used to produce gels with drug loadings between 0.01 and 2% paclitaxel. By increasing the ratio of ethoxydiglycol to water, more paclitaxel may be dissolved in the gel.

Other cell cycle inhibitors may be incorporated into the gel formulation provided they are sufficiently soluble in the active phase and in the final gel formulation. To enhance drug solubility, some or all of the ethoxydiglycol or water may be substituted with another solvent, such as ethanol or propylene glycol. The amount of substituted solvent required is determined by measuring the solubility of the selected cell cycle inhibitor in various co-solvent systems, and selecting one that provides sufficient solubility of the compound to incorporate the desired amount into the gel (up to 1%).

B: Cream Formulation

Topical creams (oil in water emulsions) can be prepared as follows. A cream base may be used to incorporate a cell cycle inhibitor (e.g., 5-fluorouracil). A 1.85% 5-fluorouracil cream is prepared as follows. An oil phase is prepared by combining stearyl alcohol (250 g) and White Petrolatum, USP (250 g) at 75° C. and melting the mixture. The oil phase is stirred at 100 rpm for 5 minutes to ensure homogeneous mixing. An active phase is prepared as follows. Methylparaben (0.25 g), propylparaben (0.15 g), sodium lauryl sulfate (10 g), propylene glycol (120 g) are dissolved in 370 g of Fluorouracil Injection, USP, by mixing the components at 75° C. with stirring at 100 rpm until a clear solution is formed. The active phase is added to the oil phase and the mixture is cooled while stirring until it congeals to form a cream.

Other water soluble cell cycle inhibitors may be incorporated into a cream by substituting an aqueous solution of the drug for Fluorouracil Injection, USP.

C: Ointment Formulation

Topical ointments can be prepared as follows. An ointment such as White Petrolatum, USP, may be used to incorporate a cell cycle inhibitor (e.g., bleomycin A₂). White petrolatum (99 g) is heated to 75° C. until it is completely melted. Bleomycin (1 g) is dissolved in 20 ml methanol with stirring for 20 minutes at 30° C. The bleomycin solution is added to the molten petrolatum phase and stirred. The mixture is maintained at 75° C. with stirring for 3 hours to evaporate the methanol, leaving a mixture of 1% bleomycin in White Petrolatum, USP. The mixture is then transferred to a vacuum oven heated to 75° C. and residual solvent is removed under reduced pressure (<5 mmHg) over a 12 hour period.

Alternatively, bleomycin may be incorporated directly into the White Petrolatum, USP by trituration and geometric dilution, without the use of a solvent. In this embodiment, 1 g of bleomycin is combined with 1 g White Petrolatum, USP at room temperature on a glass slab. Mixing is accomplished with a stainless steel spatula. The components are mixed for 5 minutes to ensure the bleomycin is evenly dispersed in the White Petrolatum, USP. An additional 2 g of White Petrolatum, USP are then added and mixed by trituration for 5 minutes. An additional 4 g of White Petrolatum, USP are then added and mixed by trituration for 5 minutes. An additional 8 g of White Petrolatum, USP are then added and mixed by trituration for 5 minutes. An additional 16 g of White Petrolatum, USP are then added and mixed by trituration for 5 minutes. An additional 69 g of White Petrolatum, USP are then added and mixed by trituration for 5 minutes. The result is 100 g of a 1% bleomycin ointment.

These topical cell cycle inhibitor-loaded formulations can be used with topical radiation in the treatment of such diseases as skin cancer, using surface molds or plaques. The formulation would be applied to the skin surface prior to the fitting of surface molds and repeated prior to each treatment.

### Example 4

### Use Of A Topically Administered Cell Cycle Inhibitor With Radiation

In various embodiments of this method of treatment, cancers are treated with a combination of radiation therapy and a topically administered cell cycle inhibitor. Table 1 lists the embodied cell cycle inhibitors, targeted cancers and the topical formulation used to deliver them. The formulations are produced in a manner similar to that described for gels, creams and ointments in the previous example. Any exceptions to the procedure are listed in Table 1 are substituted for those described in the previous example.

TABLE 1
SUMMARY OF EMBODIED CELL CYCLE INHIBITOR TOPICAL
FORMULATIONS AND THEIR METHOD OF MANUFACTURE

| | Type of | | |
|---|---|---|---|
| Cell cycle | Formu- | | Targeted |
| inhibitor | lation | Manufacturing Procedure | Cancer |
| 5- | Cream | As described in Example 3 | Cervical, Non- |
| fluor- | | | melanoma skin, |
| ouracil | | | Penile, Vulvar |
| paclitaxel | Gel | As described in Example 3 | Cervical |
| bleomycin | Ointment | As described in Example 3 | Penile |
| cisplatin | Ointment | Add cisplatin to White | Cervical, |
| | | Petrolatum, USP by trituration | Penile, Vulvar |
| | | as described in Example 3 | |
| ifosfamide | Ointment | Add cisplatin to White | Cervical |
| | | Petrolatum, USP by trituration | |
| | | as described in Example 3 | |
| ironotecan | Cream | Substitute a 10 mg/ml aqueous | Cervical |
| | | solution of ironotecan (adjusted | |
| | | to pH = 4) for the fluorouracil | |
| | | injection, USP used in | |
| | | Example 3 | |
| gemcita- | Cream | Substitute a 1 mg/ml aqueous | Cervical |
| bine | | solution of gemcitabine for the | |
| | | fluorouracil injection, USP used | |
| | | in Example 3 | |
| carmustine | Gel | Substitute carmustine or | Melanoma |
| or | | lomustine for paclitaxel used in | |
| lomustine | | Example 3. Substitute ethanol | |
| | | for ethoxydiglycol in the active | |
| | | phase and ethanol for water in | |
| | | the gum phase of the gel, as | |
| | | described in Example 3 | |
| dacar- | Cream | Substitute a 1 mg/ml aqueous | Melanoma |
| bazine | | solution of dacarbazine (adjusted | |
| | | to pH = 4) for the fluorouracil | |
| | | injection, USP used in | |
| | | Example 3 | |
| metho- | Ointment | Add methotrexate to White | Penile |
| trexate | | Petrolatum, USP by trituration | |
| | | as described in Example 3 | |
| vincristine | Cream | Substitute a 1 mg/ml aqueous | Penile |
| | | solution of vincristine for the | |
| | | fluorouracil injection, USP used | |
| | | in Example 3 | |

Treatment by this means includes the administration of the topical formulation to the target site for a prescribed period of time prior to or immediately prior to the administration of brachytherapy. Structural analogs of each compound listed Table 1 may be substituted as the active component provided they are cell cycle inhibitors.

In this example, a suitable dose of topical cell cycle inhibitor is administered prior to radiation that is administered by placing a radioactive cast or mold over the affected area. Alternately, the topical formulation may be made to contain a soluble form of radiation that decays rapidly to avoid prolonged exposure.

### Example 5

### Procedure For Producing Injectable Polymeric Pastes Containing Cell Cycle Inhibitors

A: Thermally Responsive Paste (Cold Sensitive Paste)

Five grams of polycaprolactone MW 10,000 to 20,000 (Polysciences, Warrington Penn. USA) was added to a 20 ml glass scintillation vial that was placed into a 600 ml beaker containing 50 ml of water. The beaker was gently heated to 65° C. and held at that temperature for 20 minutes until the polymer melted. A known weight (e.g., 5 g) of cell cycle inhibitor (e.g., paclitaxel, vincristine, etoposide, doxorubicin, naphthoquinone) was thoroughly mixed into the melted polymer at 65° C. The melted polymer was poured into a prewarmed mold at 60° C. or poured onto a glass slide at room temperature. The polymeric matrix was allowed to cool until it solidified. For an injectable formulation, the polymer was cut into small pieces (approximately 2 mm by 2 mm in size) and was placed into a 1 ml glass syringe.

The glass syringe was then placed upright (capped tip downwards) into a 500 ml glass beaker containing distilled water at 65° C. until the polymer melted completely. The plunger was then inserted into the syringe to compress the melted polymer into a sticky mass at the tip end of the barrel. The syringe was capped and allowed to cool to room temperature.

For application, the syringe was reheated to 60° C. and administered as a liquid that solidified when cooled to body temperature.

B: Thermally Responsive Paste (Heat Sensitive Paste)

A heat sensitive paste can be made as follows. Three and one half grams of Pluronic F127 (BASF) are added to a 20 ml glass scintillation vial. To the vial, 10 ml of a 1.3% aqueous solution are added and the vial capped. The vial is placed on a rotating mixer at 10 to 15° C. for three hours or until a homogeneous solution is formed. The final solution is a liquid containing approximately 1% fluorouracil. The liquid is loaded into syringes in 100 ml aliquots. The syringe becomes a single injection delivery system. Upon injection into or onto a target tissue, such as a tumor resection site, the liquid is warmed to body temperature it solidifies to form a semi solid paste.

C: Injectable Paste

A semi-solid paste containing a cell cycle inhibitor (e.g., paclitaxel) in a polymeric matrix was prepared by mixing solid paclitaxel into a molten sample of triblock copolymer. The triblock copolymer (2 g) was placed into a 20 ml beaker and heated to 60° C. in a constant temperature water bath. The triblock copolymer was allowed to melt and 3 g of MePEG 350 was added to the triblock copolymer. To prepare a 0.5% w/w paclitaxel paste, 25 mg of paclitaxel was added to the liquid polymer at 50° C. The components were stirred with a stainless steel spatula to mix the drug into the molten mixture. While still molten, the mixture was drawn in 100 μl aliquots into 1 ml syringes. The syringes were sealed. This formulation may be administered into the site of action by injecting it through a 21 gauge needle, a catheter or other similar delivery mechanism.

The triblock copolymer was prepared by ring opening polymerization of a 1:1 mixture of caprolactone and DL-lactide (the monomer) in the presence of polyethylene glycol (PEG) 4600 (the initiator). The ratio of monomer to initiator was 70:30. Stated in terms of components, the weight ratio was 35:35:30 caprolactone:DL-lactide:PEG 4600. The polymerization reaction proceeded at 140° C. for 6 hours with the addition of 0.5% stannous octoate as a catalyst. The formulation can be altered by the addition of varying amounts of paclitaxel, in the range of 0.1 to 5% w/w.

### Example 6

### Procedure For Producing Injectable Non-Polymeric Pastes

Semi-solid matrices containing sucrose acetate isobutyrate (SAIB), a solvent to control viscosity and a cell cycle inhibitor (e.g., paclitaxel) were prepared by combining the ingredients listed in Table 2 at 50° C. and mixing with a stainless steel spatula for 5 to 15 minutes. After a clear solution was formed, the mixtures were allowed to cool to room temperature. The result was a water insoluble, semi-solid matrix.

TABLE 2
COMPOSITIONS OF SAIB MATRIX SEMI-SOLID
FORMULATIONS OF PACLITAXEL FOR ADMINISTRATION
AS A RADIATION SENSITIZER.

| Composition | Mass of | Mass of | Mass and Type of |
|---|---|---|---|
| # | SAIB | Paclitaxel | Solvent |
| 1 | 1884 mg | 502 mg | 627 mg PEG 200 |
| 2 | 1914 mg | 500 mg | 626 mg Ethanol |

In a second embodiment, similar semi-solid matrices were made by altering the ratio of ethanol:SAIB between 40:60 and 5:95, to alter viscosity. A 10:90 ethanol:SAIB matrix was loaded with 0.5% paclitaxel in the same manner as described in the first embodiment of this example.

### Example 7

### Injection Of A Paste Formulation Containing A Cell Cycle Inhibitor Into Or Near To The Targeted Tissue

Cell cycle inhibitor-loaded pastes could be injected through a balloon or catheter to enhance the effect of intracavitary application of radioactive material. Alternatively, cell cycle inhibitor-loaded pastes could be injected through a needle into a target tissue, such as a prostate tumor. Likewise, cell cycle inhibitor-loaded pastes could be applied to organ or tissue surfaces (e.g., tumor resection sites) that will be treated with local radiation. The paste is loaded into the delivery system, such as a syringe and heated if necessary (for thermally responsive, cold sensitive pastes) to allow the material to flow. The delivery system is then situated (e.g., by injection) in the target site and the paste is administered to the target tissue.

Embodied target tissues include any solid tumor such as breast, lung, prostate and esophageal tumors or any tumor resection site. For delivery into the prostate, the paste may be injected alone or it may be loaded into a catheter or needle containing brachytherapy seeds, a mode of local radiation delivery. In this fashion, the cell cycle inhibitor loaded paste may be co-administered with the radiation source. A thermally responsive paste, or one that has an increase in viscosity in vivo could also serve to position the brachytherapy seeds contained within it.

Any cell cycle inhibitor (e.g., paclitaxel, irinotecan, doxorubicin, vincristine, carmustine, cisplatin, methotrexate, 5-fluorouracil, gemcitabine, estramustine, cyclophosphamide, ifosfamide, dacarbazine, and mitomycin C) may be incorporated into a paste as described in Examples 5 and 6 by substituting it for the paclitaxel used in that example. Structural analogs of each of these compounds may be substituted as the active component provided they are cell cycle inhibitors.

### Example 8

### Procedure For Producing Film Containing A Cell Cycle Inhibitor

The term film refers to a polymer formed into one of many geometric shapes. The film may be a thin, elastic sheet of polymer or a 2 mm thick disc of polymer, either of which may be applied to the organ or tissue surface. This film was designed to be placed on exposed tissue so that any encapsulated cell cycle inhibitor can be released from the polymer over a long period of time at the tissue site. Films may be made by several processes, including, for example, by casting and by spraying.

A: Cast Films

In the casting technique, the polymer was either melted and poured into a shape or dissolved in a solvent and poured into a shape. The polymer then either solidified as it cooled or solidified as the solvent evaporated, respectively. In one embodiment, a film containing 5% of a cell cycle inhibitor (paclitaxel) in polyethylene vinyl acetate (EVA) was prepared. Paclitaxel (5 g) and EVA (95 g) were dissolved in 500 ml of dichloromethane over a 12 hour period, with slow stirring at room temperature. 20 ml of the solution was cast onto a glass plate at room temperature using a 40 mil. Gardner Knife. The cast film is placed in a fume hood for 12 hours to allow the solvent to evaporate. The result is a 5% paclitaxel loaded film having a thickness of 100-150 μm.

In a second embodiment, similar to the first, the polymer may be a blend of two materials that serve to alter release of the cell cycle inhibitor or result in increased water uptake into the film. For example, and EVA film was made using the casting technique however an amount of Pluronic L101 or Pluronic F127 surfactant (between 5 and 25% w/w of the mass of EVA) was added to a 10% w/w EVA solution in dichloromethane. The solution was cast in the same manner described for EVA films.

In a third embodiment, the film is cast in the same manner onto a radioactive metalic substrate such as a mixture of radioactive Pd and titanium. After coating, the substrate is turned over, and the back may also be coated in the same manner or it may be coated with a radioopaque layer. This results in a device having at least one polymeric drug-loaded layer, and a metallic radioactive layer. This device may then be inserted around the target site, delivering both radiation and a cell cycle inhibitor.

In a fourth embodiment the following procedure was used. A small glass beaker with a 20 g of PCL was placed into a larger beaker containing water (to act as a water bath) and placed onto a hot plate at 70° C. until the polymer was fully melted. A known weight (1 g) of cell cycle inhibitor (camptothecin) was added to the melted polymer and the mixture stirred thoroughly. The melted polymer was poured into a mold and allowed to cool. The result was a rigid film containing 5% camptothecin in a biodegradable polymer.

B: Sprayed Films

In the spraying technique, the polymer was dissolved in solvent and sprayed onto glass, as the solvent evaporated the polymer solidified on the glass. Repeated spraying enabled a build up of polymer into a film that can be peeled from the glass.

In one embodiment of sprayed films, the following procedure was used. 400 mg of a polymer (polyurethane) was weighed directly into a 20 ml glass scintillation vial and 20 ml of dichloromethane added to achieve a 2% w/v solution. The solution was mixed to dissolve the polymer. Using an automatic pipette, a suitable volume (minimum 5 ml) of the 2% polymer solution was transferred to a separate 20 ml glass scintillation vial. Sufficient cell cycle inhibitor (e.g., paclitaxel) was added to the solution and dissolved by shaking the capped vial. To prepare for spraying, the cap of the vial was removed and the barrel of an atomizer dipped into the polymer solution. A nitrogen tank was connected to the gas inlet of the atomizer and the pressure gradually increased until atomization and spraying began. Molds were sprayed using 5 second oscillating sprays with a 15 second dry time between sprays. Spraying was continued until a suitable thickness of polymer was deposited on the mold.

Alternately, the polymer and solvent may be altered to form a more biocompatible mixture, such as ethanol and hyaluronic acid. A more biocompatible solvent will allow for the solution to be sprayed directly onto the targeted tissue.

Cell cycle inhibitor-loaded films, wraps or molds can be applied to tissue or organ surfaces that are to receive radioactive treatment. The cell cycle inhibitor-loaded polymers can be applied prior to or concurrently with application of radioactive material. Alternatively, films can be applied to the surface of radioactive sutures, wires and seeds prior to their implantation into the treatment area.

In a second embodiment of sprayed films, the therapeutic radioisotope is dissolved or dispersed in the polymer solution containing the cell cycle inhibitor (as described in the first embodiment for sprayed films). The solvent used and polymer used may be altered to form a more biocompatible mixture, such as ethanol and hyaluronic acid. A more biocompatible solvent will allow for the solution to be sprayed directly onto the targeted tissue. The resulting formulation would result in a thin layer of drug and polymer being deposited onto the tissue as the ethanol diffuses away from or into the biological surface. A water insoluble polymer may be used to cause the film to precipitate as it contacts the moist tissue surface. In this embodiment, the radiation and cell cycle inhibitor are administered together in the same device.

### Example 9

### Administration Of A Cell Cycle Inhibitor Incororated Into A Film

A cell cycle inhibitor may be administered to a target tissue from a film by placing the film in contact with that tissue. One embodiment in this example is the implantation of an EVA film containing a sufficient amount of paclitaxel (10%) at the site of a breast tumor excision prior to closure of the wound. The film is sutured to maintain its position at the excision site. After implantation of the film, local radiation is administered. A biodegradable film may be substituted for this purpose. A biodegradable film made of a blend poly(glycolic-co-lactic acid) (PLGA) and methoxypolyethylene glycol (MePEG) 350 (or another low molecular weight PEG) may be produced by film casting in the same manner described for EVA films in Example 8. To produce these films, the PLGA and MePEG are substituted for the EVA in the process. The PLGA:MePEG ratio may be altered from 60:40 to 95:5 to optimize the film properties including release kinetics of the cell cycle inhibitor, degradation lifetime of the film and pliability of the film. This formulation has been tested by implantation of a film made of 50:50 PLGA:MePEG containing 1% and 5% of a cell cycle inhibitor (paclitaxel) adjacent to a blood vessel in a rat. The film was pliable and served to deliver paclitaxel to the target site.

Other embodied treatments in this example include excision sites in head and neck, esophageal, liver and bladder cancers and placement of the film around targeted organs such as the pancreas, bile duct, and urethra. In these applications, cell cycle inhibitors other than paclitaxel may be preferred. Films containing any cell cycle inhibitor may be produced using the solvent casting process described in Example 9 with the following modification. The cell cycle inhibitor may be dissolved in the solvent (dichloromethane) in place of paclitaxel. Alternatively, if the cell cycle inhibitor has a solubility in dichloromethane lower than that of the desired loading, an alternate solvent may be employed, such as toluene, tetrahydrofuran or dimethyl acetamide. Alternately, the cell cycle inhibitor may be dispersed as solid particles in the polymer solution. This may be accomplished by milling the drug in a ball mill and sieving the resulting powder through 25 and 100 μm sieves to obtain solid particles of a defined size. The powdered drug is then dispersed with stirring into the polymer solution. A surfactant (such as Pluronic L101) may be added to the solution to facilitate a uniform dispersion of drug particles. Casting of such a solution may be accomplished in a manner similar to the one described in Example 9. Examples of cell cycle inhibitors that may be processed into films include paclitaxel, irinotecan, doxorubicin, vincristine, carmustine, cisplatin, methotrexate, 5-fluorouracil, gemcitabine, estramustine, cyclophosphamide, ifosfamide, dacarbazine, and mitomycin C). Structural analogs of each of these compounds may be substituted as the active component provided they are cell cycle inhibitors.

### Example 10

### Production Of Cell Cycle Inhibitor-Loaded Brachytherapy Seed Spacers

Spacers having a cylindrical shape and dimensions of 0.2-1 mm diameter by 5-10 mm long were prepared from polymers using the following procedures.

Composition #1, Control PCL spacer

Poly(ε-caprolactone) (PCL) was heated to 65° C. in a 20 ml beaker. Once the polymer had melted to a homogeneous liquid, a 12 μl aliquot was removed by suctioning with a pipettor into a glass capillary tube. The open end of the tube was inserted into a sealed vial through a rubber or wax septum. The capillary tube assembly was transferred to a 50° C. water bath and the polymer allowed to equilibrate to 50° C. for approximately 1 minute. The polymer was ejected from the tube as a solid rod into the sealed vial at the end of the capillary tube assembly. The rod was cut using a metal blade into 6 mm lengths. A volume of 12 μl is sufficient to produce four spacers having dimensions of 0.25 nun in diameter by 6 mm in length.

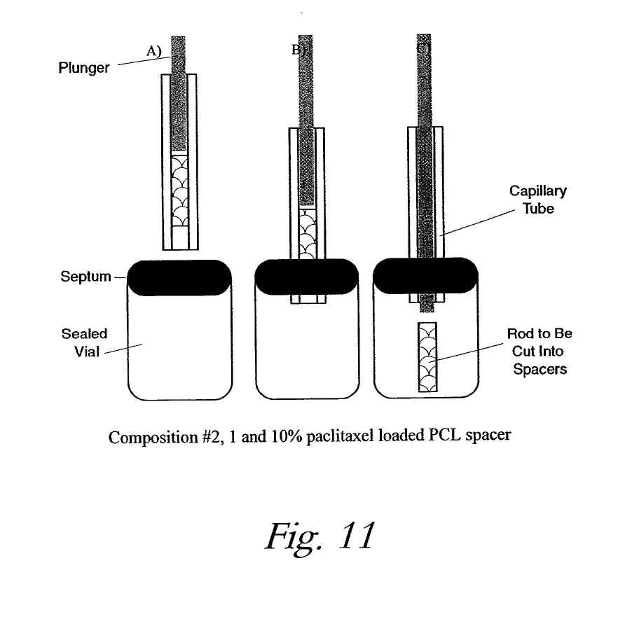

This process is summarized in FIG. 11. As shown in FIG. 11, in step A), the rod has been formed in the capillary tube, and in step B), the capillary tube is inserted through the septum. After insertion through the septum, the assembly is transferred to a water bath, typically a 50° C. water bath, in step C), the rod is ejected into the sealed vial.

Paclitaxel loaded spacers were made in the same manner as for composition #1 with the following exception. Prior to heating to 65° C., PCL was combined with paclitaxel in weight ratios of 1:99 or 10:90 for 1 and 10% loaded spacers, respectively.

Composition #3, Control polyblend spacers (25/75 and 75/25 polyblend spacers)

Control polyblend spacers were made in the same manner as for composition #1 with the following exception. Prior to heating to 65° C., PCL was combined with a diblock copolymer having a composition of 20% w/w MePEG 750 and 80% w/w PCL (total molecular weight=3750 g/mol). The PCL and diblock copolymer was combined in weight ratios of 1:3 and 3:1 to produce 25/75 and 75/25 polyblend spacers, respectively.

Composition #4, Drug loaded polyblend spacers (1 and 10% drug loaded, 25/75 and 75/25 polyblend spacers)

Paclitaxel loaded polyblend spacers were made in the same manner as for composition #3 with the following exception. Prior to heating to 65° C., the 25/75 or 75/25 polyblends were combined with paclitaxel in weight ratios of 1:99 or 10:90 for 1 and 10% loaded spacers, respectively. Other polymeric compositions may be employed. Altering the blend composition serves to alter the physical properties of the spacer including degradation lifetime, pliability and kinetics of release of the cell cycle inhibitor.

### Example 11

### Use Of Cell Cycle Inhibitor-Loaded Brachytherapy Seed Spacers

Spacers having the same dimensions as a brachytherapy seed could be easily loaded into a needle with the brachytherapy seeds. Dummy spacers (containing no cell cycle inhibitor) may also be used in conjunction with the active spacers. By alternating brachytherapy seeds, dummy spacers and drug-loaded spacers into a needle in a predetermined order, followed by injection through a template into a target tissue, for instance a prostate tumor, a precise dose of radiation and cell cycle inhibitor can be administered into a three-dimensional space. Other solid tumor types may also be acceptable target tissues, such as lung, pancreatic or brain tumors. For these four tumor types a number of cell cycle inhibitors that may be selected including etoposide, topotecan, paclitaxel, irinotecan, doxorubicin, vincristine, lomustine, cisplatin, methotrexate, 5-fluorouracil, gemcitabine, leucovorin, tamoxifen, estramustine, cyclophosphamide, ifosfamide and dacarbazine). Structural analogs of each of these compounds may be substituted as the active component provided they are cell cycle inhibitors.

### Example 12

### Coating A Cell Cycle Inhibitor Onto A Device

Non-radioactive metal wire having dimensions of 0.7-0.9 mm diameter and 70-80 mm in length were coated with polyethylene vinyl acetate containing paclitaxel using the following method. After coating the rods were cut into “dummy” seeds with length approximately 10 mm. After coating the diameter increased to 0.85-1.0 mm. The coating procedure was as follows.

Solutions were prepared by dissolving EVA into 2 ml of dichloromethane and adding paclitaxel. The solutions were mixed at room temperature to ensure a homogeneous solution. The compositions of each solution (A-D) are described in Table 3.

TABLE 3
COMPOSITIONS OF SOLUTIONS USED TO COAT
BRACHYTHERAPY SEEDS

| | | Mass of | Desired loading |
|---|---|---|---|
| | Mass of EVA/2 ml | paclitaxel/2 ml | (% w/w paclitaxel |
| Solution | dichloromethane (g) | dichloromethane (g) | in EVA) |
| A | 0.4 | 0.08 | 20 |
| B | 0.2 | 0.04 | 20 |
| C | 0.4 | 0.02 | 5 |
| D | 0.2 | 0.01 | 5 |

After complete dissolution, 1 ml of each solution was transferred to a glass tube. Metal wires were coated by successive dipping of the wire into the solutions in a three-step process. Wires coated with 20% paclitaxel loaded EVA were done by dipping the wire into solutions A, then B, then A again. Wires coated with 5% paclitaxel loaded EVA were dipped in solutions C, then D, then C again. Between each dip, the wires were allowed to dry overnight at 37° C.

Before and after coating, the wires were weighed. Based on these measurements, the amount of paclitaxel per mm was calculated. Total paclitaxel loadings were 26±9 and 41±13 μg/mm for 5 and 20% loaded seeds. For release testing, wires of both loadings having 30-36 μg/mm paclitaxel were selected and cut into lengths equivalent to 1 mg paclitaxel (26-32 mm in length).

### Example 13

### Coating A Cell Cycle Inhibitor Onto A Device

Known weight of cell cycle inhibitor is dissolved in a HPLC grade ethanol. Stent (or radioactive wire) is dipped into the above solution and dried. The stent (or radioactive wire) is further dried under vacuum conditions (−90 KPa) for at least 24 hours at room temperature.

Cell cycle inhibitor-coated radioactive stents can be used for the enhanced brachytherapy of stenosed lumens, such as blood vessels (i.e., restenosis), bile ducts and the esophagus (i.e., carcinoma). Cell cycle inhibitor-coated radioactive wires can be used for interstitial as well as surface therapy.

### Example 14

### Cell Cycle Inhibitor-Loaded Polyurethane Stent Coating

The polyether-based polyurethane is known to be susceptible to microcracking due to biological peroxidation of the ether linkage. A second generation of polyurethane is based on a polycarbonate diol that appears bio stable. Many researchers have reported minimal or no microcracking of polyurethane coating on a stent in the 60 days implantation period.

0.5 g of polycarbonate-based polyurethane with a molecular weight from 100,000 to 250,000 was dissolved in 10 ml of dichloromethane. The above solution was applied to a stent by spraying the solution evenly to its surface. The polyurethane-coated stent was generated by evaporating the dichloromethane completely. The coated stent was further dried under vacuum conditions (−90 KPa) for at least 24 hours at room temperature.

Cell cycle inhibitor-coated radioactive stents can be used in conjunction with local radiation for the treatment of stenosed lumens, such as blood vessels (i.e., restenosis), bile ducts and the esophagus (i.e., carcinoma).

Non-radioactive metal wire having dimensions of 0.18 mm in diameter and 148 mm in length were coated with polyethylene vinyl acetate containing paclitaxel using the following method.

The coating procedure was as follows. A coating solution was prepared by dissolving 0.4 g EVA into 2 ml of dichloromethane and adding 0.08 g paclitaxel. The solutions were mixed at room temperature to ensure a homogeneous solution. After complete dissolution, 1 ml of each solution was transferred to a conical hopper with an orifice at the bottom. Metal wires were coated by passing the wires from the top of the hopper containing polmyer-drug solution through the orifice. The dipping process was completed twice for each wire. Between coatings, the wire was allowed to air dry at room temperature for at least 30 minutes. For the first coat, the orifice at the bottom of the hopper was 0.64 mm. For the second coat, the orifice was 1.14 mm. The wires were drawn through the orifice at a rate sufficient to coat the 148 nun wire in 5-10 seconds.

Before and after coating, the wires were weighed. Based on these measurements, the amount of paclitaxel per cm was calculated. After coating, the wires contained a drug-polymer coating equivalent to 139±39 μg/cm of paclitaxel.

### Example 15

### Manufacture of Micorspheres Containing A Cell Cycle Inhibitor

Microspheres may be made from a number of biodegradable or non-biodegradable polymers including PCL, PLGA, poly(lactic acid) (PLA) and EVA.

In this example an organic phase containing the polymer and cell cycle inhibitor is prepared and dispersed in an aqueous phase with stirring. As the organic solvent is removed, the microspheres are formed.

The organic phase was prepared as follows. PCL (1.00 g) or PLA (1.0 g), or 0.50 g each of PLA and EVA was weighed directly into a 20 ml glass scintillation vial. Twenty milliliters of dichloromethane (DCM) was then added. The vial was capped and stored at room temperature (25° C.) for one hour with occasional shaking to ensure complete dissolution of the polymer. The solution may be stored at room temperature for at least two weeks. To the organic phase was added a sufficient amount of a cell cycle inhibitor (e.g., paclitaxel) to give a drug:polymer ratio of 5:95, 10:90, 20:80, 25:75, or 30:70.

The aqueous phase was prepared as follows. Twenty-five grams of PVA was weighed directly into a 600 ml glass beaker and 500 ml of distilled water was added, along with a 3 inch Teflon coated stir bar. The beaker was covered with glass to decrease evaporation losses, and placed into a 2000 ml glass beaker containing 300 ml of water. The PVA was stirred at 300 rpm at 85° C. (Coming hot plate/stirrer) for 2 hours or until fully dissolved. Dissolution of the PVA was determined by a visual check of solution clarity. The solution was then transferred to a glass screw top storage container and stored at 4° C. for a maximum of two months. The solution, however, must be warmed to room temperature before use or dilution.

To produce the microspheres 100 ml of the aqueous phase (PVA solution) was transferred to a 200 ml beaker. In order to control the size of microspheres, the PVA solution was diluted to a final concentration between 1 and 5% PVA in water (see Table 4A). The aqueous phase was stirred using an overhead stirrer. The stirrer setting was selected based on the desired particle size (see Table 4A). To the stirring aqueous phase, i10 ml of polymer solution containing cell cycle inhibitor was added over a period of 1 to 2 minutes. After 3 minutes the stir speed was adjusted (see Table 4), and the solution stirred for an additional 2.5 hours. The stirring blade was then removed from the microsphere preparation, and rinsed with 10 ml of distilled water so that the rinse solution drained into the microsphere preparation. The microsphere preparation was then poured into a 500 ml beaker, and the beaker washed with 70 ml of distilled water which was also allowed to drain into the microsphere preparation. The 180 ml microsphere preparation was then stirred with a glass rod, and equal amounts were poured into four polypropylene 50 ml centrifuge tubes. The tubes were then capped, and centrifuged for 10 minutes at 2000 rpm. Forty-five milliliters of the PVA solution was drawn off of each microsphere pellet.

5 ml of distilled water was then added to each centrifuge tube and vortexed to resuspend the microspheres. The 4 microsphere suspensions were then pooled into one centrifuge tube along with 20 ml of distilled water, and centrifuged for another 10 minutes (force given in Table 4). This process was repeated two additional times for a total of three washes. The microspheres were then centrifuged a final time, and resuspended in 10 ml of distilled water. After the final wash, the microsphere preparation was transferred into a preweighed glass scintillation vial. The suspension was then frozen and lyophilized to produce a freeze-dried cake of microspheres.

This same process was used to produce microspheres made from PLGA polymers containing paclitaxel in a paclitaxel:polymer ratio of 10:90 and 20:80. Several PLGA polymers having different ratios of glycolic acid to lactic acid monomer units were successfully used to produce microspheres. These PLGA polymers were characterized by their inherent viscosity and are described in Table 4B.

TABLE 4A
STIRRER SPEED SETTINGS AND PVA CONCENTRATIONS USED
IN THE MANUTACTURE OF MICROSPEHRES CONTAINING AND
CELL CYCLE INHIBITOR.

| Microsphere | Stirring Speed | |
|---|---|---|
| Size (μm) | (rpm) | PVA Concentration (%) |
| 1-10 | 2100 | 5% |
| 10-30 | 900 | 5% |
| 30-100 | 900 | 2% |

TABLE 4B
PLGA POLYMER COMPOSITIONS BASED ON WEIGHT RATIOS
OF LACTIC ACID (LA) AND GLYCOLIC ACID (GA) MONOMER
UNITS IN THE POLYMER AND THEIR CHARACTERISTIC
INHERENT VISCOSITY (IV).

| LA:GA | IV |
|---|---|
| 50:50 | 0.74 |
| 50:50 | 0.78 |
| 50:50 | 1.06 |
| 65:35 | 0.55 |
| 75:25 | 0.55 |
| 85:15 | 0.56 |

Cell cycle inhibitor-loaded microspheres could be injected through a balloon or catheter to enhance the effect of intracavitary application of radioactive material. Interstitial brachytherapy would also benefit from interstitial injection of cell cycle inhibitor microspheres prior to or together with injection of radioactive material.

### Example 16

### Production Of Solutions For Local Injection Of A Cell Cycle Inhibitor

A: Manufacture of Aqueous Solutions of Cell Cycle Inhibitors

For water soluble cell cycle inhibitors may be prepared as aqueous solutions. To aid the dissolution of the cell cycle inhibitor into the aqueous phase, the drug may first be lyophilized and excipients added such as mannitol in drug:mannitol ratios between 1:100 and 1:1. Solutions may also be adjusted to a specific pH with HCl or NaOH to optimize drug solubility and stability. Table 5 summarizes several acceptable aqueous solution of cell cycle inhibitors. Essentially, the compounds are dissolved with stirring into water at the appropriate concentration with stirring. Once a clear solution is achieved it may stored, used or lyophilized for later reconstitution.

TABLE 5
CONCENTRATIONS OF AQUEOUS SOLUTIONS OF
CELL CYCLE INHIBITORS

| | Aqueous concentration |
|---|---|
| Cell cycle inhibitor | (mg/ml) |
| Cytarabine | 100 |
| 5-fluorouracil | 50 |
| Ifosfamide | 50 |
| Doxorubicin (as HCl salt) | 2 |
| Vincristine (as SO₄ salt) | 1 |
| Cisplatin | 0.5 |
| Mitomycin | 0.5 |

B: Manufacture of Micellar (Aqueous Solution) Cell Cycle Inhibitor Formulations

Poly(DL-lactide)-block-methoxypolyethylene glycol (PDLLA-block-MePEG) with a MePEG molecular weight of 2000 and a PDLLA:MePEG weight ratio 40:60 is used as the micellar carrier for the solubilization of hydrophobic cell cycle inhibitor, such as paclitaxel. PDLLA-MePEG 2000-40/60 (polymer) is an amphiphilic diblock copolymer that dissolves in aqueous solutions to form micelles with a hydrophobic PDLLA core and hydrophilic MePEG shell. The cell cycle inhibitor is physically trapped in the hydrophobic PDLLA core to achieve the solubilization.

The polymer was synthesized from the monomers methoxypolyethylene glycol and DL-lactide in the presence of 0.5% w/w stannous octoate through a ring opening polymerization. Stannous octoate acted as a catalyst and participated in the initiation of the polymerization reaction. Stannous octoate forms a number of catalytically reactive species which complex with the hydroxyl group of MePEG and provide an initiation site for the polymerization. The complex attacks the DL-lactide rings and the rings open up and are added to the chain, one-by-one, forming the polymer. The calculated molecular weight of the polymer is 3,333 g/mol.

All reaction glassware was washed and rinsed with Sterile Water for Irrigation, USP, dried at 37° C., followed by depyrogenation at 250° C. for at least 1 hour. MePEG (240 g) and DL-lactide (160 g) were weighed and transferred to a round bottom glass flask using a stainless steel funnel. A 2 inch Teflon coated magnetic stir bar was added to the flask. The flask was sealed with a glass stopper and then immersed to the neck in a 140° C. oil bath. After the MePEG and DL-lactide melted, 2 ml of 95% stannous octoate (catalyst) was added to the flask. The flask was vigorously shaken immediately after the addition to ensure rapid mixing and then returned to the oil bath. The reaction was allowed to proceed for an additional 6 hours with heat and stirring. The liquid polymer was then poured into a stainless steel tray, covered and left in a chemical fume hood overnight (about 16 hours). The polymer solidified in the tray. The top of the tray was sealed using Parafilm®. The sealed tray containing the polymer was placed in a freezer at −20±5° C. for at least 0.5 hour. The polymer was then removed from the freezer, broken up into pieces and transferred to glass storage bottles and stored refrigerated at 2 to 8° C.

Preparation of the bulk and filling of cell cycle inhibitor/polymer matrix was accomplished essentially as follows. Reaction glassware was washed and rinsed with Sterile Water for Irrigation, USP, and dried at 37° C., followed by depyrogenation at 250° C. for at least 1 hour. First, a phosphate buffer (0.08 M, pH 7.6) was prepared. The buffer was dispensed at the volume of 10 ml per vial. The vials were heated for 2 hours at 90° C. to dry the buffer. The temperature was then raised to 160° C. and the vials dried for an additional 3 hours.

The polymer was dissolved in acetonitrile at 15% w/v concentration with stirring and heat. The polymer solution was then centrifuged at 3000 rpm for 30 minutes. The supernatant was poured off and set aside. Additional acetonitrile was added to the precipitate and centrifuged a second time at 3000 rpm for 30 minutes. The second supernatant was pooled with the first supernatant. Cell cycle inhibitor (e.g., paclitaxel) was weighed and then added to the supernatant pool. The solution was brought to the final desired volume with acetonitrile.

The cell cycle inhibitor/polymer matrix solution is dispensed into the vials containing previously dried phosphate buffer at a volume of 10 ml per vial. The vials are then vacuum dried to remove the acetonitrile. The cell cycle inhibitor/polymer matrix is then terminally sterilized by irradiation with at least 2.5 Mrad Cobalt-60 (Co-60) x-rays.

C: Manufacture of Lipophilic Solutions of Cell Cycle Inhibitors

For water insoluble cell cycle inhibitors, a solution may be prepared in a lipophilic liquid such as an oily vitamin (e.g., Vitamin E). For example, paclitaxel may be dissolved in Vitamin E by first dissolving it in ethanol

### Example 17

### Manufacture Of Spray Loaded With Cell Cycle Inhibitor And A Radioactive Source

A sufficient amount of polymer is weighed directly into a 20 ml glass scintillation vial and sufficient DCM added to achieve a 2% w/v solution. The solution is mixed to dissolve the polymer. Using an automatic pipette, a suitable volume (minimum 5 ml) of the 2% polymer solution is transferred to a separate 20 ml glass scintillation vial. Sufficient cell cycle inhibitor (e.g., paclitaxel) is added to the solution and dissolved by shaking the capped vial. Once the cell cycle inhibitor is dissolved, an appropriate amount of microparticulate radioactive source (e.g., gold grains) is added so as to achieve the desired radiation dose. To prepare for spraying, the cap of the vial is removed and the barrel of the TLC atomizer dipped into the polymer solution.

The nitrogen tank is connected to the gas inlet of the atomizer and the pressure gradually increased until atomization and spraying begins. The cell cycle inhibitor-loaded radioactive spray is then applied to the tumor resection margin. The area is sprayed until the premeasured amount of cell cycle inhibitor/microparticulate radiation source is dispensed.

### Example 18

### Release Of A Cell Cycle Inhibitor From A Device Or Formulation To Be Used Into Conjunction With Local Radiation Therapy

In vitro release profiles of a cell cycle inhibitor (e.g., paclitaxel) from brachytherapy seed spacers, injectable semi-solid pastes, coated seeds and coated wires were measured using the following method. The test articles (samples of the aforementioned devices and formulations) were weighed and transferred to test tubes containing 15 ml of phosphate buffer (pH=7.4). The test tubes were sealed and placed on a rotating rack in a 37° C. oven. At sampling intervals, the tubes were removed and the buffer was transferred from each sample tube to a new clean tube, which tubes were reserved for later analysis. To the sample tubes, 15 ml of fresh buffer were added and the tubes returned to the rotating rack in the 37° C. oven.

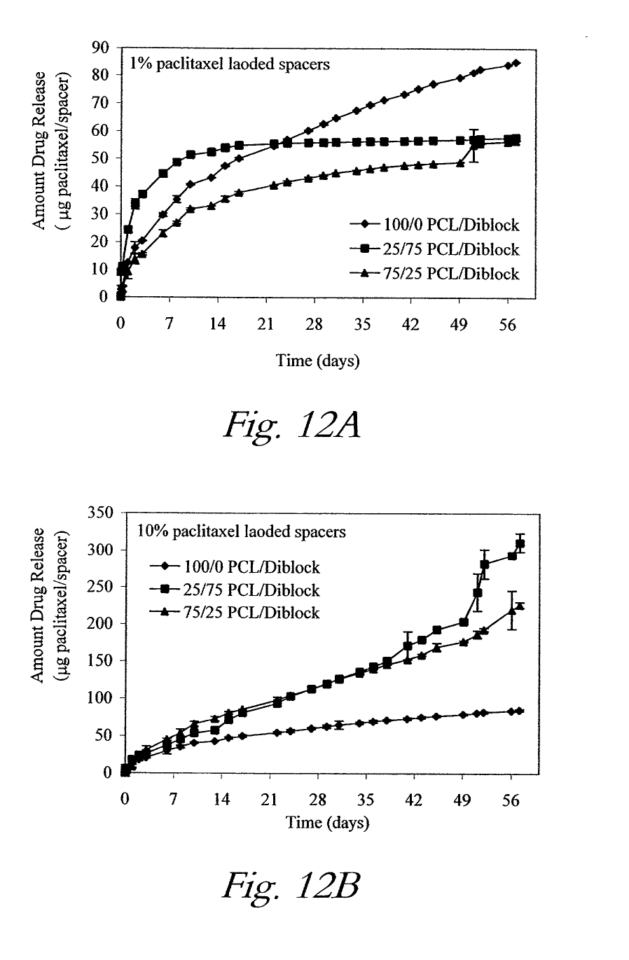

To the sampled buffer, 1 ml of dichloromethane was added and the tube mixed for 15 minutes by rotating at room temperature. The tube was then centrifuged to separate the aqueous and organic phases. The aqueous supernatant was removed and discarded and the organic extract was evaporated to dryness under nitrogen at 55° C. Immediately prior to analysis by HPLC, the dried sample was reconstituted with a 1 ml mixture of 1:1 acetonitrile and water. The sample was then analyzed by HPLC using a Hypersil ODS guard column, a 125 mm×4 mm ID 5 μm Hypersil ODS column at 28° C., a uv detector at 232 nm, and a mobile phase of 55% acetonitrile, 45% water with a flow rate of 1 ml/min. The injection volume was 10 μl and the assay run time was 15 minutes. FIGS. 12 to 15 show in vitro release profiles of paclitaxel from the various test articles.

FIGS. 12A and 12B show in vitro profiles of paclitaxel release from radiation seed spacers. Each spacer weighs 5-10 mg and contains 1 or 10% w/w paclitaxel in a polymeric matrix containing poly(E-caprolactone) (PCL) and diblock (80:20 MePEG 750:PCL).

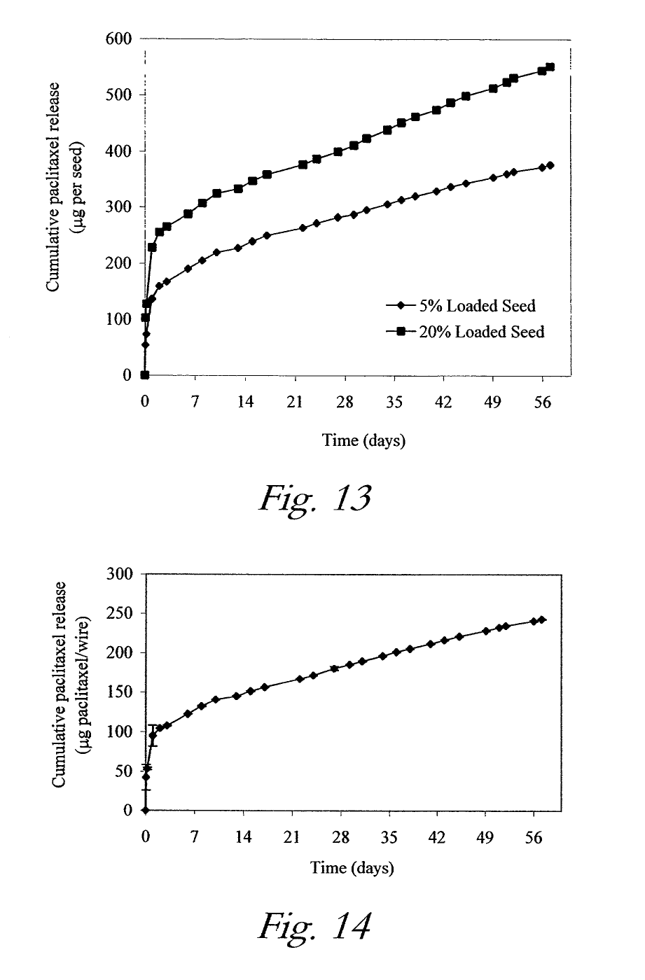

FIG. 13 shows in vitro profiles of paclitaxel release from paclitaxel coated brachytherapy seeds. Each seed is coated with 0.95 to 1.00 mg of paclitaxel in an EVA coating. The concentration of paclitaxel in EVA is 5 or 25% w/w.

FIG. 14 shows an in vitro profile of paclitaxel release from a coated wire. Each wire is coated with 1-2 mg of an EVA matrix containing 20% w/w paclitaxel.

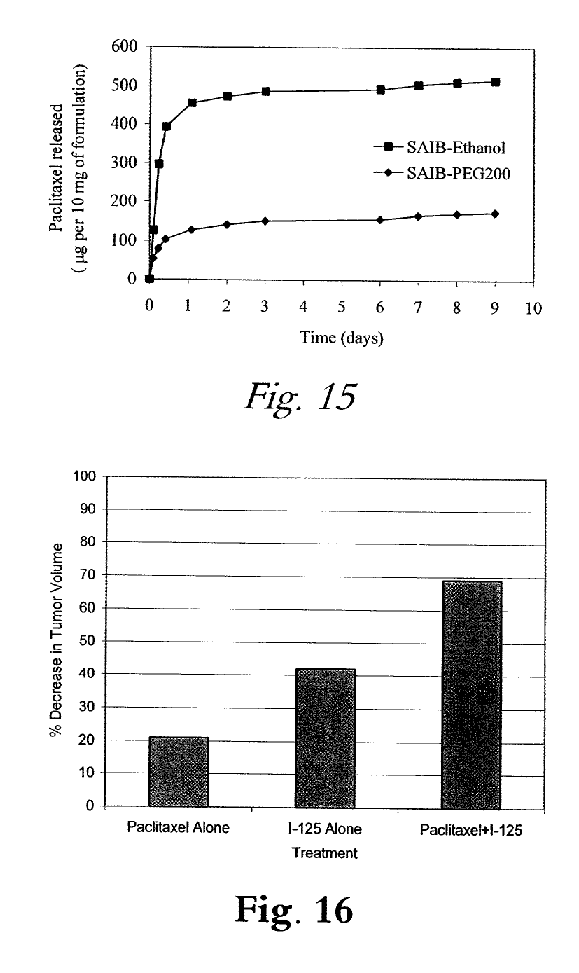

FIG. 15 shows in vitro profiles of paclitaxel release from a semi-solid injectable paste comprising sucrose acetate isobutyrate (SAIB) and a solvent, ethanol or PEG 200.

Profiles of paclitaxel release from the test articles illustrate the ability to control exposure of a cell cycle inhibitor to a target tissue using each of the embodied devices and formulations. Furthermore, the profiles illustrate the ability to alter the release rate and extent by altering the excipient properties of the device or formulations. It is also anticipated that these results will be correlated to release of drug in vivo during the normal course of their therapeutic use and that in vivo release could be controlled and/or altered through specific design of the device or formulation. It should be understood that similar data may be obtained for other cell cycle inhibitors by altering the assay conditions to accommodate compounds with different chemical characteristics.

### Example 19

### In Vivo Treatment Model Using A Locally Administered Cell Cycle Inhibitor

This animal model is used to determine the effectiveness of a locally administered cell cycle inhibitor (e.g., paclitaxel) in conjunction with a locally administered radiation source in treating a proliferative disease, specifically, a cancer. The relative change in tumor volume measured in tumor bearing mice receiving various treatments will be used to gauge the therapy's effectiveness relative to use of local radiation alone or locally administered cell cycle inhibitor alone.

The methods used are as follows. Cancer cells (specifically PC3) human prostate cells, American Type Culture Collection, Rockville Md.) are maintained in DMEM solution with 5% heat-inactivated fetal calf serum. Male SCID mice are inoculated with approximately 1×10⁶ cells subcutaneously in the flank region. The tumor injection sites are followed by visual inspection or palpation. Tumor volume is measured using calipers. The tumor is allowed to grow until it reaches a treatable volume of 100-200 mm³.

At this time the mice are treated as follows. Approximately six brachytherapy seeds are implanted adjacent to the tumor to deliver a local radiation dose of 25-40 Gy (I¹²⁵ radiation source). A polymeric paste (100 μl) containing 50 μg paclitaxel (0.5% w/w) is injected subcutaneously adjacent to or into the tumor. The following treatment groups were studied (10 mice per group). 1) Control paste without paclitaxel and non-radioactive (cold) seeds. 2) Control paste and radioactive seeds. 3) 0.5% w/w paclitaxel loaded paste and cold seeds. 4) 0.5% w/w paclitaxel loaded paste and radioactive seeds.

Tumor size is measured at twice-weekly intervals using calipers. An investigator blinded to the experimental groups will conduct the measurements. Caliper measured dimensions may be taken in two (length (L), width (W)) or three dimensions (Height (H)). Measurements are converted to tumor volumes (mm³) using either the hemi-ellipsoid formula π/6 (L×W×H) or the following formula (L×W²)/2. Tumor measurements are taken for approximately 12 weeks or until tumor volume has reached 3 cm³, which ever occurs sooner.

The animal data are analyzed as follows. The means and standard deviations of the tumor measurements are determined and plotted from the initial day of caliper measurement until the final measurement. Comparisons are made of control versus paclitaxel-paste treatment alone to determine the effect of drug alone and control versus radiation treatment alone to determine the effect of radiation on tumor growth. (If there are significant reductions in tumor development in either group, the dose of either or both drug and radiation should be titrated down and an additional experiment performed.) Finally comparisons are made of tumor growth in the radiation group versus the drug and radiation group. A reduction in tumor size over the course of the experiment following the drug radiation treatment relative to radiation alone illustrates the effectiveness of this therapy.

This animal model may be used to identify therapeutic compounds to be used in this therapy, to establish correlation between in vivo efficacy and in vitro release data (refer to Example 18), or to study dose response relationships. In should be understood that these key parameters may be altered in the following ways in order to answer specific experimental questions regarding this therapy. 1) The dose of radiation can be altered by using hotter or colder seeds (greater or lesser rate of radioactive decays per second, respectively), or by using a different radiation source. 2) The number of seeds used can be altered. 3) The type or amount of cell cycle inhibitor loaded into the paste can be altered. 4) The exact composition of the paste may be altered with the proviso that the paste must serve to deliver the cell cycle inhibitor locally by a subcutaneous injection. 5) A different cell type may be used, with the proviso that the cells will result in a measurable tumor mass after implantation. The doses of cell cycle inhibitor and radiation may be predetermined from preliminary experiments as those which exhibit minimal but observable effects on tumor growth, or just below that dose which causes observable reduction in tumor growth.

FIG. 16 shows representative data obtained using this method. The data show that the tumor volume is decreased one week after treatment with locally administered radiation (I-125) and locally administered paclitaxel (n=9; per treatment). The percent reduction is greatest when these two treatments are given in combination whereas a lesser reduction is observed in animals given only one of the two treatments (radiation of paclitaxel alone).

### Example 20

### Synthesis Of A Radioactive Polymer From Bipyridine-Diol

Bipyridine-diol is combined with methacryloyl chloride in a mole ratio of 1:1 dissolved in anhydrous dichloromethane. The mixture is transferred to a round bottom flask and heated to reflux. A substitution reaction is allowed to proceed for 2-3 hours. The result is a (bipyridine-diol) methacrylate of the type shown in FIG. 1.

FIG. 1. A (bipyridine-diol) methacrylate.

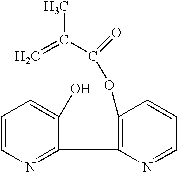

The (bipyridine-diol) methacrylate (FIG. 1) is polymerised with methylmethacrylate to form a poly(methylmethacrylate-co-(bipyridine-diol)methacrylate) as follows. Methylmethacrylate and (bipyridine-diol)methacrylate are combined in a mole ratio of 1:10, dissolved 15% in dry toluene with 1% VAZO67 and degassed by bubbling UHP N₂ through the solution. After degassing the reaction vessel is sealed and heated to 65° C. for 18 hours. After 18 hours, the reaction solution is transferred to lOx the volume of methanol (25° C.) to precipitate the polymer. The polymer is dissolved in dichloromethane (10% w/v) with excess radioactive ¹⁰³PdCl₄ and refluxed for 36 hours. The polymer is then precipitated in lOx the volume of methanol (25° C.). The solid product is dried to constant weight at 25° C. under high vacuum. The product is a radioactive polymer having a structure shown in FIG. 2.

FIG. 2. Radioactive polymer.

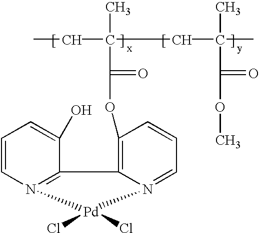

### Example 21

### A Radioactive Fibre

A radioactive fibre that is suitable for implantation is prepared by extrusion of a radioactive polymer from Examples 1 or 2 to produce a fibre. The polymer is well pulverized prior to extrusion using a high-speed, water-cooled grinder. The pulverized polymer is loaded into the hopper and extruded at a temperature above its Tm, which is determined by differential scanning calorimetry prior to the preparation of fibres. For polymers containing a low percentage of bipyridine monomers compared to methylmethacrylate monomers, the Tm will be around 220° C. The polymer is extruded, drawn and spun into fibres suitable for further processing into forms such as sutures or fabrics.

The fibre can be drug loaded (e.g., with paclitaxel) by pre-treating the polymer as follows. The polymer and paclitaxel are dissolved in dichloromethane in a weight ratio of 9:1 polymer to paclitaxel. The solvent is removed from the mixture by drying under vacuum to constant weight at 40° C. A dry matrix has less then a 1% change in weight in three consecutive measurements of mass after 6 hours of drying time.

### Example 22

### A Ring-Shaped Brachytherapy Device

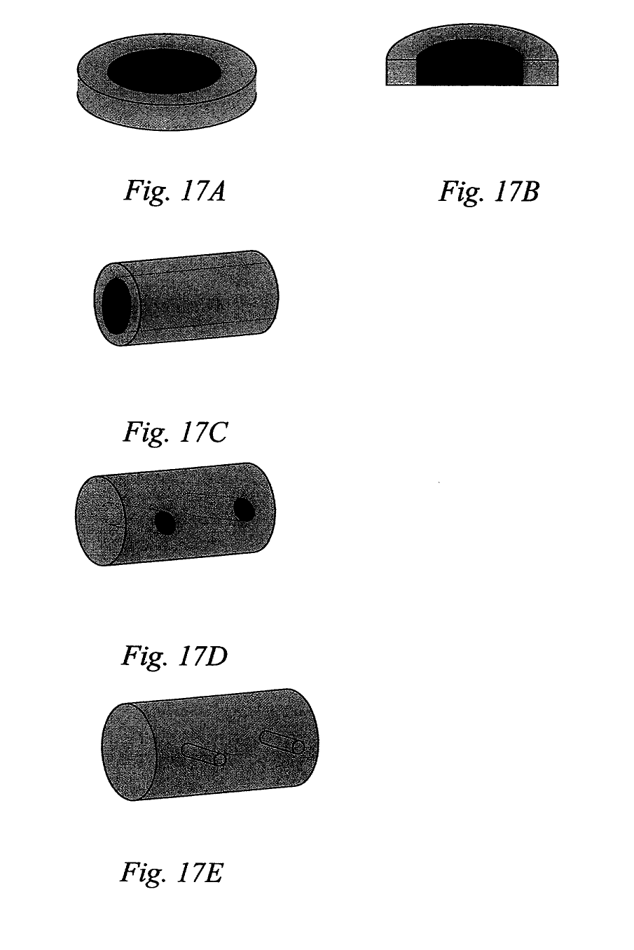

A brachytherapy device is made in the shape of a ring by extruding a radioactive polymer as a pipe. The extrusion temperature will be set above the Tg of the polymer, which is determined by DSC prior to the manufacture of the pipe. As the pipe is extruded, it is cooled and then cut into rings. The shape of such a ring is shown in FIG. 17A. The inner and outer diameter of the ring may be set by adjusting the extrusion arpetures. A typical dimension would be ID: 0.4″, OD: 0.5″. The ring shaped device may be cut in half to produce a “horseshoe” shaped device, shown in FIG. 17B.

### Example 23

### A Hollow Tube Brachytherapy Device

A brachytherapy device is made in the shape of a ring by extruding a radioactive polymer as a pipe. The extrusion temperature will be set above the Tg of the polymer, which is determined by DSC prior to the manufacture of the pipe. As the pipe is extruded, it is cooled and then cut into appropriate lengths. The shape of such a tube is shown in FIG. 17C. The inner and outer diameter of the tube may be set by adjusting the extrusion arpetures. A typical dimension would be ID: 0.08″, OD: 0.1″, Length: 0.4″.

Alternately the tube may function as a drug delivery device by filling the hollow space in with a drug release matrix, such as a solution of paclitaxel 5% in polyethylene glycol, M.W. 2000. The drug matrix is prepared by dissolving paclitaxel and polyethylene glycol in tetrahydrofuran.

### Example 24

### A Rod With Holes Perpendicular To The Axis For Use As A Brachytherapy Device

A brachytherapy device is made in the shape of a rod with holes perpendicular to the axis by extruding a radioactive polymer as a rod. The extrusion temperature will be set above the Tg of the polymer, which is determined by DSC prior to the manufacture of the pipe. As the pipe is extruded, it is cooled and then cut into appropriate lengths. After cutting to length, holes are drilled perpendicular to the axis, either using a conventional mechanical drill bit, e.g. a Dremel toolbit for larger holes or using a laser to drill very fine holes. The shape of such a rod is shown in FIG. 17D. The outer diameter of the rod may be set by adjusting the extrusion arpetures. A typical dimension would be OD: 0.15″, Length: 0.4″, hole diameter: 0.05″.

### Example 25

### A Rod With Protrusions Perpendicular To The Axis For Use As A Brachytherapy Device

A brachytherapy device is made in the shape of a rod with protrusions perpendicular to the axis by first preparing a rod with holes perpendicular to the axis as described in Example 8. The holes are filled by inserting rods with an outer diameter that matches the hole diameter. The rods to be inserted have a length greater than the diameter of the device so that they extend as protrusions out from the device. The protrusions are fixed in place by bonding the seams by lightly spraying them with acetone to dissolve the interface. The outer diameter of the rod may be set by adjusting the extrusion arpetures. A typical dimension would be OD: 0.15″, Length: 0.4″, protrusion diameter: 0.05″, length of protrusion: 0.0.5″. A representative example is shown in FIG. 17E.

### Example 26

### A Method Of Deviloping A Therapeutic Plan For The Admionistration of Drug Loaded Devices In 3-D Space

The method involves obtaining a 3-D image of the target tissue, measuring the diffusion gradient of drug from the drug implant in the target tissue and creating a 3-D map having the outer bounds being the same as the target tissue and points in the confined space such that each area of the target tissue receives a minimum required dose of the drug within the therapeutic life of the device.

In this example, the 3-D image is collected using a conventional ultrasound probe and software used to convert the ultrasound data to a 3-D image. The diffusion gradient of the drug (e.g. paclitaxel) delivered from a device (e.g. a polycaprolactone brachytherapy spacers loaded with 10% paclitaxel) is determined by collecting two types of data, which include the following: (1) in vitro release data are collected (see, e.g., Example 18); and (2) in vivo biodistribution data. These data are collected by loading the device with 9% paclitaxel and 1% ³H-paclitaxel (total activity: 100 μCi per implant). The implant is inserted into a dog prostate and drug allowed to release over a period of 7 days. The animal is sacrificed and the prostate removed, frozen and sectioned into cubes having a dimension of 5 mm. Each cube is referenced by its distance in 3 dimensions from the implant in the prostate. Each cube of tissue is homogenized and the amount of ³H in each sample is analyzed. From this biodstribution study and in vitro release study, the lifetime of the device and the diffusion gradient from the device in the prostate are determined. The therapeutic plan can then be made by allowing for drug-loaded implants to be adequately spaced so that at the midpoint between the implants, the drug level is expected to be at the prescribed concentration.

### Example 27

### Microspheres Made From A Radioactive Polymer.

Microspheres are made by dissolving a radioactive polymer as synthesised in Examples 1 and 2 in dichloromethane at a concentration of 1 g in 10 ml. The polymer is allowed to dissolve with mild agitation. After a clear solution is formed the polymer solution is added at a rate of 1 ml/min to 100 ml of a stirring solution of 2% polyvinyl alcohol (PVA) in water. Stirring is maintained at 1000 rpm for 2 hours until microspheres form and solidify. After preparation of the microspheres, the suspension is centrifuged at 1000 rpm for 5 minutes to separate the microspheres from the PVA solution. The PVA solution is decanted and the microspheres are resuspended in 50 ml water to rinse off any residual PVA. The microspheres are centrifuged again and the water is decanted. The microspheres are allowed to dry in a vacuum oven at ambient temperature and high vacuum for 48 hours.

### Example 28

### Preparation Of A Porous Poly(Methyl Methacrylate) Brachytherapy Seed Spacer.

6.0 g PMMA was added to 20 mL THF in a glass screw top vial. The sample was slowly rotated at 37° C. until dissolved. 20 g NaCl that had been milled and sieved (85-125 um) was then added to the dissolved PMMA solution. The solution was mixed until a homogenous mixture was obtained. The solution was then loaded into a syringe. The mixture was then injected into a piece of Teflon tubing (ID=approx. 1 mm). The tubing was then placed overnight in a forced air oven at 37° C. The remaining solvent was removed by placing the tubing under vacuum overnight. Using a scalpel blade, the Teflon tubing was cut into segments that were approx. 3 mm in length. The PMMA was then removed from the Teflon tubing using a metal push rod. The NaCl was leached from the PMMA segments but stirring them in 200 mL deionized water for 18 hours. The water was changed 3 times during this period. The porous PMMA segments were removed from the water by filtration, rinsed with fresh deionized water and dried overnight under vacuum.

### Example 29

### Preparation Of A Porous Poly(Methyl Methacrylate) Coated Metal Brachytherapy Seed Spacer.

6.0 g PMMA was added to 20 mL THF in a glass screw top vial. The sample was slowly rotated at 37° C. until dissolved. 20 g NaCl that had been milled and sieved (85-125 um) was then added to the dissolved PMMA solution. The solution was mixed until a homogenous mixture was obtained. The open vial was then placed in the fumehood until a viscous solution was obtained. A metal spacer was then dipped into the viscous solution, dried in the forced air oven (4 hours, 37° C.) and further dried overnight under vacuum. The coated spacer was then placed in 100 mL deionized water for 18 hours. The water was changed 3 times during this period. The coated seeds were removed from the water by filtration, rinsed with fresh deionized water and dried overnight under vacuum.

### Example 30

### Incorporation Of Paclitaxel Into A Porous Poly(Methyl Methacrylate) Brachytherapy Seed Spacer.

A 1% (w/v) paclitaxel (Hauser) solution was prepared by dissolving 100 mg paclitaxel in 10 mL acidified methanol. A porous PMMA spacer, prepared in example 29, was placed into the paclitaxel solution. The solution was placed in an ultrasonic bath for 30 sec. The paclitaxel—PMMA spacer solution was stirred at room temperature for 1 hour. The spacers were removed from paclitaxel solution, placed on a glass slide and were dried for 3 hours in a forced air oven at 37° C. The paclitaxel-loaded spacers were further dried by placing under vacuum overnight.

### Example 31

### Biodergradable Spacer With A Biodegradable Echogenic Coating—Dissolution Method.

A 50/50 PLG polymer is extruded through a die such that a solid rod of approx. 0.80- 0.85 mm is produced. A solution of 50/50 PLG is prepared by dissolving 1 g 50/50 PLG in 10 mL ethyl acetate. Five hunderd milligrams finely powdered (<25 um) sucrose is added to the PLG solution. The PLG rod is dipped into the sucrose/PLG solution and then withdrawn such that a thin layer of the PLG/sucrose coated the PLG rod. The solvent is removed using a forced air oven (60° C.) oven. The coated rod is then immersed in a water bath for 10 min. to ensure dissolution of the embedded sucrose. The samples are then dried under vacuum. Optionally, the rod is then dipped into a viscous solution of PLG in ethyl acetate such that a top coat of PLG is formed over the porous coating. The samples are dried under vacuum.

### Example 32

### Biodegradable Spacer With A Biodegradable Echogenic Coating—Rapit Evaporation Method.

A 50/50 PLG polymer is extruded through a die such that a solid rod of approx. 0.80- 0.85 mm is produced. A solution of 50/50 PLG is prepared by dissolving 1 g 50/50 PLG in 10 mL dichloromethane/ethyl acetate. The PLG rod is dipped into the PLG solution and then withdrawn such that a thin layer of the PLG coated the PLG rod. These coated rods are rapidly transferred to a forced-air oven (80° C). The rapid evaporation of the solvent resulted in air bubbles being trapped in the coating layer. This resulted in enhanced echogenic properties of the PLG rod. The rod is dried under vacuum to remove the residual solvent. The rod is then cut into pieces of approx. 5.5 mm.

### Example 33

### Non-Degradable Spacer With A Non-Degradable Echogenic Coating—Gas Bubbles.

A polyethylene rod of diameter 0.83 mm is prepared by an extrusion method. The polymer rod is then dipped into a solution consisting of an acrylic polymer, a polyolefin/acrylic co-polymer, and isocyanate, dissolved in a mixture of tetrahydrofuran and cyclohexanone, and cured. The rod is then dip coated in a base coat solution consisting of cellulose ester, an acrylic polymer, and a polyurethane resin, dissolved in a mixture of solvents including cyclohexanone, tetrahydrofuran, ethyl acetate, and benzyl alcohol, and cured. This rod is then coated with an echogenic coating solution comprising 20% isocyanate pre-polymer dissolved in a mixture of 50 percent (w/w) dimethylsulfoxide in tetrahydrofuran. The coating is then partially dried at room temperature for 3 to 5 minutes to allow some of the TMF (which is the more volatile solvent) to evaporate. The isocyanate pre-polymer polymerizes on exposure to water and gives off carbon dioxide. The device is dipped in water at room temperature for three minutes to cause the polymerization reaction to occur quickly, trapping bubbles of carbon dioxide and forming pores and craters ranging from about 1 to about 70 microns diameter in the coating. The coating is then dried. The rod is then cut into pieces of approx. 5.5 mm in length.

### Example 34

### Non-Degradable Spacer With A Non-Degradable Echogenic Coating—Metal Particles.

A polyethylene rod of diameter 0.83 mm is prepared by an extrusion method. A 20% w/v solution of a polyurethane (ChronoFlex) in THF is prepared. Tungsten powder (Aldrich, 12 micron) is added to the polyurethane solution. The solution is stirred until a homogeneous mixture is obtained. The amount of tungsten powder added is adjusted such that a uniform polymer/tungsten powder coating is obtained when the polymer rod is dipped into the solution. The coated rod is then placed in a forced-air oven to remove the solvent. Optionally the coated rod can be dipped into a second polyurethane/THF solution such that a polyurethane coating is added over the polyurethane/tungsten powder coating. The solvent is removed using a forced-air oven (50°). The rod is then dried overnight in a vacuum oven, and then cut into pieces of approx 5.5 mm in length.

### Example 35

### Non-Degradable Spacer With Metallic Particles

One hundred grams of polyethylene pellets are mixed with 1 g tungsten powder (Aldrich, 12 micron). The mixture is then extruded in the form of a rod with a diameter of approx 0.83 mm. The extruded rod is then cut into pieces of approx. 5.5 mm.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A therapeutic device, comprising a device which locally administers radiation, and a cell-cycle inhibitor.

2. The device according to claim 1 wherein said device is a radioactive stent.

3. The device according to claim 1 wherein said device is a radioactive rod.

4. The device according to claim 1 wherein said device is a radioactive disk.

5. The device according to claim 1 wherein said device is a radioactive seed.

6. The device according to claim 1 wherein said device is a radioactive suture.

7. The device according to claim 1 wherein said device further comprises a polymer.

8. The device according to claim 7 wherein said cell-cycle inhibitor is released by said polymer.

9. The device according to claim 1 wherein said radiation is released from a polymer.

10. The device according to claim 7 or 9 wherein said polymer is a non-biodegradable polymer.

11. The device according to claim 7 or 9 wherein said polymer is a biodegradable polymer.

12. The device according to claim 7 or 9 wherein said polymer is echogenic or radiopaque.

13. The device according to claim 1, wherein said device is echogenic or radiopague.

14. A therapeutic device, comprising: a radioactive source sized to be positioned into the tissue of a patient adjacent to a site to be treated by locally administered radiation from the radioactive source; and a cell-cycle inhibitor positioned adjacent to the radioactive source.

15. A therapeutic device, comprising: a radioactive source sized to be positioned into a pre-existing or created body cavity of a patient adjacent to a site to be treated by locally administered radiation from the radioactive source; and a cell-cycle inhibitor positioned adjacent to the radioactive source.

16. A therapeutic device, comprising: a radioactive source; a capsule containing the radioactive source, the capsule being sized to be positioned into a pre-existing or created body cavity of a patient adjacent to a site to be treated by locally administered radiation from the radioactive source; and a cell-cycle inhibitor.

17. A therapeutic device, comprising: a radioactive source; a body contact material carrying the radioactive source, the body contact member being applied to a pre-existing or created surface site of a patient's body to be treated by locally administered radiation from the radioactive source; and a cell-cycle inhibitor.

18. The device according to claims 14, 15, or 16 wherein said device is ecbogenic or radiopaque.

19. A composition, comprising a radioactive source and a cell-cycle inhibitor.

20. The composition according to claim 18 wherein said composition is echogenic or radiopaque.

20. A method for treating a hyperproliferative disease of the prostate, comprising administering to the prostate a cell cycle inhibitor and a radioactive source, such that said hyperproliferative disease of the prostate is treated.

21. A method for treating a hyperproliferative disease of the anorectum, comprising administering to the anorectum a cell cycle inhibitor and a radioactive source, such that said hyperproliferative disease of the anorectum is treated.

22. A method for treating a hyperproliferative disease of the bladder or urinary tract, comprising administering to the bladder or urinary tract a cell cycle inhibitor and a radioactive source, such that said hyperproliferative disease is treated.

23. A method for treating a hyperproliferative disease of the eye, comprising administering to the eye a cell cycle inhibitor and a radioactive source, such that said hyperproliferative disease is treated.

24. A method for treating a hyperproliferative disease of the brain, comprising administering to the brain a cell cycle inhibitor and a radioactive source, such that said hyperproliferative disease is treated. 25. A method for treating a hyperproliferative disease of the breast, comprising administering to the breast a cell cycle inhibitor and a radioactive source, such that said hyperproliferative disease of the breast is treated.

25. A method for treating a hyperproliferative disease of the esophagus, comprising administering to the esophagus a cell cycle inhibitor and a radioactive source, such that said hyperproliferative disease is treated.

26. A method for treating a hyperproliferative disease of the uterus or cervix, comprising administering to the uterus or cervix a cell cycle inhibitor and a radioactive source, such that said hyperproliferative disease is treated.

27. A method for treating a hyperproliferative disease of the liver or bile duct, comprising administering to the liver or bile duct a cell cycle inhibitor and a radioactive source, such that said hyperproliferative disease is treated.

28. A method for treating a hyperproliferative disease of the lung, comprising administering to the lung a cell cycle inhibitor and a radioactive source, such that said hyperproliferative disease is treated.

29. A method for treating a hyperproliferative disease of the pancreas, comprising administering to the pancreas a cell cycle inhibitor and a radioactive source, such that said hyperproliferative disease is treated.

30. A method for treating soft-tissue sarcomas, comprising administering to a soft-tissue sarcoma a cell cycle inhibitor and a radioactive source, such that sarcoma is treated.

31. A method for treating a hyperproliferative disease of the skin, comprising administering to the skin a cell cycle inhibitor and a radioactive source, such that said hyperproliferative is treated.

32. A method for treating a hyperproliferative disease of the head or neck, comprising administering to the head or neck a cell cycle inhibitor and a radioactive source, such that said hyperproliferative disease is treated.

33. The method according to any one of claims 20 to 32 wherein said cell-cycle inhibitor or radioactive source is echogenic or radiopaque.

34. The method according to any one of claims 20 to 32 wherein said cell-cycle inhibitor or radioactive source further comprise an echogenic or radipaque coating.

35. A radioactive polymer comprising a radioactive monomer and a non-radioactive monomer.

36. The radioactive polymer according to claim 35, wherein said polymer is echogenic.

37. The radioactive polymer according to claim 35, wherein said radioactive polymer further comprises an echogenic polymer.
